SEQUENCE LISTING (3 sequences)

SEQ ID NO: 1 - DNA, 23 bp, Artificial Sequence
  Synthetic oligonucleotide primer for PBGD.
aagggattcactcaggctctttc

SEQ ID NO: 2 - DNA, 20 bp, Artificial Sequence
  Synthetic oligonucleotide primer for PBGD.
ggcatgttcaagctccttgg

SEQ ID NO: 3 - DNA, 26 bp, Artificial Sequence
  Synthetic oligonucleotide probe for PBGD.
ccggcagattggagagaaaagcctgt

(19) United States
(12) Patent Application Publication
(10) Pub. No.: US 2010/0292232 A1
(43) Pub. Date: Nov. 18, 2010

(54) NON-NUCLEOSIDE REVERSE TRANSCRIPTASE INHIBITORS

(76) Inventors: Daniel Elleder, San Diego, CA (US); John A.T. Young, San Diego, CA (US); Thomas J. Baiga, San Marcos, CA (US); Joseph P. Noel, San Diego, CA (US)

Correspondence Address: KLARQUIST SPARKMAN, LLP, 121 S.W. SALMON STREET, SUITE 1600, PORTLAND, OR, 97204, US

(21) Appl. No.: 12/741,189

(22) PCT Filed: Nov. 5, 2008
(86) PCT No.: PCT/US08/82531

§ 371 Date: May 3, 2010

Related U.S. Application Data
(60) Provisional application No. 60/986,990, filed on Nov. 9, 2007.

(51) Int. Cl. A61K 31/437; C07D 513/04; C07D 471/04; A61K 31/429; A61K 31/5377; A61K 31/497; A61P 31/18

(52) U.S. Cl. 514/233.2; 546/121; 548/154; 548/151; 544/133; 544/135; 544/350; 435/184; 514/303; 514/368; 514/366; 514/249

(57) ABSTRACT

Disclosed herein are antiviral agents, in particular non-nucleoside reverse transcriptase inhibitors (NNRTIs) of the formula

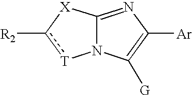

Also disclosed are methods of making the NNRTIs, as well as compositions that include such NNRTIs and methods of their use for treating viral infections, in particular retroviral infections, such as HIV infection.

### CROSS REFERENCE TO RELATED APPLICATION

This application claims the benefit of U.S. Provisional Application No. 60/986,990 filed Nov. 9, 2007, herein incorporated by reference.

### FIELD

This application concerns non-nucleoside reverse transcriptase inhibitors (NNRTIs), methods of making the NNRTIs, as well as compositions that include such NNRTIs and methods of their use for treating HIV infection.

### BACKGROUND

Resistance of the human immunodeficiency virus (HIV) to currently available HIV drugs continues to be a major cause of therapy failure. This has led to the introduction of combination therapy employing two or more anti-HIV agents usually having a different activity profile. Significant progress was made by the introduction of HAART therapy (Highly Active Anti-Retroviral Therapy), which has resulted in a significant reduction of morbidity and mortality in HIV patient populations treated therewith. HAART involves various combinations of nucleoside reverse transcriptase inhibitors (NRTIs), non-nucleoside reverse transcriptase inhibitors (NNRTIs) and protease inhibitors (PIs). Current guidelines for antiretroviral therapy recommend such triple combination therapy regimen for initial treatment. However, these multi-drug therapies do not completely eliminate HIV and long-term treatment usually results in multi-drug resistance. In particular, half of the patients receiving anti-HIV combination therapy do not respond fully to the treatment, mainly because of resistance of the virus to one or more drugs used. Furthermore, resistant virus is carried over to newly infected individuals, resulting in severely limited therapy options for these drug-naive patients.

Therefore, there is continued need for new antiviral agents and active combinations of antiviral agents that are effective against HIV. New types of effective anti-HIV active ingredients, differing in chemical structure and activity profile, are useful in new types of combination therapy. Finding such new active ingredients, therefore, is highly desirable.

### SUMMARY

Disclosed herein are chemical compounds, pharmaceutical compositions including such compounds as well as methods for their preparation and use. In one aspect, the compounds are reverse transcriptase inhibitors. Exemplary compounds disclosed herein are represented by the formula:

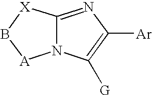

wherein A is N, O, S or CR¹;

B is N or CR²;

A and B are bonded by a single or double bond;

X is CH; CR, where R is H, cyano, halogen, aliphatic, particularly haloalkyl and lower aliphatic, such as lower alkyl, —OR₉, —NR¹⁰R¹¹; or is an atom or atoms in a fused ring; O; or S;

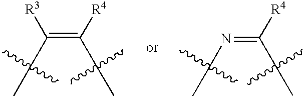

R¹, R², R³ and R⁴ independently are selected from H; cyano; halogen; aliphatic, particularly haloalkyl and lower aliphatic, such as lower alkyl; —OR₉; and —NR¹⁰R¹¹; and two of R¹, R², R³ and R⁴ together may optionally form, or be atoms in, a fused ring;

Ar is a 5 or 6 membered aromatic ring of the formula

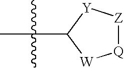

Y is S; N or CR⁵;

Z is S; N; CR⁶;

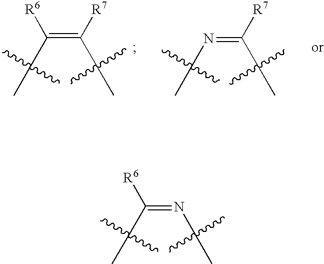

Q is S; N or CR⁸

W is S; N or CR⁹

R⁵-R⁹ independently are selected from H; cyano; halogen; haloalkyl; lower alkyl; —OR¹⁰; —SR¹¹; —NR¹²R¹³; and wherein two of R⁵-R⁹ together optionally may form a fused ring;

R¹⁰, R¹¹, R¹² and R¹³ independently are H, alkyl or acyl;

G is selected from —NR¹⁴R¹⁵ or —N═R¹⁶;

R¹⁴ and R¹⁵ independently are selected from H; aralkyl; lower alkyl; aryl; acyl;

—C(O)OR¹⁷; —C(O)NR¹⁸R¹⁹; —S(O)₂R²⁰; or together with one of R¹, R² or R³ forms a ring;

R¹⁶ is aralkyl and optionally together with one of R¹, R² or R³ forms a ring;

R¹⁷ is lower alkyl, aralkyl or aryl;

R¹⁸ and R₁₉ independently are selected from H; aralkyl; lower alkyl and aryl; and

R²⁰ is aryl.

Also disclosed herein are salts of the disclosed compounds as well as pharmaceutical compositions and methods for their use.

Methods are provided for using the disclosed compounds as antiviral agents. The disclosed compounds and their pharmaceutically acceptable salts and prodrugs thereof as described herein are particularly useful for the treatment or prophylaxis of HIV infection, including HIV that is resistant to one or more reverse transcriptase inhibitors due to a reverse transcriptase mutation.

The foregoing and other objects and features of the disclosure will become more apparent from the following detailed description, which proceeds with reference to the accompanying figures.

### BRIEF DESCRIPTION OF THE DRAWINGS

FIG. 1 illustrates synthesis of inhibitor compounds via the Groebke reaction, a variation of the Ugi multicomponent condensation.

FIG. 2 illustrates preparing exemplary 3-aminoimidazo[1,2-a]pyridine inhibitors using the reaction illustrated in FIG. 1.

FIG. 3 is a plate map illustrating the components used to assemble CBPL-08-006 as described in Example 1 below.

FIG. 4A and FIG. 4B illustrates exemplary 3-aminoimidazo[1,2-a]pyridine inhibitors prepared via the Groebke reaction.

FIG. 5A and FIG. 5B illustrates additional exemplary inhibitors prepared via the Groebke reaction.

FIG. 6 illustrates an overview of the synthesis of exemplary 3-aminoimidazo[1,2-a]pyrazine inhibitors prepared via the Groebke reaction.

FIG. 7A and FIG. 7B illustrates exemplary 3-aminoimidazo[1,2-a]pyrazine inhibitors.

FIG. 8A and FIG. 8B illustrates additional exemplary 3-aminoimidazo[1,2-a]pyrazine inhibitors.

FIG. 9 is a plate map illustrating the components used in the synthesis of 96 inhibitors via Groebke reaction to prepare library CBPL-08-004-034.

FIG. 10 is an additional plate map illustrating the components used in the synthesis of 96 additional inhibitors via Groebke reaction to prepare library CBPL-08-004-035.

FIG. 11 is an additional plate map illustrating the components used in the synthesis of 96 additional inhibitors via Groebke reaction to prepare library CBPL-08-004-036.

FIG. 12 is an additional plate map illustrating the components used in the synthesis of 96 additional inhibitors via Groebke reaction to prepare library CBPL-08-004-037.

FIG. 13 illustrates exemplary biologically active reverse transcriptase inhibitors identified from the synthesis described in FIGS. 9-12.

FIG. 14 is a graph of the results of the cell based infectivity assay using the VSV-G pseudotyped HIV-1 vector demonstrating inhibition of HIV-1 vector infectivity by compound 08-006-F2.

FIG. 15A is an isobologram plot of inhibition for combinations of AZT and the presently disclosed inhibitor 08-006-F2 at the 50% fractional inhibitory concentrations for single compounds, demonstrating synergistic inhibition of HIV-1 infectivity in combination with AZT.

FIG. 15B is an isobologram plot of inhibition for combinations of AZT and the presently disclosed inhibitor 08-006-F2 at the 90% fractional inhibitory concentrations for single compounds, demonstrating synergistic inhibition of HIV-1 infectivity in combination with AZT.

FIG. 16 is a dose-response curve for inhibition of purified HIV-1 reverse transcriptase activity by compound 08-006-F2 (IC₅₀ 2.8 μM, 95% confidence interval 2.3-3.5 μM).

FIG. 17A is an isobologram plot of inhibition for combinations of AZT and the presently disclosed inhibitor 08-006-F2 at the 50% fractional inhibitory concentrations for single compounds, demonstrating synergistic inhibition of purified HIV-1 reverse transcriptase in combination with AZT.

FIG. 17B is an isobologram plot of inhibition for combinations of AZT and the presently disclosed inhibitor 08-006-F2 at the 90% fractional inhibitory concentrations for single compounds, demonstrating synergistic inhibition of purified HIV-1 reverse transcriptase in combination with AZT.

FIG. 18 includes four bar graphs illustrating the effect of compound 08-006-F2 on the synthesis of early and late HIV-1 reverse transcription viral DNA products.

FIG. 19 is a graph charting the dose-dependent inhibition of late HIV-1 reverse transcription viral DNA product synthesis by compound 08-006-F2.

FIG. 20 illustrates the components used to assemble a library of exemplary compounds using the Groebke reaction.

FIG. 21 includes five dose response graphs charting the inhibitory concentration of five different disclosed inhibitor compounds in a cell-based infectivity assay against wild type and four HIV-1 mutant strains having variant reverse transcriptase enzymes.

FIG. 22 includes five dose response graphs charting the inhibitory concentration of three different disclosed inhibitor compounds in a cell-based infectivity assay against wild type and four HIV-1 mutant strains having variant reverse transcriptase enzymes.

FIG. 23 includes five dose response graphs for three control compounds (08-006-F2, 04-035-E1 and Nevirapine (NVP)) against the wild type and four mutant HIV strains used to generate the data in FIGS. 21 and 22.

FIG. 24 includes two graphs (left) charting the toxicity of several disclosed inhibitors, demonstrating that the compounds are substantially nontoxic to human cells and (right) showing inhibition of wild-type and NNRTI mutant viruses by another control compound, AZT.

FIG. 25A and FIG. 25B illustrates several representative compounds and provides their IC₅₀ values against exemplary HIV strains.

FIG. 26 is a plate map illustrating the components used in the synthesis of 88 compounds via Groebke reaction to prepare library CBPL-08-100.

FIG. 27 is a plate map illustrating the synthesis of 88 compounds via Groebke reaction to prepare library CBPL-08-101.

FIG. 28 is a plate map illustrating the synthesis of 88 compounds via Groebke reaction to prepare library CBPL-08-115.

FIG. 29 is a plate map illustrating the synthesis of 88 compounds via Groebke reaction to prepare library CBPL-08-116.

FIG. 30 is a plate map illustrating the synthesis of 88 compounds via Groebke reaction to prepare library CBPL-08-117.

FIG. 31 is a plate map illustrating the synthesis of 88 compounds via Groebke reaction to prepare library CBPL-08-118.

FIG. 32 is a plate map illustrating the synthesis of 88 compounds via Groebke reaction to prepare library CBPL-08-119.

### SEQUENCE LISTING

The nucleic acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NOS: 1 and 2 are primers that can be used to amplify PBGD.

SEQ ID NO: 3 is a probe that can be used to amplify PBGD.

### DETAILED DESCRIPTION

### I. Terms and Abbreviations

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. “Optional” or “optionally” means that the subsequently described event or circumstance can but need not occur, and that the description includes instances where said event or circumstance occurs and instances where it does not. The term “comprises” means “includes.” The abbreviation, “e.g.” is derived from the Latin exempli gratia, and is used herein to indicate a non-limiting example. Thus, the abbreviation “e.g.” is synonymous with the term “for example.” Although methods and materials similar or equivalent to those described herein can be used in the practice or testing of this disclosure, suitable methods and materials are described below. In addition, the materials, methods, and examples are illustrative only and not intended to be limiting.

To facilitate review of the various embodiments of this disclosure, the following explanations of specific terms are provided.

AIDS: acquired immune deficiency syndrome

ART: antiretroviral therapy

AZT: zidovudine

HAART: highly-active antiretroviral therapy

HIV: human immunodeficiency virus

NNRTI: non-nucleoside reverse transcriptase inhibitor

NRTI: nucleoside/nucleotide reverse transcriptase inhibitor

PI: protease inhibitor

SIV: simian immunodeficiency virus

Acquired immune deficiency syndrome or acquired immunodeficiency syndrome (AIDS or Aids): A collection of symptoms and infections resulting from injury to the immune system caused by HIV in humans, and similar viruses in other species (e.g., SIV and FIV).

Acyl: A group of the formula RC(O)— wherein R is an organic group.

Administration: The introduction of an agent, such as one or more of the newly-identified NNRTIs provided herein, into a subject by a chosen route, including both oral and parenteral administration. Generally, parenteral formulations are those that are administered through any possible mode except ingestion. This term also refers to injections, whether administered intravenously, intrathecally, intramuscularly, intraperitoneally, intraarticularly, or subcutaneously, and various surface applications including intranasal, inhalational, intradermal and topical application, for instance. In a specific example, the newly-identified NNRTIs provided herein are administered orally.

Alkyl: An optionally substituted branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms. The terms “halogenated alkyl” or “haloalkyl group” refer to an alkyl group as defined above with one or more hydrogen atoms present on these groups substituted with a halogen (F, Cl, Br, I). The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom in the ring such as, but not limited to, nitrogen, oxygen, sulfur, or phosphorous. In contrast with heterocyclocycloalkyl groups, the term “alicyclic” refers to a group that is both aliphatic and cyclic. Such groups contain one or more all-carbon rings which may be either saturated or unsaturated, but do not have aromatic character. Alkyl groups, including cycloalkyl groups and alicyclic groups optionally may be substituted. The nature of the substituents can vary broadly. Typical substituent groups useful for substituting alkyl groups in the presently disclosed compounds include halo, fluoro, chloro, alkyl, alkylthio, alkoxy, alkoxycarbonyl, arylalkyloxycarbonyl, aryloxycarbonyl, cycloheteroalkyl, carbamoyl, haloalkyl, dialkylamino, sulfamoyl groups and substituted versions thereof.

Alkenyl: An alkenyl group is an optionally substituted hydrocarbon group of 2 to 24 carbon atoms and structural formula containing at least one carbon-carbon double bond.

Alkoxy: An alkoxy group is represented by the formula —OR, wherein R can be an alkyl group, optionally substituted with an alkenyl, alkynyl, aryl, aralkyl, cycloalkyl, halogenated alkyl, or heterocycloalkyl group as described herein. A particular example of an alkoxy group includes, without limitation, methoxy (—OMe).

Alkynyl: An optionally substituted hydrocarbon group of 2 to 24 carbon atoms and a structural formula containing at least one carbon-carbon triple bond.

Aliphatic: Moieties including alkyl, alkenyl, alkynyl, halogenated alkyl and cycloalkyl groups as described above, including optionally substituted variants of these moieties. A “lower aliphatic” group is a branched or unbranched aliphatic group having from 1 to 10 carbon atoms.

Amine or amino: A group of the formula —NRR′, where R and R′ can be, independently, hydrogen or an alkyl, alkenyl, alkynyl, aryl, aralkyl, cycloalkyl, halogenated alkyl, heteroaryl or heterocycloalkyl group described herein. Particular examples of amino groups include, without limitation, —NH-tBu, —NH-nBu and —NMe₂.

Amide: A group represented by the formula —C(O)NRR′, where R and R′ independently can be a hydrogen, alkyl, alkenyl, alkynyl, aryl, aralkyl, cycloalkyl, halogenated alkyl, or heterocycloalkyl group described above.

Antiretroviral therapy (ART): A treatment that can suppress or inhibit a retrovirus, such as HIV or SIV. In some examples such a treatment substantially reduces or inhibits retroviral replication or infection in a mammalian cell. In particular examples, includes administration of one or more agents that interfere with either host or viral mechanisms necessary for the formation or replication of a retrovirus in a mammal, such as one or more NNRTIs, NRTIs, protease inhibitors, fusion inhibitors, RNAse H inhibitors, maturation inhibitors, portmanteau inhibitors, and integrase inhibitors.

Aryl: Refers to any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted. The term “alkyl amino” refers to alkyl groups as defined above where at least one hydrogen atom is replaced with an amino group.

Aralkyl: An aryl group having an alkyl group, as defined above, attached to the aryl group. An example of an aralkyl group is a benzyl group.

Carbonyl: A group of the formula —C(O)—. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

Carboxyl: A —COO— group, which may be either —COOH or —COOR, which also may be referred to as substituted carboxyl, wherein R is aliphatic, heteroaliphatic, alkyl, heteroalkyl, aralkyl, aryl or the like.

CD4: Cluster of differentiation factor 4 polypeptide, a T-cell surface protein that mediates interaction with the MHC class II molecule. CD4 also serves as the primary receptor site for HIV on T-cells during HIV infection (e.g., wild-type HIV-1 infection).

Contacting: Placement in direct physical association; includes both in solid and liquid form. Contacting can occur in vitro with isolated cells or in vivo by administering an agent (such as one or more of the disclosed NNRTIs) to a subject.

Control: A reference standard. For example, a control can be a known value or range of values indicative of a successful or an unsuccessful anti-HIV therapy, such as an expected level of wild-type or mutant HIV-1, HIV-2, or SW RNA present in plasma following administration of one or more anti-HIV compounds.

A control value can be compared to an experimental value, for example to determine the efficacy of a particular NNRTI or anti-HIV therapy, such as a therapy using one or more of the NNRTIs described herein. A difference between a test sample and a control can be an increase or a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%.

Derivative: A compound or portion of a compound that is derived from or is theoretically derivable from a parent compound.

Detect: To determine if an agent is present or absent. In one example, HIV-1, HIV, or SIV virions or RNA is detected in a sample obtained from a subject, such as a subject infected or suspected of being infected with HIV or SW. In some examples, this can further include quantification.

Halide or Halo: Refers to a fluoro, chloro, bromo or iodo substituent, and most typically in the present compounds refers to a fluoro or chloro group.

Highly active antiretroviral therapy (HAART): A treatment that includes a combination of several (such as two, three, four, five or more) anti-retroviral agents, thereby suppressing or inhibiting a retrovirus, such as HIV or SIV. Particular HAART therapies currently in use include (i) efavirenz+zidovudine+lamivudine; (ii) efavirenz+tenofovir+emtricitabine; (iii) lopinavir boosted with ritonavir+zidovudine+lamivudine; and (iv) lopinavir boosted with ritonavir+tenofovir+emtricitabine. One skilled in the art will appreciate that the disclosed NNRTIs can be used in a HAART.

In particular examples disclosed, HAART includes administration of at least one NNRTI disclosed herein along with other agents that target HIV, such as an additional agent that targets HIV-1 reverse transcriptase, such as at least one other NNRTI (e.g., another NNRTI disclosed herein, efavirenz, nevirapine or delavirdine) and one or two different NRTIs (e.g., lamuvidine and zidovudine). In some examples, HAART includes administration of one protease inhibitor (PI) and two NRTIs along with a presently disclosed compound. In a specific example, HAART includes administration of one or more of the newly-identified NNRTIs provided herein in combination with one or more NRTIs (such as zidovudine).

Human immunodeficiency virus (HIV): A retrovirus that causes immunosuppression in humans (HIV disease) and leads to a disease complex known as the acquired immunodeficiency syndrome (AIDS). Reference herein to “HIV” can include reference to the two species of HIV that infect humans, namely, HIV-1 and HIV-2, as well as subtypes thereof, as well as wild-type viruses and variants or mutants thereof. In some examples, the HIV is not a wild-type virus but is instead a mutant form. Mutant forms of HIV include, but are not limited to, those that are not replication competent (e.g., have a functional deletion in the envelope gene), those having a mutant reverse transcriptase sequence (e.g, those that have a mutant RT sequence, such as those that are associated with NNRTI resistance for example L74V, V75I, A98G, L100I, K101E/D/C, K103N, V106A/M, V1081/M, E138K, Q145M, Y181C/I, Y188L/C/H, G190S/A/E, M230L, P225H, P236L, Y318F, N348I or combinations thereof).

“HIV disease” refers to a well-recognized constellation of signs and symptoms (including the development of opportunistic infections) in persons who are infected by an HIV virus, for example as determined by antibody or western blot studies. Laboratory findings associated with this disease include a progressive decline in T-helper cells.

A “resistant strain of HIV” refers to an HIV that retains at least some biological activity (in vitro or in vivo), such as the ability to infect or replicate, when treated with an HIV inhibitor or anti-HIV therapy, such as a reverse transcriptase (RT) inhibitor. One example of an HIV resistant strain is one that retains at least some detectable RT activity when treated with a therapeutically effective amount of an RT inhibitor (such as a currently commercially available NNRTI, for example evirapine, delaviradine and efavirenz), for example retains at least 50%, at least 75%, at least 80%, at least 90%, or even 100% of such RT activity in the presence of an RT inhibitor relative to the absence of the inhibitor. In some examples, such resistant strains of HIV are less resistant or not resistant to the NNRTIs disclosed herein, that is, RT activity of the HIV is significantly decreased (e.g., a decrease of at least 20%, at least 50%, at least 75%, at least 80%, at least 90%, at least 99%, or at least 100%) in the presence of the therapeutically effective amount of the compound.

Hydroxyl: A moiety represented by the formula —OH.

Hydroxyalkyl: An alkyl group that has at least one hydrogen atom substituted with a hydroxyl group. The term “alkoxyalkyl group” is defined as an alkyl group that has at least one hydrogen atom substituted with an alkoxy group described above. Where applicable, the alkyl portion of a hydroxyalkyl group or an alkoxyalkyl group can be substituted with aryl, optionally substituted heteroaryl, aralkyl, halogen, hydroxy, alkoxy, carboxyalkyl, optionally substituted cycloalkyl, optionally substituted cycloalkenyl and/or optionally substituted heterocyclyl moieties.

Inhibit: To decrease, limit or block the action or function of a molecule. For example, NNRTIs of the present disclosure reduce or inhibit, with certain embodiments substantially reducing or inhibiting the activity of HIV-1 or HIV-2 reverse transcriptase, thereby reducing HIV infection, HIV replication, or both.

Isolated: An “isolated” biological component (such as a nucleic acid, peptide or protein) has been substantially separated, produced apart from, or purified away from other biological components in the cell of the organism in which the component naturally occurs, such as, other chromosomal and extrachromosomal DNA and RNA, and proteins. Nucleic acids, peptides and proteins which have been “isolated” thus include nucleic acids and proteins purified by standard purification methods for example from a biological sample.

Non-nucleoside reverse transcriptase inhibitor (NNRTI): Non-nucleosides and analogues thereof that significantly reduce or inhibit the activity of HIV reverse transcriptase (e.g., HIV-1 reverse transcriptase), the enzyme which catalyzes the conversion of viral genomic HIV RNA into proviral HIV DNA. Newly-identified NNRTIs are provided herein. However, other NNRTIs are known, such as nevirapine, delaviradine and efavirenz.

Nucleoside/nucleotide reverse transcriptase inhibitor (NRTI): Nucleosides, nucleotides, and analogues thereof that significantly reduce or inhibit the activity of HIV reverse transcriptase (e.g., HIV-1 reverse transcriptase). Exemplary NRTIs include but are not limited to zidovudine (AZT), lamivudine (3TC), and zalcitabine (ddC).

Optionally substituted: Refers to groups, e.g. “substituted alkyl,” such as an alkyl group, that optionally may have from 1-5 substituents, typically from 1-3 substituents, selected from alkoxy, optionally substituted alkoxy, acyl, acylamino, acyloxy, amino, aminoacyl, aminoacyloxy, aryl, carboxyalkyl, optionally substituted cycloalkyl, optionally substituted cycloalkenyl, optionally substituted heteroaryl, optionally substituted heterocyclyl, hydroxy, thiol and thioalkoxy.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition (1995), describes compositions and formulations suitable for pharmaceutical delivery of the fusion proteins herein disclosed. Such carriers can be used with the NNRTIs and HAART provided herein.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Protease inhibitor (PI): Inhibitors of HIV-1 or HIV-2 protease, an enzyme required for the proteolytic cleavage of viral polyprotein precursors (e.g., viral GAG and GAG Pol polyproteins), into the individual functional proteins found in infectious HIV-1. Examples include, but are not limited to, enfuvirtide, saquinavir and nelfnavir.

Retroviruses: RNA viruses wherein the viral genome is RNA. When a host cell is infected with a retrovirus, the genomic RNA is reverse transcribed into a DNA intermediate which is integrated into the chromosomal DNA of infected cells. The integrated DNA intermediate is referred to as a provirus. The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-1 and HIV-2), simian immunodeficiency virus (SIV), and feline immunodeficiency virus (FIV).

Reverse transcriptase (RT): An enzyme that can transcribe single-stranded RNA into single-stranded DNA. This enzyme is used by reverse-transcribing RNA viruses, such as retroviruses, to reverse-transcribe their RNA genomes into DNA, which is then integrated into the host genome and replicated along with it.

An exemplary reverse transcriptase is a wild-type HIV-1 or HIV-2 RT. HIV-1 RT includes a p66/p51 heterodimer. The p66 domain includes the polymerase (residues 1-315), connection (residues 316-437), and RNase H domains (residues 438-560). In some examples, the RT is an HIV mutant RT, such as those traditionally associated with NNRTI resistance (for example mutations at HIV-1 RT position 74, 75, 98, 100, 101, 103, 106, 108, 138, 145, 181, 188, 190, 230, 225, 236, 318 or 348, such as the mutants L74V, V75I, A98G, L100I, K101E/D/C, K103N, V106A/M, V108I/M, E138K, Q145M, Y181C/I, Y188L/C/H, G190S/A/E, M230L, P225H, P236L, Y318F, N348I or combinations thereof, such as K103N in combination with another mutations, for example L100I and K103N, K101D and K103N, oK103N and Y181C, K103N and V1081, or K103N and K101E/C). Position numbers are based on wild-type HIV-1 RT (polymerase regions) from pNL4-3 genome (GenBank Accession number AAK08484) and HXB-2 genome (GenBank Accession number AAC82598) (the sequences publicly available on Nov. 5, 2008 are herein incorporated by reference). In some examples, one or more of the disclosed novel NNRTIs can substantially reduce or inhibit HIV RT activity (such as HIV-1 or HIV-2 RT) in one or more HIV RT mutants resistant to one or more other NNRTIs, such as those mutants listed above.

Subject: Living multi-cellular vertebrate organisms, a category that includes human and non-human mammals. The methods and compounds disclosed herein have equal applications in medical and veterinary settings. Therefore, the general term “subject” is understood to include all animals, including, but not limited to, humans or veterinary subjects, such as other primates and felines. In a particular example, a subject is a primate model for HIV, such as a macaque infected with SIV or a chimeric SIV. In one example, the subject is infected with HIV-1, HIV-2, SIV, or FIV.

Sulfide: A moiety represented by the formula —SR, wherein R can be an alkyl group, optionally substituted with an alkenyl, alkynyl, aryl, aralkyl, cycloalkyl, halogenated alkyl, or heterocycloalkyl group as described above. The term sulfhydryl is used to refer to the formula —SR wherein R is H.

Sulfonyl: A group of the formula —SO₂—. The sulfonyl group can be further substituted with a variety of groups to form, for example, sulfonic acids, sulfonamides, sulfonate esters and sulfones.

T-Cell: A white blood cell critical to the immune response. T-cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T-cells carry the “cluster of differentiation 8” (CD8) marker. In one embodiment, a CD8 T-cell is a cytotoxic T lymphocyte. In another embodiment, a CD8 cell is a suppressor T-cell.

Therapeutically Effective Amount: An amount of a therapeutic agent (e.g., the disclosed NNRTIs) that alone, or together with an additional therapeutic agent(s) (for example other antiviral agents such as NNRTIs, NRTIs, and PIs) induces the desired response (e.g., substantial reduction or inhibition of HIV infection or replication). In one example, a desired response is to significantly reduce or inhibit HIV replication in a cell to which the therapy is administered. HIV replication does not need to be completely eliminated for the composition to be effective. For example, a composition can decrease HIV replication by a desired amount, for example by at least 10%, at least 20%, at least 50%, at least 60%, at least 70%, at least 80%, at least 90%, at least 95%, at least 98%, or even about 100% (elimination of HIV), as compared to HIV replication in the absence of the composition, can be therapeutically effective.

In another example, a desired response is to inhibit HIV infection. The HIV infected cells do not need to be completely eliminated for the composition to be effective. For example, a composition can decrease the number of HIV infected cells by a desired amount, for example by at least 10%, at least 20%, at least 50%, at least 60%, at least 70%, at least 80%, at least 90%, at least 95%, at least 98%, or even about 100% (elimination of detectable HIV infected cells), as compared to the number of HIV infected cells in the absence of the composition.

A therapeutically effective amount of at least one of the disclosed NNRTIs can be administered in a single dose, or in several doses, for example daily or several times daily (e.g., in 2, 3, or 4 divided doses), during a course of treatment. However, the therapeutically effective amount can depend on the subject being treated, the severity and type of the condition being treated, and the manner of administration. In one non-limiting example, a therapeutically effective amount of an NNRTI provided herein can vary from greater than zero mg per day orally, such as from about 100 to about 2000 mg per day orally. Such doses can be administered in one dose, or divided over several doses per day (such as two, three, or four separate doses).

Treating a disease: A therapeutic intervention that ameliorates a sign or symptom of a disease or pathological condition in a mammalian subject, such as a sign or symptom of HIV infection, SIV infection, acquired immune deficiency syndrome (AIDS), or combinations thereof. Treatment can also induce remission or cure of a condition, such as substantial reduction or elimination of detectable HIV or SIV infected cells, HIV or SIV RNA, or HIV or SIV virions. In particular examples, treatment includes preventing a disease, for example by inhibiting the full development of a disease, such as AIDS, for example by substantially reducing or inhibiting HIV or SIV replication or infection. The beneficial effect can be evidenced, for example, by a delayed onset of clinical symptoms of the disease in a susceptible subject, a reduction in severity of some or all clinical symptoms of the disease, a slower progression of the disease, a reduction in viral replication, an improvement in the overall health or well-being of the subject, or by other parameters well known in the art that are specific to the particular disease. Treatment of a disease does not require a total absence of disease. For example, a decrease of at least 10%, at least 20% or at least 50% can be sufficient.

### II. NNRTIs

Disclosed herein are novel compounds that, in one aspect function as reverse transcriptase inhibitors. Certain embodiments of such compounds can be represented by the formula:

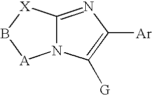

wherein A is N, O, S or CR¹;

B is N or CR², with A and B being bonded by a single or double bond;

X is O; S;

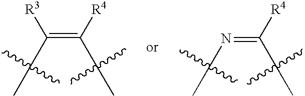

R¹, R², R³ and R⁴ independently are selected from H; cyano; halogen; haloalkyl; lower aliphatic; —OR₉; and —NR¹⁰R¹¹; and two of R¹, R², R³ and R⁴ together may optionally form a fused ring;

Ar is a 5 or 6 membered aromatic ring of the formula

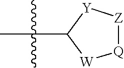

Y is S; Nor CR⁵;

Z is S; N; CR⁶;

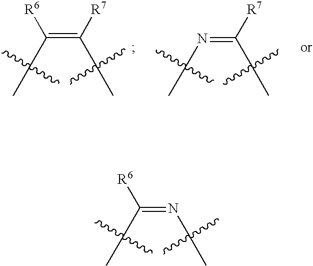

Q is S; N or CR⁸

W is S; N or CR⁹

R⁵-R⁹ independently are selected from H; cyano; halogen; haloalkyl; lower alkyl; —OR¹⁰; —SR¹¹; —NR¹²R¹³; and wherein two of R⁵-R⁹ together optionally may form a fused ring;

R¹⁰, R¹¹, R¹² and R¹³ independently are H, alkyl or acyl;

G is selected from —NR¹⁴R¹⁵ or —N═R¹⁶;

R¹⁴ and R¹⁵ independently are selected from H; aralkyl; lower alkyl; aryl; acyl;

—C(O)OR¹⁷; —C(O)NR¹⁸R¹⁹; —S(O)₂R²⁰; or together with one of R¹, R² or R³ forms a ring;

R¹⁶ is aralkyl and optionally together with one of R¹, R² or R³ forms a ring;

R¹⁷ is lower alkyl, aralkyl or aryl;

R¹⁸ and R₁₉ independently are selected from H; aralkyl; lower alkyl and aryl; and

R²⁰ is aryl.

In certain examples such compounds can be represented by one or more of the

the following seven formulas

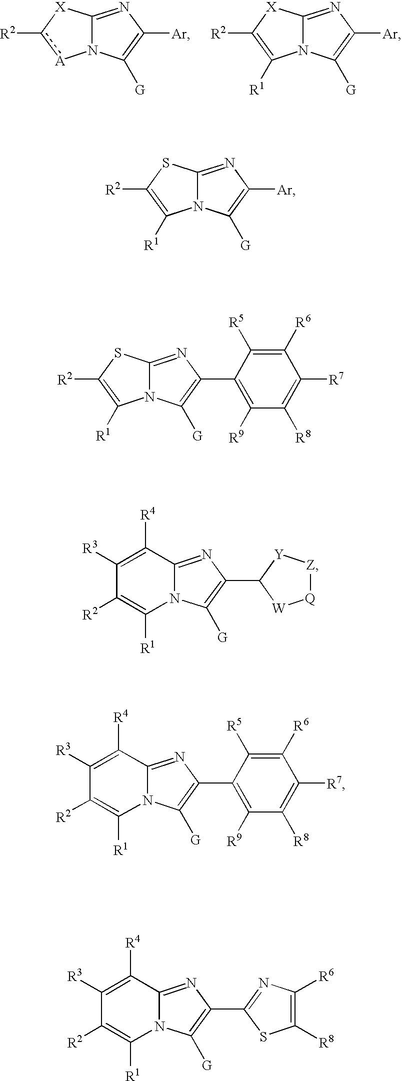

wherein the variable groups A, B, X, Y, Z, W, Q, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ are as set forth above.

In further embodiments, the disclosed compounds include those of the formula

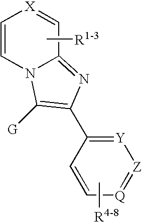

wherein X, Y, Z and Q independently are selected from N or C;

R¹-R³ independently are selected from H; cyano; halogen; haloalkyl; lower alkyl; —OR⁹; and —NR¹⁰R¹¹;

R⁴-R⁸ independently are selected from H; cyano; halogen; haloalkyl; lower alkyl; —OR⁹; —NR¹⁰R¹¹; and wherein two of R⁴-R⁸ together optionally may form a fused ring;

R⁹, R¹⁰ and R¹¹ independently are alkyl or acyl;

G is selected from —NR¹²R¹³ or N═R¹⁴;

R¹² and R¹³ independently are selected from H; aralkyl; lower alkyl; aryl; acyl;

—C(O)OR¹⁵; —C(O)NR¹⁶R¹⁷; —S(O)₂R¹⁸; or together with one of R¹, R² or R³ forms a ring;

R¹⁴ is aralkyl and optionally together with one of R¹, R² or R³ forms a ring;

R¹⁵ is lower alkyl, aralkyl or aryl;

R¹⁶ and R¹⁷ independently are selected from H; aralkyl; lower alkyl and aryl;

R¹⁸ is aryl; or

a salt thereof.

In certain embodiments, disclosed reverse transcriptase inhibitors are represented by the formula

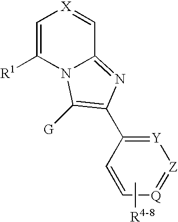

wherein R₁ is halogen; haloalkyl; lower alkyl; —OR¹¹; and —NR¹²R¹³.

Additional disclosed reverse transcriptase inhibitors are disclosed having the formula

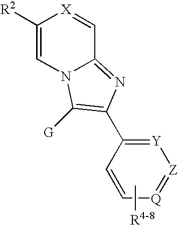

wherein R² is halogen; haloalkyl; lower alkyl; —OR¹¹; and —NR¹²R¹³.

Still other exemplary compounds can be represented by the formula

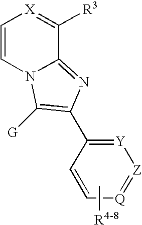

wherein R₃ is halogen; haloalkyl; lower alkyl; —OR¹¹; and —NR¹²R¹³.

Other embodiments of the disclosed reverse transcriptase inhibitors are represented by the formula

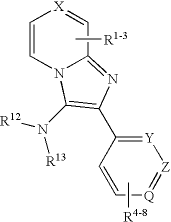

wherein X, Y, Z, Q, R¹, R², R³, R⁴, R⁵, R⁶, R⁷, R⁸, R¹² and R¹³ are as set forth immediately above.

As is apparent from the chemical formulas presented above, the disclosed compounds are not nucleosides, and thus can be categorized as non-nucleoside reverse transcriptase inhibitors or NNRTIs.

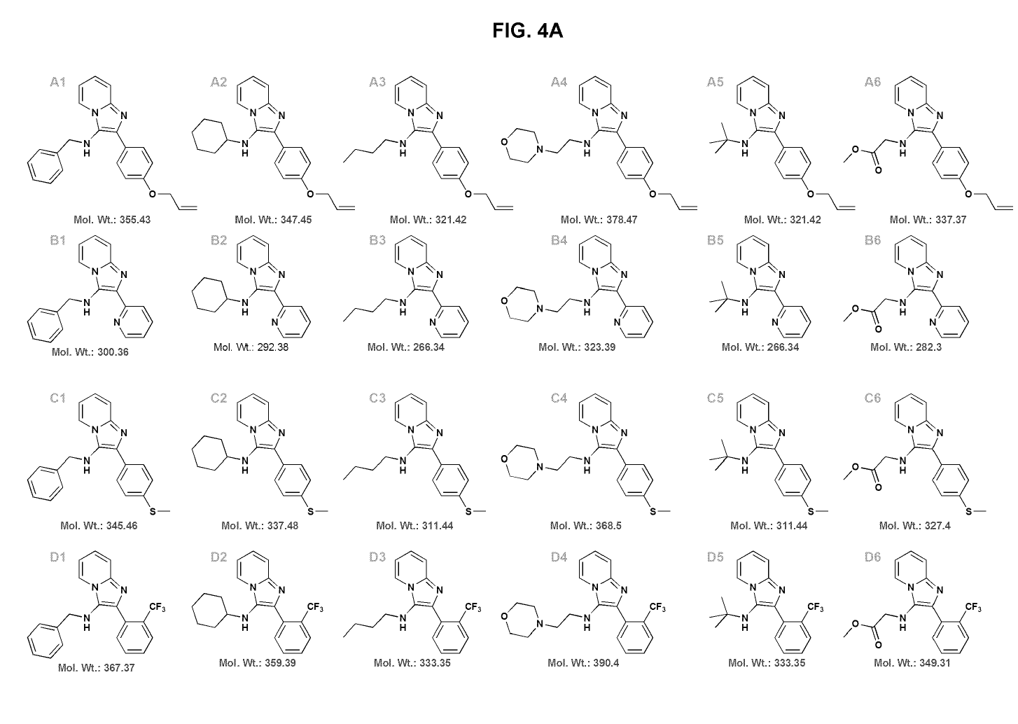

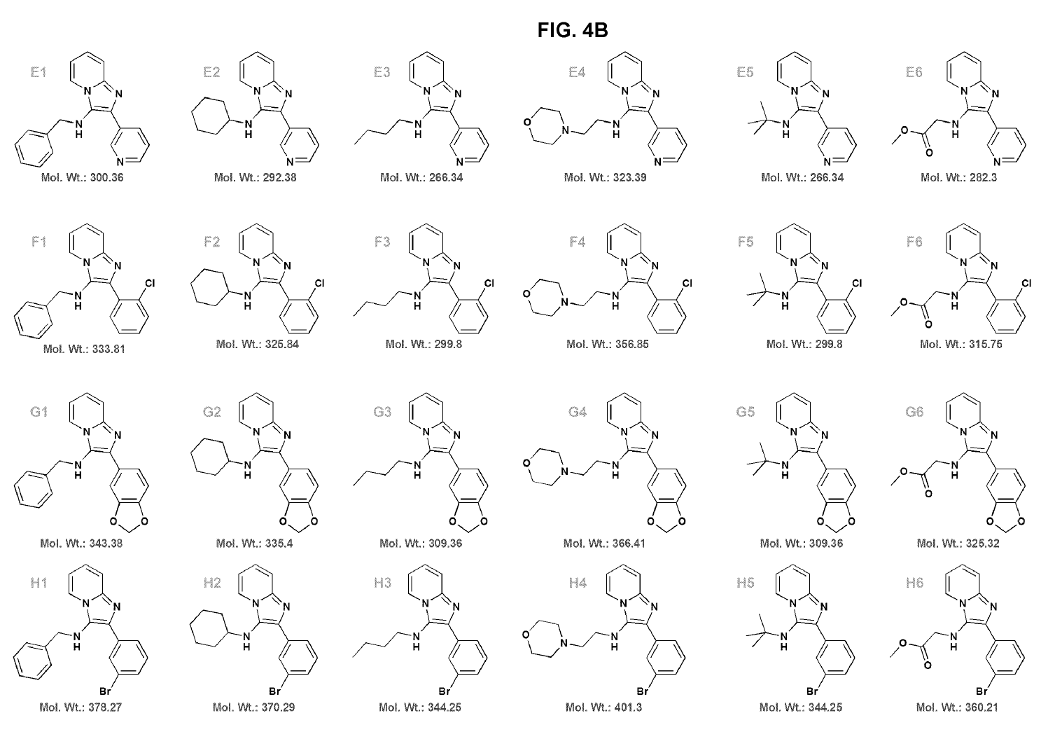

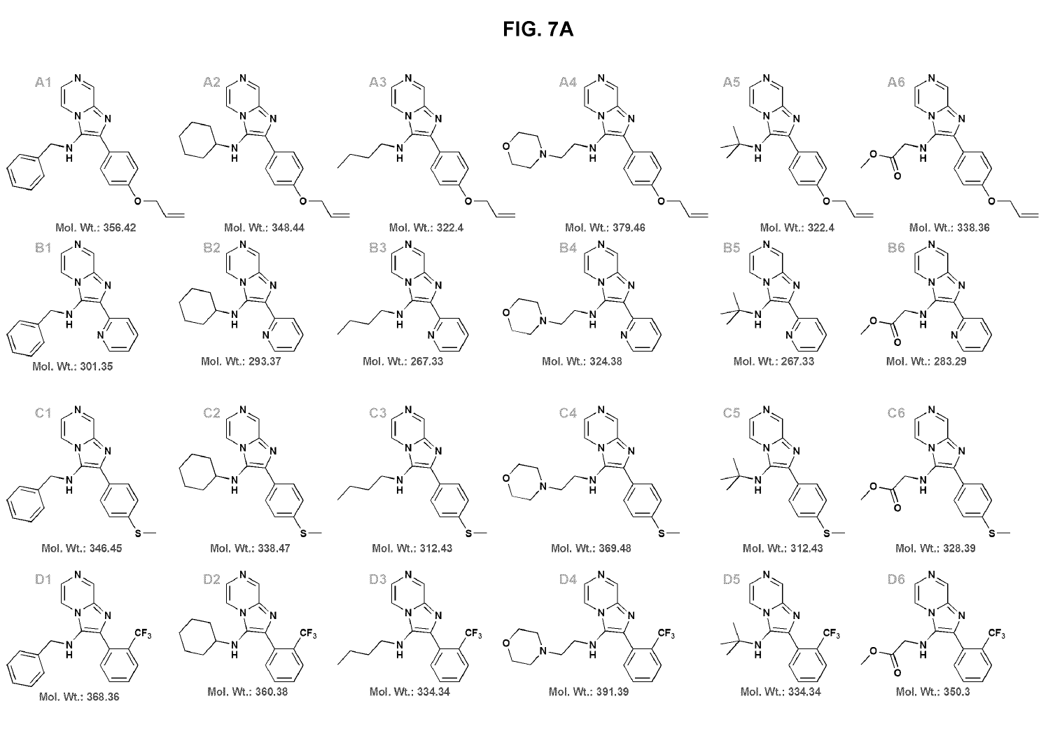

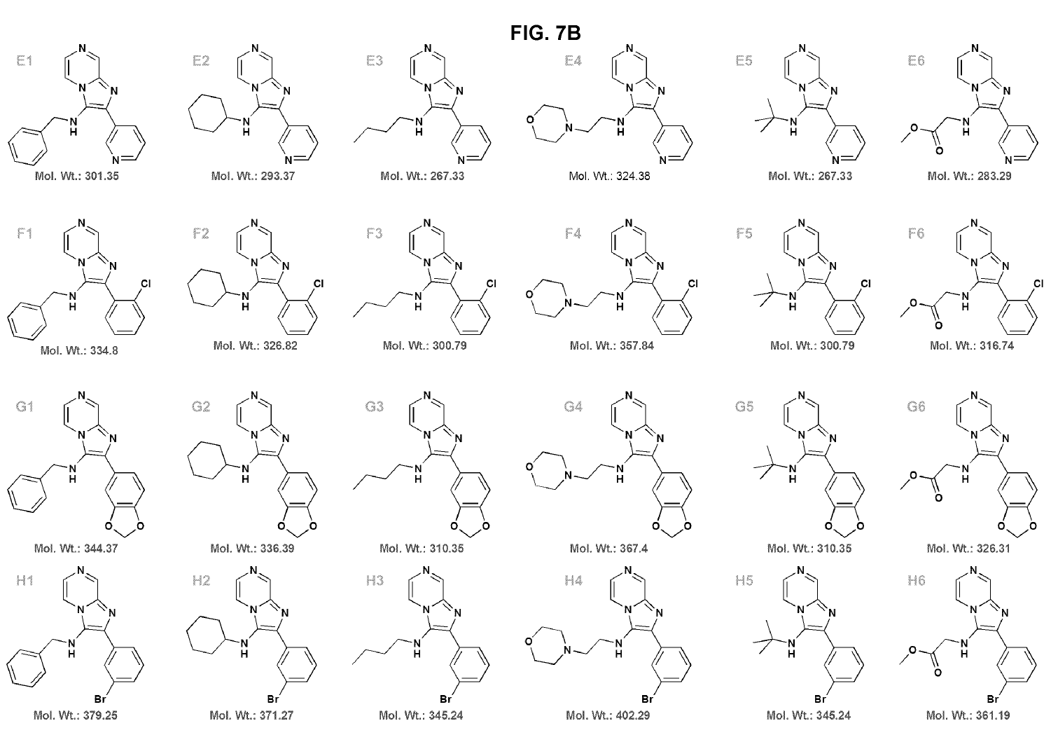

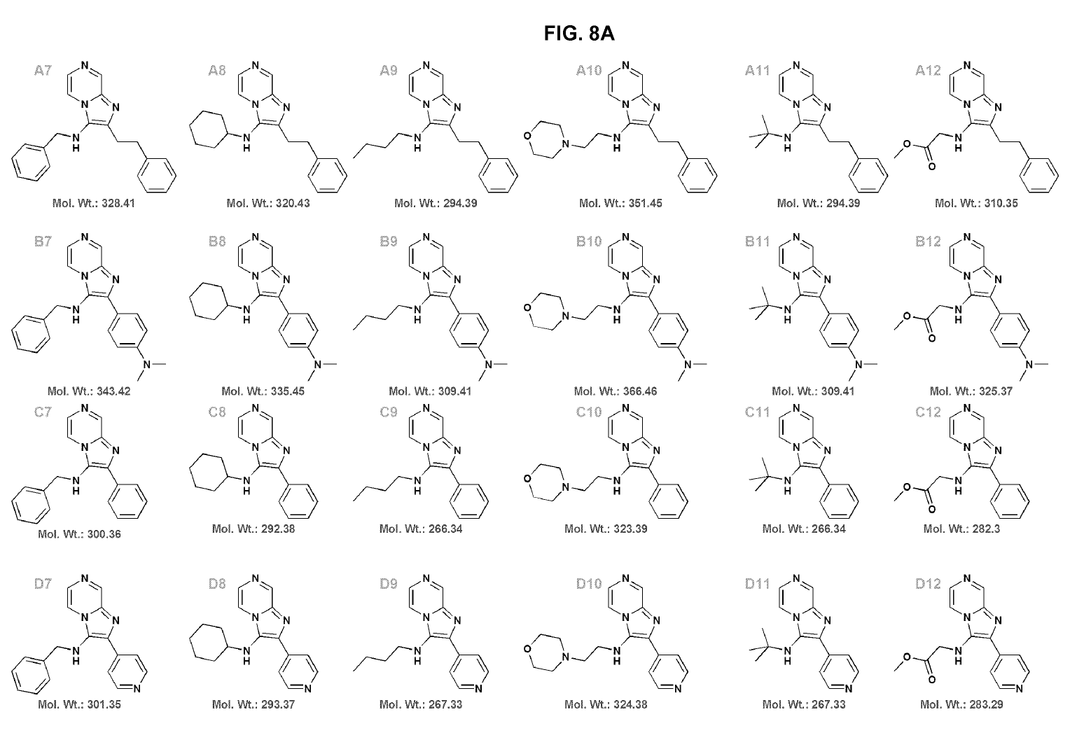

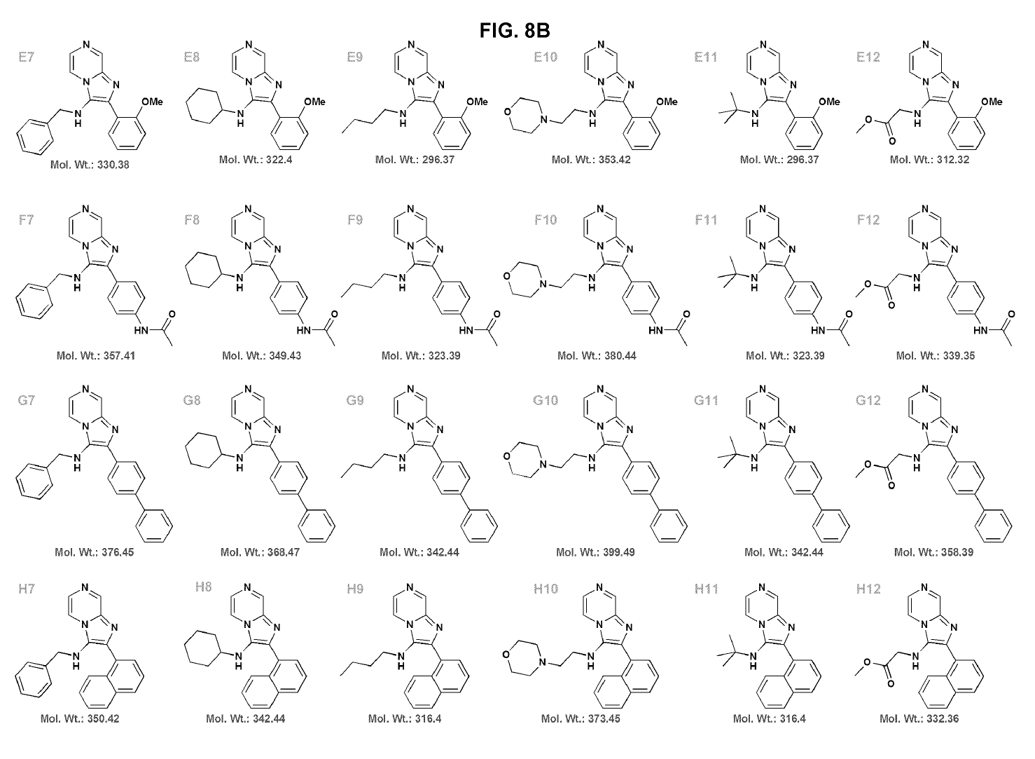

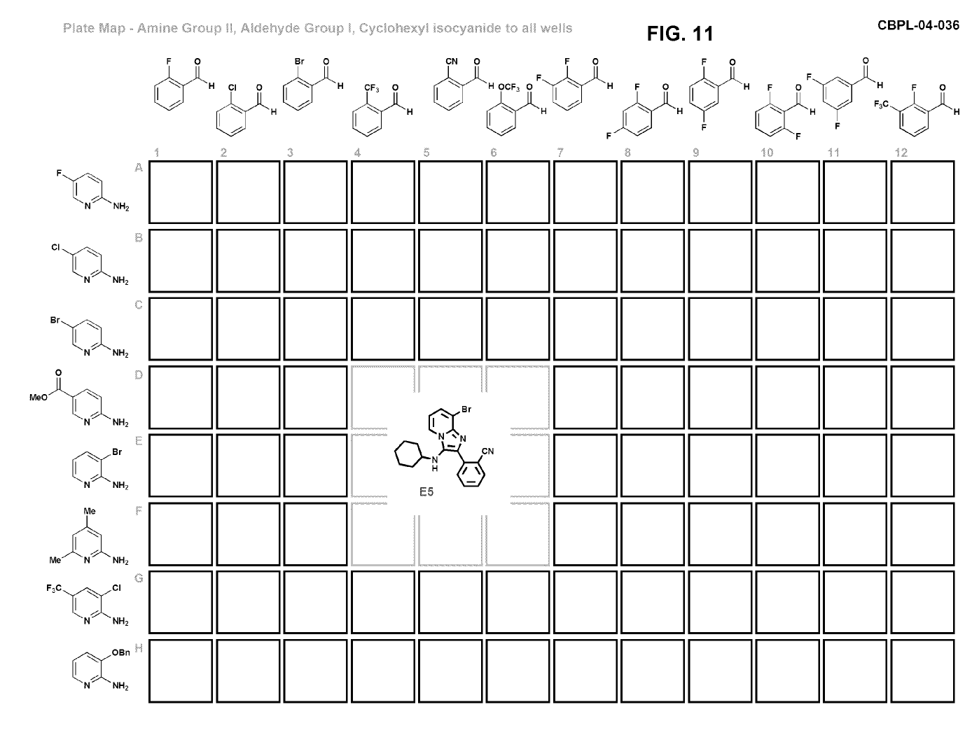

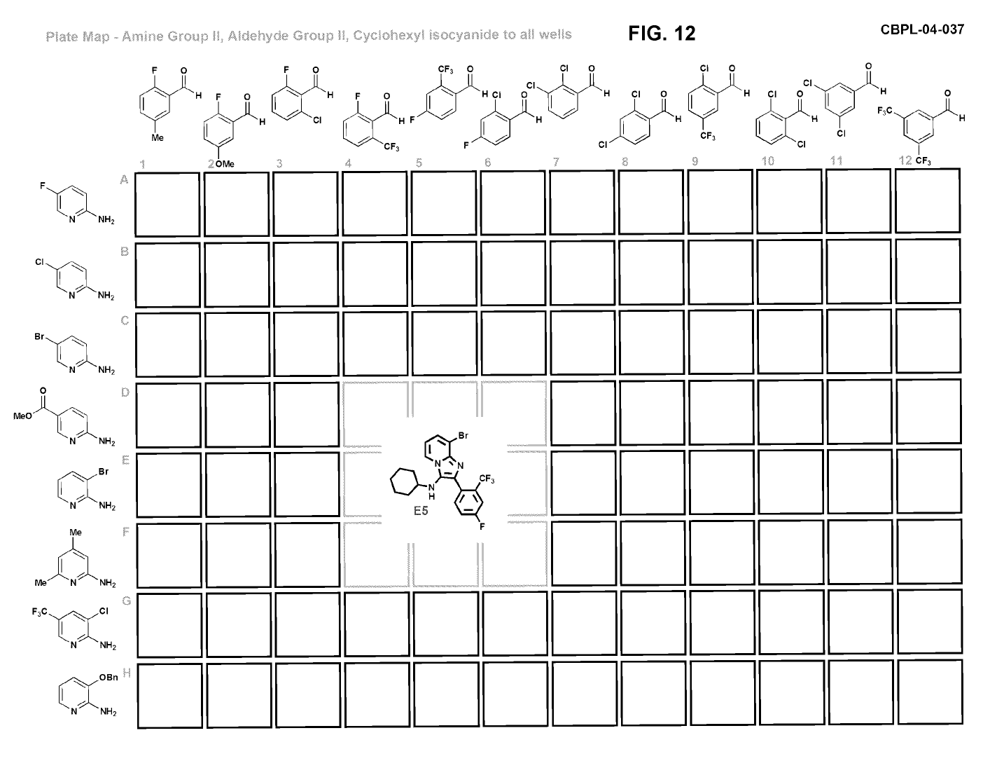

Examples of representative NNRTI compounds disclosed herein and encompassed by formulas presented above are provided throughout this disclosure, including the figures, for example in FIGS. 4, 5, 7-13 and 25. These examples and the illustrative preparations illustrated in the figures and examples below are provided to enable those skilled in the art to more clearly understand and to make and use the disclosed compounds.

### Methods for Making the Disclosed Compounds

The inhibitors disclosed herein can be prepared as set forth herein or by other methods that will be apparent to those of skill in the art of organic synthesis upon consideration of the present disclosure. The disclosed compounds can be prepared by a variety of methods depicted in the illustrative synthetic reaction schemes and examples shown and described herein. The starting materials and reagents used in preparing these compounds generally are either available from commercial suppliers, such as Sigma-Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. Disclosed synthetic schemes are merely illustrative of some methods by which the compounds disclosed herein can be synthesized, and various modifications to these synthetic reaction schemes can be made and will be suggested to one skilled in the art having referred to the information contained in this disclosure.

One method for making exemplary inhibitors follows Scheme 1:

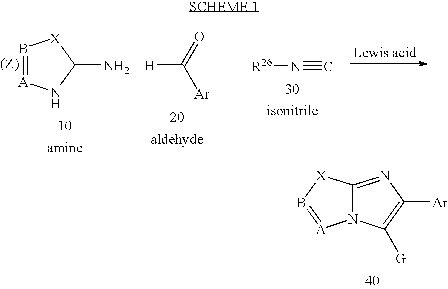

wherein the moiety G in product 40 is derived from isonitrile 30 and thus product 40 also can be represented by the formula

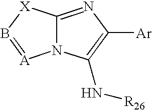

In Scheme 1, as disclosed above, A is N, O, S or CR¹;

B is N or CR²

X is O; S;

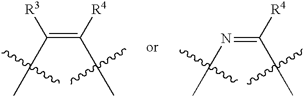

R¹, R², R³ and R⁴ independently are selected from H; cyano; halogen; haloalkyl; lower aliphatic; —OR₉; and —NR¹⁰R¹¹; and two of R¹, R², R³ and R⁴ together may optionally form a fused ring;

Ar is a 5 or 6 membered aromatic ring of the formula

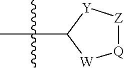

Y is S; Nor CR⁵;

Z is S; N; CR⁶;

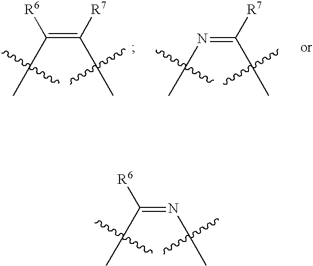

Q is S; N or CR⁸

W is S; N or CR⁹

R⁵-R⁹ independently are selected from H; cyano; halogen; haloalkyl; lower alkyl; —OR¹⁰; —SR¹¹; —NR¹²R¹³; and wherein two of R⁵-R⁹ together optionally may form a fused ring;

R¹⁰, R¹¹, R¹² and R¹³; independently are H, alkyl or acyl;

G is selected from —NR¹⁴R¹⁵ or —N═R¹⁶;

R¹⁴ and R¹⁵ independently are selected from H; aralkyl; lower alkyl; aryl; acyl;

—C(O)OR¹⁷; —C(O)NR¹⁸R¹⁹; —S(O)₂R²⁰; or together with one of R¹, R² or R³ forms a ring;

R¹⁶ is aralkyl and optionally together with one of R¹, R² or R³ forms a ring;

R¹⁷ is lower alkyl, aralkyl or aryl;

R¹⁸ and R₁₉ independently are selected from H; aralkyl; lower alkyl and aryl; and

R²⁰ is aryl.

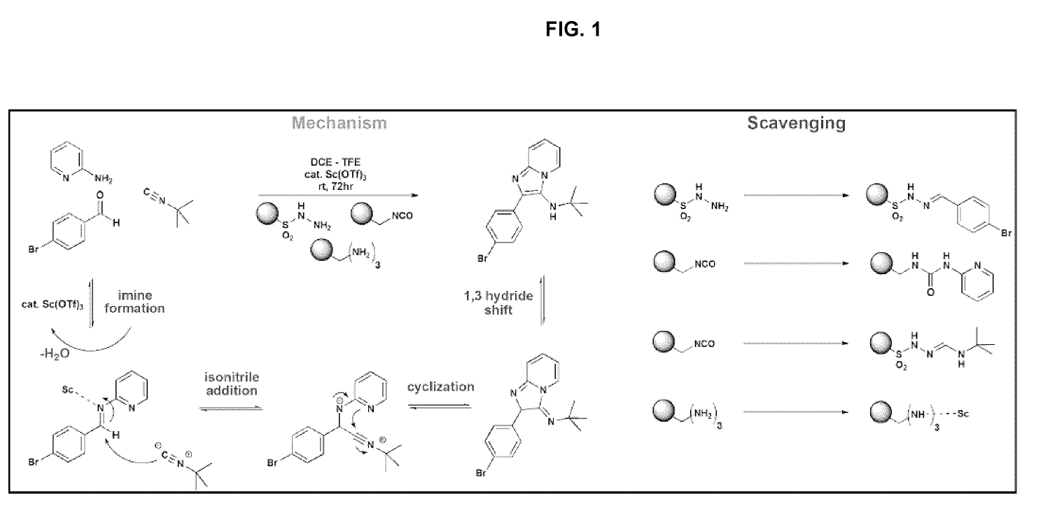

With reference to Scheme 1, this reaction is a Groebke (Ugi) three-component condensation, which without being limited to any particular mechanism or theory, currently is believed to follow the mechanism set forth in FIG. 1. In particular, and with continued reference to Scheme 1, amine 10 condenses with aldehyde 20 and isonitrile 30 in the presence of an acid catalyst, such as a Lewis acid, to yield the desired compound 40. The catalyst may be any suitable acid, but typically is a Lewis acid, such as a lanthanide-based Lewis acid, for example and without limitation scandium triflate (Sc(OTf)₃).

A second Groebke reaction-based scheme for synthesizing exemplary compounds is illustrated below in Scheme 2:

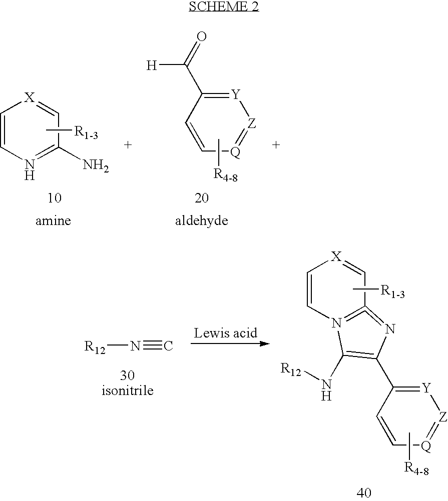

wherein X, Y, Z and Q independently are selected from N or C;

R₁-R₃ independently are selected from H; cyano; halogen; haloalkyl; lower alkyl; —OR₉; and —NR₁₀R₁₁;

R₄-R₈ independently are selected from H; cyano; halogen; haloalkyl; lower alkyl; —OR₉; —NR₁₀R₁₁; and wherein two of R₄-R₈ together optionally may form a fused ring;

R₉, R₁₀ and R₁₁ independently are alkyl or acyl;

R₁₂ is selected from H; aralkyl; lower alkyl; aryl; acyl;

—C(O)OR₁₅; —C(O)NR₁₆R₁₇; —S(O)₂R₁₈; or together with one of R₁, R₂ or R₃ forms a ring;

R₁₄ is aralkyl and optionally together with one of R₁, R₂ or R₃ forms a ring;

R₁₅ is lower alkyl, aralkyl or aryl;

R₁₆ and R₁₇ independently are selected from H; aralkyl; lower alkyl and aryl; and

R₁₈ is aryl.

Isonitriles useful for synthesizing the disclosed are known to those of skill in the art and can be used to synthesize isonitriles used in examples described herein, as well as in the preparation of additional isonitriles that can be used, in combination with the disclosed aldehydes and amines to synthesize additional compounds. Isonitriles used herein may be prepared, for example from the corresponding amines by reaction with chloroform and a strong bases, such as, for example, an alkali metal hydroxide (carbylamine reaction) or from the corresponding formamides by reaction with phosphorus oxychloride (POCl₃) or phosgene in the presence of nitrogenous bases. Examples of these protocols are described by Ugi et al. in: “Isonitrile Chemistry”, I. Ugi (ed.), Academic Press, New York, 1971; and in Angew. Chem. 1965, 77, 492; both of these references are incorporated herein by reference.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

The following schemes set forth additional exemplary amines, aldehydes and isonitriles for making the disclosed inhibitor compounds.

SCHEME 3
Exemplary compounds listed by amine and isonitrile

| R—CHO | 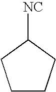 | 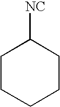 | |
|---|---|---|---|
| 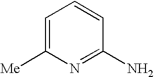 | 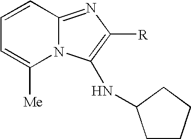 | 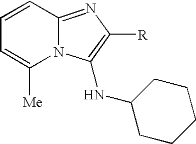 | |
| 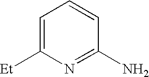 | 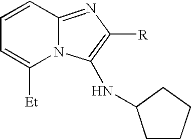 | 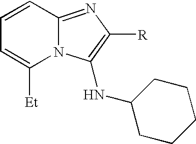 | |
| 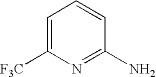 | 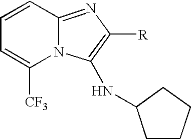 | 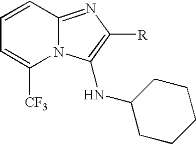 | |
| 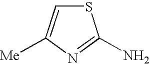 | 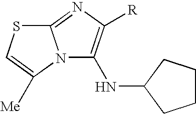 | 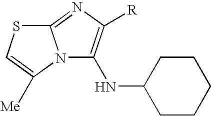 | |
| 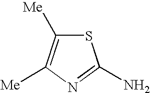 | 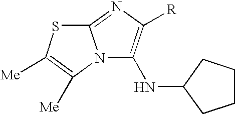 | 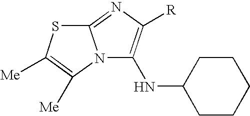 | |
| 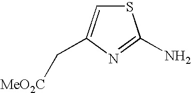 | 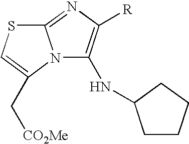 | 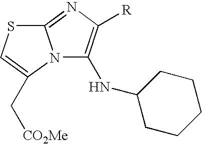 | |
| 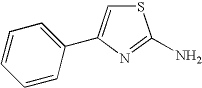 | 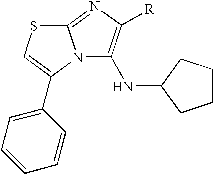 | 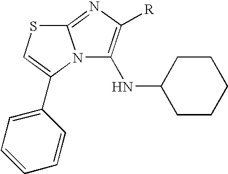 | |
| 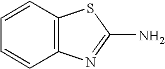 | 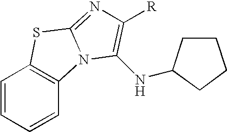 | 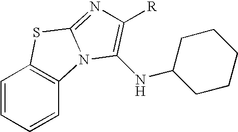 | |
| R—CHO | 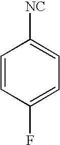 | 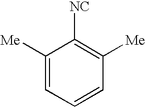 | |
| 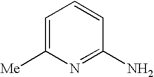 | 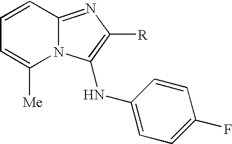 | 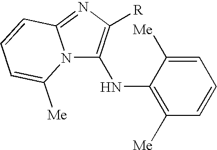 | |
| 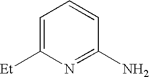 | 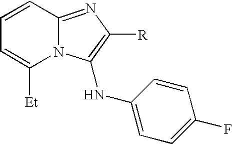 | 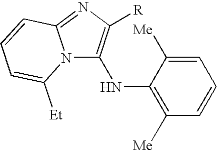 | |
| 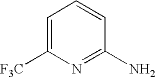 | 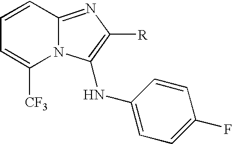 | 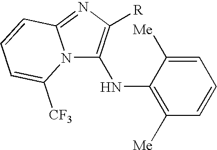 | |
| 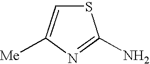 | 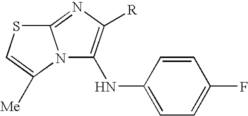 | 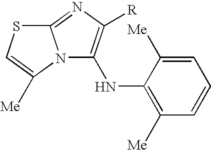 | |
| 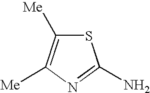 | 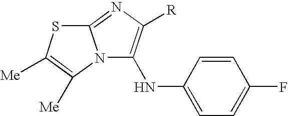 | 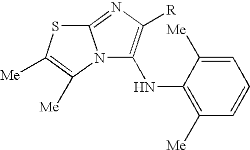 | |
| 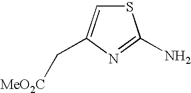 | 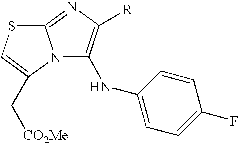 | 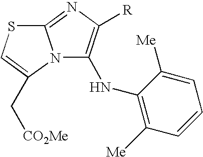 | |
| 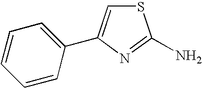 | 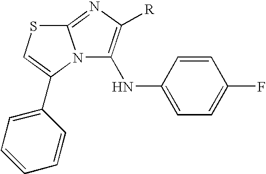 | 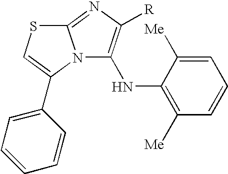 | |
| 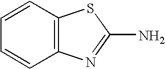 | 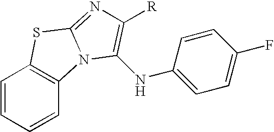 | 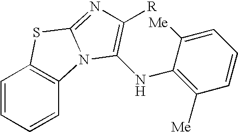 | |
| | R—CHO | | 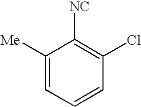 |
| | 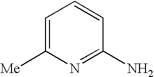 | | 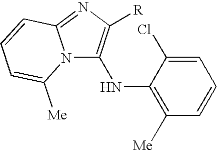 |
| | 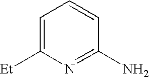 | | 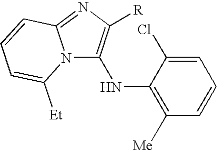 |
| | 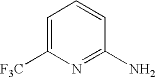 | | 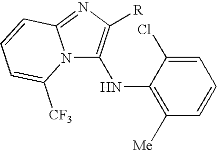 |
| | 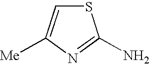 | | 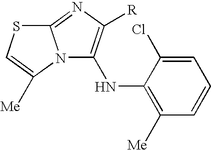 |
| | 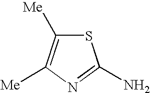 | | 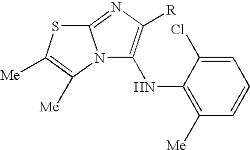 |
| | 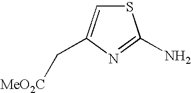 | | 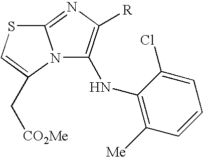 |
| | 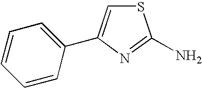 | | 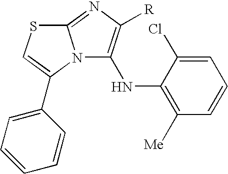 |
| | 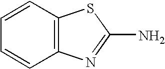 | | 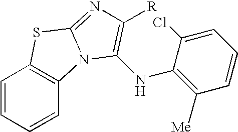 |

SCHEME 4

| R—CHO | 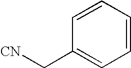 | 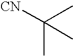 | |
|---|---|---|---|
| 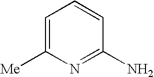 | 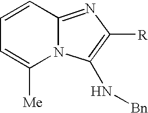 | 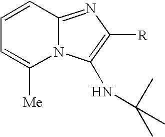 | |
| 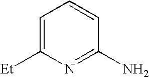 | 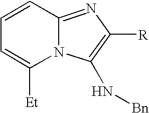 | 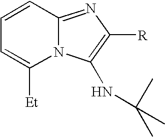 | |
| 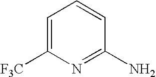 | 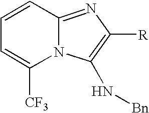 | 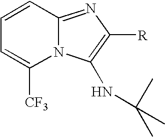 | |
| 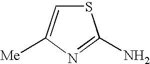 | 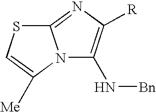 | 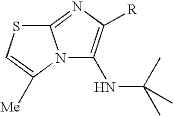 | |
| 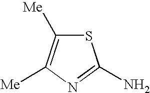 | 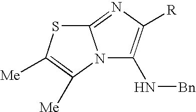 | 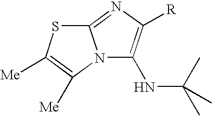 | |
| 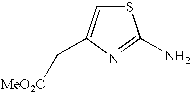 | 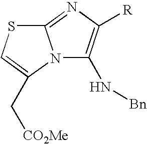 | 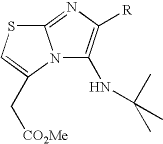 | |
| 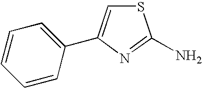 | 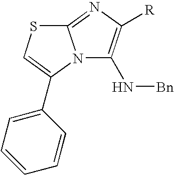 | 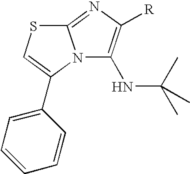 | |
| 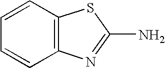 | 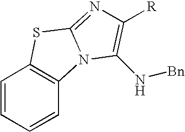 | 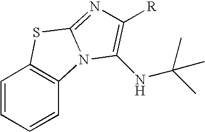 | |
| R—CHO | 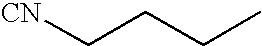 | 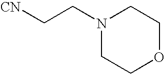 | |
| 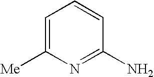 | 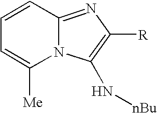 | 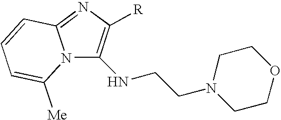 | |
| 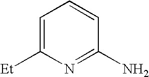 | 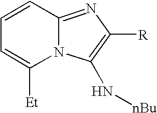 | 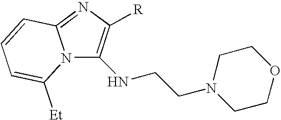 | |
| 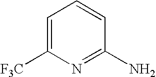 | 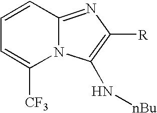 | 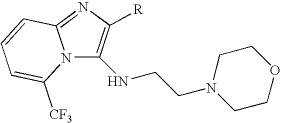 | |
| 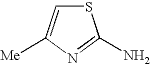 | 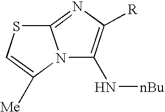 | 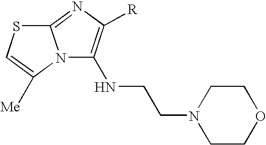 | |
| 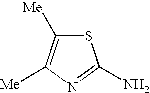 | 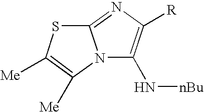 | 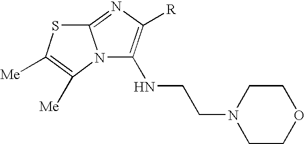 | |
| 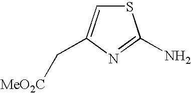 | 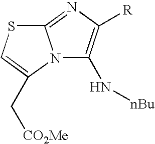 | 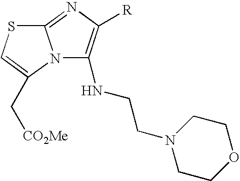 | |
| 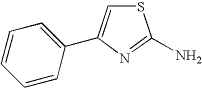 | 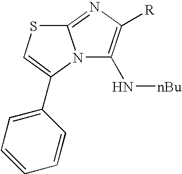 | 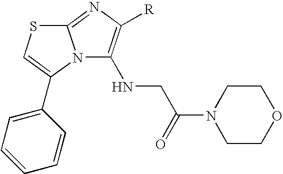 | |
| 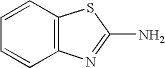 | 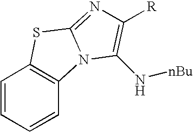 | 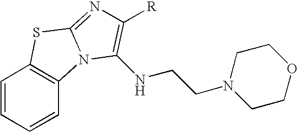 | |
| | R—CHO | | 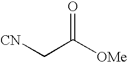 |
| | 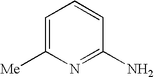 | | 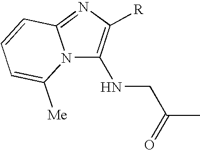 |
| | 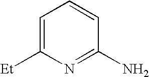 | | 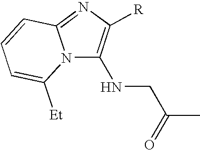 |
| | 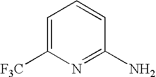 | | 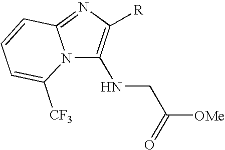 |
| | 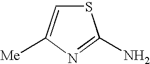 | | 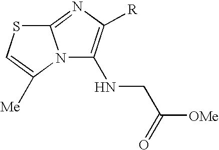 |
| | 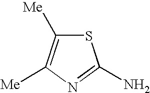 | | 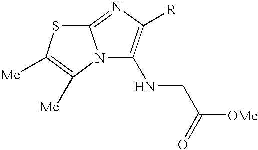 |
| | 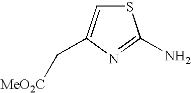 | | 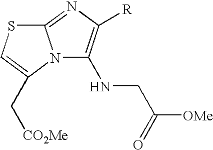 |
| | 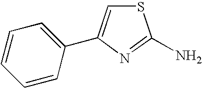 | | 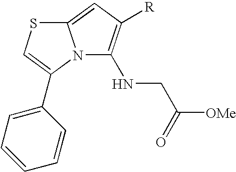 |
| | 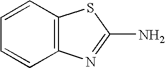 | | 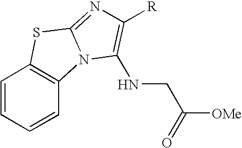 |

SCHEME 5

| R—CHO | 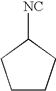 | 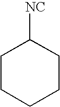 | |
|---|---|---|---|
| 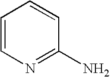 | 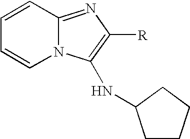 | 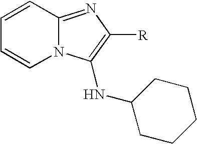 | |
| 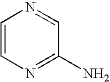 | 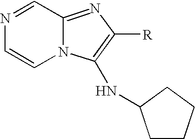 | 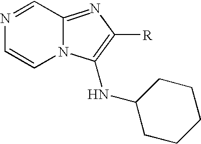 | |
| 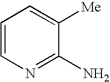 | 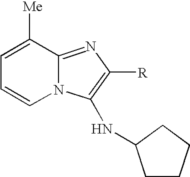 | 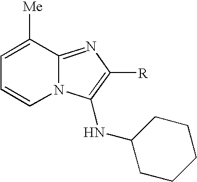 | |
| 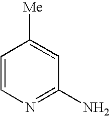 | 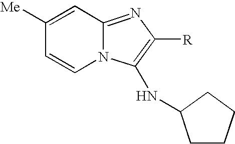 | 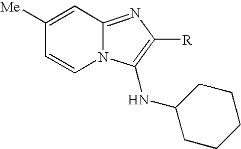 | |
| 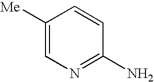 | 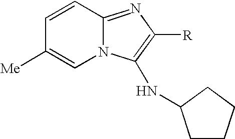 | 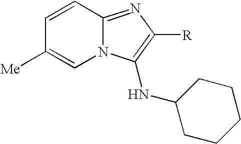 | |
| 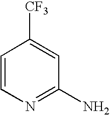 | 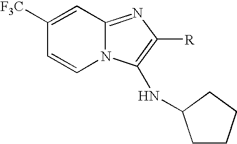 | 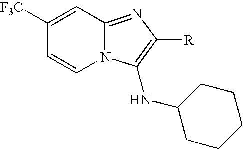 | |
| 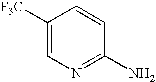 | 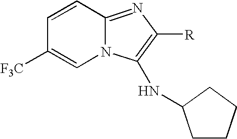 | 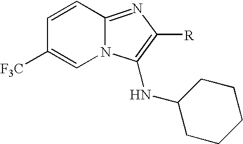 | |
| 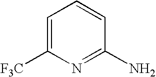 | 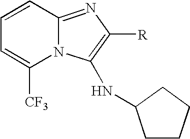 | 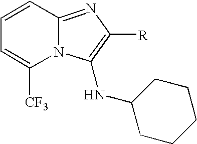 | |
| R—CHO | 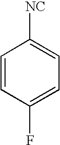 | 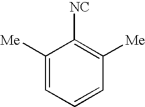 | |
| 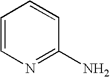 | 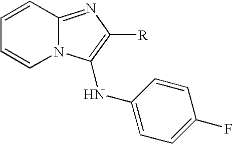 | 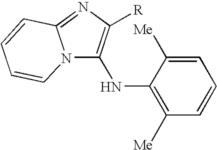 | |
| 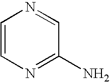 | 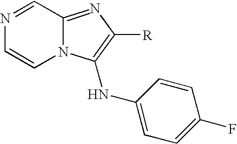 | 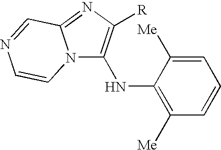 | |
| 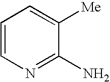 | 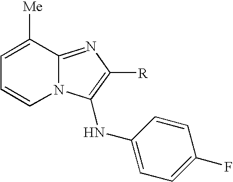 | 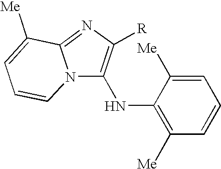 | |
| 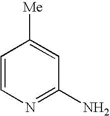 | 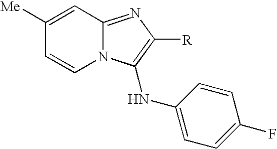 | 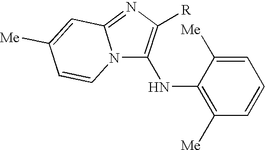 | |
| 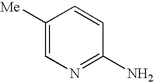 | 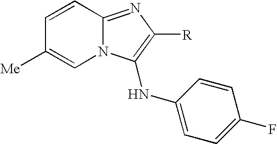 | 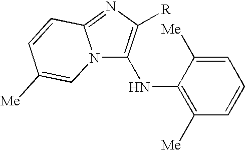 | |
| 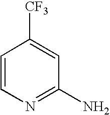 | 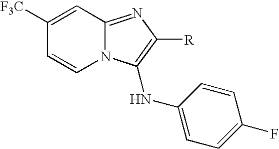 | 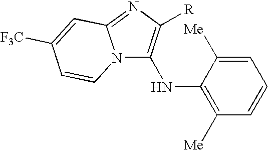 | |
| 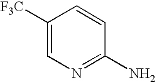 | 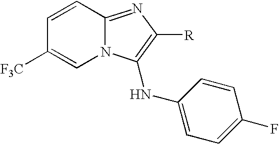 | 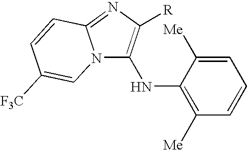 | |
| 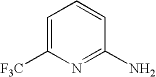 | 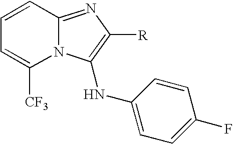 | 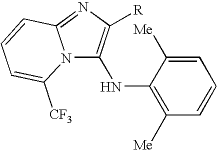 | |
| | R—CHO | | 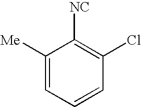 |
| | 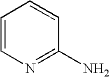 | | 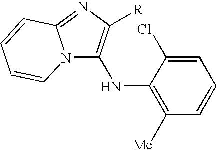 |
| | 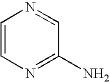 | | 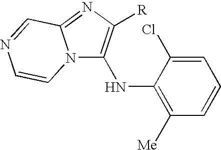 |
| | 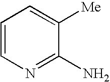 | | 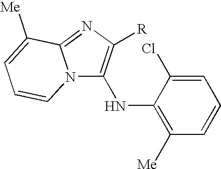 |
| | 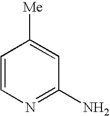 | | 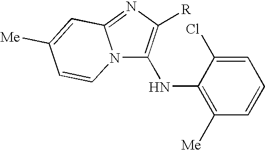 |
| | 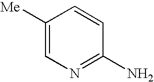 | | 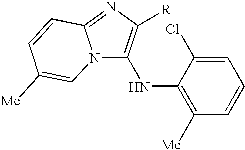 |
| | 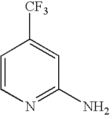 | | 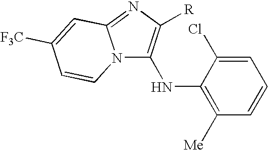 |
| | 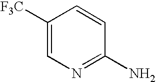 | | 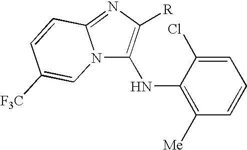 |
| | 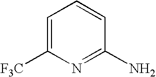 | | 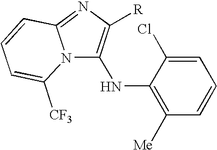 |

SCHEME 6

| R—CHO | 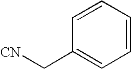 | 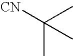 |
|---|---|---|
| 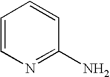 | 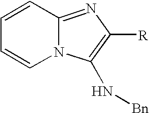 | 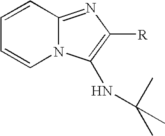 |
| 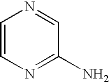 | 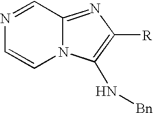 | 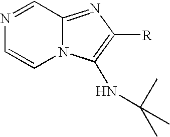 |
| 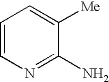 | 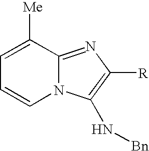 | 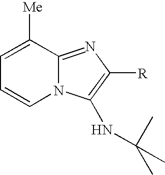 |
| 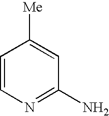 | 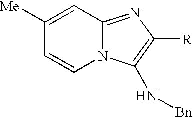 | 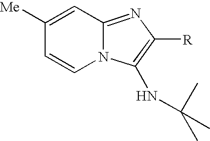 |
| 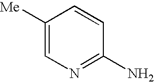 | 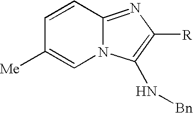 | 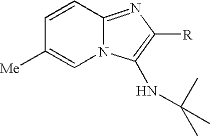 |
| 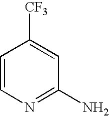 | 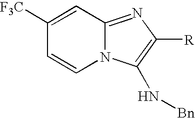 | 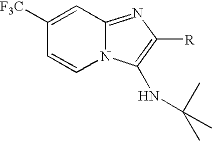 |
| 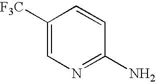 | 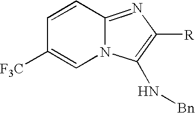 | 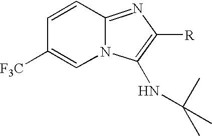 |
| 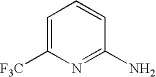 | 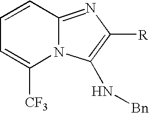 | 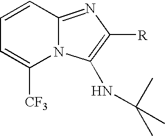 |
| R—CHO | 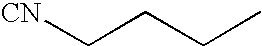 | 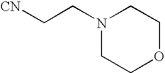 |
| 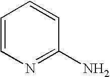 | 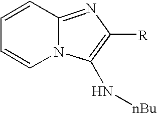 | 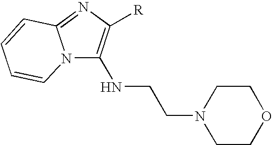 |
| 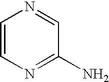 | 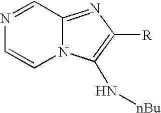 | 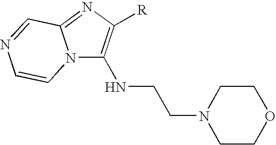 |
| 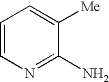 | 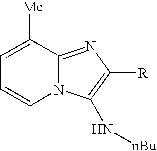 | 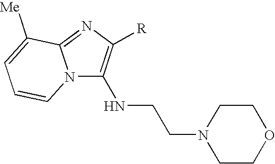 |
| 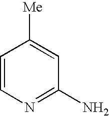 | 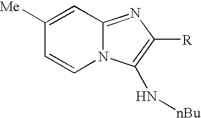 | 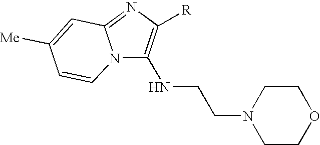 |
| 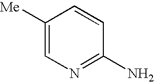 | 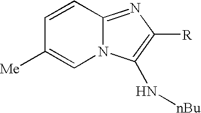 | 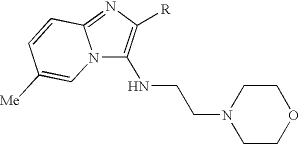 |
| 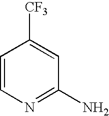 | 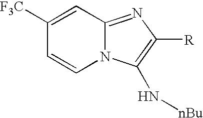 | 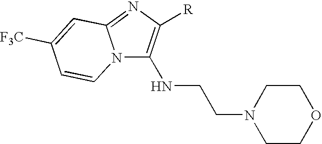 |
| 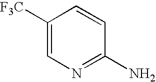 | 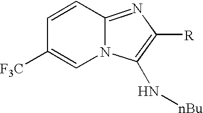 | 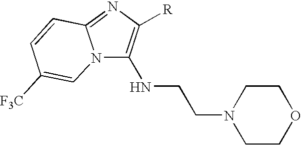 |
| 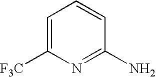 | 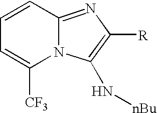 | 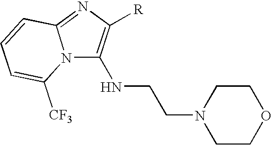 |
| | R—CHO | 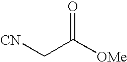 |
| | 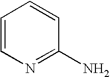 | 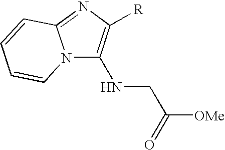 |
| | 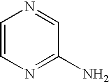 | 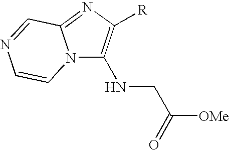 |
| | 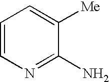 | 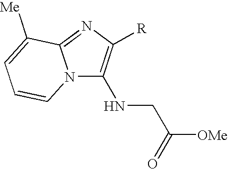 |
| | 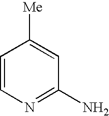 | 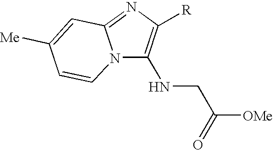 |
| | 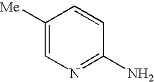 | 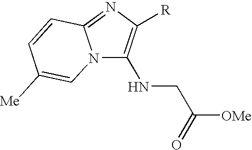 |
| | 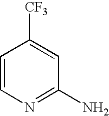 | 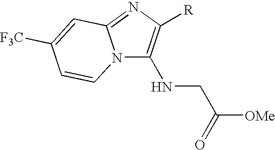 |
| | 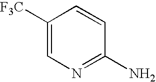 | 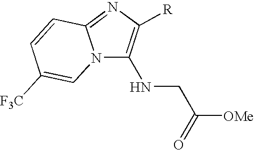 |
| | 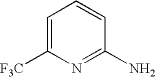 | 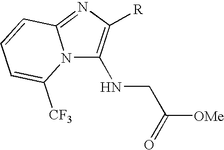 |

SCHEME 7

| R—CHO | 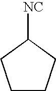 | 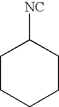 | |
|---|---|---|---|
| 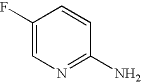 | 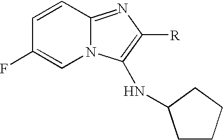 | 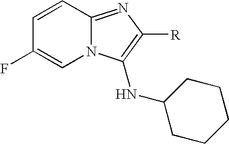 | |
| 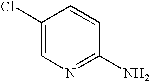 | 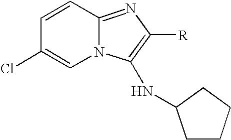 | 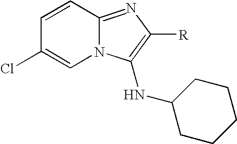 | |
| 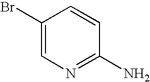 | 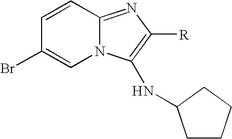 | 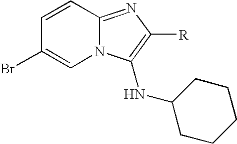 | |
| 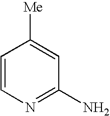 | 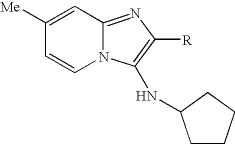 | 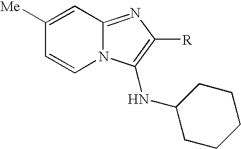 | |
| 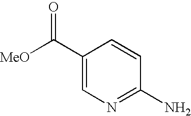 | 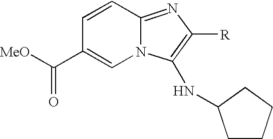 | 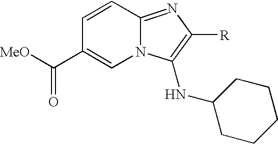 | |
| 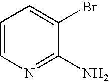 | 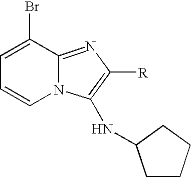 | 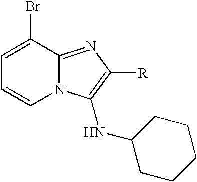 | |
| 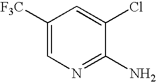 | 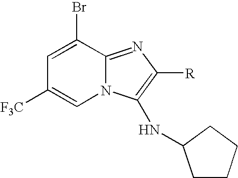 | 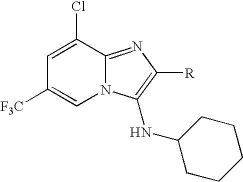 | |
| 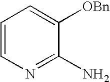 | 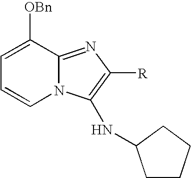 | 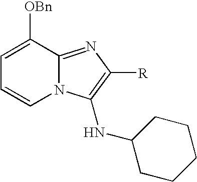 | |
| R—CHO | 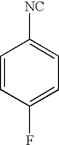 | 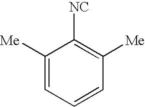 | |
| 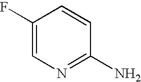 | 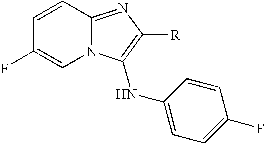 | 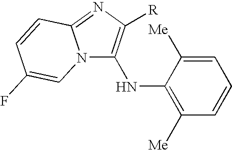 | |
| 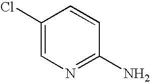 | 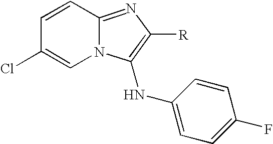 | 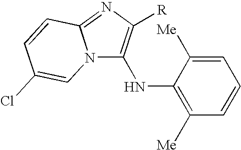 | |
| 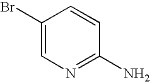 | 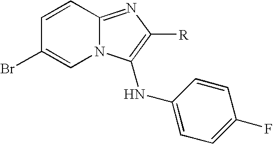 | 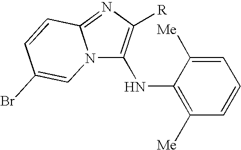 | |
| 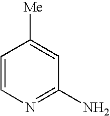 | 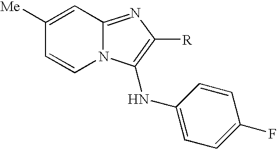 | 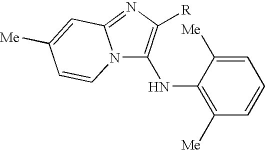 | |
| 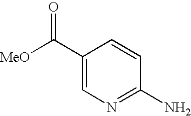 | 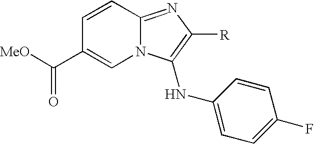 | 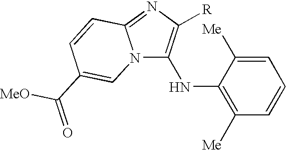 | |
| 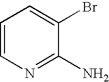 | 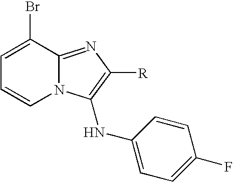 | 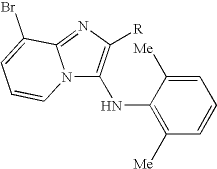 | |
| 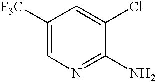 | 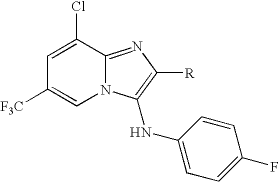 | 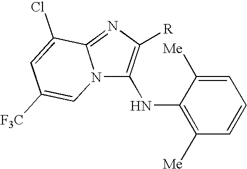 | |
| 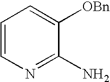 | 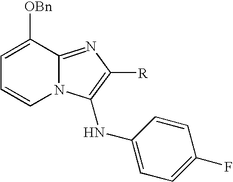 | 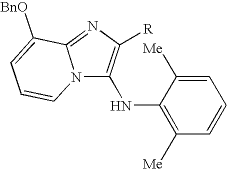 | |
| | R—CHO | | 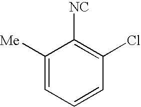 |
| | 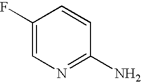 | | 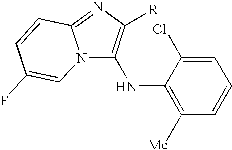 |
| | 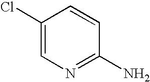 | | 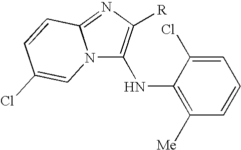 |
| | 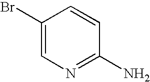 | | 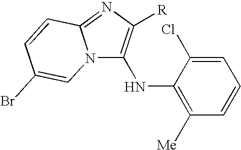 |
| | 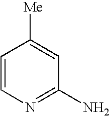 | | 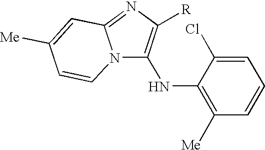 |
| | 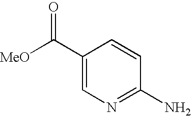 | | 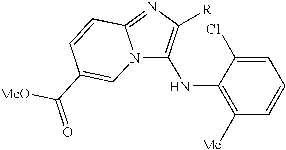 |
| | 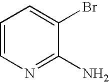 | | 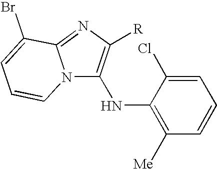 |
| | 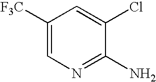 | | 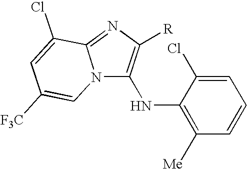 |
| | 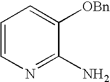 | | 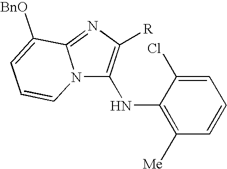 |

SCHEME 8

| R—CHO | 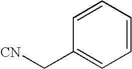 | 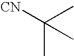 |
|---|---|---|
| 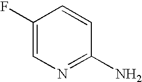 | 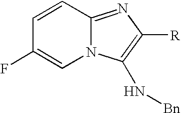 | 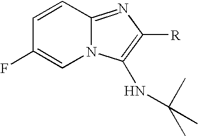 |
| 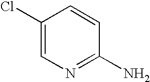 | 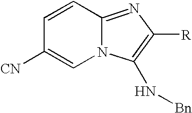 | 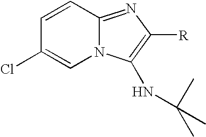 |
| 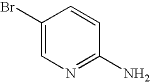 | 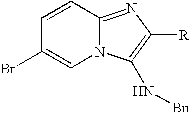 | 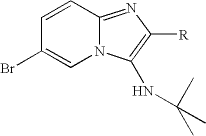 |
| 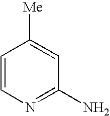 | 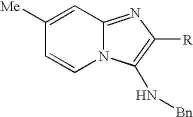 | 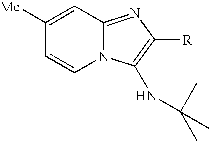 |
| 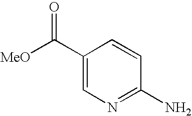 | 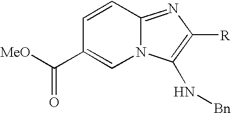 | 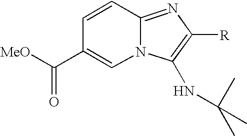 |
| 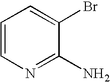 | 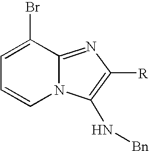 | 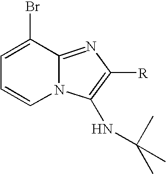 |
| 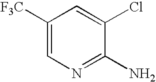 | 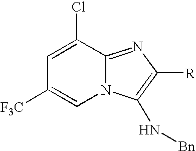 | 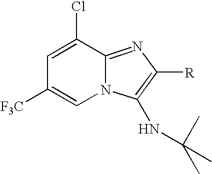 |
| 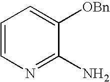 | 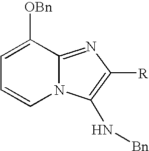 | 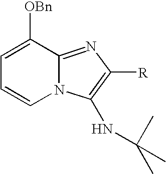 |
| R—CHO | 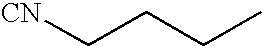 | 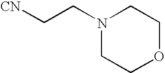 |
| 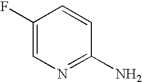 | 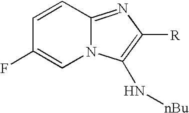 | 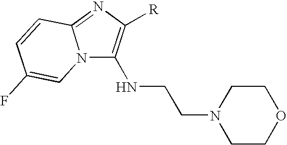 |
| 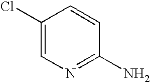 | 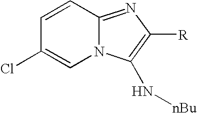 | 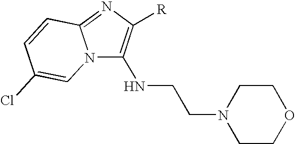 |
| 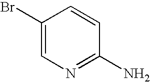 | 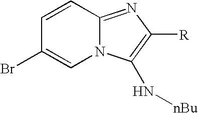 | 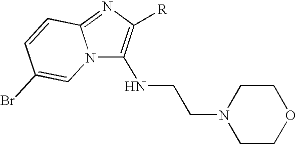 |
| 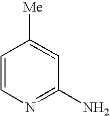 | 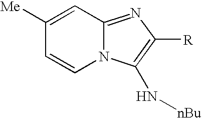 | 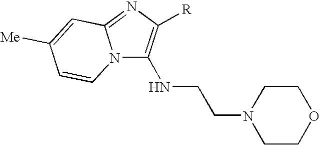 |
| 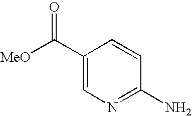 | 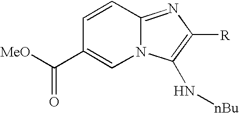 | 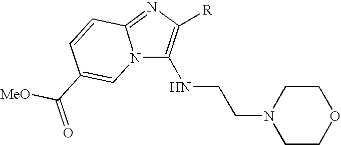 |
| 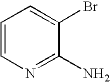 | 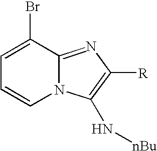 | 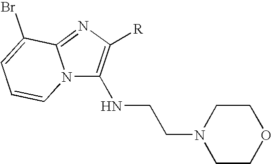 |
| 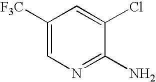 | 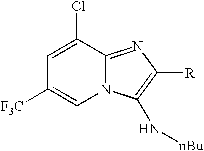 | 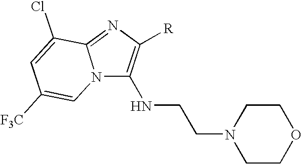 |
| 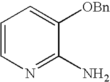 | 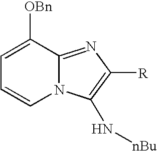 | 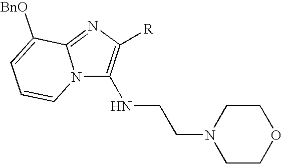 |
| | R—CHO | 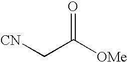 |
| | 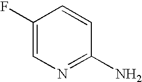 | 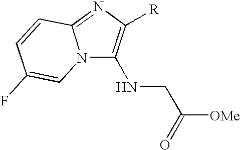 |
| | 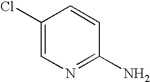 | 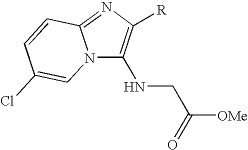 |
| | 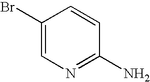 | 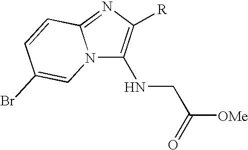 |
| | 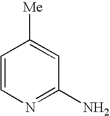 | 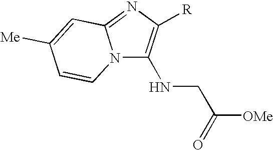 |
| | 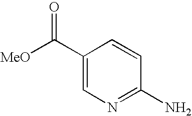 | 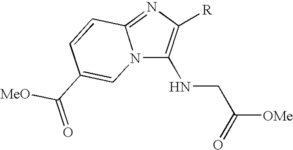 |
| | 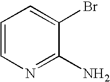 | 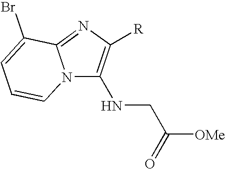 |
| | 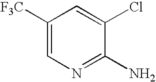 | 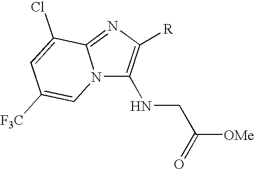 |
| | 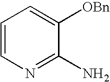 | 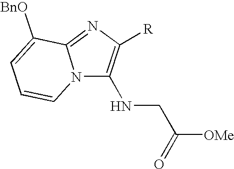 |

SCHEME 9

| R—CHO | 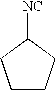 | 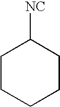 | |
|---|---|---|---|
| 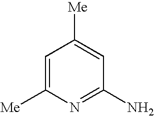 | 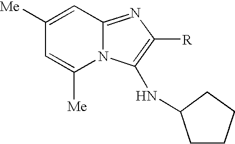 | 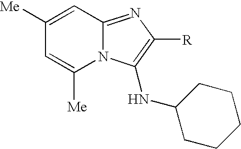 | |
| 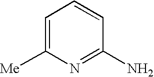 | 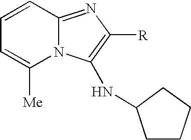 | 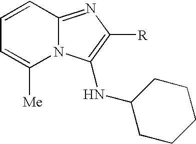 | |
| 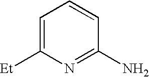 | 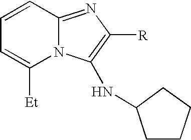 | 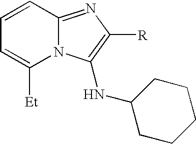 | |
| 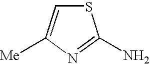 | 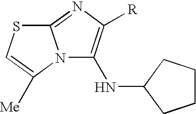 | 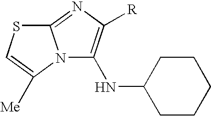 | |
| 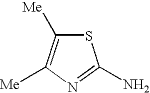 | 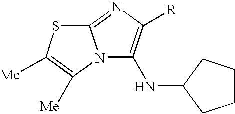 | 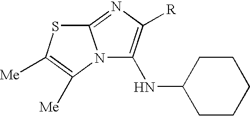 | |
| 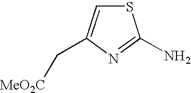 | 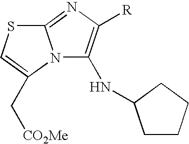 | 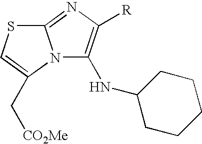 | |
| 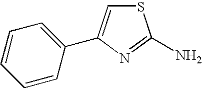 | 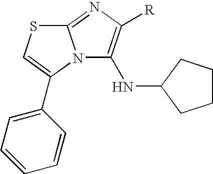 | 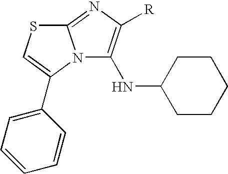 | |
| 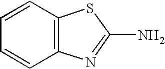 | 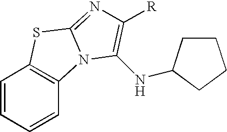 | 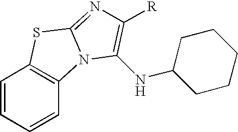 | |
| R—CHO | 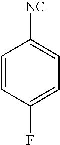 | 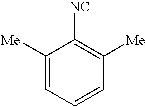 | |
| 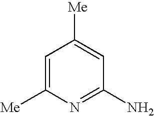 | 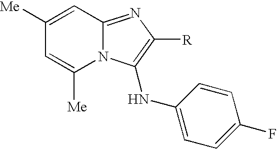 | 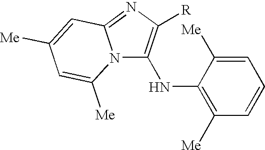 | |
| 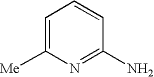 | 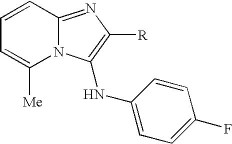 | 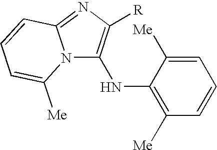 | |
| 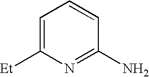 | 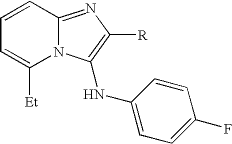 | 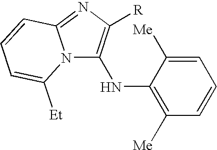 | |
| 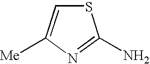 | 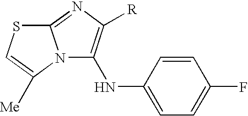 | 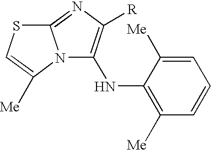 | |
| 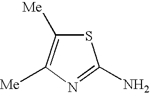 | 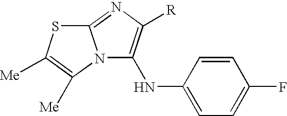 | 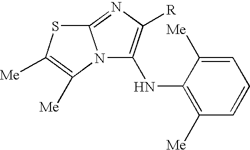 | |
| 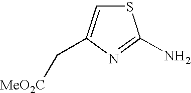 | 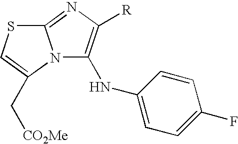 | 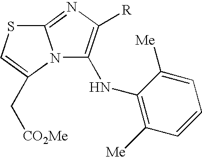 | |
| 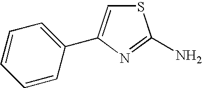 | 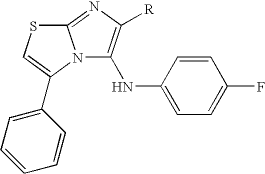 | 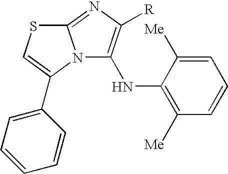 | |
| 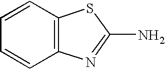 | 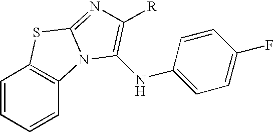 | 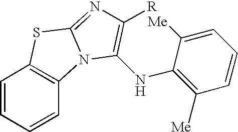 | |
| | R—CHO | | 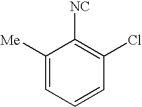 |
| | 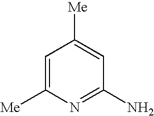 | | 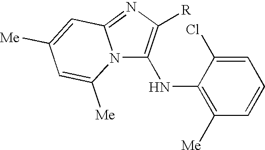 |
| | 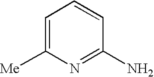 | | 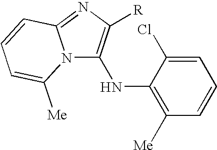 |
| | 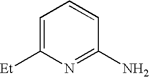 | | 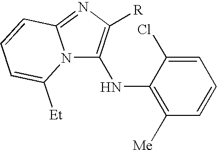 |
| | 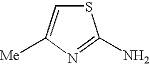 | | 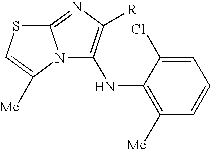 |
| | 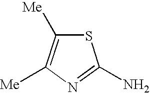 | | 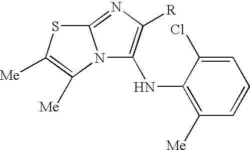 |
| | 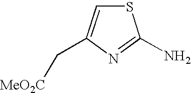 | | 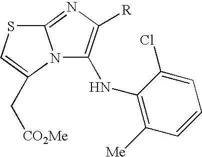 |
| | 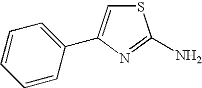 | | 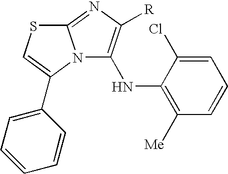 |
| | 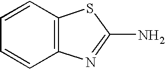 | | 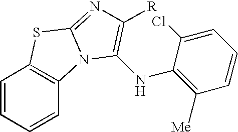 |

SCHEME 10

| R—CHO | 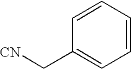 | 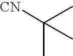 | |
|---|---|---|---|
| 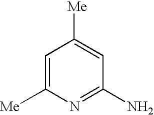 | 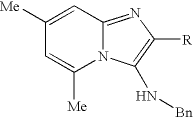 | 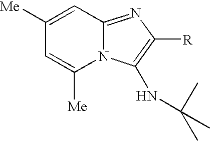 | |
| 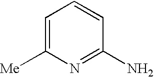 | 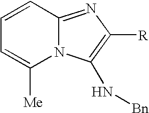 | 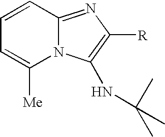 | |
| 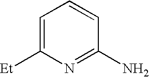 | 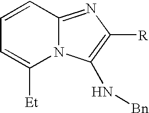 | 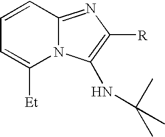 | |
| 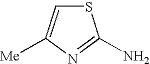 | 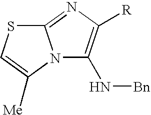 | 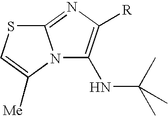 | |
| 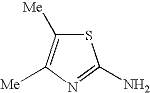 | 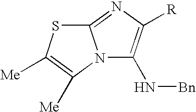 | 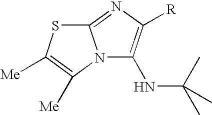 | |
| 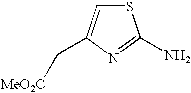 | 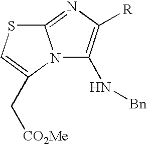 | 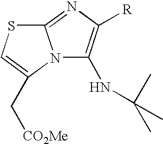 | |
| 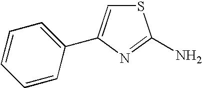 | 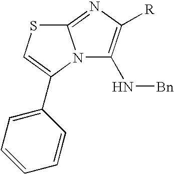 | 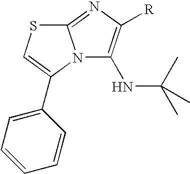 | |
| 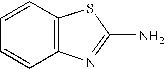 | 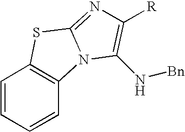 | 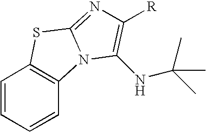 | |
| R—CHO | 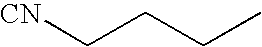 | 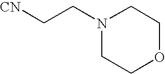 | |
| 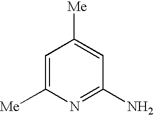 | 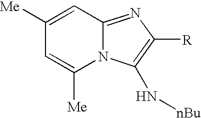 | 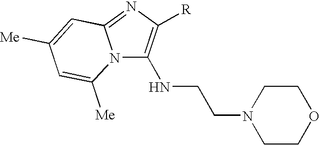 | |
| 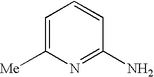 | 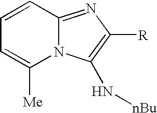 | 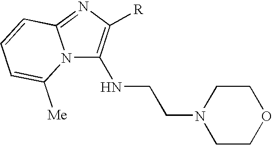 | |
| 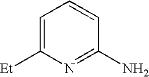 | 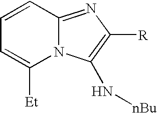 | 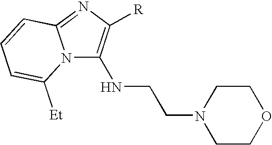 | |
| 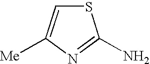 | 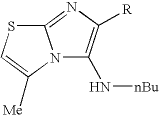 | 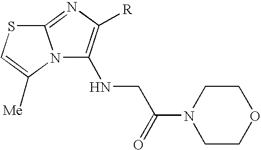 | |
| 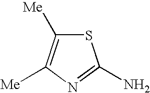 | 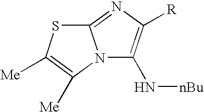 | 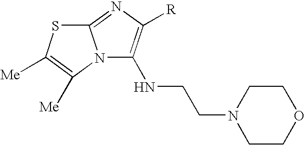 | |
| 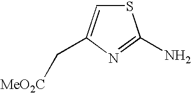 | 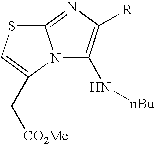 | 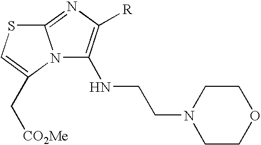 | |
| 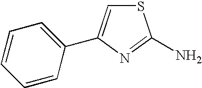 | 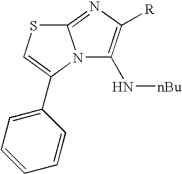 | 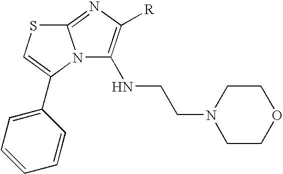 | |
| 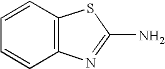 | 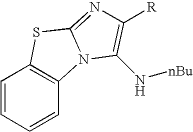 | 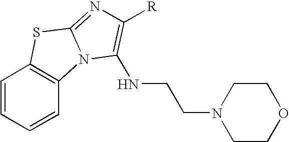 | |
| | R—CHO | | 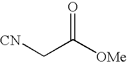 |
| | 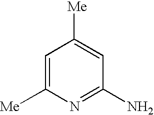 | | 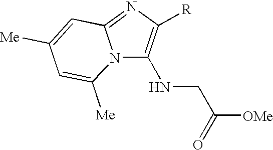 |
| | 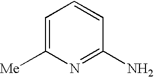 | | 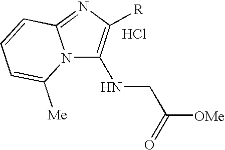 |
| | 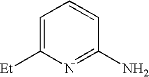 | | 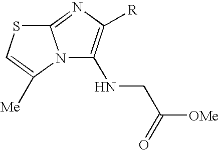 |
| | 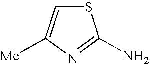 | | 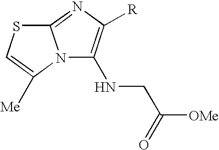 |
| | 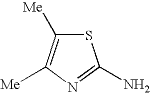 | | 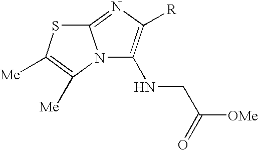 |
| | 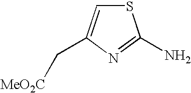 | | 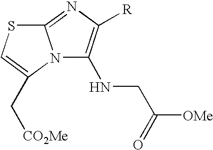 |
| | 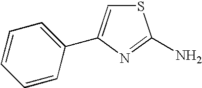 | | 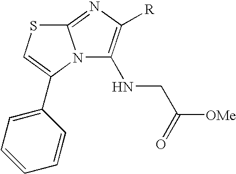 |
| | 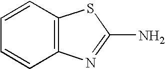 | | 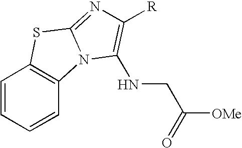 |

SCHEME 11

| R—CHO | 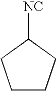 | 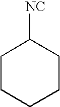 | |
|---|---|---|---|
| 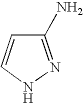 | 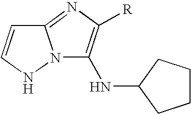 | 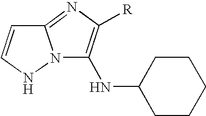 | |
| 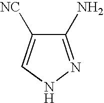 | 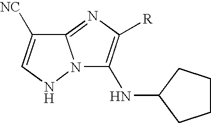 | 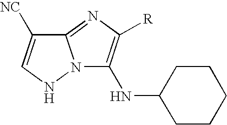 | |
| 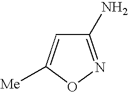 | 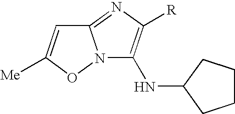 | 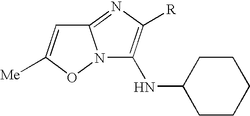 | |
| 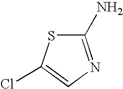 | 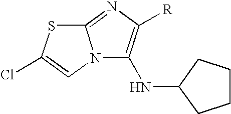 | 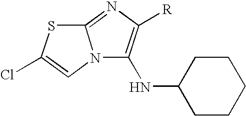 | |
| 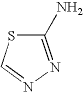 | 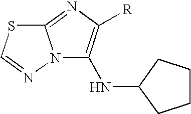 | 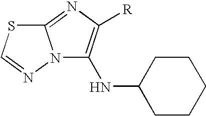 | |
| 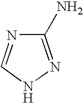 | 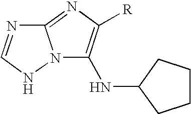 | 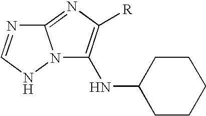 | |
| 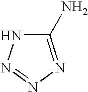 | 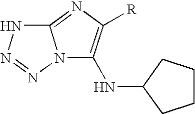 | 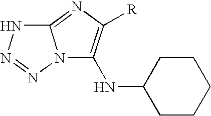 | |
| R—CHO | 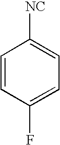 | 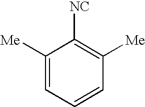 | |
| 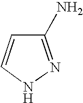 | 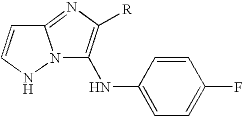 | 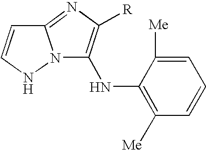 | |
| 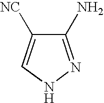 | 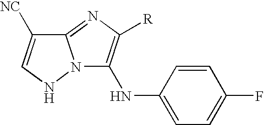 | 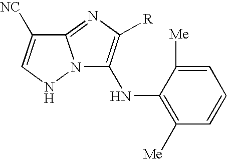 | |
| 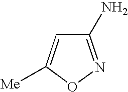 | 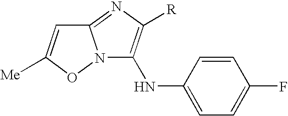 | 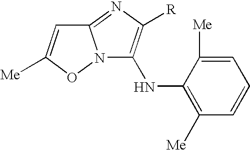 | |
| 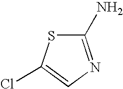 | 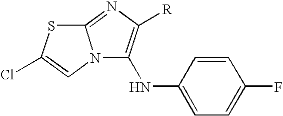 | 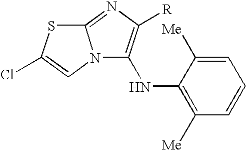 | |
| 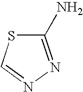 | 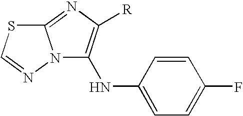 | 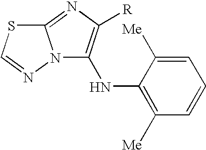 | |
| 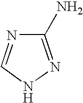 | 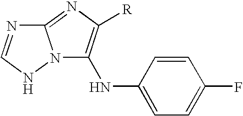 | 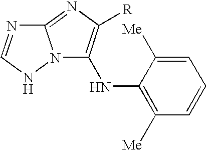 | |
| 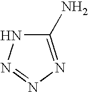 | 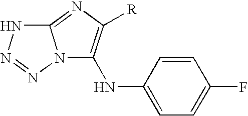 | 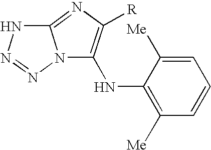 | |
| | R—CHO | | 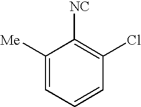 |
| | 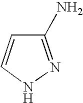 | | 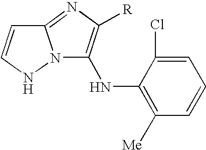 |
| | 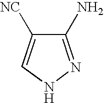 | | 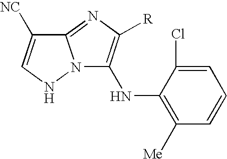 |
| | 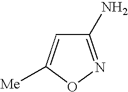 | | 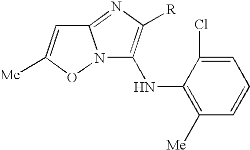 |
| | 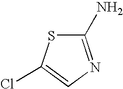 | | 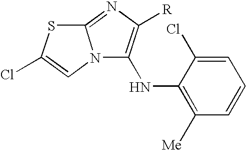 |
| | 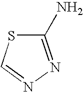 | | 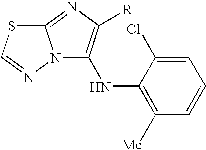 |
| | 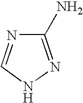 | | 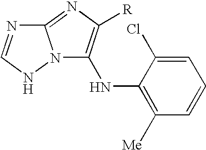 |
| | 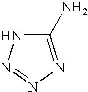 | | 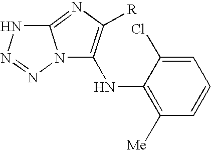 |

SCHEME 12

| R—CHO | 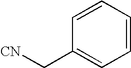 | 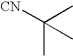 |
|---|---|---|
| 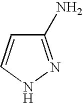 | 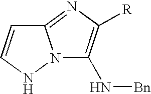 | 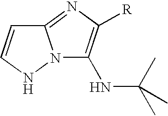 |
| 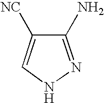 | 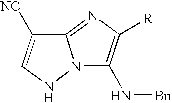 | 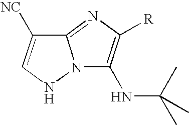 |
| 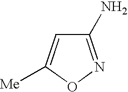 | 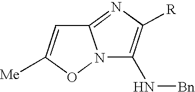 | 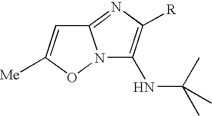 |
| 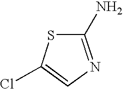 | 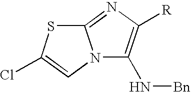 | 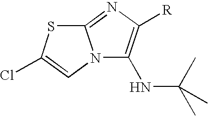 |
| 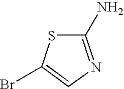 | 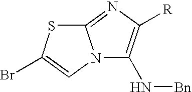 | 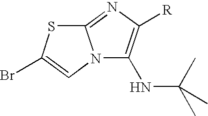 |
| 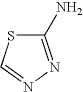 | 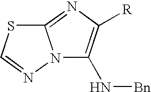 | 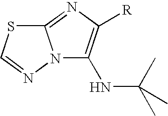 |
| 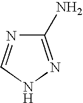 | 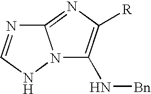 | 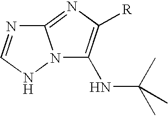 |
| 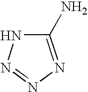 | 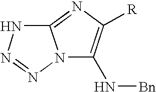 | 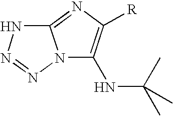 |
| R—CHO | 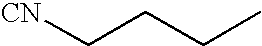 | 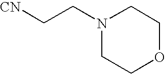 |
| 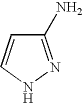 | 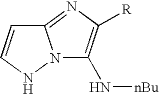 | 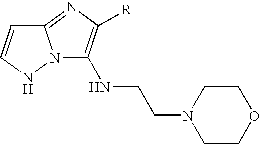 |
| 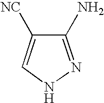 | 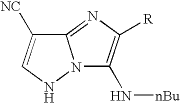 | 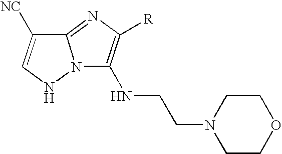 |
| 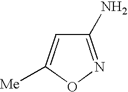 | 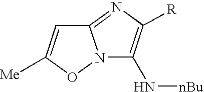 | 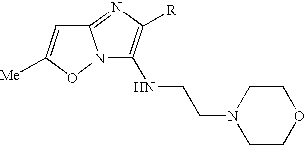 |
| 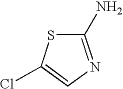 | 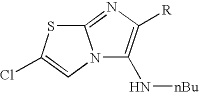 | 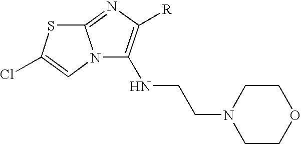 |
| 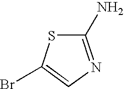 | 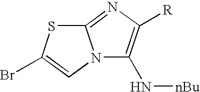 | 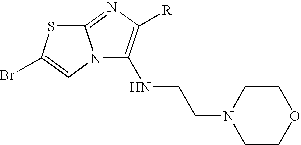 |
| 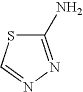 | 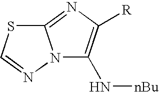 | 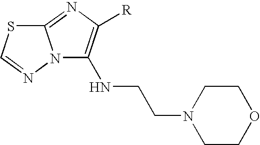 |
| 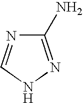 | 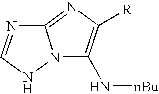 | 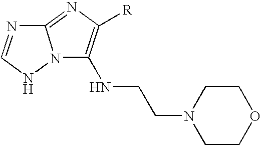 |
| 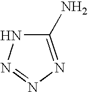 | 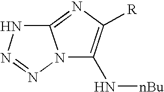 | 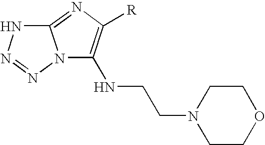 |
| | R—CHO | 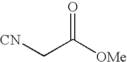 |
| | 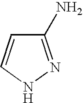 | 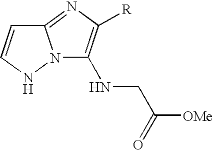 |
| | 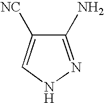 | 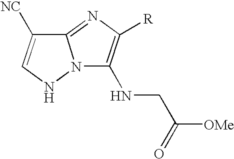 |
| | 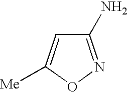 | 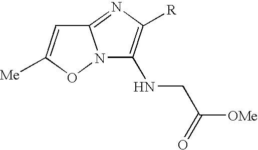 |
| | 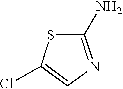 | 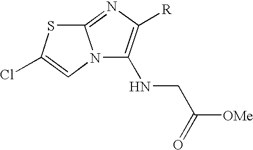 |
| | 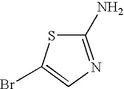 | 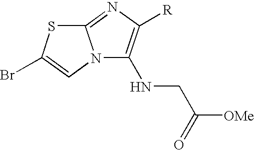 |
| | 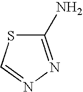 | 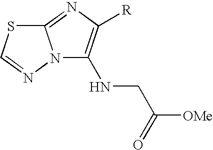 |
| | 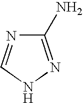 | 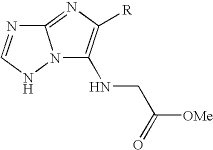 |
| | 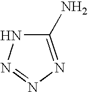 | 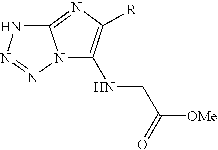 |

SCHEME 13

| R—CHO | 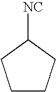 | 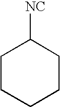 | |
|---|---|---|---|
| 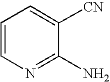 | 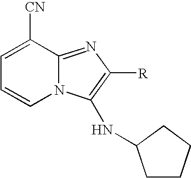 | 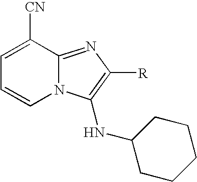 | |
| 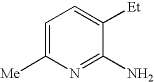 | 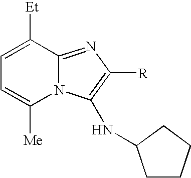 | 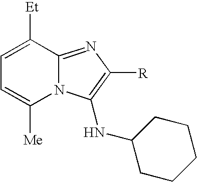 | |
| 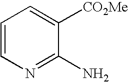 | 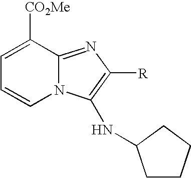 | 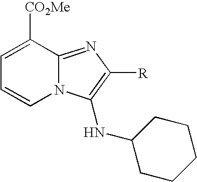 | |
| 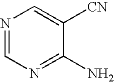 | 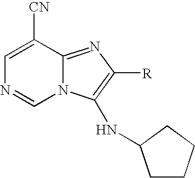 | 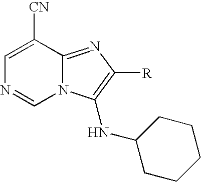 | |
| 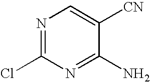 | 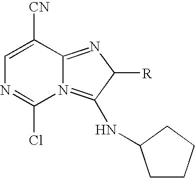 | 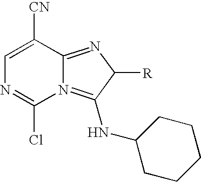 | |
| 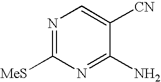 | 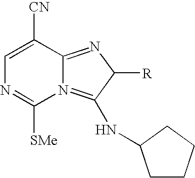 | 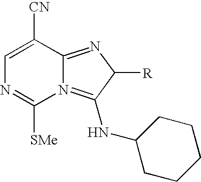 | |
| 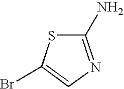 | 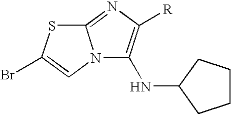 | 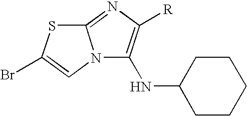 | |
| 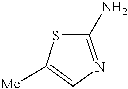 | 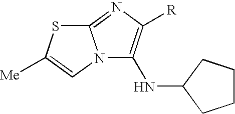 | 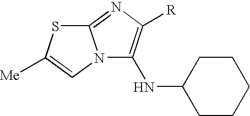 | |
| 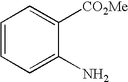 | 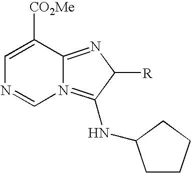 | 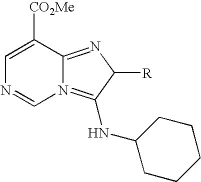 | |
| R—CHO | 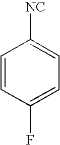 | 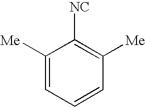 | |
| 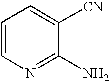 | 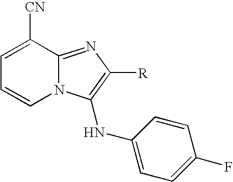 | 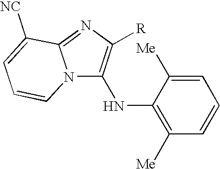 | |
| 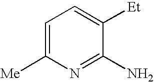 | 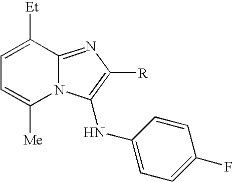 | 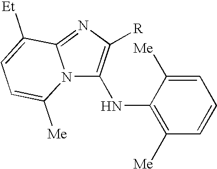 | |
| 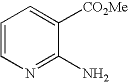 | 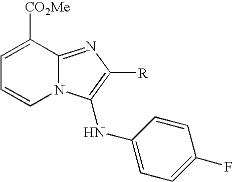 | 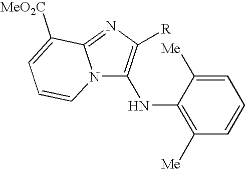 | |
| 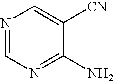 | 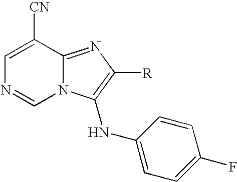 | 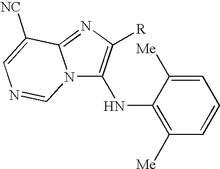 | |
| 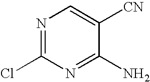 | 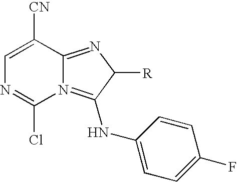 | 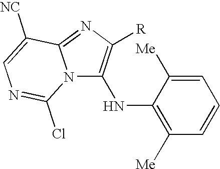 | |
| 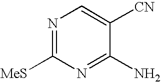 | 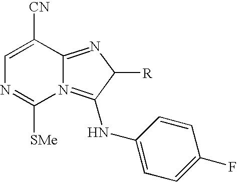 | 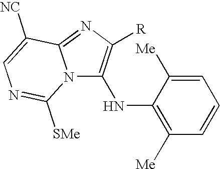 | |
| 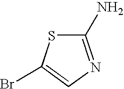 | 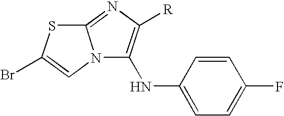 | 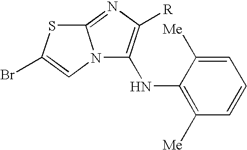 | |
| 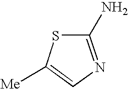 | 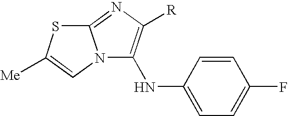 | 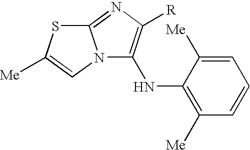 | |
| 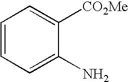 | 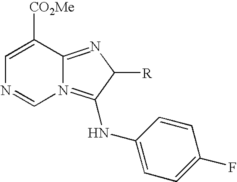 | 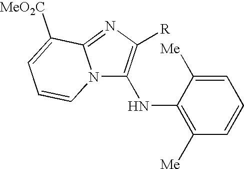 | |
| | R—CHO | | 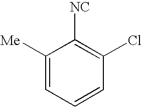 |
| | 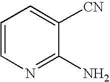 | | 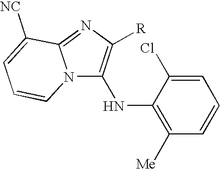 |
| | 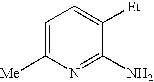 | | 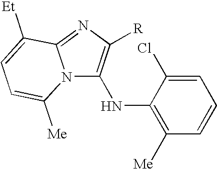 |
| | 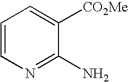 | | 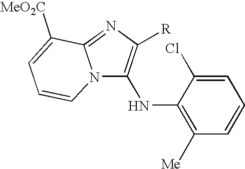 |
| | 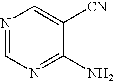 | | 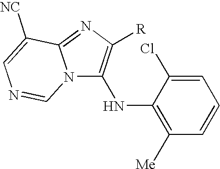 |
| | 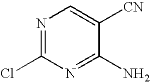 | | 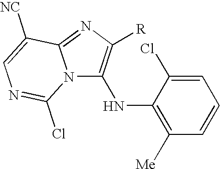 |
| | 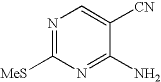 | | 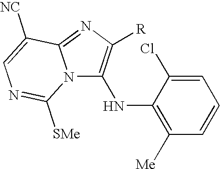 |
| | 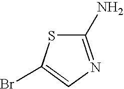 | | 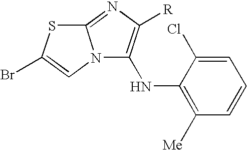 |
| | 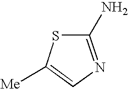 | | 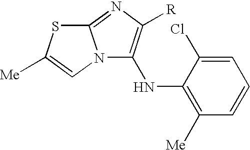 |
| | 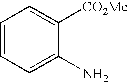 | | 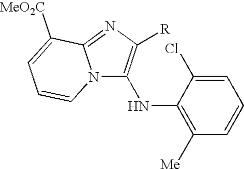 |

SCHEME 14

| R—CHO | 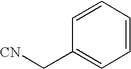 | 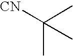 |
|---|---|---|
| 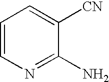 | 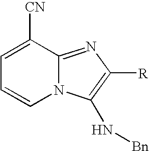 | 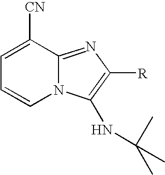 |
| 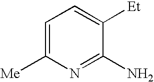 | 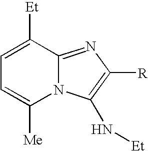 | 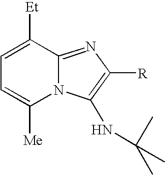 |
| 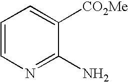 | 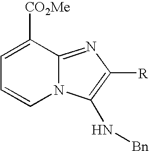 | 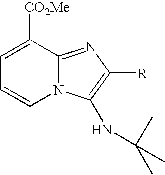 |
| 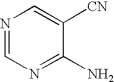 | 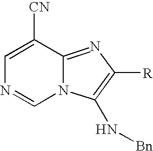 | 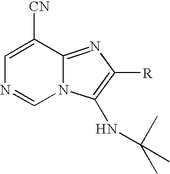 |
| 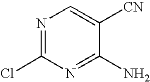 | 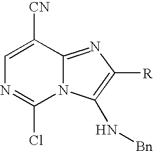 | 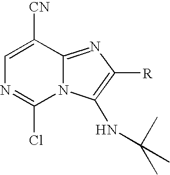 |
| 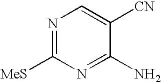 | 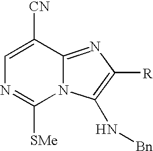 | 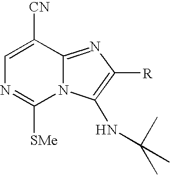 |
| R—CHO | 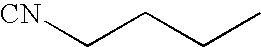 | 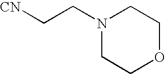 |
| 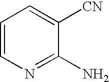 | 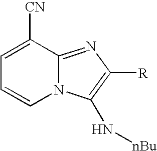 | 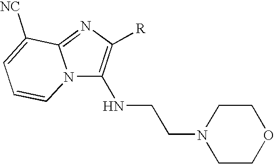 |
| 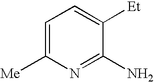 | 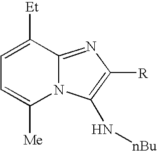 | 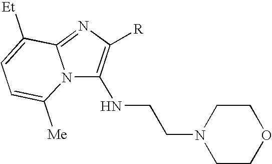 |
| 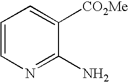 | 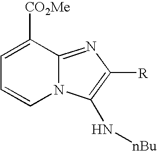 | 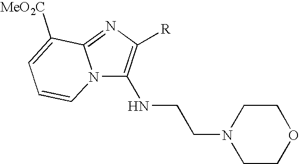 |
| 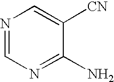 | 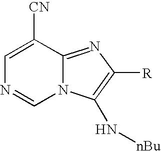 | 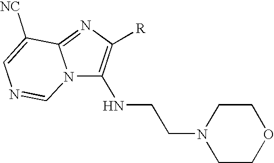 |
| 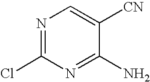 | 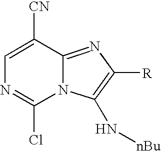 | 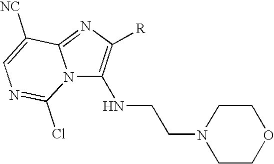 |
| 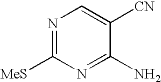 | 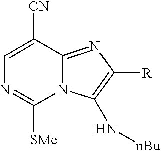 | 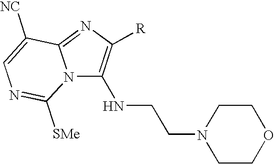 |
| | R—CHO | 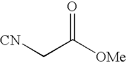 |
| | 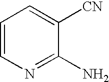 | 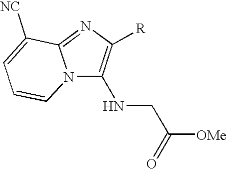 |
| | 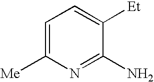 | 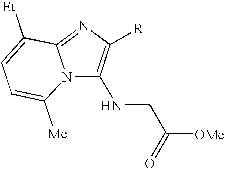 |
| | 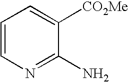 | 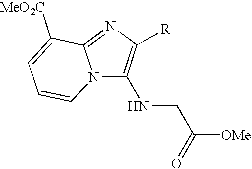 |
| | 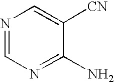 | 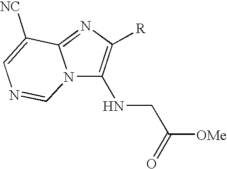 |
| | 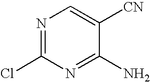 | 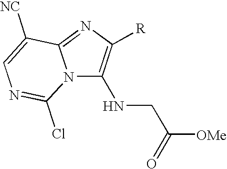 |
| | 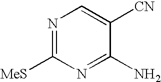 | 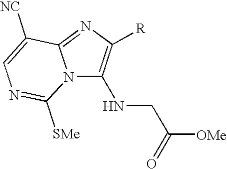 |

With reference to Schemes 3-14, representative aldehydes (R—CHO) for condensation with the amines and isonitriles set forth include, without limitation, the following:

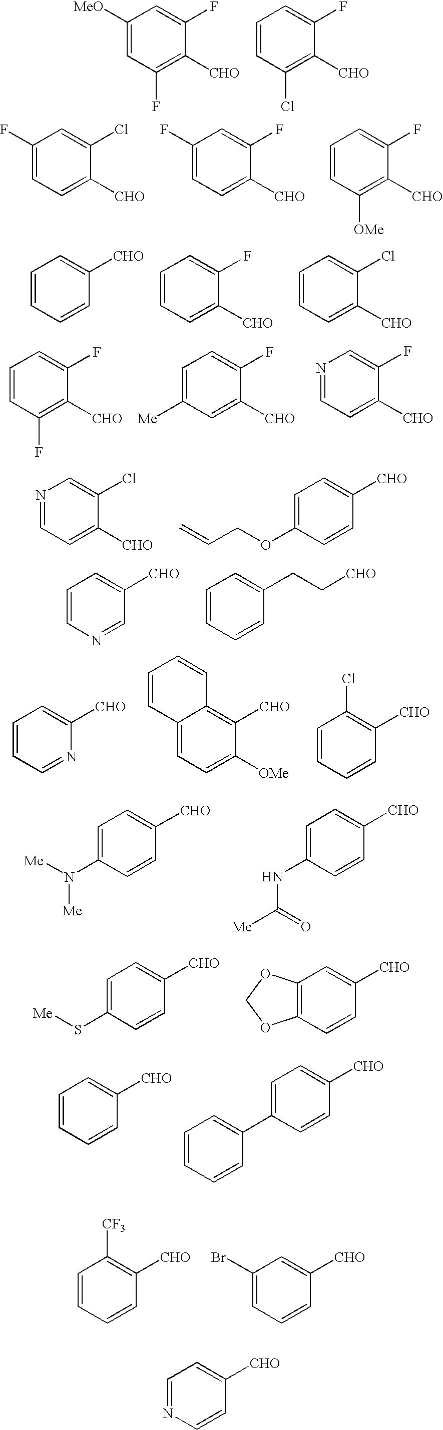

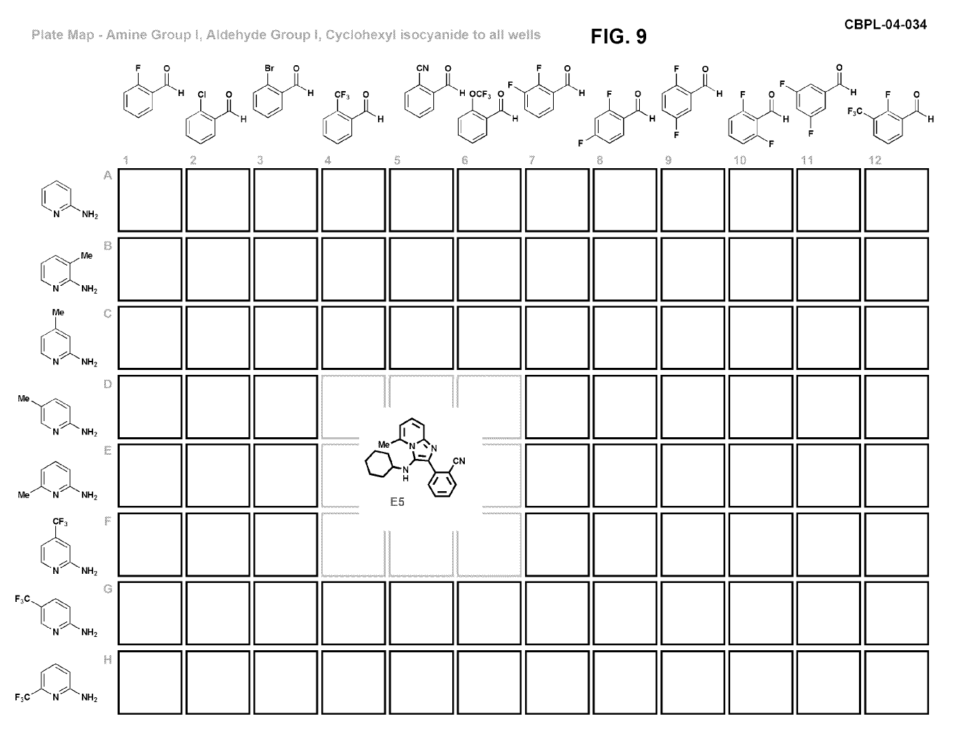

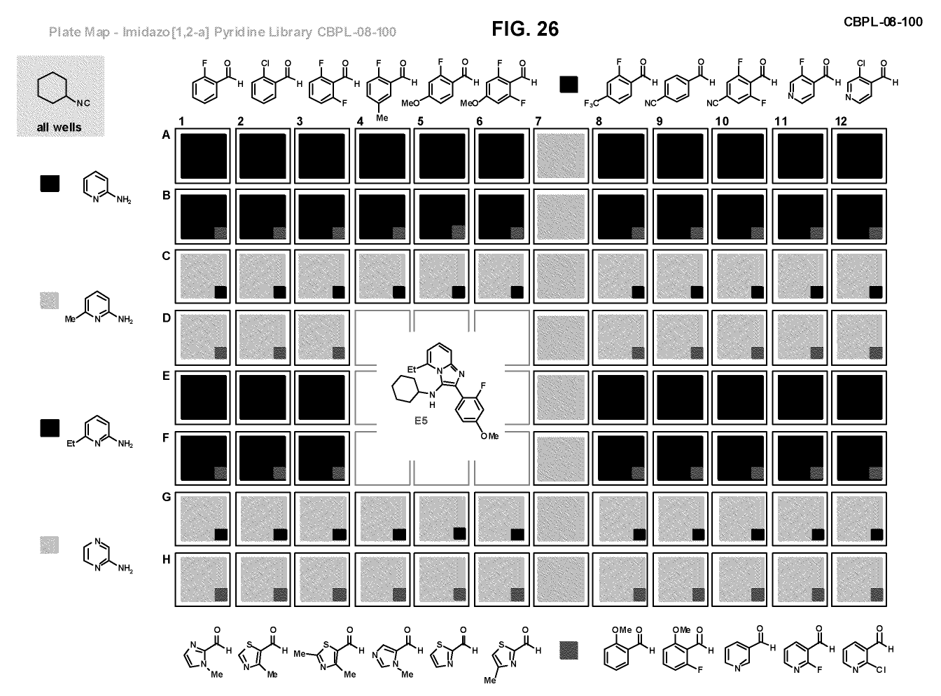

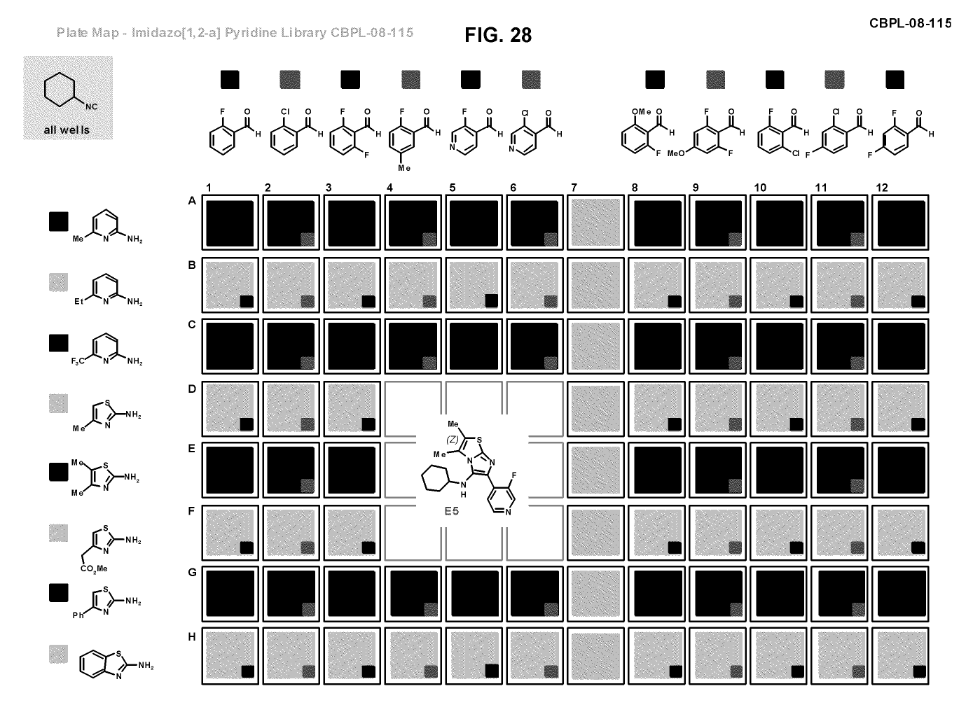

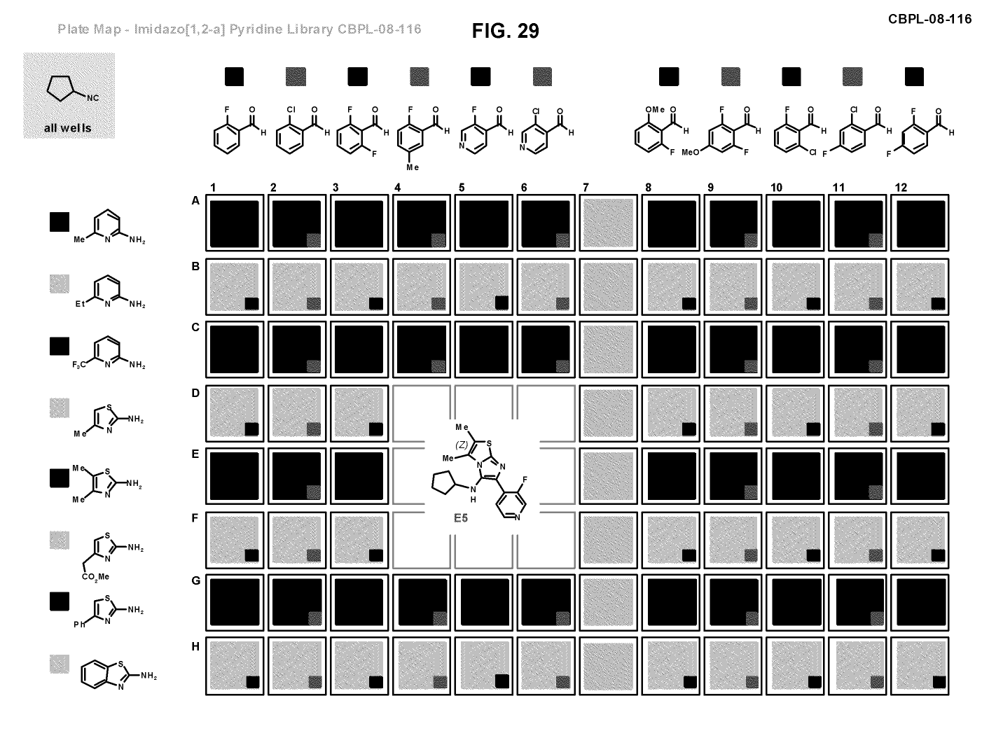

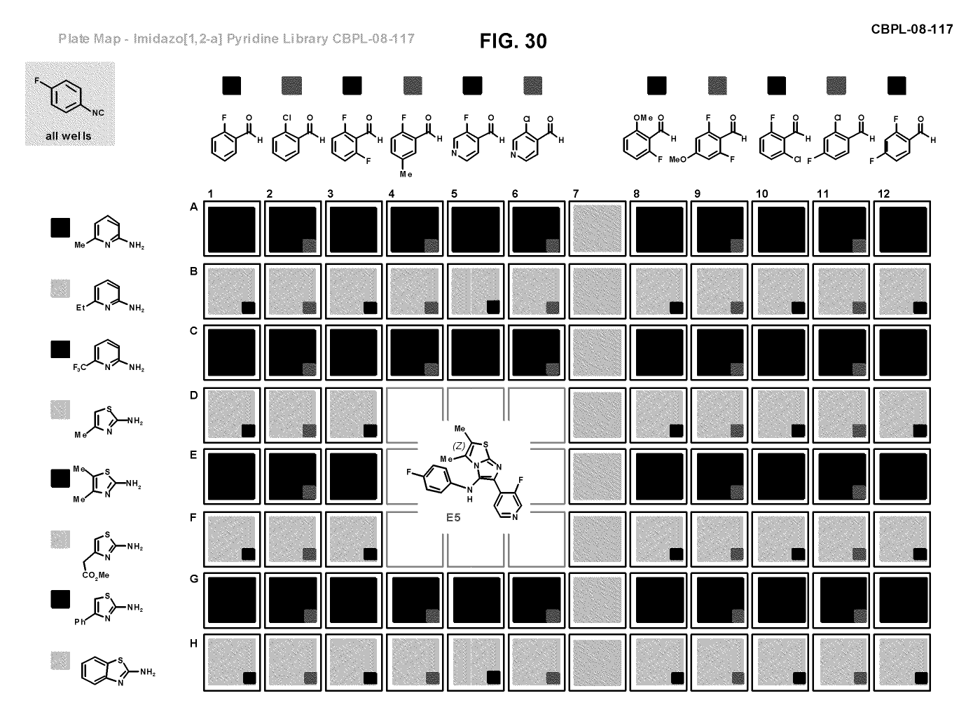

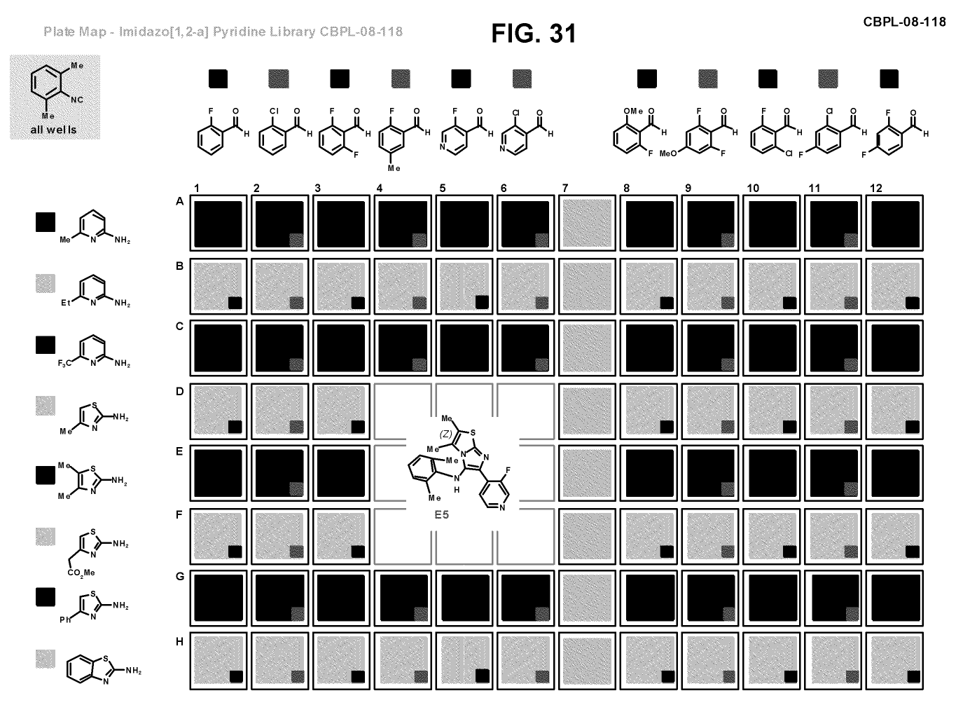

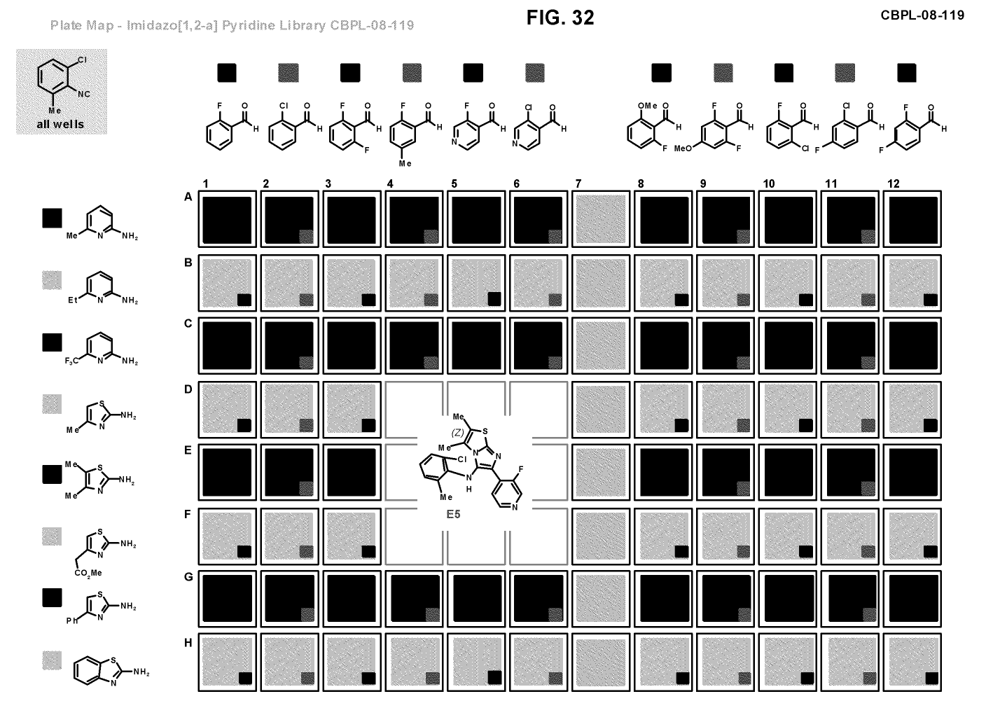

Additional compounds synthesized and evaluated as inhibitors as described herein are illustrated by the plate maps in FIGS. 9-12 and 26-32. For example, plate map 9 illustrates 88 compounds that are synthesized by the three component Groebke reaction of the aldehydes on the x-axis of the map and the amines on the y-axis with cyclohexyl isocyanide. As is known to those of ordinary skill in the art, the amine, aldehyde and/or isonitrile component can be varied to include others described herein or known to those of skill in the art. In particular, other isonitriles, including those disclosed herein can be combined with the specific aldehydes and amines illustrated in the plate maps. Similarly, FIG. 26 illustrates 22 aldehydes on the x-axis and four amines on the y-axis that, in the presence of cyclohexyl isocyanide form 88 different compounds. Of course, the components used in the illustrated three-component reaction can be varied as is known to those of skill in the art. In particular many other isonitriles, including those specifically described herein can be combined with the illustrated aldehydes and amines to produce additional compounds.

Exemplary inhibitor compounds, including those which may be synthesized as set forth above, include, without limitation:

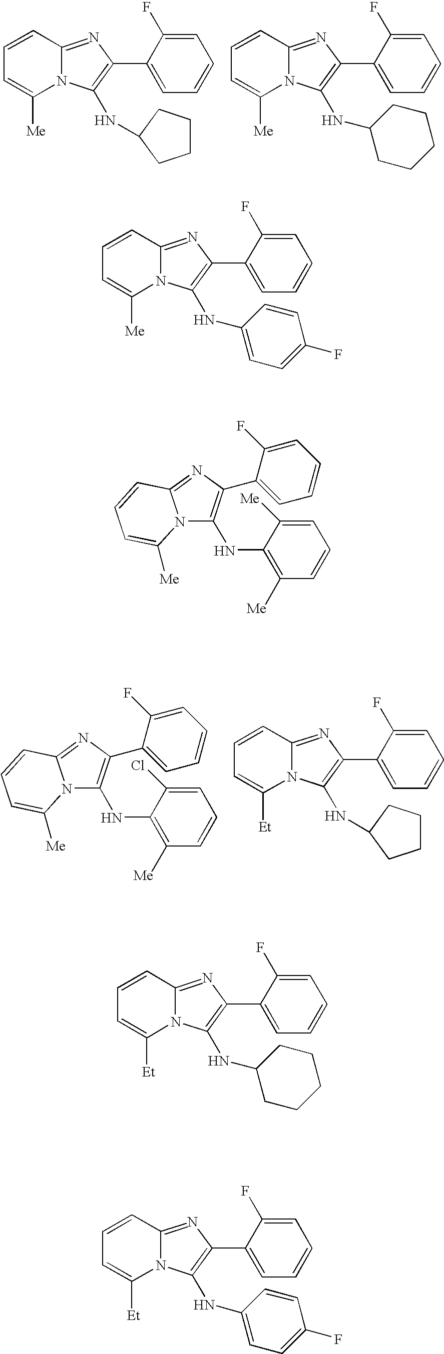

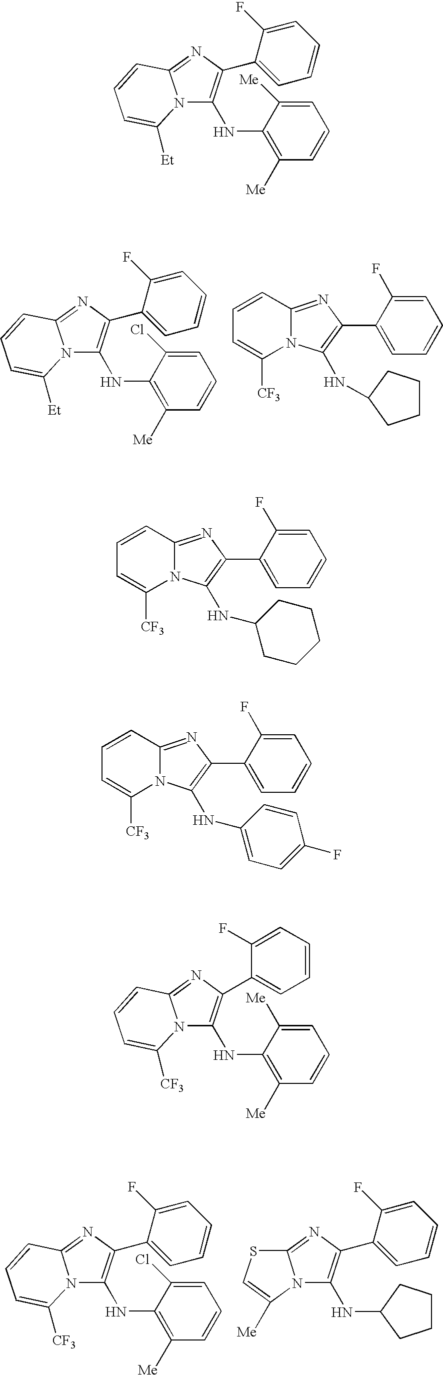

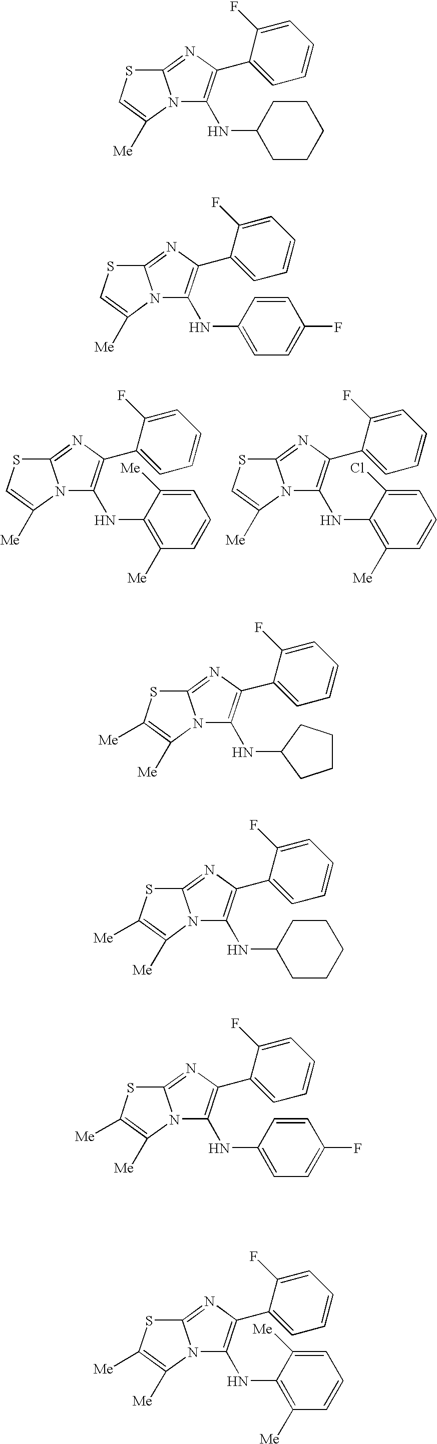

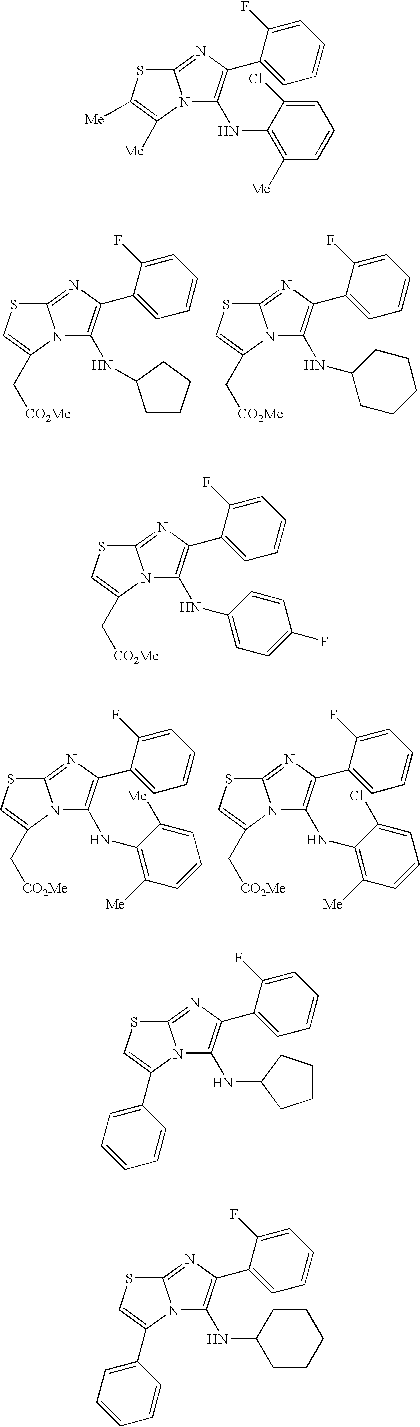

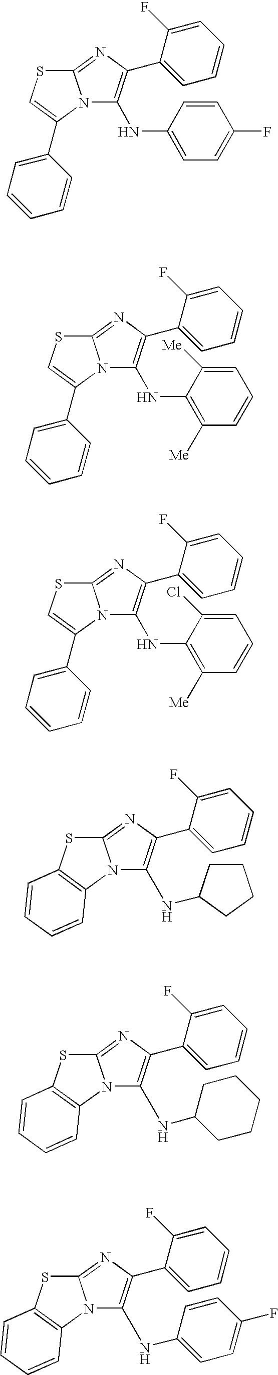

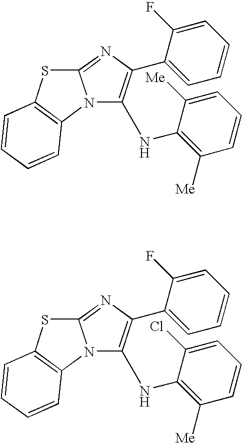

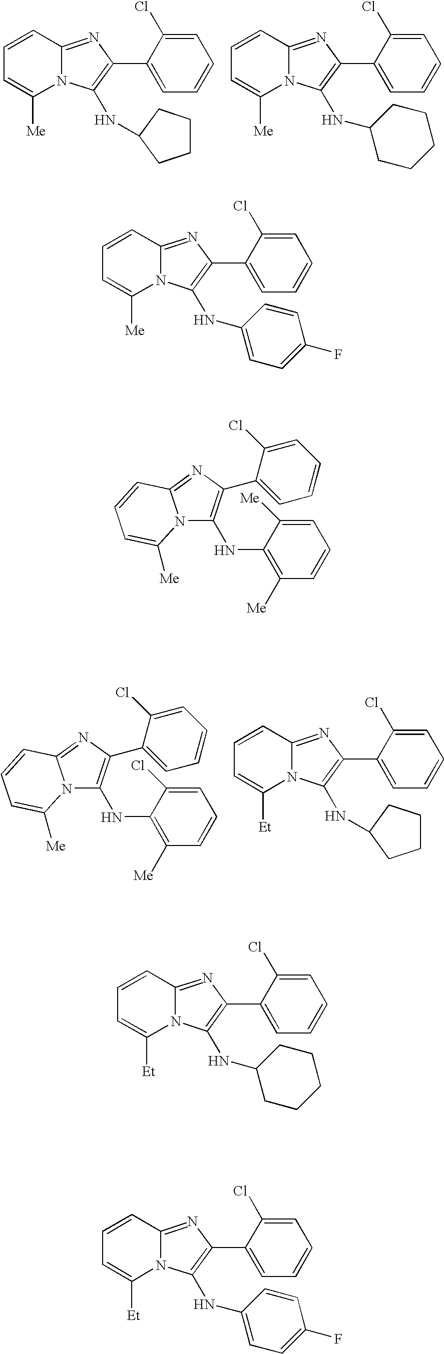

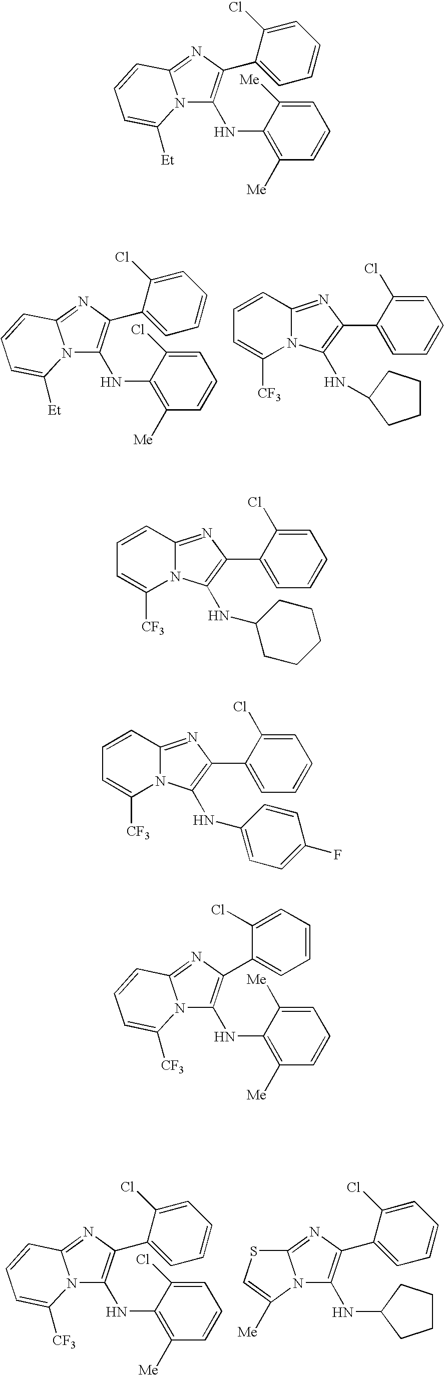

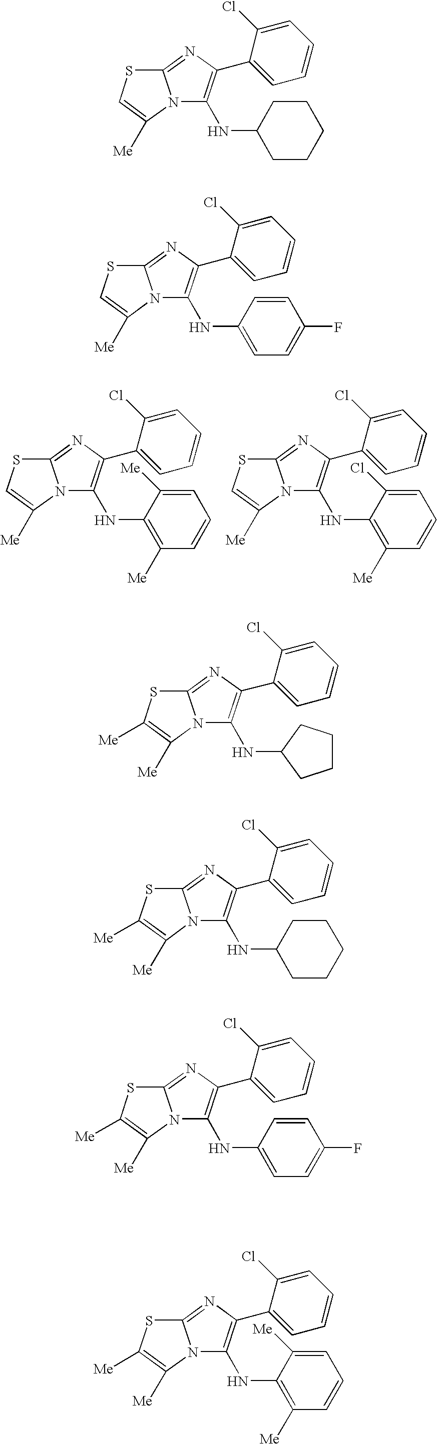

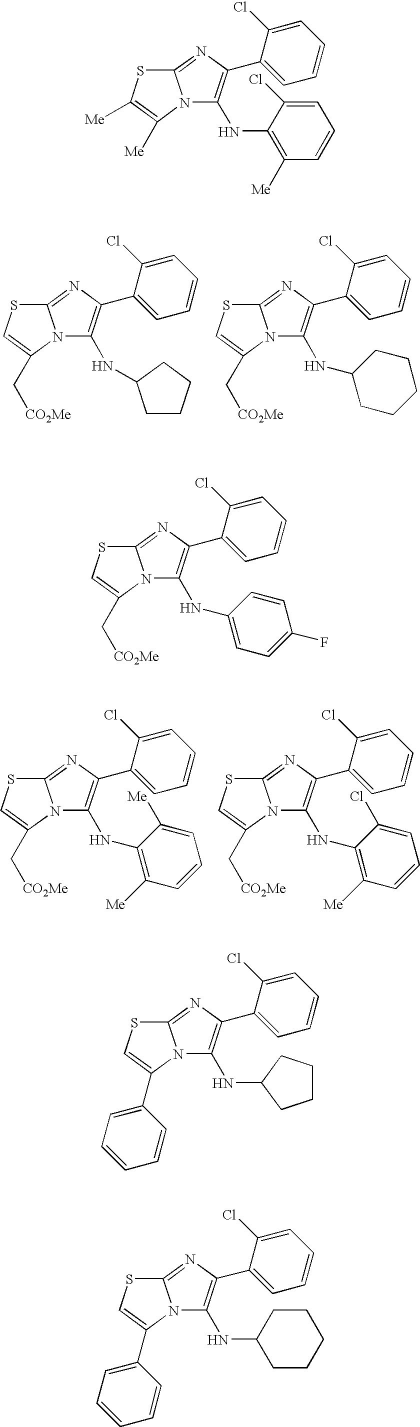

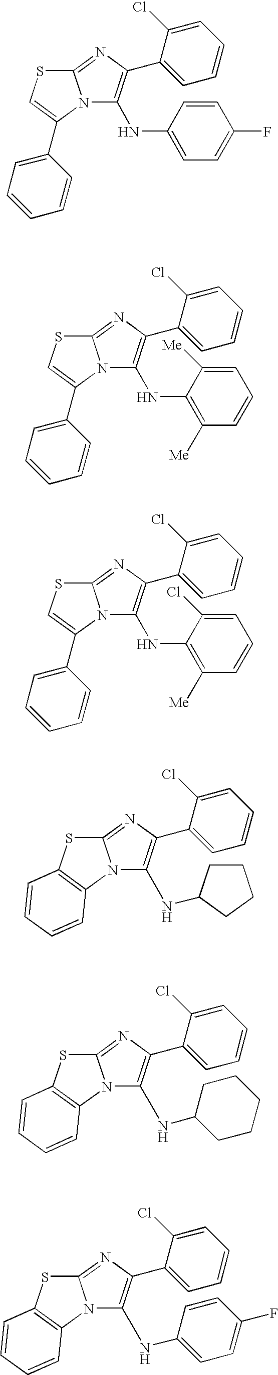

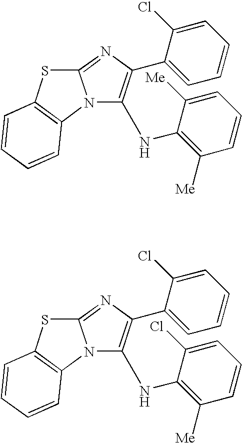

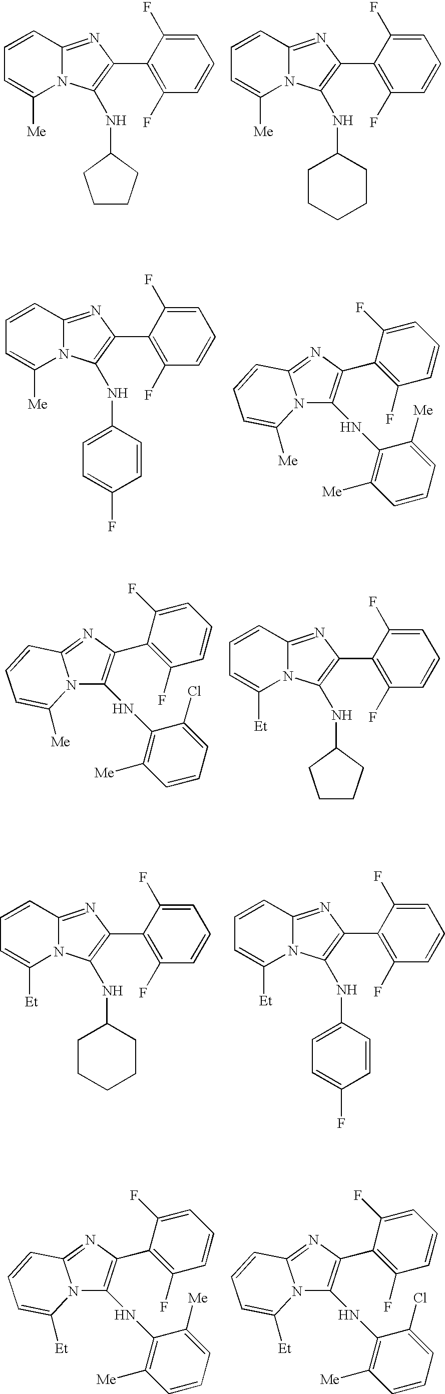

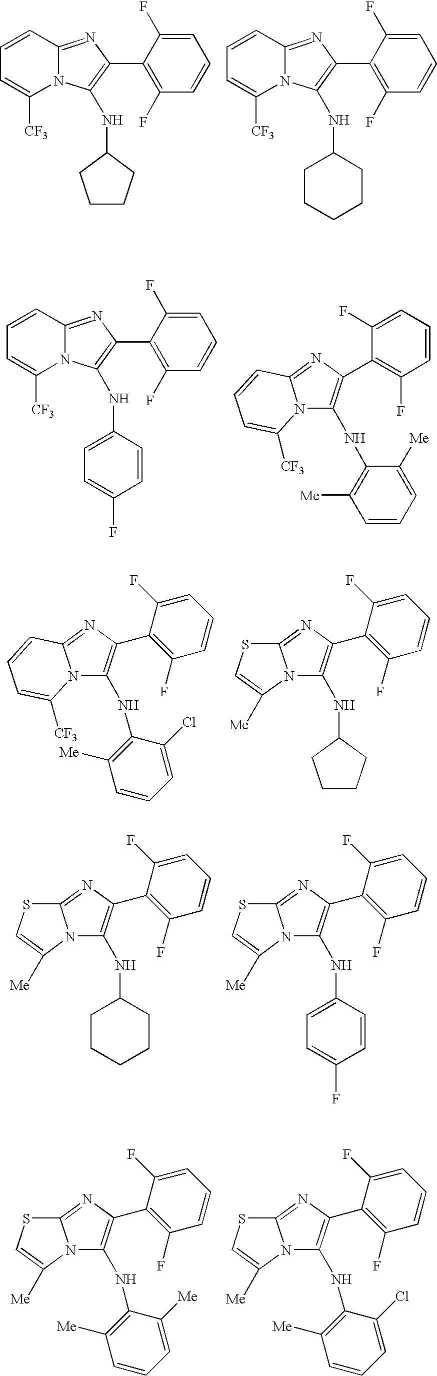

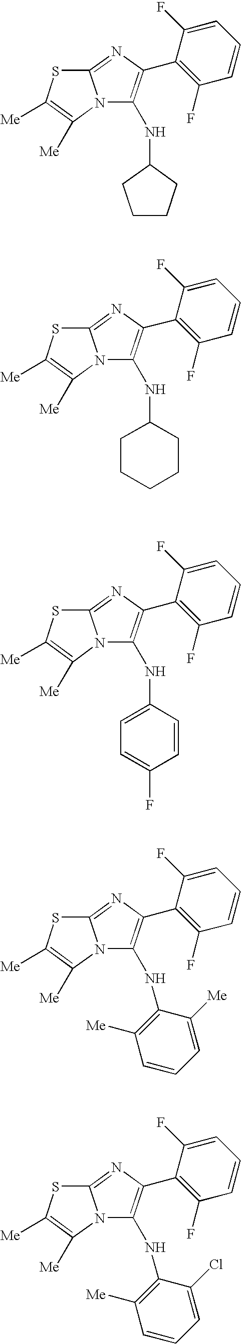

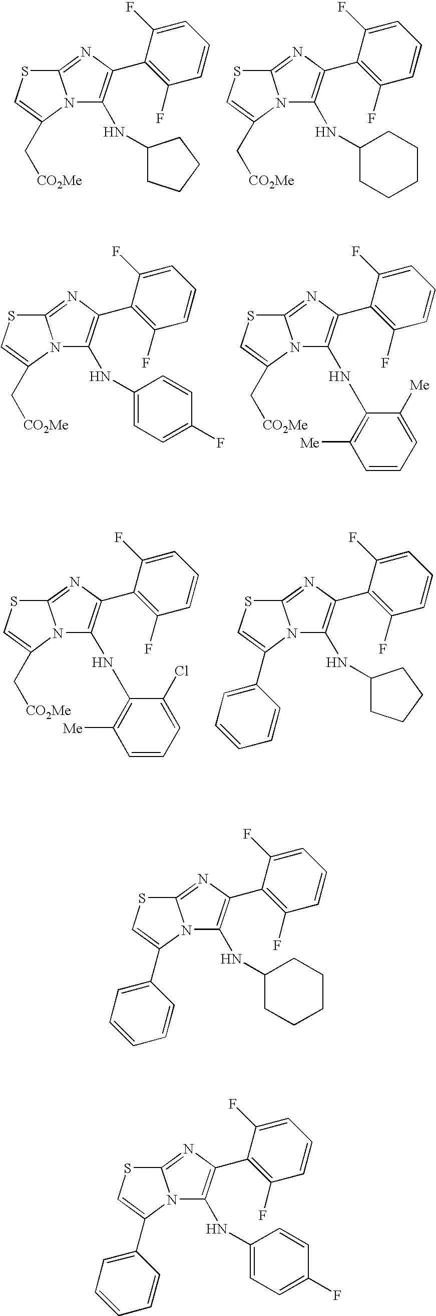

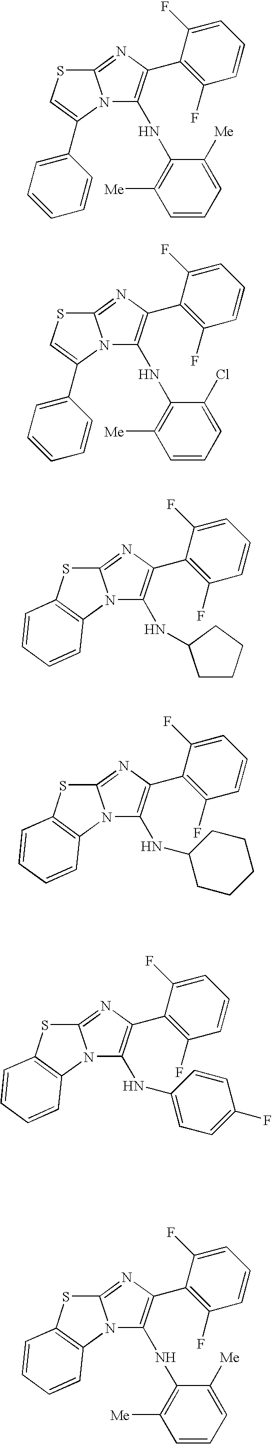

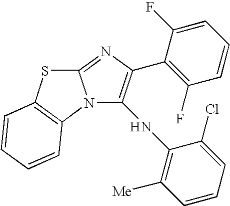

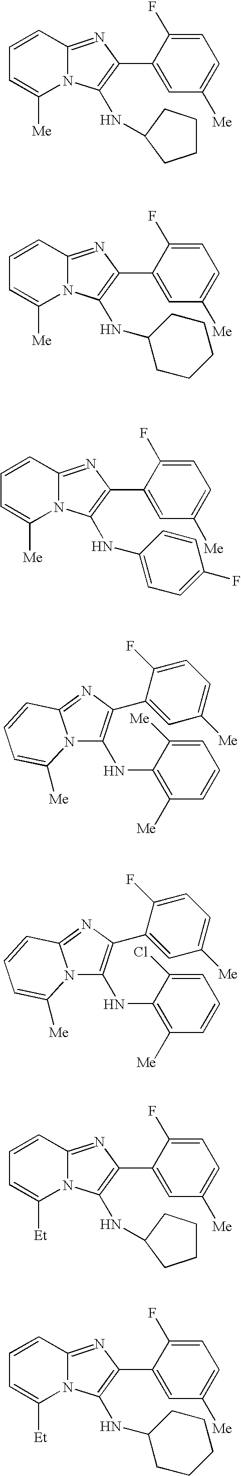

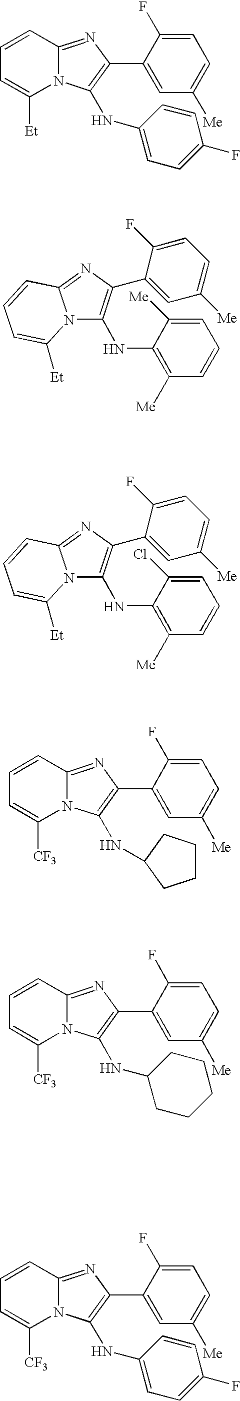

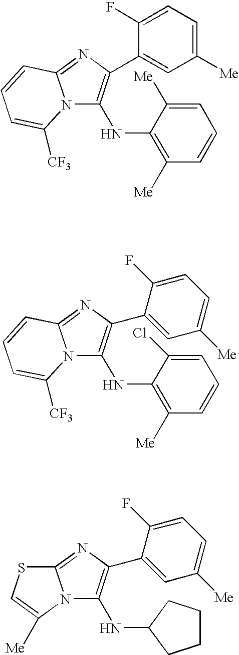

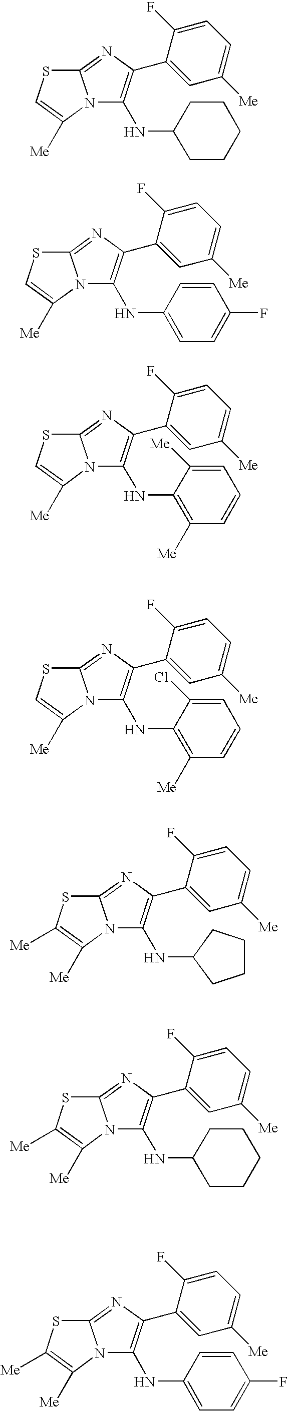

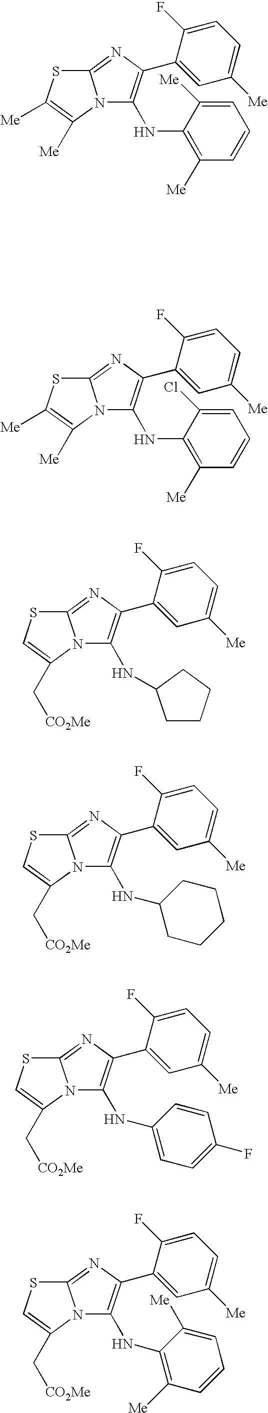

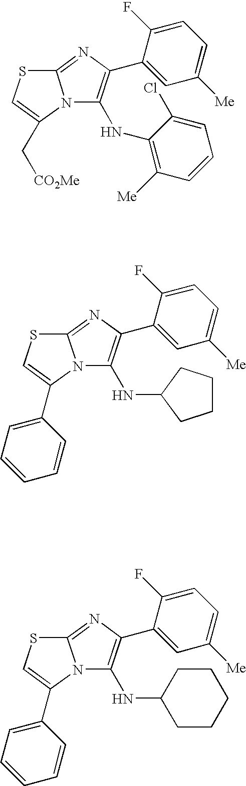

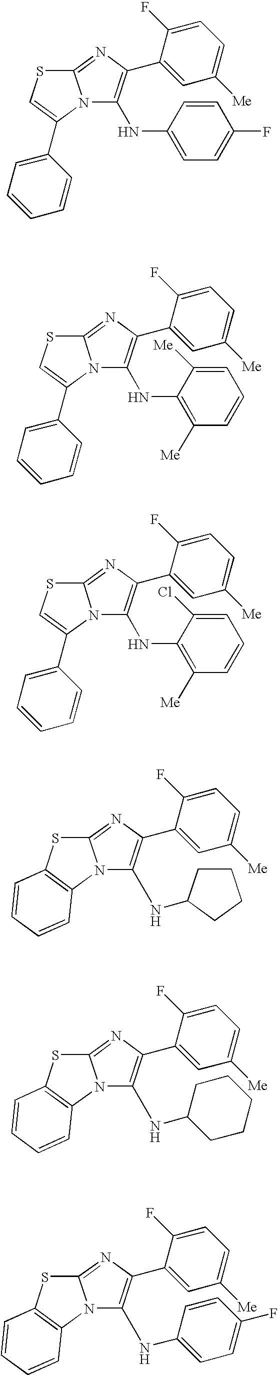

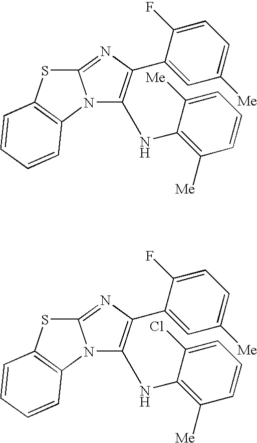

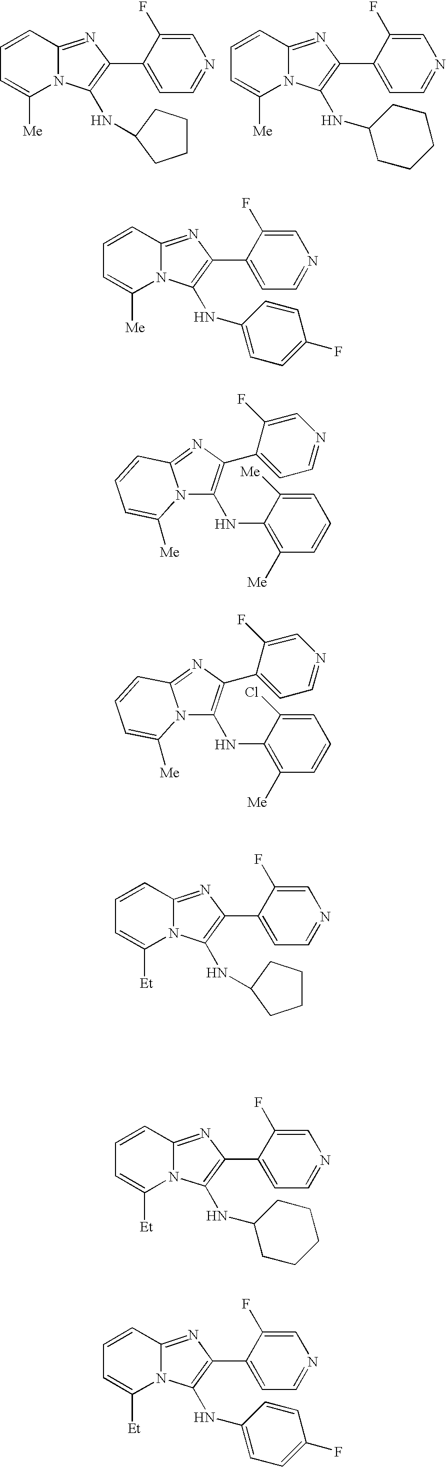

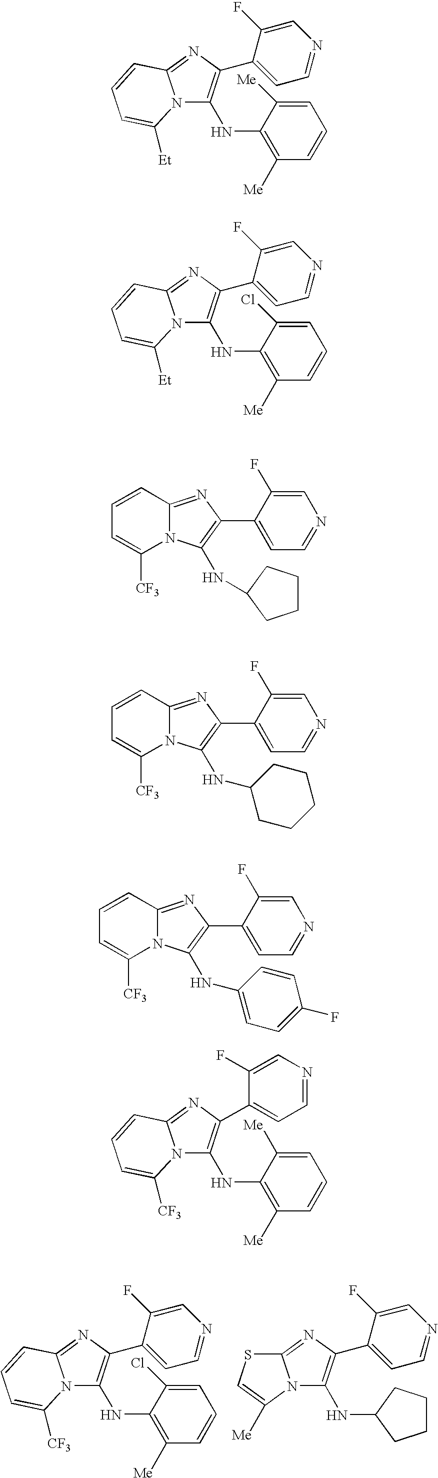

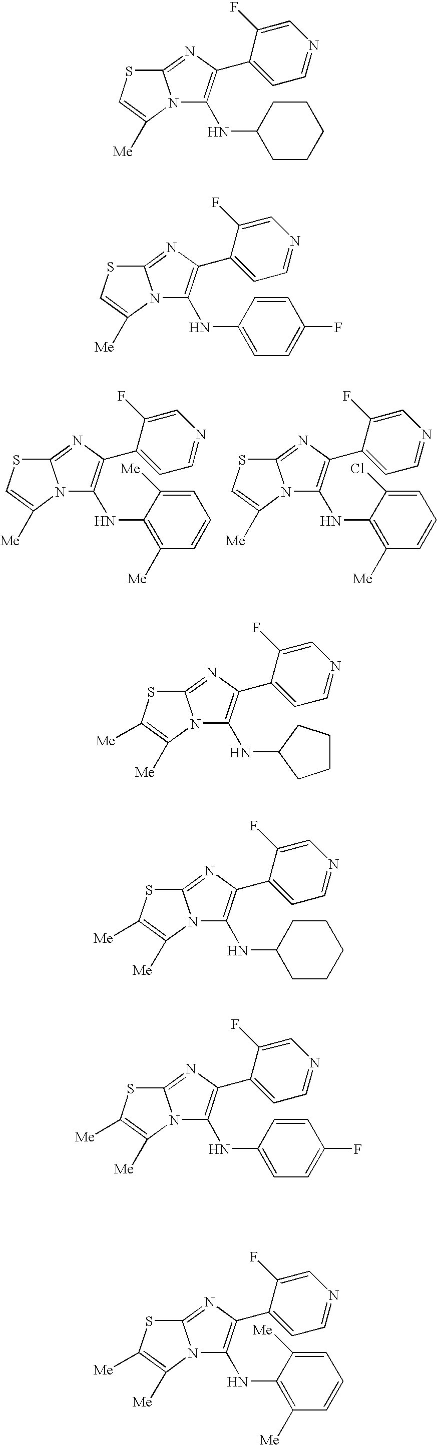

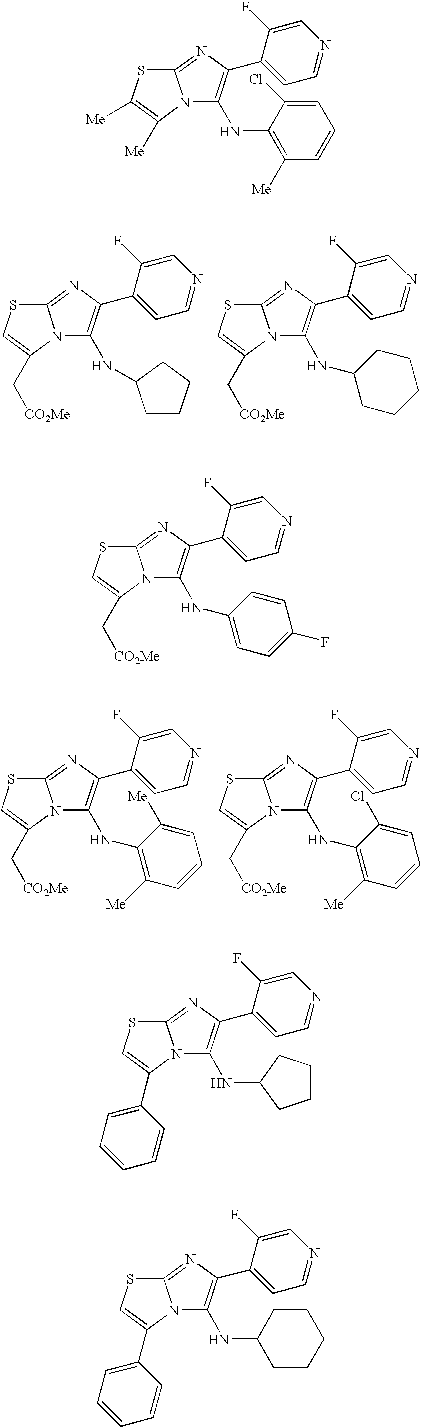

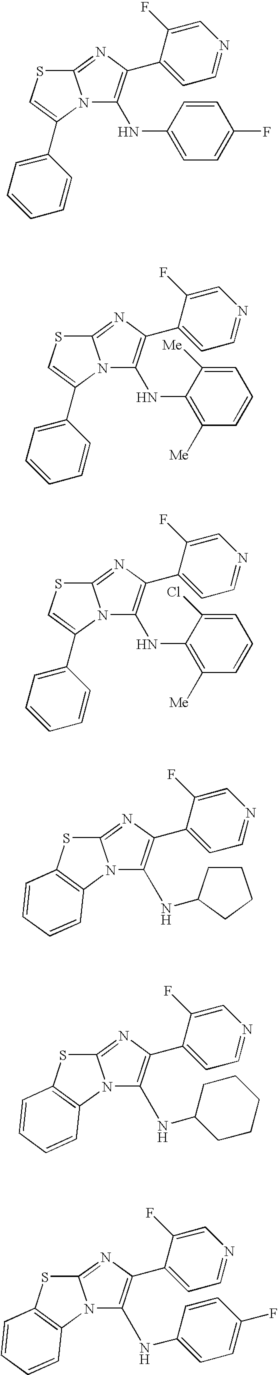

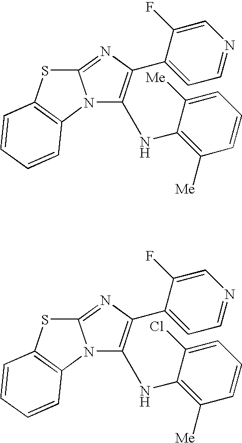

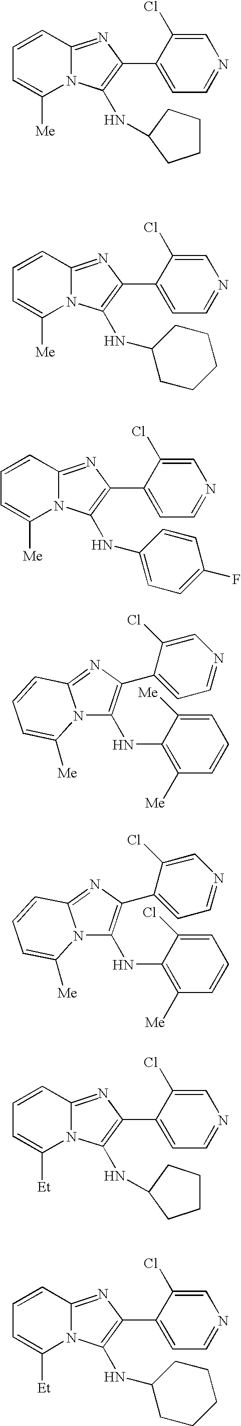

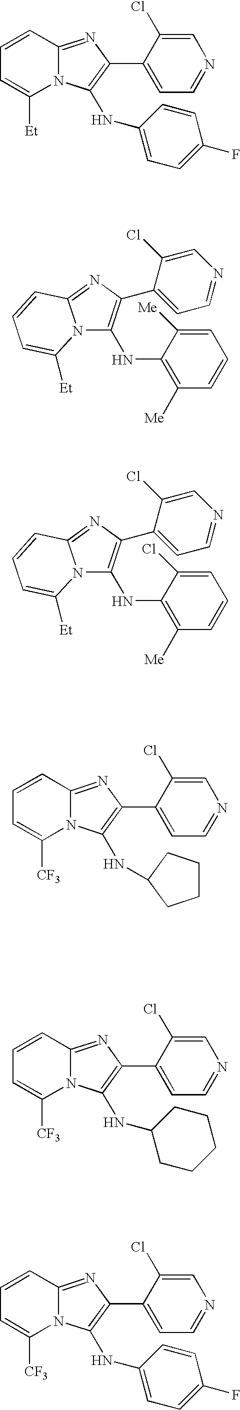

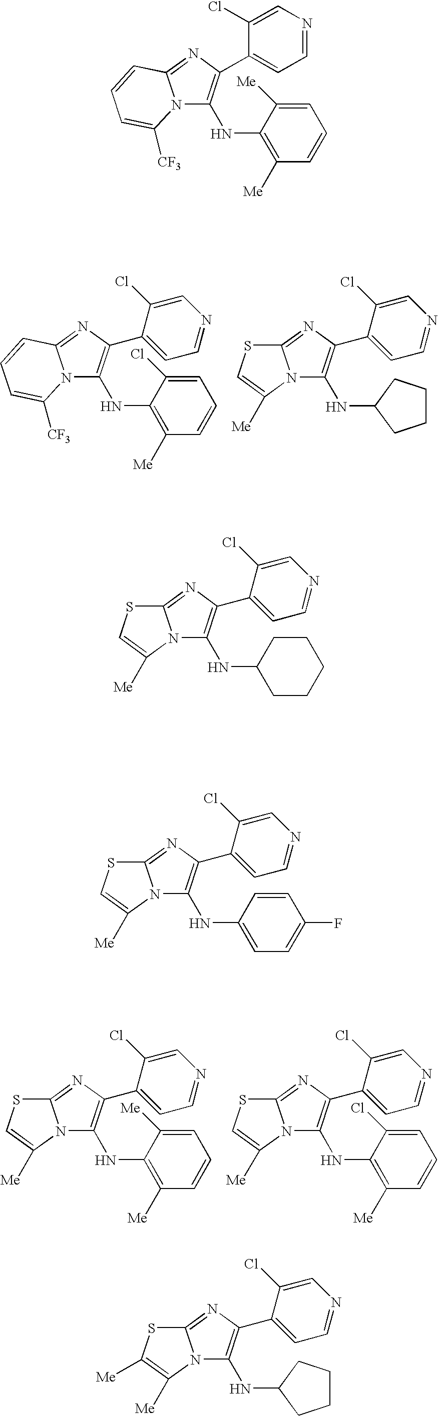

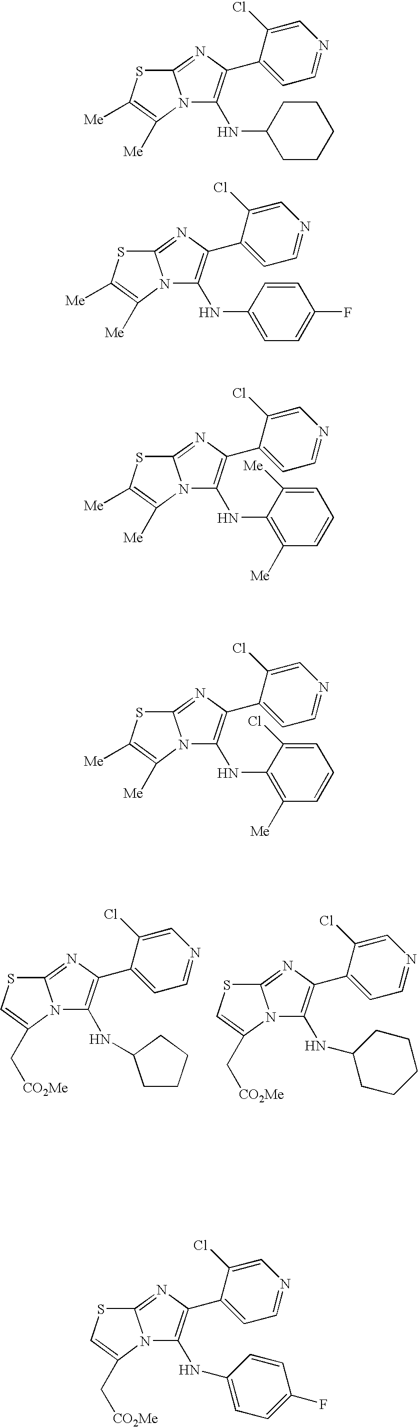

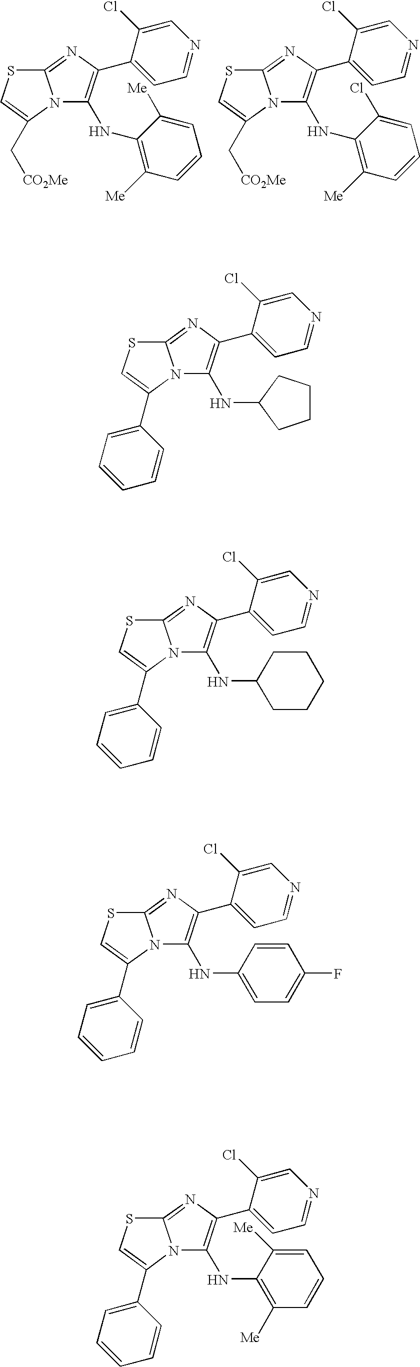

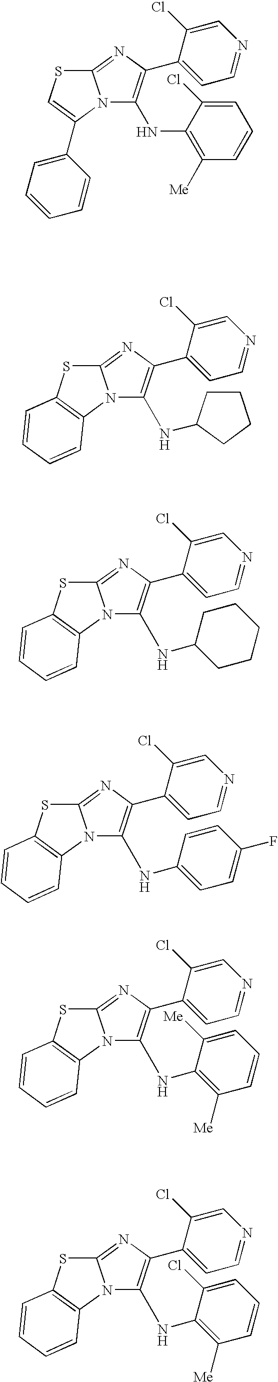

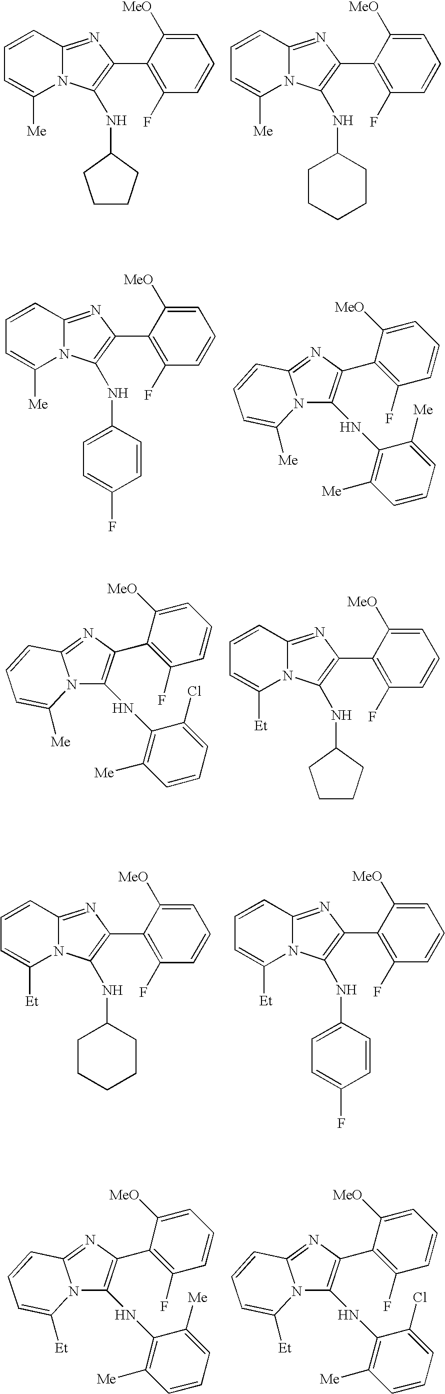

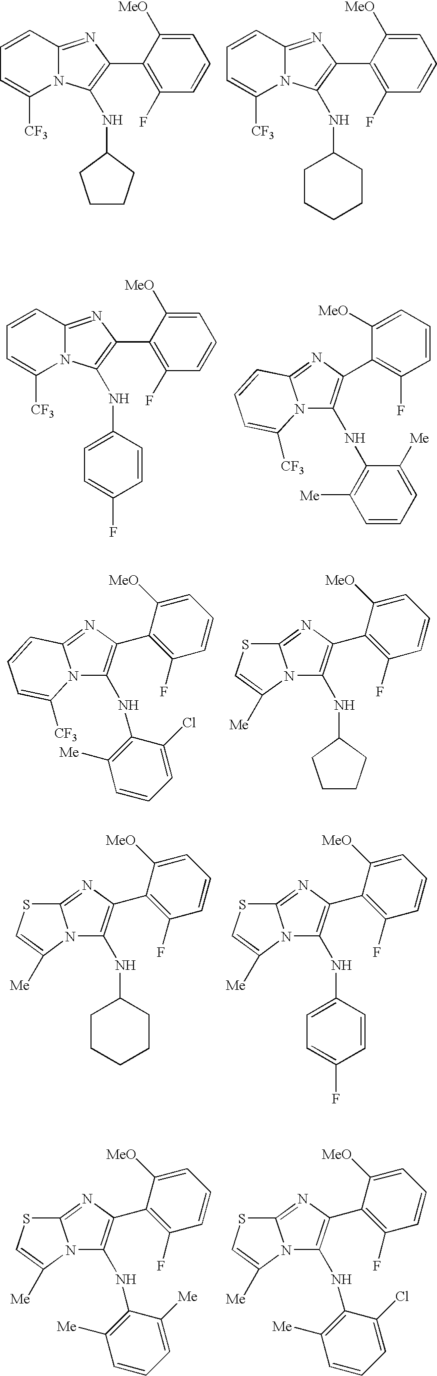

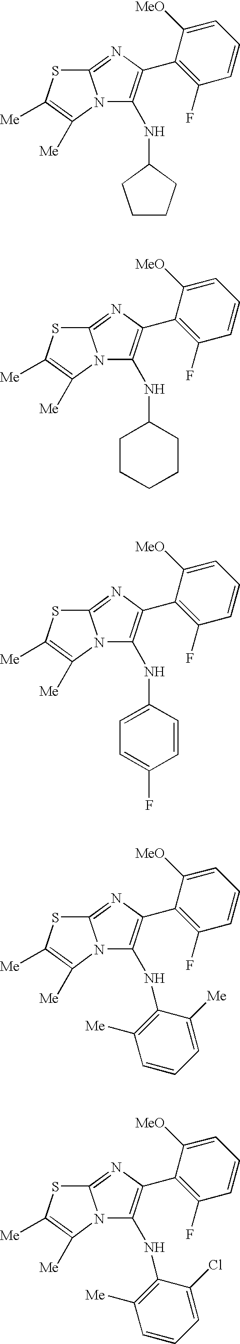

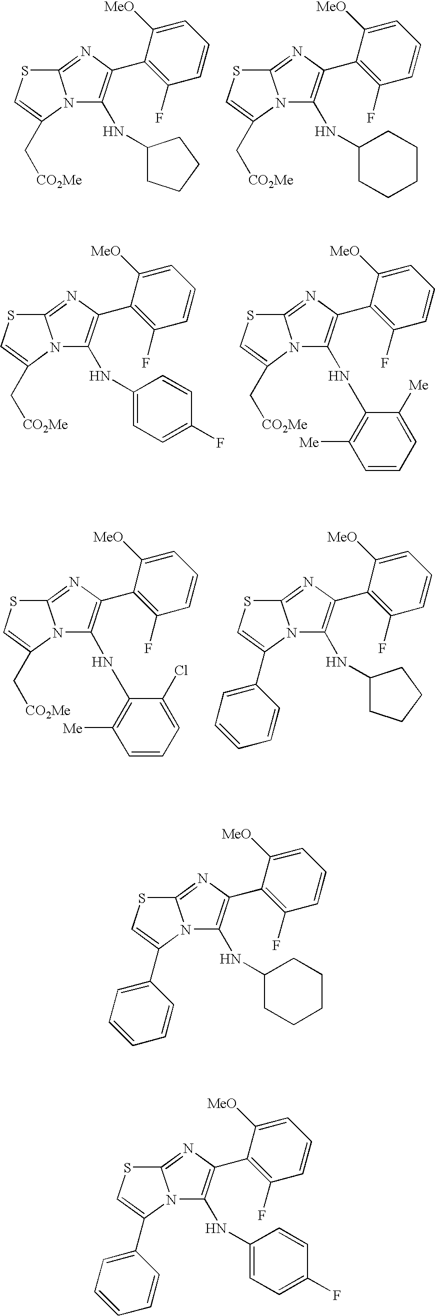

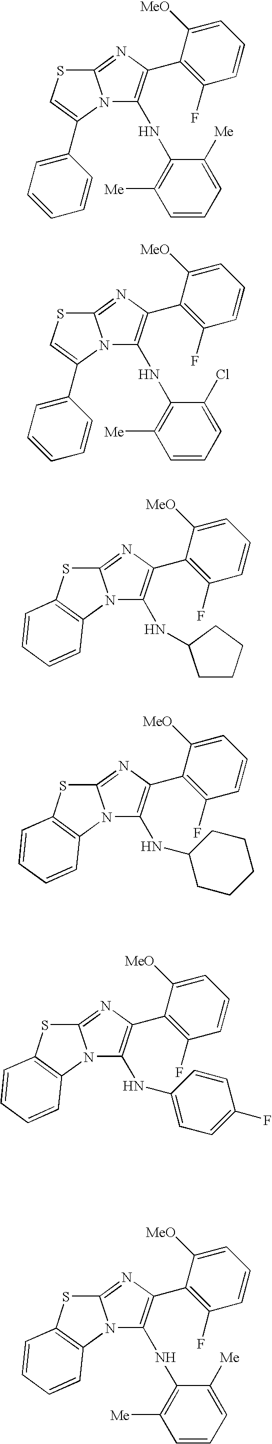

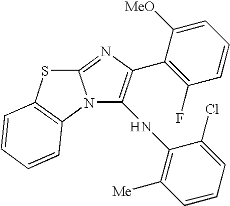

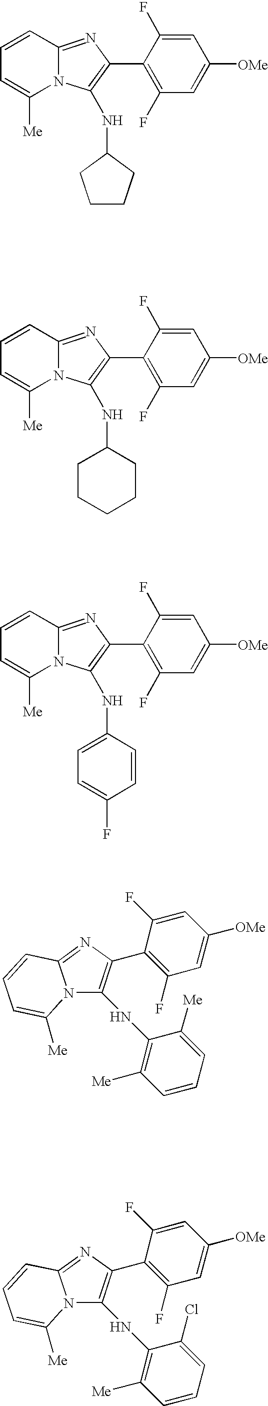

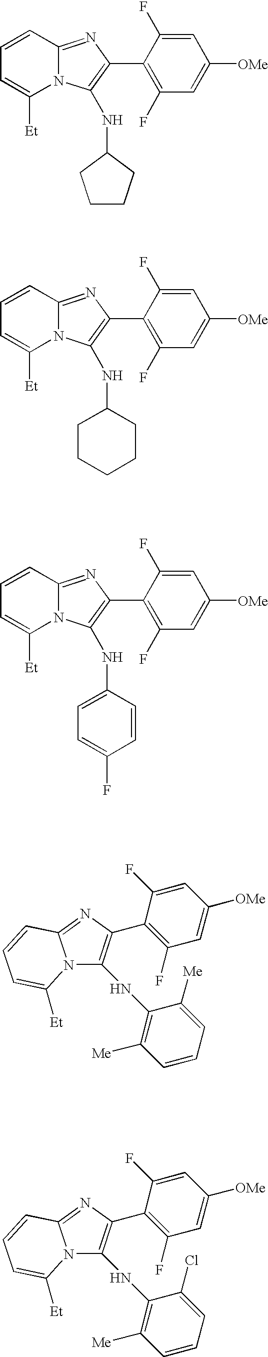

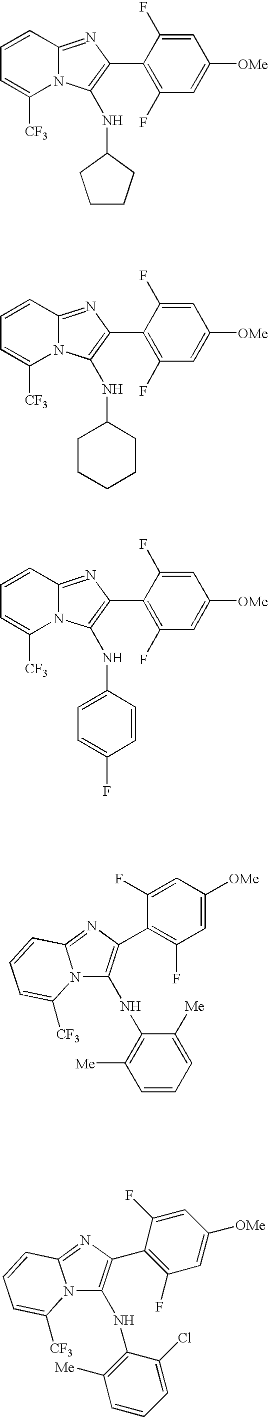

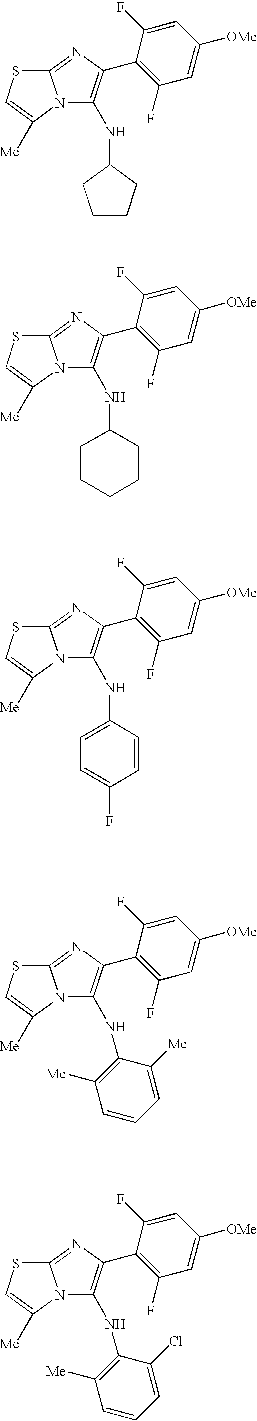

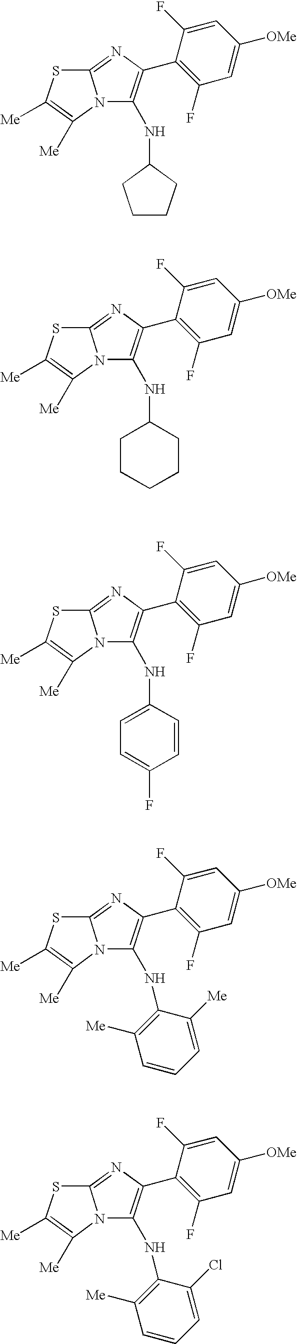

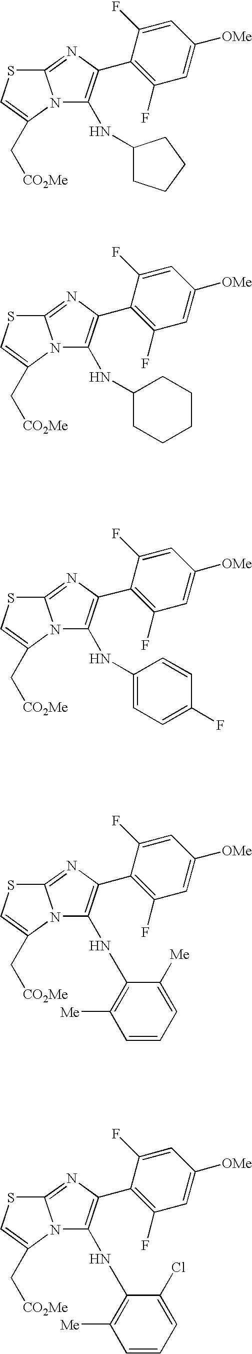

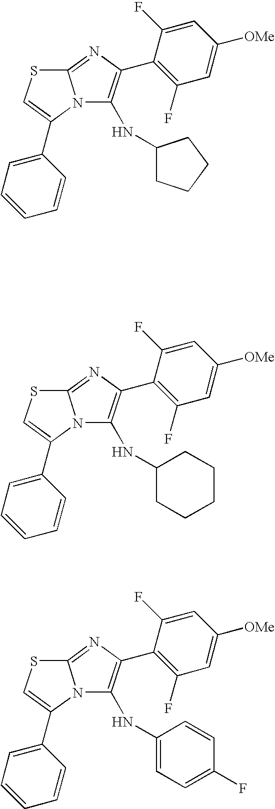

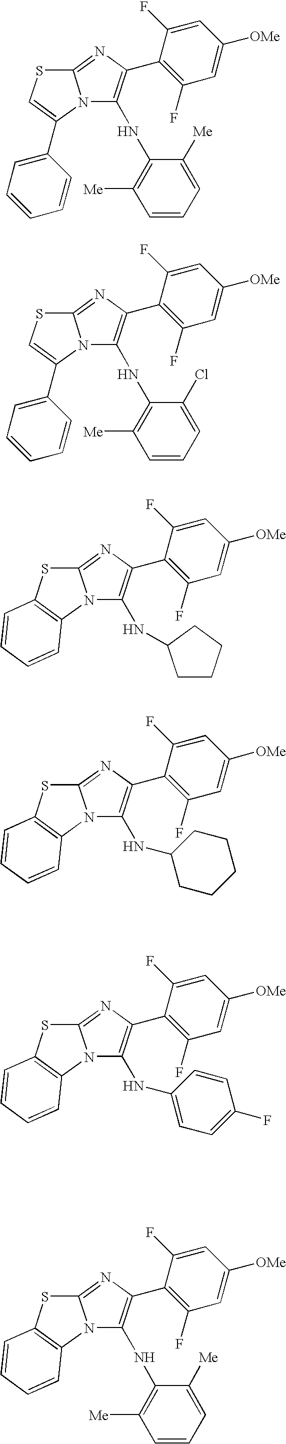

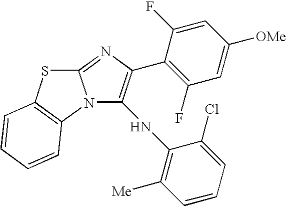

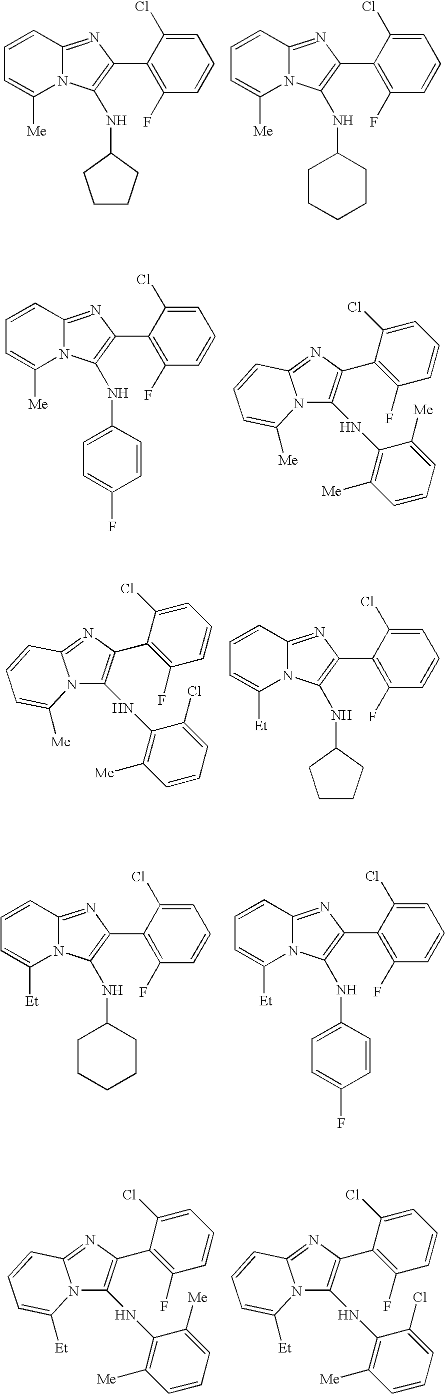

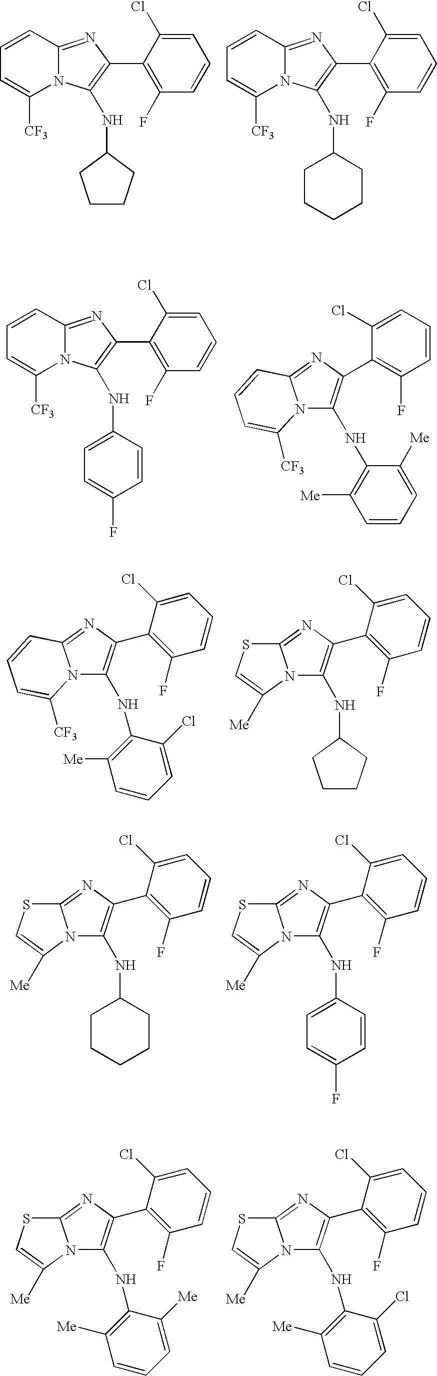

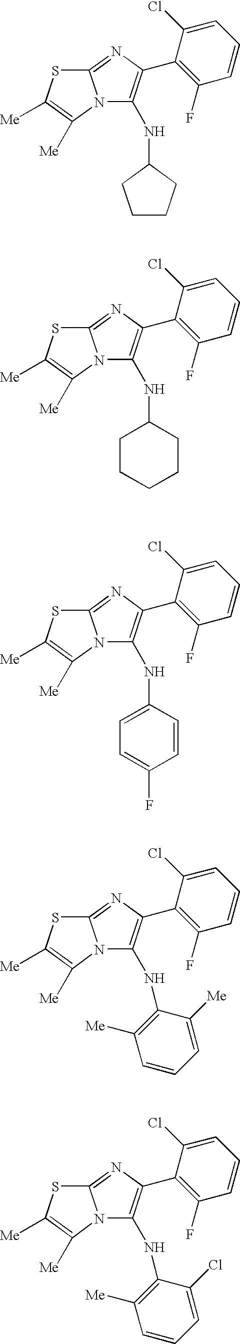

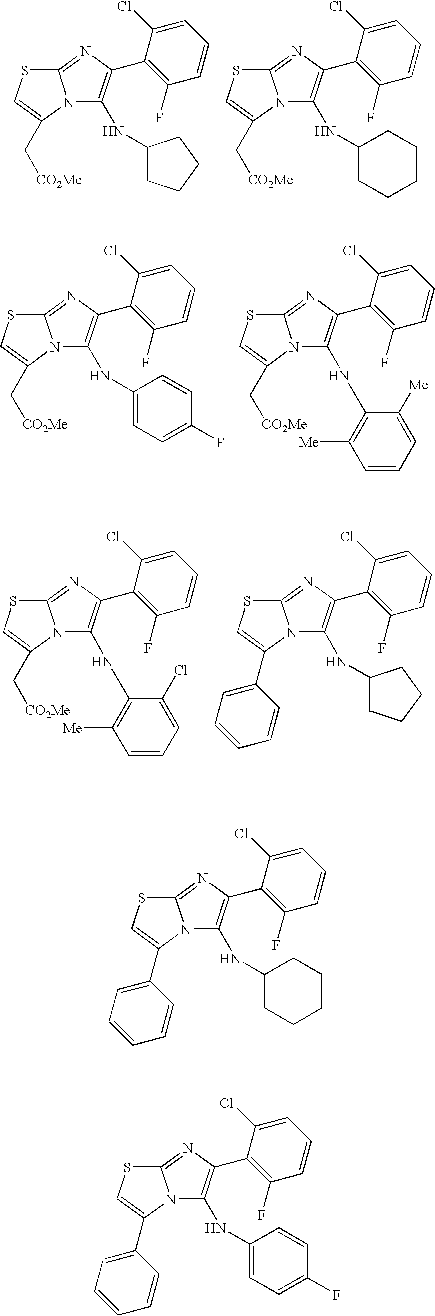

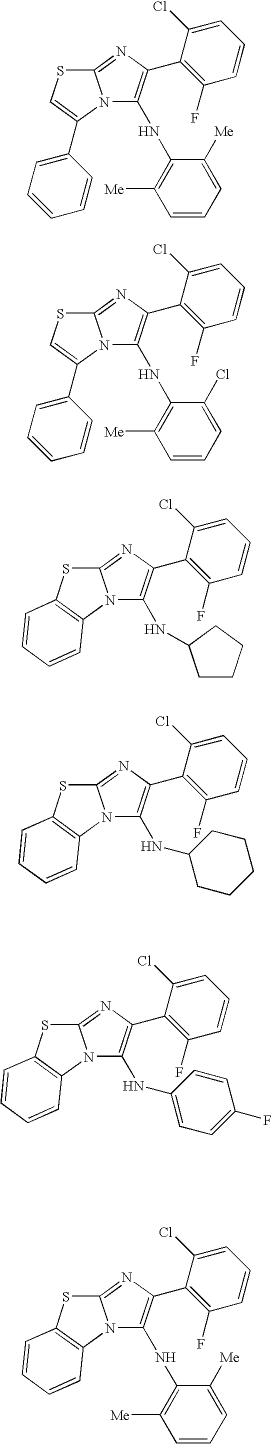

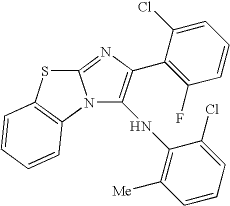

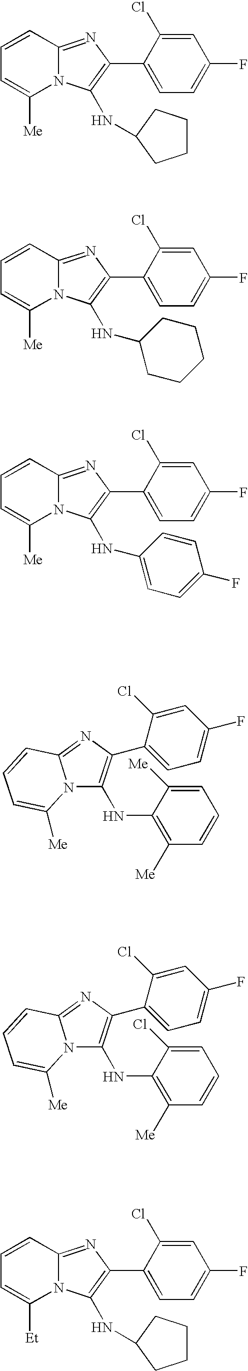

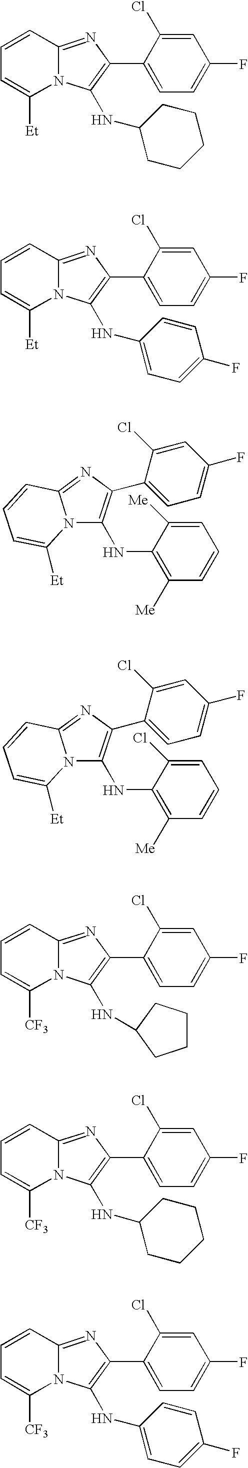

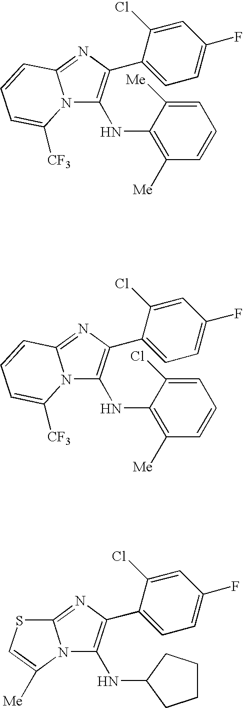

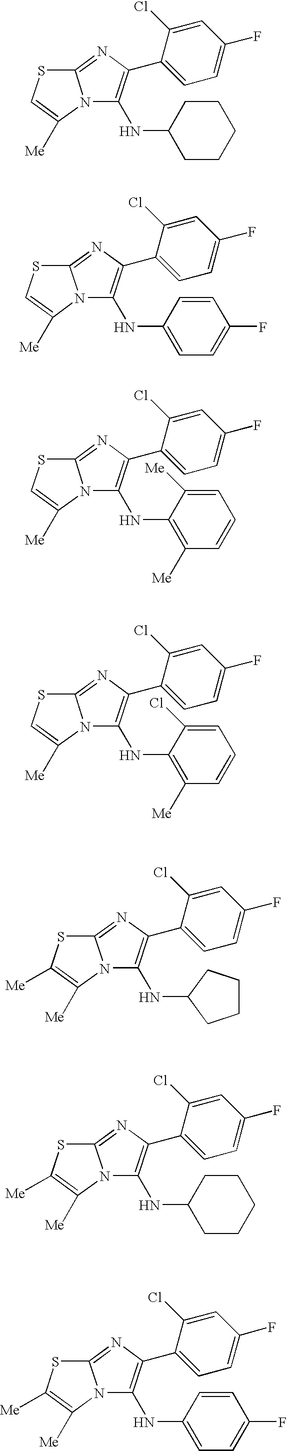

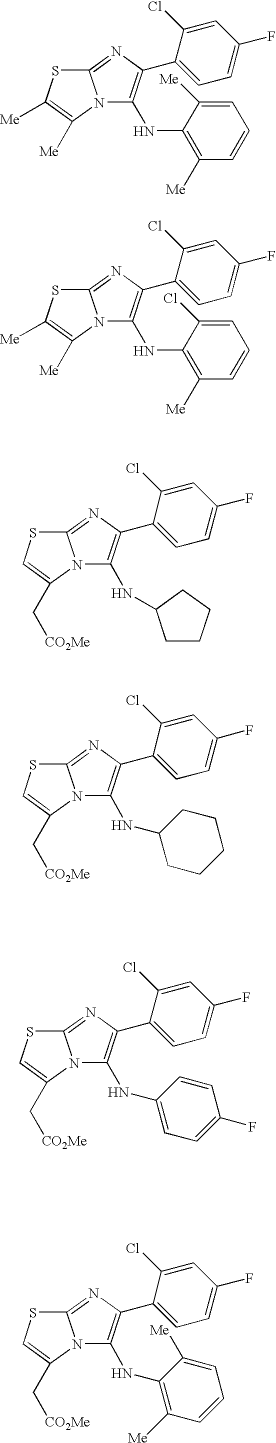

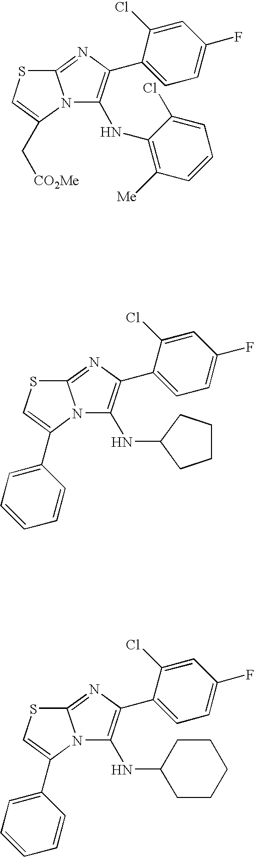

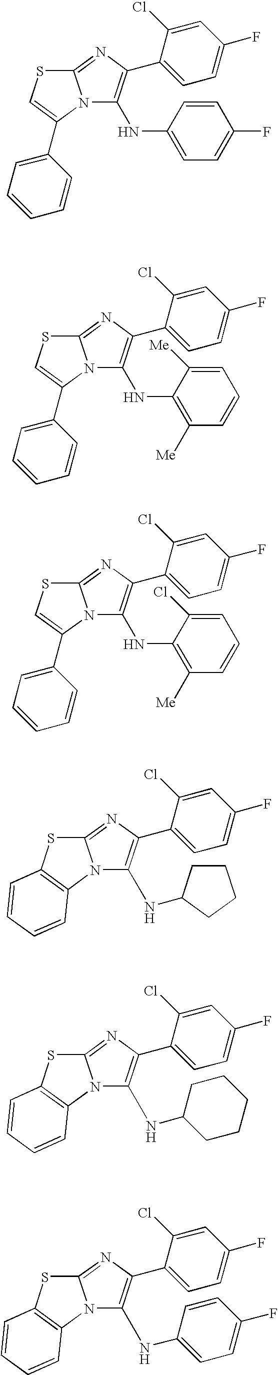

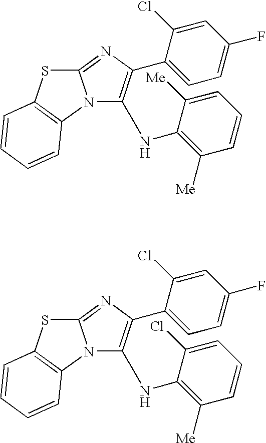

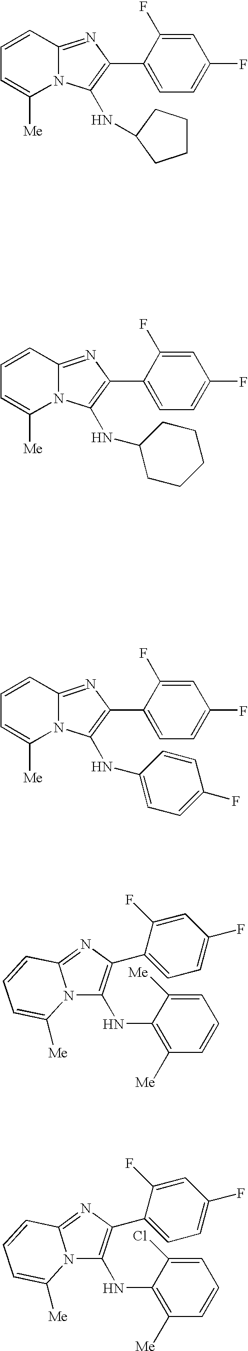

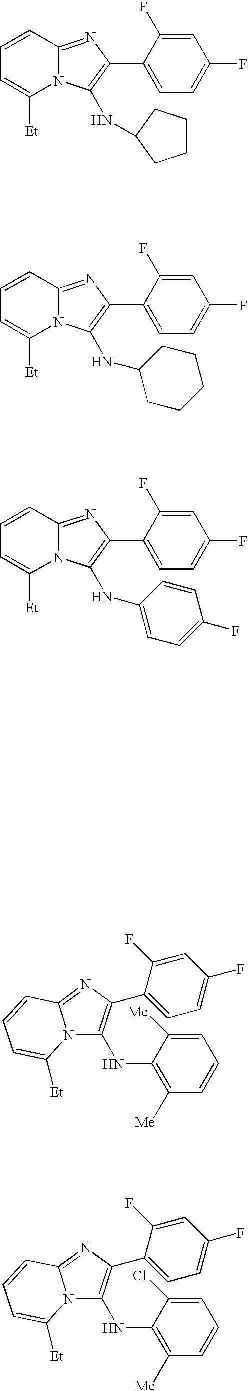

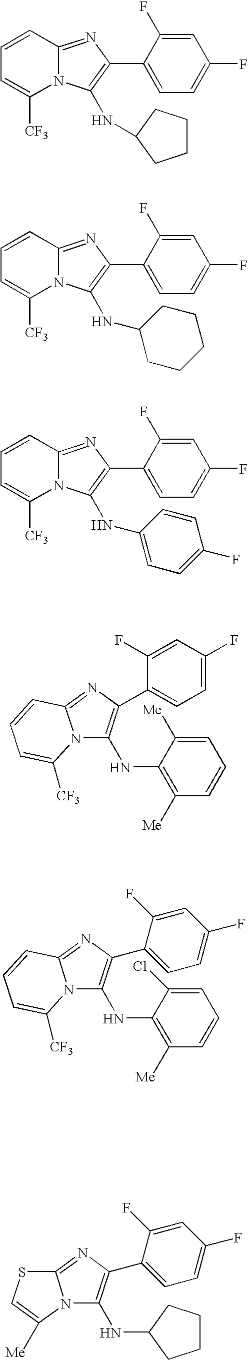

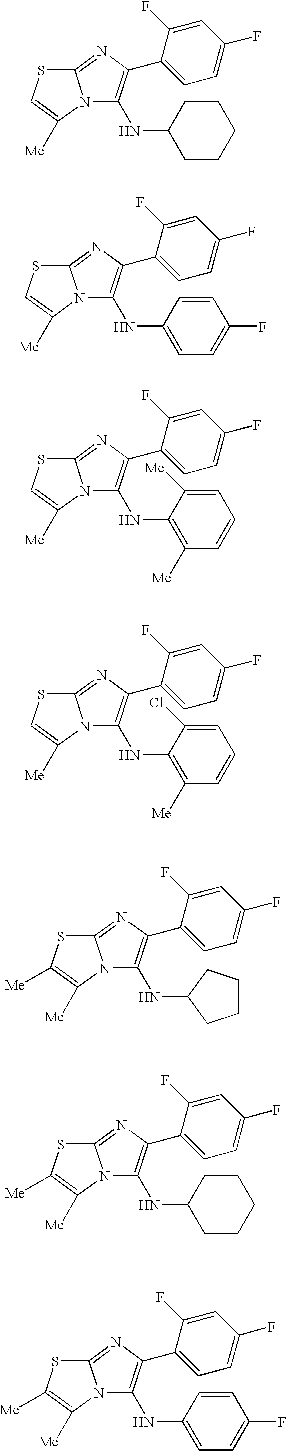

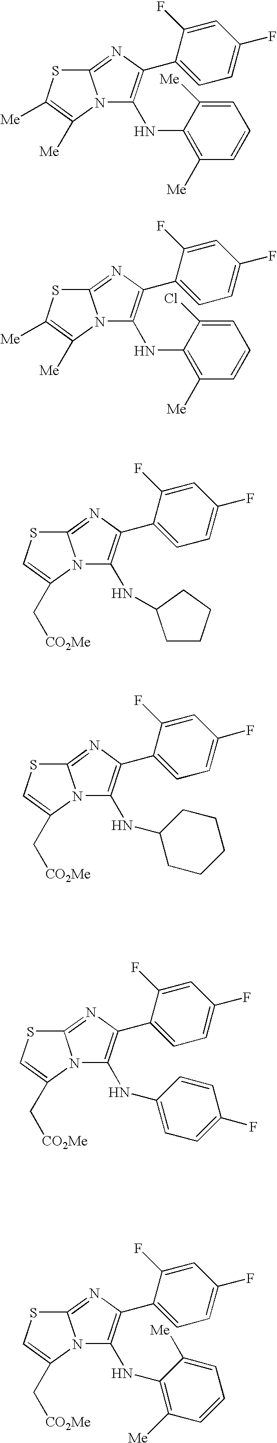

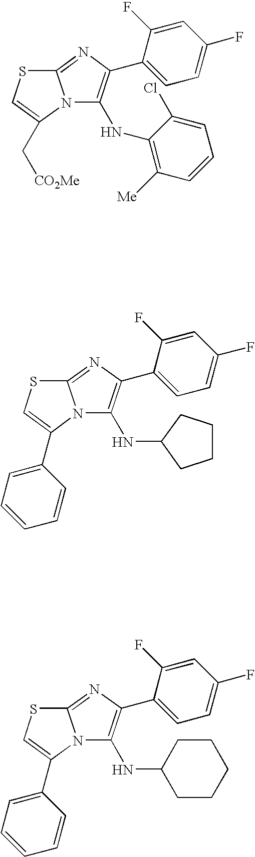

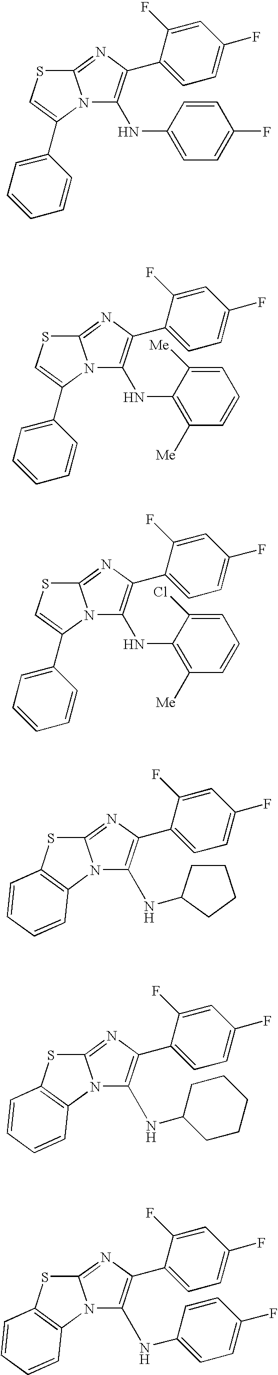

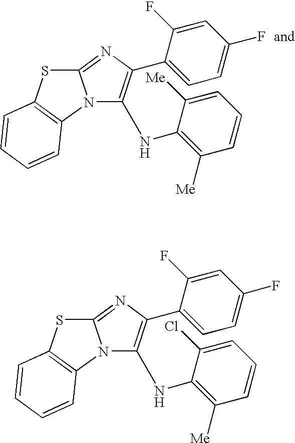

### Solvates, Salts and Pharmaceutical Compositions

Solvates of the presently disclosed antiviral agents are specifically contemplated herein. The term “solvate” refers to a compound physically associated with one or more solvent molecules. This physical association involves varying degrees of ionic and covalent bonding, including by way of example covalent adducts and hydrogen bonded solvates. In certain instances the solvate will be capable of isolation, for example when one or more solvent molecules are incorporated in the crystal lattice of the crystalline solid. “Solvate” encompasses both solution-phase and isolable solvates. Representative solvates include ethanol associated compounds, methanol associated compounds, and the like. “Hydrate” is a solvate wherein the solvent molecule(s) is/are H₂O.

Inhibitors and pharmaceutical compositions containing the inhibitors disclosed herein include those formed from pharmaceutically acceptable salts and/or solvates of the disclosed compounds. Pharmaceutically acceptable salts include those derived from pharmaceutically acceptable inorganic or organic bases and acids. Particular disclosed compounds possess at least one basic group that can form acid-base salts with acids. Examples of such basic groups present in exemplary inhibitors include, but are not limited to, amino and imino groups. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art.

Certain compounds include at least one acidic group that can form an acid-base salts with an inorganic or organic base. Examples of salts formed from inorganic bases include salts of the presently disclosed compounds with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. Of the aliphatic amines, the acyclic aliphatic amines, and cyclic and acyclic di- and tri-alkyl amines are particularly suitable for use in the disclosed compounds. In addition, quaternary ammonium counterions also can be used.

Particular examples of suitable amine bases (and their corresponding ammonium ions) for use in the present compounds include, without limitation, pyridine, N,N-dimethylaminopyridine, diazabicyclononane, diazabicycloundecene, N-methyl-N-ethylamine, diethylamine, triethylamine, diisopropylethylamine, mono-, bis- or tris-(2-hydroxyethyl)amine, 2-hydroxy-tert-butylamine, tris(hydroxymethyl)methylamine, N,N-dimethyl-N-(2-hydroxyethyl)amine, tri-(2-hydroxyethyl)amine and N-methyl-D-glucamine. For additional examples of “pharmacologically acceptable salts,” see Berge et al., J. Pharm. Sci. 66:1 (1977).

Compounds disclosed herein can be crystallized and can be provided in a single crystalline form or as a combination of different crystal polymorphs. As such, the compounds can be provided in one or more physical form, such as different crystal forms, crystalline, liquid crystalline or non-crystalline (amorphous) forms. Such different physical forms of the compounds can be prepared using, for example different solvents or different mixtures of solvents for recrystallization. Alternatively or additionally, different polymorphs can be prepared, for example, by performing recrystallizations at different temperatures and/or by altering cooling rates during recrystallization. The presence of polymorphs can be determined by X-ray crystallography, or in some cases by another spectroscopic technique, such as solid phase NMR spectroscopy, IR spectroscopy, or by differential scanning calorimetry.

Also disclosed are prodrugs of the presently disclosed compounds. “Prodrug,” as used herein, means a compound which is convertible in vivo by metabolic means (e.g., by hydrolysis) to an active reverse transcriptase inhibitor.

Various forms of prodrugs are known in the art, for example, as discussed in Bundgaard, (ed.), Design of Prodrugs, Elsevier (1985); Widder et al. (ed.), Methods in Enzymology, vol. 4, Academic Press (1985); Krogsgaard-Larsen et al., (ed). Design and Application of Prodrugs, Textbook of Drug Design and Development, Chapter 5, 113 191 (1991), Bundgaard, et al., Journal of Drug Delivery Reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975).

### III. Methods of Treatment

NNRTIs are provided herein that can substantially reduce or inhibit HIV infection and replication. These NNRTIs can be used alone, or in combination with other therapies, to substantially inhibit or reduce the biological activity of a reverse transcriptase, such as an HIV-1 reverse transcriptase. In some examples, such a method can be used to inhibit HIV infection or replication, for example to treat HIV-1 or other similar retrovirus infection (e.g., SW or FIV), or diseases associated with such infection (such as AIDS in a primate or feline).

Methods of substantially reducing or inhibiting the biological activity of a reverse transcriptase (RT), such as an HIV-1 RT are provided. The methods include contacting the RT with a therapeutically effective amount of any of the disclosed NNRTIs, alone or in combination with a therapeutically effective amount of other antiviral agents (such as an NRTI or PI). For example, the disclosed NNRTIs can be incubated with a cell culture, or administered to a subject (e.g., orally). The activity of the RT need not be reduced by 100% for the therapy to be considered effective. For example, a reduction of at least 20%, at least 50%, at least 75%, at least 90%, or at least 95% can be considered effective. Methods of measuring RT activity are known in the art, and can include detecting HIV-1 nucleic acids (e.g., DNA) or proteins present in a sample using routine methods.

The ability to substantially reduce or inhibit the biological activity of a RT, thereby enables a method for inhibiting or treating HIV infection (or infection with a similar retrovirus such as SIV or FIV). In particular methods, HIV infection is inhibited by contacting a cell with a therapeutically effective amount of one or more of the disclosed NNRTIs, thereby inhibiting HIV infection. For example, the NNRTIs can be added to culture medium in which cells are cultured, or administered to a mammalian subject using routine methods. HIV infection or replication need not be reduced by 100% for the therapy to be considered effective. For example, a reduction of at least 20%, at least 50%, at least 75%, at least 90%, at least 95%, at least 98%, or even at least 100% (elimination of detectable HIV infected cells or nucleic acids) can be considered effective. Methods of measuring HIV infection and replication are known in the art, and can include detecting HIV-1 nucleic acids (e.g., DNA) or proteins present in a sample using routine methods (e.g., PCR, Western blotting, and flow cytometry). In some examples, treatment of an SIV or HIV infection, reduces the HIV or SIV RNA viral load to below the detectable limit. In some example, this the detectable limit is less than 200, less than 100, or less than 50 copies of HIV or SIV RNA per ml of plasma of the subject (e.g., 200 to 10 or 200 to 1 copies per ml of plasma), for example as measured by quantitative, multi-cycle reverse transcriptase PCR.

The identified NNRTIs provided herein can be used in combination with other antiviral agents as part of an antiretroviral therapy, such as a highly active antiretroviral therapy (HAART). Antiviral therapies can be used to treat HIV infections (e.g., HIV-1 or HIV-2) in humans or SIV infections in other primates (e.g., macaques), HIV infections in felines, and in some examples are useful for treating a subject with one or more symptoms of AIDS. Although the newly-identified NNRTIs provided herein can be used alone, they can also be used as part of multi-drug combination therapies, such as the HAART triple and quadruple combination therapies. Exemplary suitable multi-drug combination therapies include at least two anti-HIV-1 drugs selected from NNRTIs and PIs, triple therapies such as (i) two NRTIs and one PI; (ii) two NRTIs and one NNRTI, (iii) one NRTI, one NNRTI, and one PI, (iv) two PIs and one NNRTI; and quadruple combination therapies such as (i) two NRTIs, one PI and a second PI or one NNRTI. Thus the disclosed NNRTIs can be used as an NNRTI in these combination therapies.

In particular examples, the NNRTIs are administered to a subject infected with HIV, SIV, or FIV. In some examples, the subject has AIDS. In some examples, the subject is asymptomatic with less than 200 CD4+ T cells/0, asymptomatic with CD4+ T cell counts of 201-350 cells/μl; asymptomatic patients with CD4+ T cell of greater than 350 cells/μl and plasma HIV RNA greater than 100,000 copies/ml. In particular examples, subjects having CD4+ T cell counts of greater than 350 cells/μl and plasma HIV RNA less than 100,000 copies/mL do not receive the disclosed therapies.

A. Combination Therapies

The disclosed NNRTIs can be used in combination with other retroviral inhibitors, for example in combination with highly active antiretroviral therapy (HAART). In some examples, the disclosed NNRTIs are used in combination with one or more reverse transcriptase inhibitors (e.g., nucleoside/nucleotide reverse transcriptase inhibitors (NRTIs) and non-nucleoside reverse transcriptase inhibitor (NNRTIs)), protease inhibitors (PIs), viral entry or fusion inhibitors, RNAse H inhibitors, integrase inhibitors, maturation inhibitors, or combinations thereof.

Reverse transcriptase inhibitors (RTIs) target generation of viral DNA by inhibiting activity of reverse transcriptase. There are two subtypes of RTIs with different mechanisms of action: NIRIs are incorporated into the viral DNA leading to chain termination, while NNRTIs distort the binding potential of the reverse transcriptase enzyme.

NRTIs include nucleosides/nucleotides and analogs thereof that substantially reduce or inhibit the activity of HIV-1 reverse transcriptase. Typical suitable NRTIs that can be used in combination with the disclosed newly-identified NNRTIs, for example in a HAART, include zidovudine (AZT; e.g., at a dose of 100 to 1000 mg two to three times a day, such as 300 mg two to three times a day); didanosine (ddI; e.g., on empty stomach, at least 30 minutes before or 2 hours after eating, for patients weighing 132 pounds or more, the recommended doses are 200 mg twice a day (tablets), 250 mg twice a day (buffered powder), or 400 mg once a day (enteric-coated capsules). For adults weighing less than 132 pounds, the recommended doses are 125 mg twice a day (tablets), 167 mg twice a day (buffered powder), or 250 mg once a day (enteric-coated capsules); zalcitabine (ddC; e.g., at a dose of 0.10 mg to 1.5 mg every 8 hours, such as 0.750 mg every 8 hours); lamivudine (3TC; e.g., at a dose of 50 to 600 mg/day, such as 300 mg once daily, or 150 mg twice a day); abacavir (e.g., for adults at a dose of 50 to 600 mg twice a day, such as 300 mg 2 times a day and for people less than 17 years 3.6 mg per lb. of body weight twice a day, up to a maximum of 300 mg in each dose); emtricitabine (e.g., at a dose of 50 to 500 mg/day, such as 200 mg as a capsule or 240 mg (24 ml) as an oral solution once a day); tenofovir disoproxil fumarate (e.g., at a dose of 50 to 900 mg/day, such as 300 mg/day), adefovir dipivoxil [bis(POM)-PMEA]; lobucavir; stavudine (e.g., at a dose of 10 to 100 mg/day, such as 40 mg twice a day for subjects weighing 132 lbs or more and 30 mg twice a day for individuals weighing less than 132 lbs); apricitabine (e.g., at a dose of 400 to 1,600 mg twice per day); elvucitabine (e.g., at a dose of 5 and 10 mg once daily or 20 mg once every other day); KP-1461 (also known as SN1461 and SN1212); racivir; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and iodenosine (FddA), 9-(2,3-dideoxy-2-fluoro-b-D-threo-pentofuranosyl)adenine. All of these NRTIs can be administered orally, for example in combination with one of the disclosed NNRTIs.

NNRTIs include non-nucleosides and analogs thereof that substantially reduce or inhibit the activity of HIV-1 reverse transcriptase. Typical suitable NNRTIs that can be used in combination with the disclosed newly-identified NNRTIs include nevirapine (e.g., at a dose of 50 to 1000 mg/day, such as 200 mg once a day for the first 14 days, then 200 mg twice a day); delaviradine (e.g., at a dose of 200 to 1000 mg 3 times per day, such as 400 mg 3 times per day); efavirenz (e.g., at a dose of 200 to 1000 mg/day, such as 600 mg/day); etravirine (e.g., at a dose of 100 to 500 mg twice a day, such as 200 mg twice a day); TMC278 (e.g., at a dose of 75, 125, or 150 mg daily); PNU-142721; 5-(3,5-dichlorophenyl)-thio-4-isopropyl-1-(4-pyridyl)methyl-1H-imidazol-2-ylmethyl carbonate; MKC-442 (1-(ethoxy-methyl)-5-(1-methylethyl)-6-(phenylmethyl)-(2,4(1H,3H)-pyrimidinedione); and (+)-calanolide A (NSC-675451) and coumarin derivatives disclosed in U.S. Pat. No. 5,489,697. All of these NNRTIs can be administered orally, for example in combination with one of the disclosed NNRTIs.

Protease inhibitors (PIs) target viral assembly by inhibiting the activity of protease, an enzyme used by HIV and other retroviruses to cleave nascent proteins for final assembly of new virons. Typical suitable PIs that can be used in combination with the disclosed newly-identified NNRTIs include compounds having a peptidomimetic structure, high molecular weight (7600 daltons) and substantial peptide character (e.g., indinavir), as well as nonpeptide protease inhibitors (e.g., nelfnavir). Exemplary PIs include atazanavir (e.g., at a dose of 200 to 800 mg/day, such as 300 or 400 mg/day); darunavir (e.g., at a dose of 200 to 1000 mg twice per day, such as 600 mg (two 300-mg tablets) taken with ritonavir 100 mg twice a day with food); fosamprenavir (dosage can depends on whether a patient has been treated for HIV before or if this is part of the first anti-HIV drug combination for the patient; adult patients on their first anti-HIV drug combination, there are three ways to dose fosamprenavir: 1) 1,400 mg twice daily without ritonavir, 2) 1,400 mg once daily plus ritonavir 200 mg once daily, or 3) 700 mg twice daily plus ritonavir 100 mg twice daily, for adult patients who have already taken anti-HIV drugs, the recommended dose of fosamprenavir is 700 mg twice daily plus ritonavir 100 mg twice daily); lopinavir/ritonavir (e.g., at a dose of 200-1000 mg/day lopinavir and 50-500 mg/day ritonavir, such as 400 mg/100 mg twice daily with or without food); saquinavir (e.g., at a dose of 200 to 2000 mg 3 times per day, such as 400 mg, 1000 mg, or 1200 mg 3 times per day, for example in combination with 100 mg of ritonavir two times a day); saquinavir mesylate (e.g., at a dose of 500 to 2000 mg 2 times per day with 50 to 100 mg ritonavir, such as 1,000 mg with 100 mg of ritonavir two times a day); ritonavir (e.g., at a dose of 100 to 1200 mg twice per day, such as 100 or 600 mg twice per day); indinavir (e.g., at a dose of 200 to 1200 mg every 8 hours, such as 800 mg (two 400-mg capsules) every 8 hours); nelfnavir (e.g., at a dose of 600 to 3000 mg twice per day, such as 1,250 mg (five 250 mg tablets or two 625 mg tablets) twice a day or 750 mg (three 250 mg tablets) three times a day); amprenavir (e.g., at a dose of 500 to 2400 mg twice per day, such as 1200 mg (twenty-four 50 mg capsules) twice a day); tipranavir (e.g., at a dose of 200 to 1000 mg twice daily, such as 500 mg taken with ritonavir 200 mg twice daily); lasinavir; ABT-378; and AG-1549 an orally active imidazole carbamate. All of these PIs can be administered orally, for example in combination with one of the disclosed NNRTIs.

RNAse H inhibitors include agents that substantially reduce or inhibit the activity of HIV-1 RNase H. Typical suitable RNAse H inhibitors that can be used in combination with the disclosed newly-identified NNRTIs include N-acyl hydrazones and aryl hydrazones, such as dihydroxy benzoyl napthyl hydrazone and analogs thereof.

Integrase inhibitors are a class of antiretroviral drug that blocks the action of integrase, an enzyme that integrates viral DNA into the DNA of the infected cell. Examples include raltegravir (e.g., at a dose of 200 to 800 mg twice a day, such as 400 mg twice a day); elvitegravir (e.g., at a dose of 200 to 800 mg twice a day or 50 and 800 mg once daily, for example with 100 mg ritonavir), and JTK-303. All of these integrase inhibitors can be administered orally, for example in combination with one of the disclosed NNRTIs.

Entry or fusion inhibitors block HIV-1 from the host cell by binding CCR5, a molecule on the host membrane termed a co-receptor that HIV-1 normally uses for entry into the cell together with a primary receptor. Entry inhibitors that can be used in combination with the disclosed newly-identified NNRTIs include maraviroc (e.g., at a dose of 100 to 600 mg twice a day, such as 300 mg twice daily) and enfuvirtide (e.g., at a dose of 10 to 200 mg twice a day, such as 2 mg/kg with a maximum of 90 mg twice daily), as well as AMD070; PRO140 (e.g. iv infusion from 0.1 to 5 mg/kg); SCH-D; and TNX-355 (e.g., via iv infusion). These entry inhibitors can be administered orally or iv, for example in combination with one of the disclosed NNRTIs.

Maturation inhibitors inhibit the last step in gag processing in which the viral capsid polyprotein is cleaved, thereby blocking the conversion of the polyprotein into the mature capsid protein (p24). Because these viral particles have a defective core, the virions released consist mainly of non-infectious particles. Bevirimat (e.g., at a dose of 25 to 600 mg/day orally) is a maturation inhibitor that can be used in combination with the disclosed newly-identified NNRTIs. These maturation inhibitors can be administered orally, for example in combination with one of the disclosed NNRTIs.

Other antiviral agents that can be used in combination with the disclosed new NNRTIs include hydroxyurea, ribavirin, IL-2, IL-12, pentafuside, ribavirin, 1β-D-ribofuranosyl-1H-1,2,4-triazole-3-carboxamide, and hydroyurea (Droxia) a ribonucleoside triphosphate reductase inhibitor. IL-2 is available as a lyophilized powder for intravenous (iv) infusion or subcutaneous (sc) administration upon reconstitution and dilution with water at a dose of 1 to 20 million IU/day sc or a dose of 15 million IU/day. IL-12 can be administered sc in a dose of about 0.5 μg/kg/day to about 10 μg/kg/day, for example sc. Pentafuside (e.g., FUZEON®) is a 36-amino acid synthetic peptide (see U.S. Pat. No. 5,464,933) that can be administered at a dose of 3-100 mg/day (such as 100 mg/day) as a continuous sc infusion or injection together with efavirenz and 2 PI's to HIV-1 positive patients refractory to a triple combination therapy.

In treatment of naive patients, an anti-HIV treatment can include a triple combination therapy, such as (i) a new NNRTI disclosed herein, one NRTI and one PI, (ii) a new NNRTi disclosed herein and two NRTIs, (iii) one new NNRTI disclosed herein, one known NNRTi, and one NRTI, (iv) two new NNRTI disclosed herein, and one NRTI, (v) one new NNRTI disclosed herein, one known NNRTi, and one PI, or (vi) two new NNRTI disclosed herein, and one PI. The CD4⁺ and HIV-1-RNA plasma levels can be monitored every 3-6 months. If there is an observed viral load plateau, a fourth drug, e.g., one PI, one NNRTI, or one NRTI can be added to the treatment regimen.

B. Administration and Dosing

The newly-identified NNRTIs provided herein can be contacted with a cell, for example to substantially reduce infection of the cell by HIV or decrease detectable HIV DNA or RNA in the cell. In particular examples, one or more of the disclosed NNRTIs is administered to a mammalian subject, such as a primate, for example a human or macaque, to treat an HIV or SW infection, for example to treat a subject with AIDS.

Methods of administering the disclosed NNRTIs to a subject (such as a mammal) is routine. Any form of administration can be used, and the appropriate route of administration can be determined by a skilled clinician. For example, the disclosed NNRTIs can be administered orally, via injection, or via transdermal delivery. In some examples, the disclosed NNRTIs are administered to a subject using known methods for administering other NNRTIs, such as orally in the form of a pill/tablet or liquid. For example, the NNRTIs can be administered to a subject orally.

The dosage and timing of administration of the anti-HIV compounds (including the new NNRTIs provided herein) employed in the disclosed methods can be varied depending upon the requirements of the subject and the severity of the condition being treated. Determination of the proper dosage regimen, that is, a therapeutically effective amount, for a particular situation is within the skill of the art. In addition, the present application provides methods that can be used to identify therapeutically effective amounts. For convenience, the total daily dosage may be divided and administered in portions during the day as required.

In a specific example, the therapeutically effective amount of an NNRTI is about 100 to 2000 mg orally, one, two, or three times per day. In certain examples a therapeutically effective amount of a disclosed NNRTI is from about 150 to about 1500 mg, such as from about 80 to about 800 mg or from about 250 to about 1200 mg of the NNRTI administered orally one, two or three times per day. The anti-HIV therapies provided herein can be administered to a mammalian subject for at least two consecutive days, 10 consecutive days, and so forth, for example for a period of weeks, months, or years, such as the lifetime of the subject.

For combination treatment with more than one therapeutic agent, where the active agents (e.g., NNRTI, NRTI, and PI) are in separate dosage formulations, the active agents may be administered separately or in conjunction. In addition, the administration of one therapeutic agent may be prior to, concurrent to, or subsequent to the administration of the other therapeutic agent.

C. Pharmaceutical Compositions

In some examples, the newly identified NNRTIs provided herein are formulated into a composition for administration. Actual methods for preparing administrable compositions will be known or apparent to those skilled in the art and are described in more detail in such publications as Remington's Pharmaceutical Sciences, by E. W. Martin, Mack Publishing Co., Easton, Pa., 19th Edition (1995).

For example, the disclosed NNRTIs can be formulated into a composition with acceptable pharmaceutically acceptable carriers. In one example, the disclosed NNRTIs are dissolved in an aqueous carrier, for example for a carrier suitable for injection, transdermal delivery, or oral delivery. A variety of aqueous carriers can be used, for example, buffered saline and the like. These solutions are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of disclosed NNRTIs in these formulations can vary, and can be selected primarily based on fluid volumes, viscosities, body weight and the like in accordance with the particular mode of administration selected and the subject's needs.

The compositions that include one or more of the disclosed NNRTIs can further include one or more biologically active or inactive compounds (or both), such as anti-viral agents and conventional non-toxic pharmaceutically acceptable carriers, respectively.

In a particular example, a therapeutic composition that includes a therapeutically effective amount of one or more disclosed NNRTIs further includes one or more biologically inactive compounds. Examples of such biologically inactive compounds include, but are not limited to: carriers, thickeners, diluents, buffers, preservatives, and carriers. In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can include minor amounts of non-toxic auxiliary substances, such as wetting or emulsifying agents, preservatives, and pH buffering agents and the like, for example sodium acetate or sorbitan monolaurate.

Oral administration of the disclosed antiviral agents (e.g., an NNRTI, NRTI, or PI), can be in the form of tablets, coated tablets, dragees, hard and soft gelatine capsules, solutions, emulsions, syrups, or suspensions. Solid form preparations include powders, tablets, pills, capsules, and cachets. A solid carrier can include substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions can be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

NNRTIs and other therapeutic agents provided herein, as well as their pharmaceutically useable salts, together with one or more conventional carriers, can be placed into the form of pharmaceutical compositions and unit dosages. The pharmaceutical compositions and unit dosage forms can include conventional ingredients in conventional proportions, with or without additional active compounds or principles, and the unit dosage forms may contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

NNRTIs and other therapeutic agents provided herein can be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing or dispersing agents. Alternatively, the active ingredient can be in powder form, obtained by aseptic isolation of sterile solid or by lyophilisation from solution for constitution before use with a suitable vehicle (e.g., sterile, pyrogen-free water).

Formulations can be prepared with enteric coatings adapted for sustained or controlled release administration of the active ingredient. For example, NNRTIs and other therapeutic agents provided herein can be formulated in transdermal or subcutaneous drug delivery devices. These delivery systems can be used to achieve sustained release of the compound. Compounds in transdermal delivery systems can be attached to a skin-adhesive solid support. The compound of interest (e.g., NNRTIs provided herein) can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

### IV. Examples

The foregoing disclosure is further explained by the following non-limiting examples.

### Example I

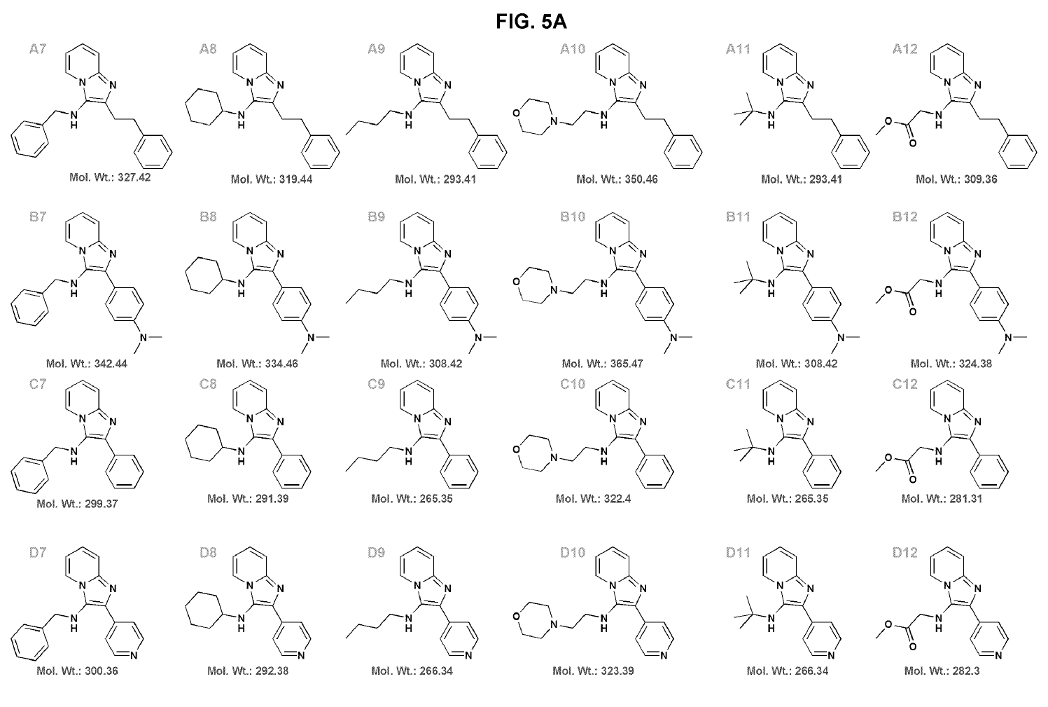

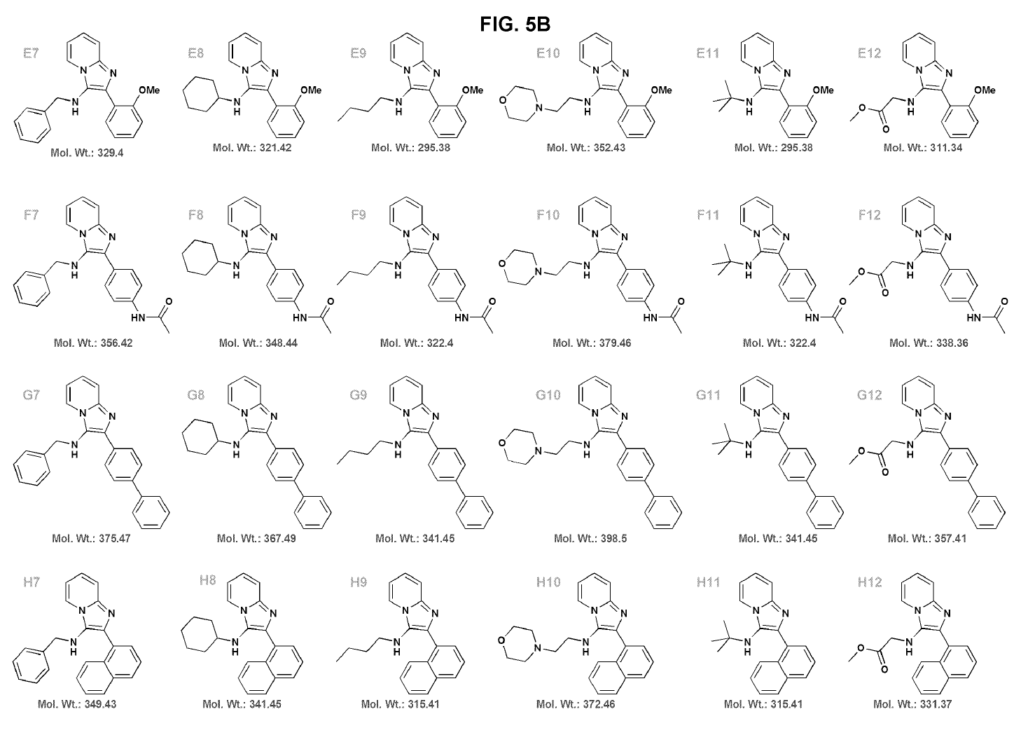

This example describes the preparation of a representative inhibitor library, referred to herein as CBPL-08-006, based on the 3-amino imidazo[1,2-a]pyridine scaffold as produced via the Multicomponent Condensation Reaction (MCR) known as the Groebke reaction. This is a special case of the MCR known as the Ugi Reaction. An overview of the mechanism of this reaction is provided in FIG. 1, and FIGS. 4 and 5 illustrate specific exemplary members of library CBPL-08-006. Compounds in the libraries described herein are referred to herein by the library number, e.g., CBPL-08-006, followed by a letter and a number corresponding to the coordinates of the reaction well on a plate map in which the specific compound was assembled.

Briefly, the Groebke reaction, an example of which is illustrated in FIG. 1, is carried out by combining 3 building blocks in a substantially equimolar ratio, with or without an acid catalyst and allowing the reaction to progress at various effective temperatures ranging from 25-140° C. over time periods effective to form desired compounds, such as from about 5 minutes to about 5 days. These broad time and temperature ranges reflect normal ambient temperature reaction conditions and completed over the period of days to microwave accelerated conditions carried out at the higher temperatures and completed in a matter of minutes.

Library CBPL-08-006 was carried at 25° C. and allowed to react for 3 days. The reaction mixtures were carried out in a Charybdis Technologies (CT) Calypso Reaction Block, sealed under a N₂ atmosphere and continuously agitated on a CT 4× Shaker, at 600 RPM for 5 days. The reaction blocks were Calypso 96-well Solution Blocks, but alternatively SBS Deep Well 96 Well Microplates can be substituted for the CT Teflon Microplate. To each well was added (in no particular order of addition requirement) stock solutions of 2-amino pyridine, aldehyde, isonitrile (isocyanide), and Lewis acid catalyst scandium [III] triflate (ScOTf₃).

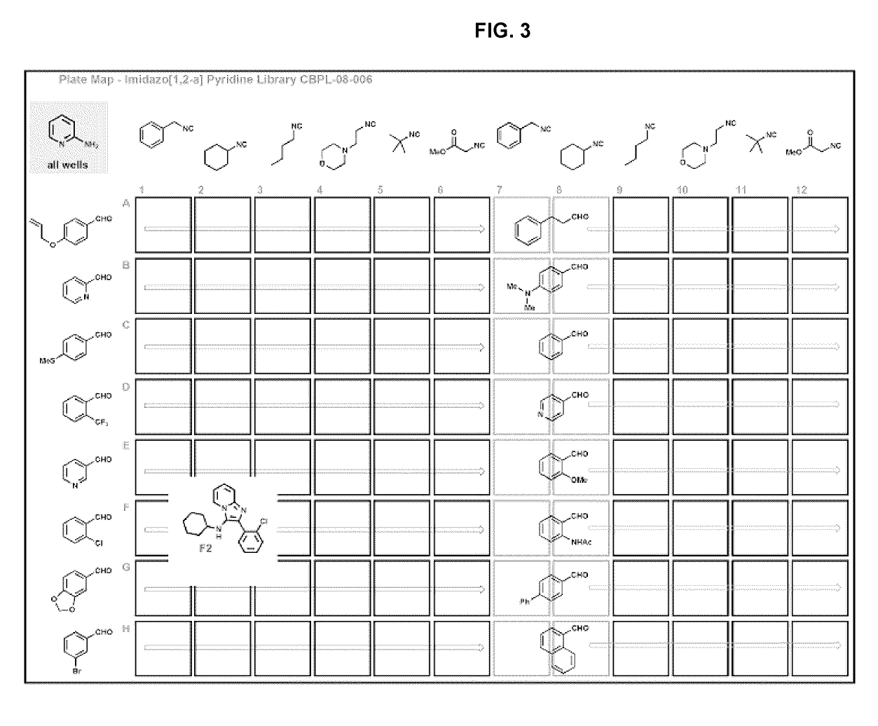

The assembly of the library components followed the plate map illustrated in FIG. 3. Specifically, 2-amino pyridine (Sigma-Aldrich) was prepared as a 0.5M stock solution in 1,2-dichloroethane (DCE) and 200 μL (0.1 mmol reaction scale) was added to all wells. Alternatively this stock solution could employ solvents such as DCM, chloroform, methanol, TFE, THF or Room Temperature Ionic Liquid (RTIL). Next, 0.5M stock solutions of 16 aldehydes (Sigma-Aldrich) in 2,2,2-trifluoroethanol (TFE), and 200 μL (0.1 mmol reaction scale) were added to all wells where, with reference to FIG. 3: Aldehyde 1 was added to Row A, wells 1 thru 6; Aldehyde 2 was added to Row B, wells 1 thru 6; Aldehyde 3 was added to Row C, wells 1 thru 6; Aldehyde 4 was added to Row D, wells 1 thru 6; Aldehyde 5 was added to Row E, wells 1 thru 6; Aldehyde 6 was added to Row F, wells 1 thru 6; Aldehyde 7 was added to Row G, wells 1 thru 6; Aldehyde 8 was added to Row H, wells 1 thru 6; Aldehyde 9 was added to Row A, wells 7 thru 12; Aldehyde 10 was added to Row B, wells 7 thru 12; Aldehyde 11 was added to Row C, wells 7 thru 12; Aldehyde 12 was added to Row D, wells 7 thru 12; Aldehyde 13 was added to Row E, wells 7 thru 12; Aldehyde 14 was added to Row F, wells 7 thru 12; Aldehyde 15 was added to Row G, wells 7 thru 12; Aldehyde 16 was added to Row H, wells 7 thru 12. Alternatively these stock solutions could employ solvents such as DCM, chloroform, methanol, TFE, THF or Room Temperature Ionic Liquid (RTIL). Next, 0.5M stock solutions of 6 isocyanides (Sigma-Aldrich) in DCE, and 200 μL (0.1 mmol reaction scale) was added to all wells where: Isocyanide 1 was added to Columns 1 and 7; Isocyanide 2 was added to Columns 2 and 8; Isocyanide 3 was added to Columns 3 and 9; Isocyanide 4 was added to Columns 4 and 10; Isocyanide 5 was added to Columns 5 and 11; Isocyanide 6 was added to Columns 6 and 12. Alternatively these stock solutions could employ solvents such as DCM, Chloroform, Methanol, TFE, THF or Room Temperature Ionic Liquid (RTIL). Finally, a stock solution of 0.025M ScOTf₃ (Sigma-Aldrich), and 200 μL (0.1 mmol reaction scale, 5 mol % catalyst) was added to all wells. Alternatively this stock solution could employ solvents such as DCM, Chloroform, Methanol, TFE, THF or Room Temperature Ionic Liquid (RTIL).

### Example 2

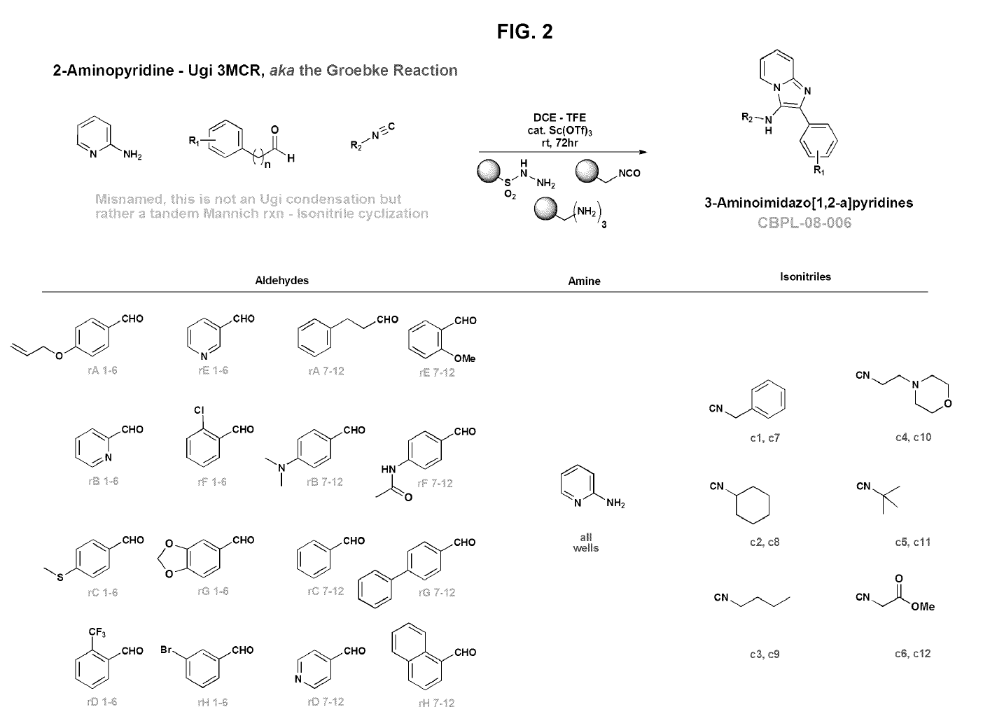

This example describes a second embodiment of a method for making disclosed compounds, and in particular describes the synthesis of a lead compound (CBPL-08-006 F2) identified in screening of the library described in FIG. 2. The resynthesis was carried out employing microwave accelerated organic synthesis (MAOS) techniques. The protocol was as follows: To a 20 mL microwave process vial (Biotage) was added 2-aminopyridine (470.6 mg, 5.0 mmol, 1.0 eq) and 2-chlorobenzaldehyde (563 μL, 5.0 mmol, 1.0 eq) and let to stir for 5 minutes. Neat amine and aldehyde were mixed to preform the intermediate imine or Schiff's base. To this imine was added 5 mL of DCE and 5 mL of TFE followed by solid ScOTf₃ (123 mg, 0.25 mmol, 0.05 eq or 5 mol %). The mixture was stirred to effect the dissolution of the Lewis acid catalyst and to this mixture was added cyclohexyl isocyanide (621.7 μL, 5.0 mmol, 1.0 eq). The vial was flushed with N₂ (g) and sealed with a Biotage crimp seal. The reaction was run for 20 min. at 120° C., transferred to a round bottom flask and the solvent was removed in vacuo. The crude product was redissolved in EtOAc and flash chromatographed thru a plug TLC grade silica gel with Hexanes-EtOAC (3:1) to afford 1.47 g (90%) of viscous yellow-brown oil. LCMS determined a single peak (@ 254 nm) with a mass of 326.8 (M⁺) in ESI+ mode corresponding to expected compound. 1H NMR (500 MHz, CDCl₃): δ 8.14 (d, J=Hz, 1H), 7.67 (d, J=Hz, 1H), 7.54 (d, J=Hz, 1H), 7.46 (d, J=Hz, 1H), 7.35 (t, J=Hz, 1H), 7.33 (dd, J=Hz, 1H), 7.13 (t, J=Hz, 1H), 6.80 (t, J=Hz, 1H), 3.26 (br d, NH, 1H), 2.67 (m, 1H), 1.66 (m, 2H), 1.56 (m, 2H), 1.46 (m, 1H), 1.04 (m, 5H). ¹³C NMR (125 MHz, CDCl₃): δ 141.6 (C), 135.0 (C), 134.0 (C), 132.5 (CH), 132.5 (C), 129.4 (CH), 129.1 (CH), 126.9 (CH), 126.3 (C), 123.7 (CH), 122.8 (CH), 117.5 (CH), 111.5 (CH), 56.3 (CH), 33.8 (CH2), 25.6 (CH2), 24.5 (CH2).

### Example 3

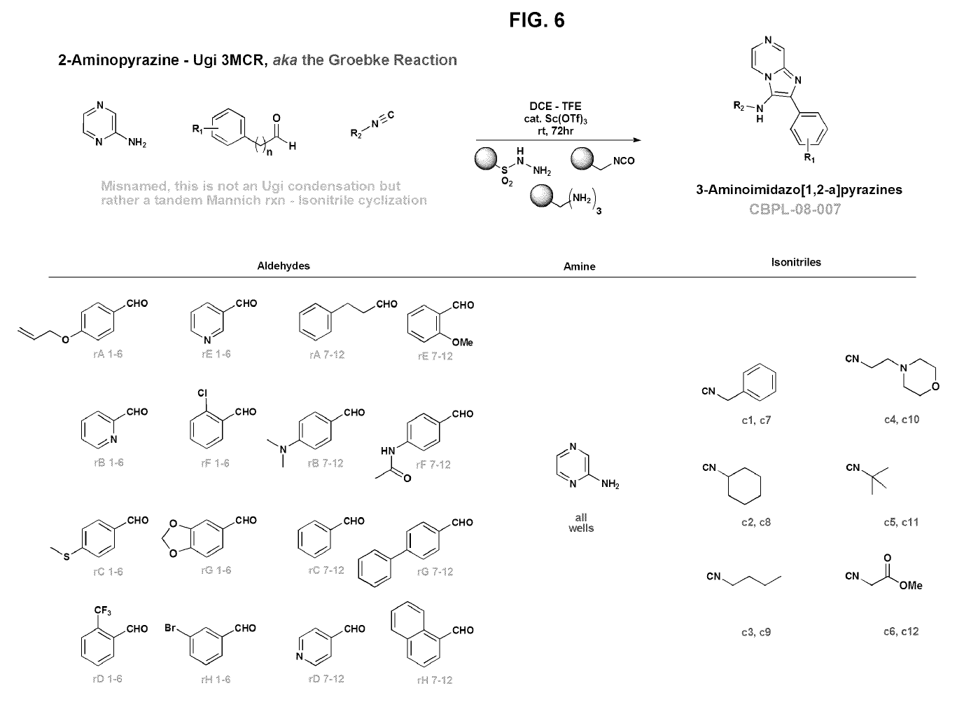

This example describes the preparation of a library of inhibitor compounds based on CBPL-08-006 F2. A 384-membered library was prepared according to the general schemes illustrated in FIGS. 1, 2 and 6. This library consisted of 4, 96 well plates that were designated CBPL-04-034 thru CBPL-04-037. The components used in the synthesis of this library are illustrated in FIGS. 9-12. With continued reference to the plate map of FIGS. 9-12, the library was prepared by Groebke condensation of the amines listed on the left hand side of the plate map with the aldehydes listed on the top of the plate map in the presence of cyclohexyl isocyanide. For example, compound CBPL-04-034-A2 of FIG. 9 is synthesized according to the scheme

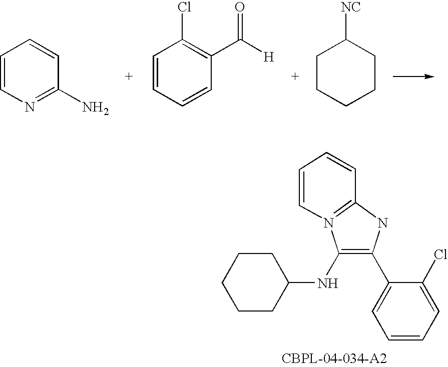

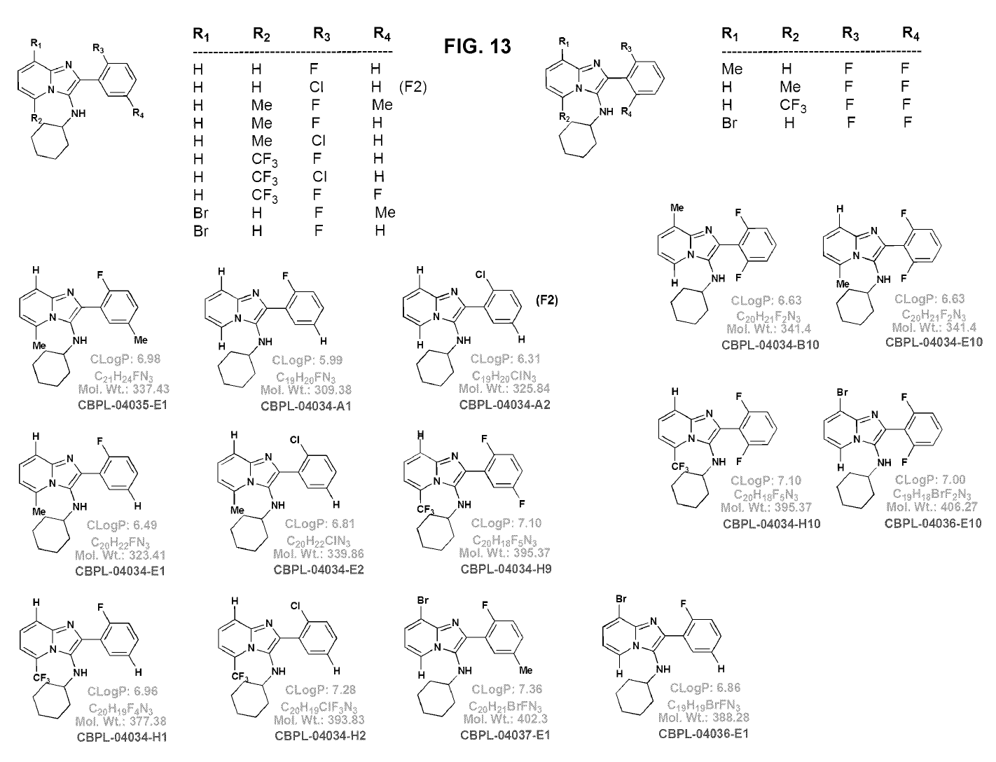

These reactions were carried out via MAOS techniques where substantially equal volume amounts of solutions containing amine, aldehyde, isocyanide and Lewis acid catalyst were added together. Specifically to each well was added: 200 μL of amine as a 0.25M solution in 1:1 DCE-TFE, 200 μL of aldehyde as a 0.25M solution in TFE, 200 μL of cyclohexyl isocyanide as a 0.25M solution in DCE, and 200 μL of ScOTf₃ as a 0.0125M solution in 1:1 TFE-THF. Using individual 2 mL microwave reaction vials, each vial was heated twice to 120° C. for 10 minutes, for a total of 20 minutes. Following the reaction completion of each set of 96 reactions, the vial contents were transferred to CT filtration plate and eluted through a short plug of Si-Tris Amine (Tris Amine Silica Gel, Biotage) with 2×0.5 mL Hexanes-EtOAc (1:1) and collected in a deep well microplate (total volume of 1.8 mL per well). Each plate was concentrated to dryness in vacuo and resolved in 100% DMSO to a 100 mM solution assuming an average reaction yield of 50%. For example, given that the reaction scale was 0.05 mmol/well (theoretical yield), the assumed yield was 0.025 mmol/well and therefore 250 μL of DMSO was used to redissolve the dried down compound to create the 100 mM Master Plate concentrations. Daughter plates consisting of 10 mM compound in 100% DMSO were created (10-to-1 dilution with 100% DMSO). These daughter plates were used for screening in the HIV infection assay described below. Exemplary active compounds identified from this library include those listed in FIG. 13.

### Example 4

This example describes the characterization of compound libraries produced as described in the examples above. Libraries were analyzed using HPLC/MS to confirm compound purity and molecular weight. Analytical HPLC was carried out on an Agilent 1100 HPLC and LCT Trap MS, outfitted with Phenomenex Gemini analytical column, 4.6×150 mm. Typical parameters were (A) (Water, 0.1% Formic Acid) and (B) (MeCN, 0.1% Formic Acid) where (A:B) initial gradient of (95:5) to (0:100) over 25 minutes at a flow rate of 0.5 ml/min were used. MS scanned both >ESI+/−modes (200-1000 amu) and in conjunction with diode array UV spectra (190-400 nm). Alternatively, a Waters Autopurification LCMS System was employed for both analytical analysis and preparative HPLC purification. For analytical purposes, Waters XBridge columns (4.6×50 mm) were used. Typical parameters were (A) (Water, 0.1% Formic Acid) and (B) (MeCN, 0.1% Formic Acid) where (A:B) initial gradient of (95:5) to (0:100) over 6 minutes at a flow rate of 3.0 ml/min were used. MS scanned both ESI+/−modes (200-1000 amu) and in conjunction with dual wavelength UV spectra (215 and 254 nm) and Evaporative Light Scattering Detection (ELSD). Positive conformation of desired compound identity and purity were judged by mass identity (typically ESI+ mode for these compounds) and UV area at 254 nm. Yields were assessed by either co-injection of internal standards or isolation of desired compound. Using the synthetic schemes described herein, compounds were typically found to be produced in high yield and purity. Master libraries were quality checked by sampling a cross section of the array (A1, B2, C3, D4, etc.). Significant hits were sampled to verify identity and purity. Further structural confirmation was carried by resynthesis at a larger scale, followed by preparative HPLC-MS purification and NMR spectroscopy analyses (1H, 13C, gCOSY and DEPT).

### Example 5

This example describes methods for screening and evaluating the disclosed compounds for reverse transcriptase inhibition. Initial screening employed a plate-based assay to identify those that inhibit infection of human 293T cells by a VSV-G pseudotyped HIV-1 vector encoding firefly luciferase (pNL4-3LucR+E-, described by Connor et al. Virology. 206:935-44, 1995; which is incorporated herein by reference as to the assay). Vpr is required for efficient replication of human immunodeficiency virus type-1 in mononuclear phagocytes.

This assay recapitulates and monitors the so-called early phase of HIV-1 infection, including i) early and late viral DNA synthesis by viral reverse transcriptase ii) nuclear import of the viral preintegration complex iii) viral DNA integration, and iv) proviral gene expression. Luciferase activity serves as a reporter of infectivity.

Briefly, the screen employed a single cycle HIV-1 reporter virus encoding firefly luciferase. The virus was generated by transient transfection of human 293T cells with plasmids pNL4-3LucR+E- (Connor et al. Virology 206:935-44, 1995) and pMD.G plasmid that expresses the VSV-G glycoprotein. Ten thousand human 293T cells were plated in 80 μL medium in each well of 96-well tissue culture plate. The next day, 10 μL of each diluted compound was added to reach a final concentration of 1 μM, and incubated at 37° C. for 1 hour. A 10 μL aliquot of medium containing the VSV-G pseudotyped HIV-1 vector (25 pg of p24 antigen) was then added to each well. Twenty-four hours after viral challenge, 50 μL of the medium was removed and 50 μL of Bright-Glo reagent (Promega, Madison, Wis.) was added to lyse the cells and provide the luciferin substrate for virus-encoded firefly luciferase. After several minutes the luminescence associated with each sample was measured, and served as readout to quantify virus infectivity in each well. The best fitted curves and IC₅₀ values were calculated using Prism 4 software (GraphPad Software, San Diego).

The combined effects of the screened compounds and AZT (Sigma, St. Louis, Mo.), which in certain embodiments were synergistic was determined by testing the compounds in the infectivity assay individually and in combinations at a fixed molar ratio over a range of serial dilutions (Murga et al., Antimicrob. Agents Chemother. 50(10):3289-96, 2006, which is incorporated herein by reference with respect to evaluating synergy). The data were then analyzed by the isobologram technique, which evaluates the compound interactions by a dose-oriented geometric method (Richman, D et al., Antimicrob. Agents Chemother. 35(2): 305-8; 1991 and Chou and Talalay, Adv. Enzyme Regul. 22:27-55, 1984; both references are incorporated herein by reference with respect to calculating compound interactions). Cytotoxicity of the compounds was measured 24 hours after treatment of mock-infected cells by adding an equal volume of CellTiter-Glo (Promega, Madison, Wis.) and reading luminescence. Exemplary results obtained using this assay are summarized in Tables 1 and 2.

TABLE 1
Activity of selected compounds listed in FIGS. 4 and 5 in the cell based
infection assay using the VSV-G pseudotyped HIV-1 vector.

| Compound | IC₅₀ (μM)^{a} |
|---|---|
| 08-006-A2 | 9.7 (6.1-15.4) |
| 08-006-B2 | 13.6 (6.8-27.0) |
| 08-006-C2 | 16.3 (12.3-21.3) |
| 08-006-D2 | 13.5 (9.1-20.0) |
| 08-006-E2 | 6.2 (4.0-9.7) |
| 08-006-G2 | 9.2 (5.3-15.9) |
| 08-006-H2 | 11.9 (9.0-15.7) |
| 08-006-F1 | 16.0 (7.2-35.5) |
| 08-006-F2 | 0.8 (0.6-1.0) |
| 08-006-F3 | 3.0 (2.3-3.8) |
| 08-006-F5 | 7.6 (4.9-11.7) |

^{a}IC₅₀ values and 95% confidence intervals (in parentheses)

TABLE 2
Activity of selected compounds from FIGS. 9-13 in the cell based
infection assay using the VSV-G pseudotyped HIV-1 vector.

| Compound | IC₅₀ (nM)^{a} |
|---|---|
| 04-034-A1 | 185.3 (116.0-296.1) |
| 04-034-B10 | 143.9 (111.8-185.1) |
| 04-034-E1 | 127.8 (104.4-156.4) |
| 04-034-E2 | 115.1 (88.6-149.4) |
| 04-034-E10 | 226.0 (154.8-330.1) |
| 04-034-H1 | 177.2 (111.9-280.6) |
| 04-034-H2 | 180.5 (105.3-309.3) |
| 04-034-H9 | 238.8 (168.8-337.9) |
| 04-034-H10 | 134.5 (81.0-223.3) |
| 04-035-E1 | 85.8 (28.2-260.9) |
| 04-036-E1 | 191.9 (107.7-341.9) |
| 04-036-E10 | 154.8 (123.2-194.5) |
| 04-037-E1 | 254.5 (130.9-494.8) |

^{a}IC₅₀ values and 95% confidence intervals (in parentheses)

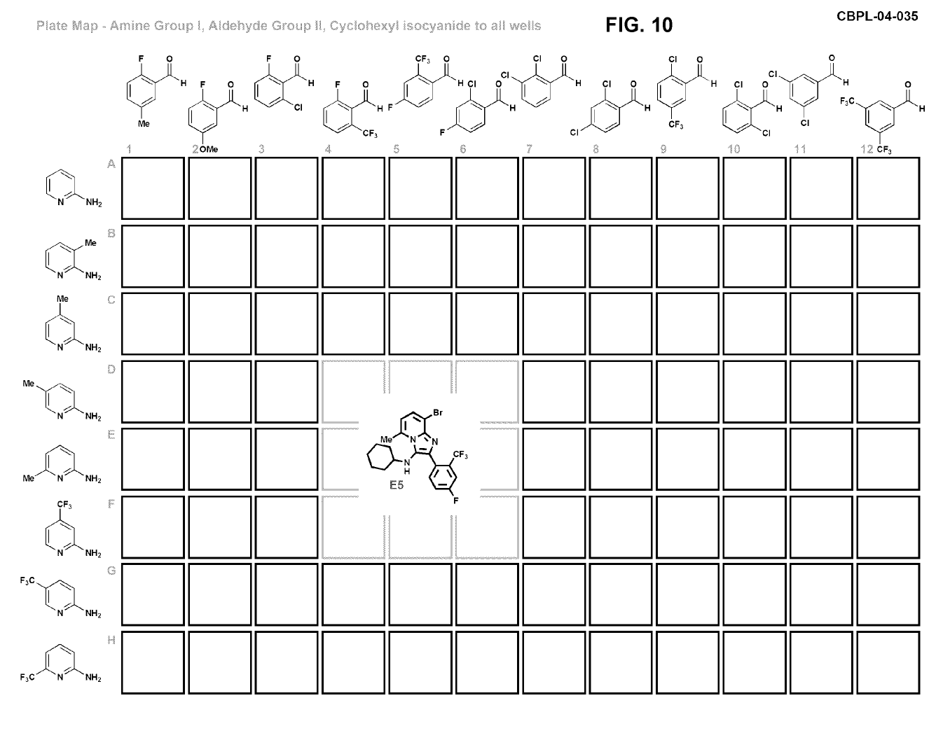

Inhibitor 04-035-E1 (FIGS. 10 and 13), was resynthesized, characterized and evaluated in additional repeated assays. In these assays, the IC₅₀ values of resynthesized 04-035-E1 ranged between 0.12 μM and 0.17 μM. Performing the HIV-1 infection assay using other cell types in addition to 293T cells yielded similar values: IC₅₀ μM for human osteosarcoma (HOS) cells and 0.15 μM for human T lymphoblast (CEM) cells (data not shown).

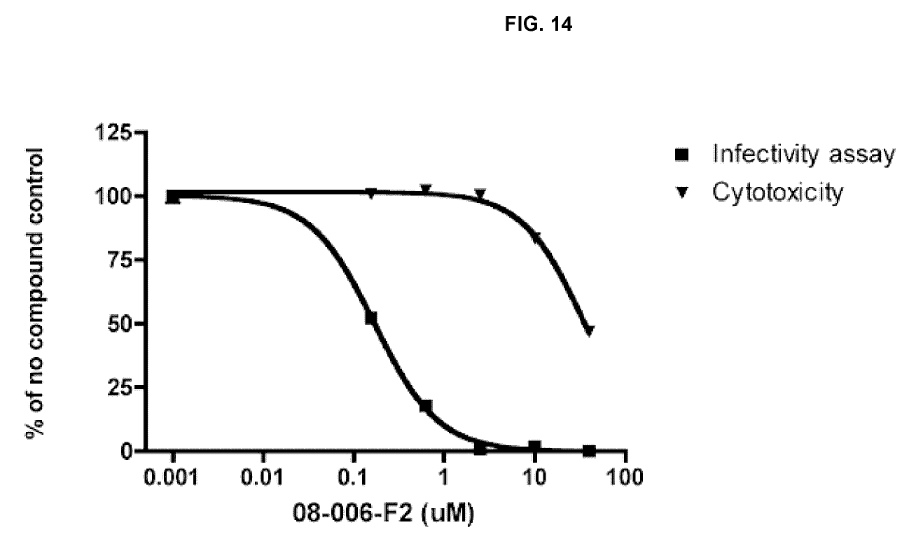

Results for compound 08-006-F2 in the cell-based infectivity assay using the VSV-G pseudotyped HIV-1 vector are charted in FIG. 14, which shows a dose-response curve for inhibition of HIV-1 vector infectivity (IC₅₀ 0.17 μM, 95% confidence interval 0.15 to 0.19 μM) and for cytotoxicity (CC₅₀ 35.0 μM, 95% confidence interval 32.2 to 38.0 μM).

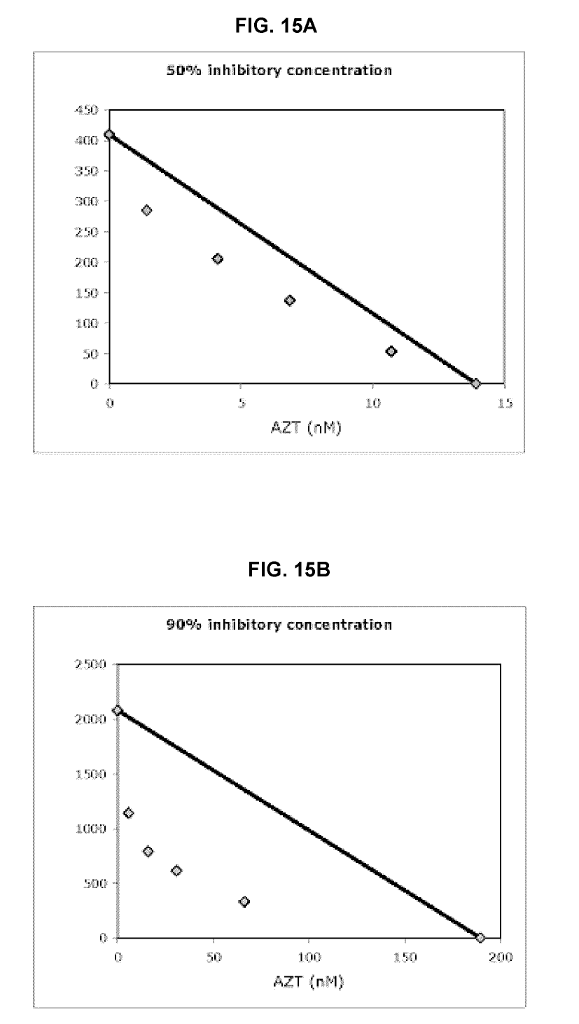

With reference to FIGS. 15A and 15B, examples of the disclosed inhibitors exhibit synergy in combination with nucleoside reverse transcriptase inhibitors as demonstrated by the isobologram plots of inhibition for combinations of AZT and 08-006-F2. The line between the 50% or 90% fractional inhibitory concentrations for single compounds indicates the values at which additive effects would occur. Values below and to the left of the lines indicate synergistic effects for inhibition when AZT and 08-006-F2 are used in combination.

Anti-HIV-1 reverse transcriptase activity of the disclosed inhibitor compounds was assayed in vitro using an assay adapted from King et al. (Antimicrob. Agents Chemother. 46(6): 1640-46; 2002) and Telesnitsky et al. (Methods Enzymol. 262:347-62; 1995). HIV-1 RT (0.5 units; Ambion, Austin, Tex.) was incubated with various concentrations of compounds for 5 minutes at room temperature. Then a template-primer mixture was added to reach final concentration of 5 μg/mL oligo(dT)₂₀, 10 μg/mL poly(rA), 1.25 μM [α-32P]dTTP and 10 μM dTTP. The mixture was incubated at 37° C. for 60 minutes. Aliquots of the reaction were spotted on DEAE paper, washed twice with 2×SSC buffer (300 mM NaCl, 30 mM sodium citrate) and once with 95% ethanol, dried and exposed to PhosphorImager screen (Molecular Dynamics, Sunnyvale, Calif.). The screens were scanned by FLA-5100 instrument (Fujifilm Life Science, Stamford, Conn.) and the amount of incorporated labeled phosphate was used to quantify the RT activity. The best fitted curves and IC₅₀ values were calculated using Prism 4 software (GraphPad Software, San Diego). The degree of synergism between screen compounds and AZT were determined by testing the compounds in the RT assay individually and in combinations at a fixed molar ratio over a range of serial dilutions (Murga et al. Antimicrob. Agents Chemother. 50(10):3289-96, 2006). The data were then analyzed by the isobologram technique, which evaluates the compound interactions by a dose-oriented geometric method (Richman et al. Antimicrob. Agents Chemother., 35(2):305-8, 1991 and Chou and Talalay, Adv. Enzyme Regul., 22:27-55 1984). For the in vitro studies, a triphosphate form of AZT (EMD Biosciences, San Diego, Calif.) was used.

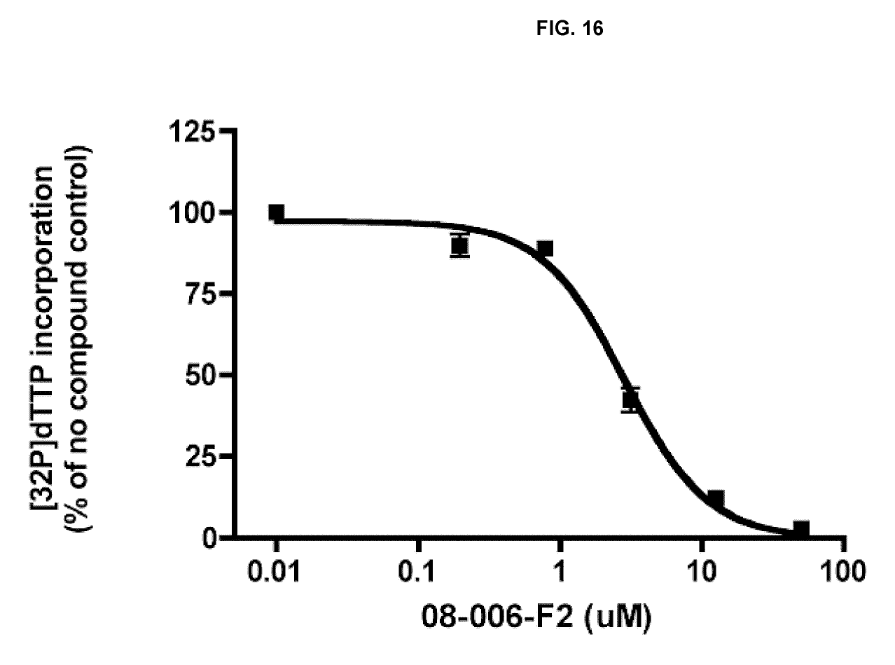

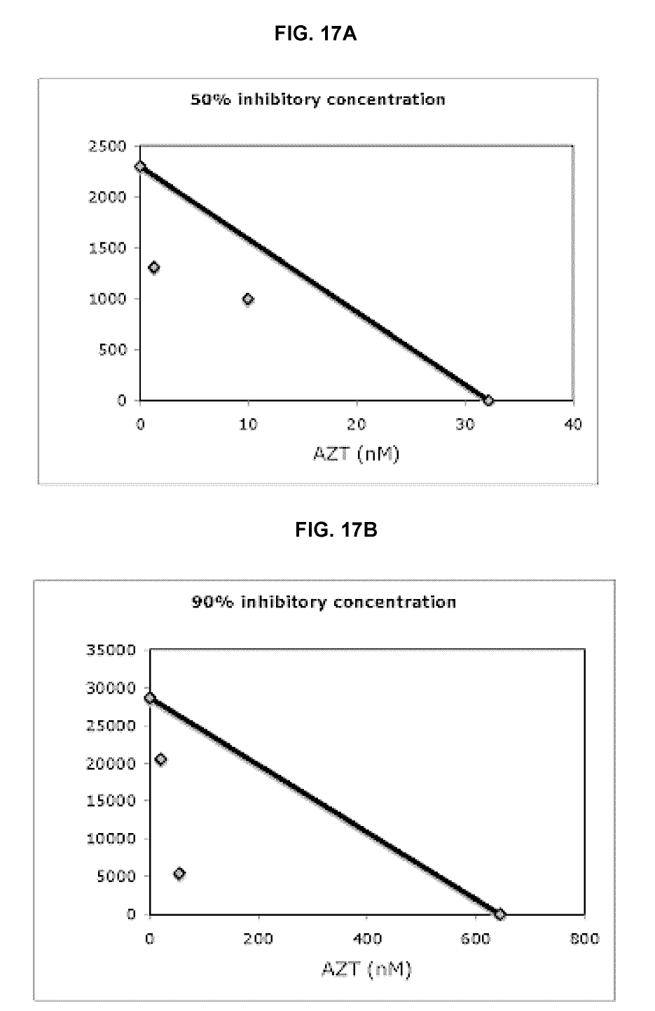

The results of a typical assay in vitro assay of the activity of the disclosed compounds against purified HIV-1 reverse transcriptase is illustrated in the graph of FIG. 16. Specifically, FIG. 16 is a dose-response curve for inhibition of HIV-1 reverse transcriptase activity by inhibitor 08-006-F2 (IC₅₀ 2.8 μM, 95% confidence interval 2.3-3.5 μM). FIGS. 17A and 17B are isobologram plots of inhibition in the purified HIV-1 reverse transcriptase assay for combinations of AZT and 08-006-F2 demonstrating the synergistic effect of these compounds. The line between the 50% or 90% fractional inhibitory concentrations for single compounds indicates the values at which additive effects would occur. Values below and to the left of the lines indicate synergy.

Real-time quantitation of viral DNA was accomplished using PCR as follows. The total cellular DNA was harvested 24 hours post infection with the AccuPrep genomic DNA extraction kit (Bioneer Life Science Corp., Rockville, Md.). The amount of viral DNA products was quantified by real-time PCR on ABI Prism 7900 Sequence Detection System (Applied Biosystems, Foster City, Calif.) with the following primers and probes: early HIV-1 reverse transcripts with primers ert2f, ert2r, probe ERT2 (Munk et al., Proc. Natl. Acad. Sci. 99(21):13843-48; 2002, herein incorporated by reference as to the sequence of the primers), late HIV-1 reverse transcripts with primers MH531, MH532, probe LRT-P, 2-LTR circular DNA with primers MHH535, MH536, probe MH603 (Butler et al., Nat. Med. 7(5):631-4; 2001, herein incorporated by reference as to the sequence of the primers and probes). To control the number of cell equivalent DNA in the samples, a single-copy locus in the porphobilinogen deaminase (PBGD) gene (Buckman et al., J. Virol. 77(2):1469-80; 2003) was amplified with primers PBGD1 (5′-AAGGGATTCACTCAGGCTCTTTC; SEQ ID NO: 1) and PBGD2 (5′-GGCATGTTCAAGCTCCTTGG; SEQ ID NO: 2) and probe PBGD-P (5′-VIC-CCGGCAGATTGGAGAGAAAAGCCTGT-MGBNFQ; SEQ ID NO: 3).

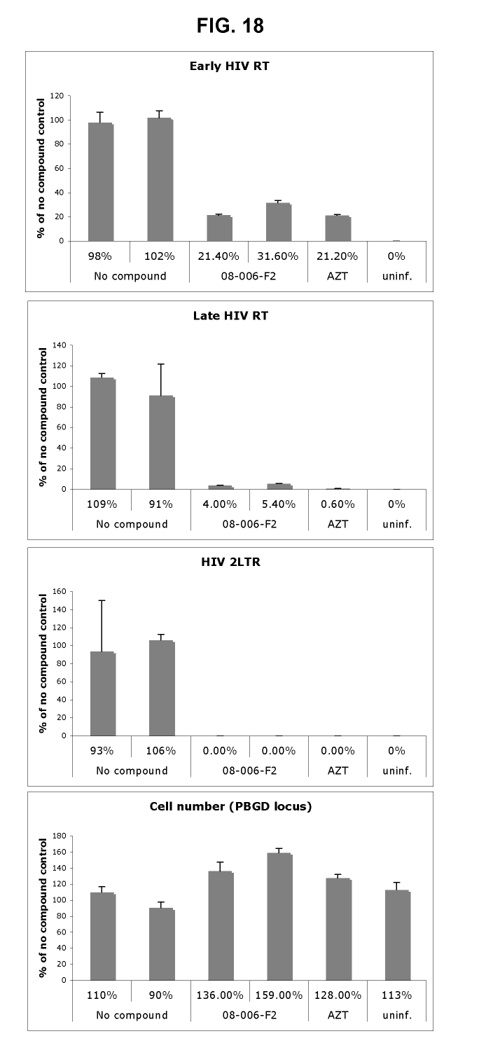

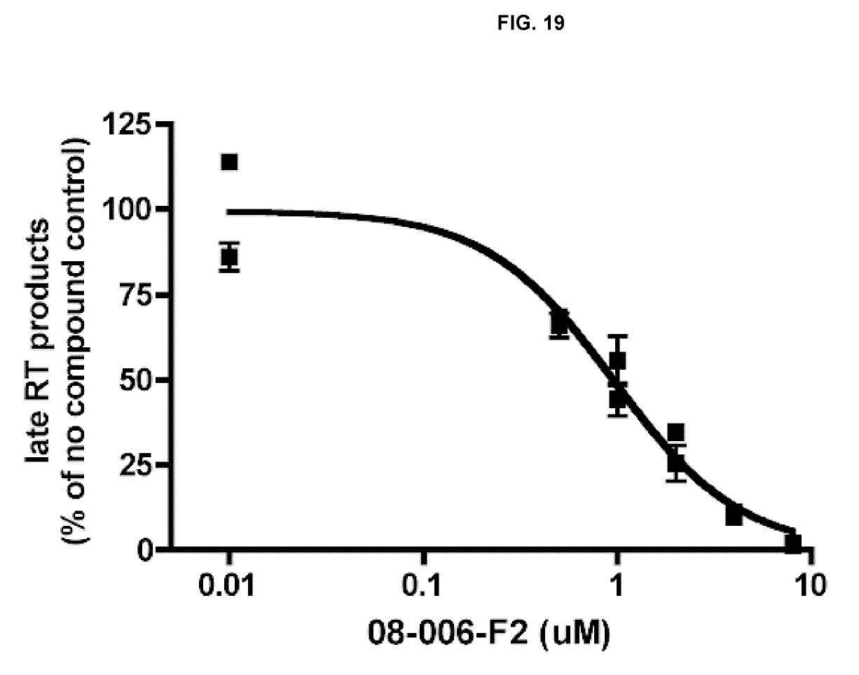

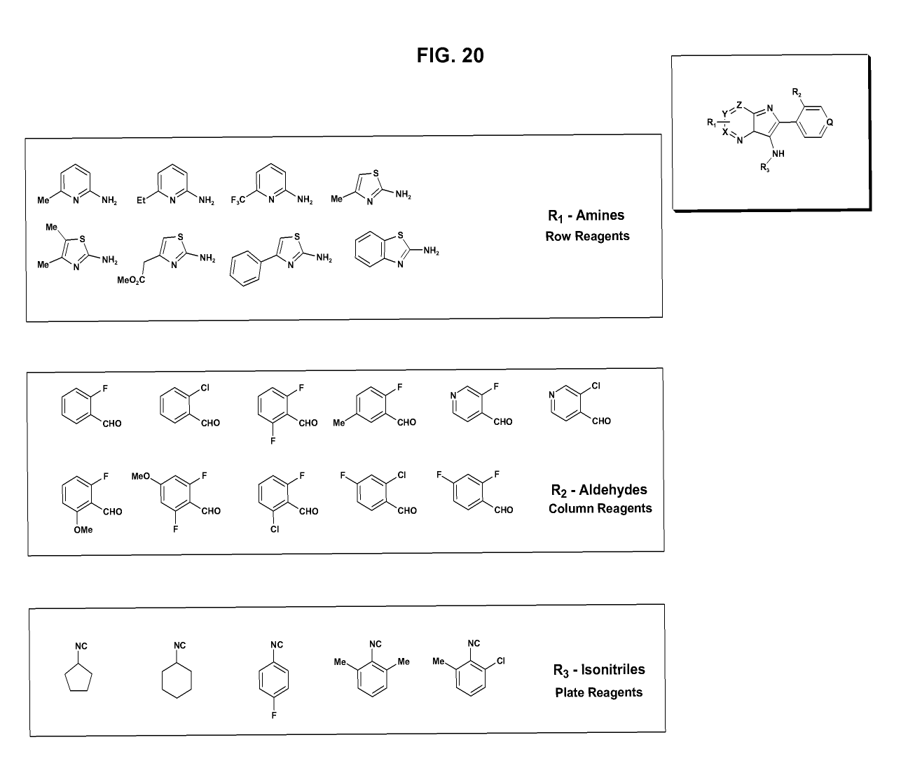

The quantitative real-time PCR-based approach described above was used to determine whether the presently disclosed compounds, such as 08-006-F2, block HIV infection at a step prior to the synthesis of late viral DNA products. Using different specific primers and probes, the amount of early viral DNA products, late viral DNA products, and 2LTR-containing circular forms of viral DNA, were quantified 24 hours post infection. Similarly to AZT, which was used as a reference compound, 08-006-F2 blocked the synthesis of early and late viral DNA, confirming the effect on the viral reverse transcription process (FIG. 18). As shown in F1G. 18, the effect of 5 μM compound 08-006-F2 and 5 μM AZT on the synthesis of viral DNA products was measured by quantitative real-time PCR. Quantitation of early and late HIV-1 reverse transcription products is illustrated, as is quantitation of 2-LTR containing circular DNA forms (the circular DNA forms serve as a surrogate marker for nuclear translocation of viral DNA). Therefore, 08-006-F2 and AZT block at the reverse transcription step. A dose-dependent inhibition of HIV-1 late RT product formation by 08-006-F2 is shown in FIG. 19. The amplification of porphobilinogen deaminase (PBGD) gene serves as a control of cellular equivalents in the samples.

It also was investigated whether 08-006-F2 exerts an additional effect on provirus gene expression. This effect was not observed, the compound was found to have no significant influence on cells that express luciferase from established HIV-1 proviral DNA (data not shown).

### Example 6

This example describes methods for screening and evaluating the disclosed compounds for reverse transcriptase inhibition, including the ability to inhibit reverse transcriptase in HIV mutants known to be resistant to other NNRTIs.

Initial screening employed a plate-based assay to identify those that inhibit infection of human 293T cells as described in Example 5, except that in addition to a wild-type HIV vector, vectors with mutant HIV were also used. The mutant HIV-1 vectors, based on the same HIV-1 NL4-3 strain described in Example 5, had one of four mutations introduced into the RT region (Y188L, Y181C, V106A, or K103N) and a deletion in the Env gene to make them not replication competent (see Julias et al., Virology, 322:13-21, 2004, herein incorporated by reference as to the vectors). The four mutants assayed are the most commonly encountered resistance mutants for the current NNRTIs. One skilled in the art will appreciate that other RT mutants can be screened using these methods, such as G190E or the double mutant K103N/Y181I.

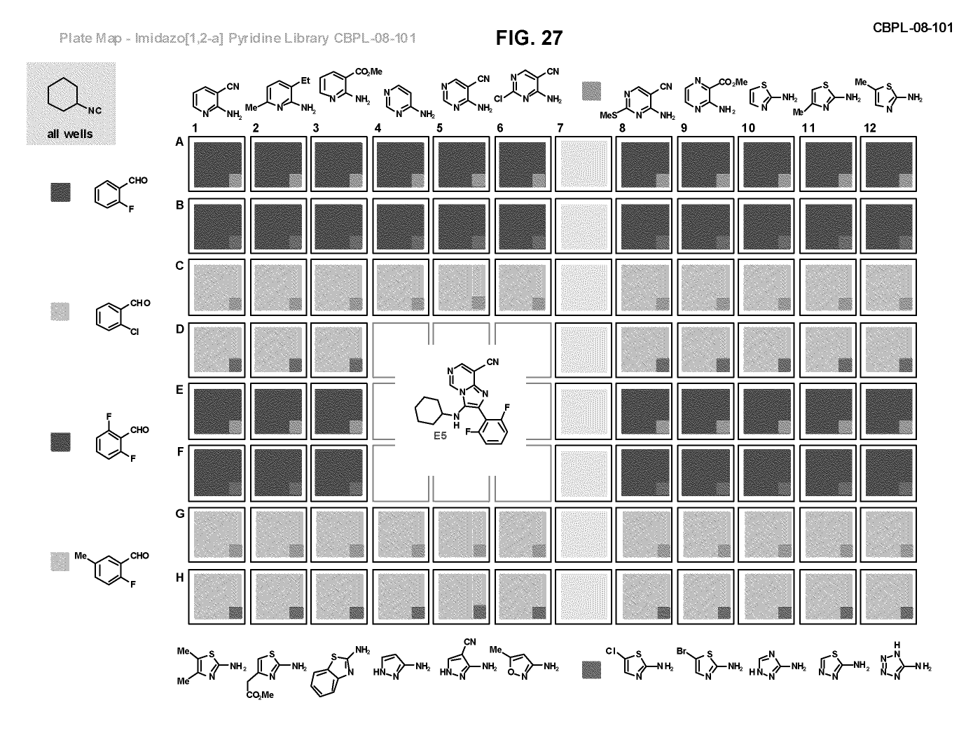

Briefly, 10,000 human 293T cells were plated in 80 μL medium in each well of 96-well tissue culture plate. The next day, 10 μL of each diluted compound shown in FIGS. 26 and 27 was added to reach a final concentration of 2 μM, and incubated at 37° C. for 1 hour. Controls included 08-006-F2, 04-035-E1, nevirapine (NVP), and AZT (each control added to reach a final concentration of 2 μM). A 10 μL aliquot of medium containing the wild-type (pNL4-3LucR+E-) or mutant HIV-1 vector (p24 antigen) was then added to each well. The mutant HIV-1 vectors (pNLNgoMIV R+E-.luc) are described in Julias et al. (Virol. 322:31-21, 2004). Twenty-four hours after viral challenge, 50 μL of the medium was removed and 50 μL of Bright-Glo reagent (Promega, Madison, Wis.) was added to lyse the cells and provide the luciferin substrate for virus-encoded firefly luciferase. After several minutes the luminescence associated with each sample was measured, and served as readout to quantify virus infectivity in each well.

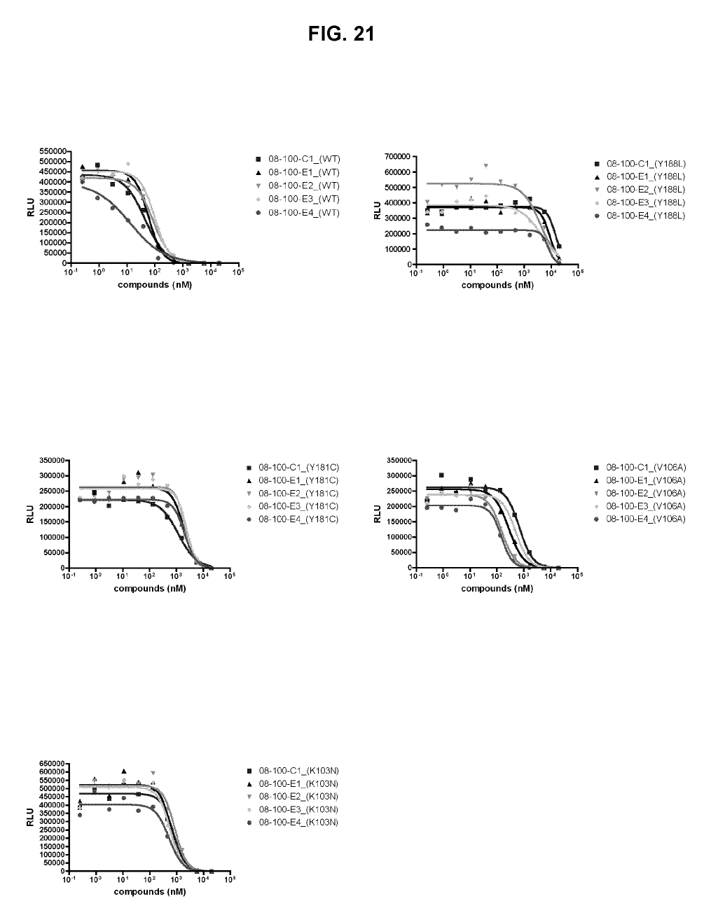

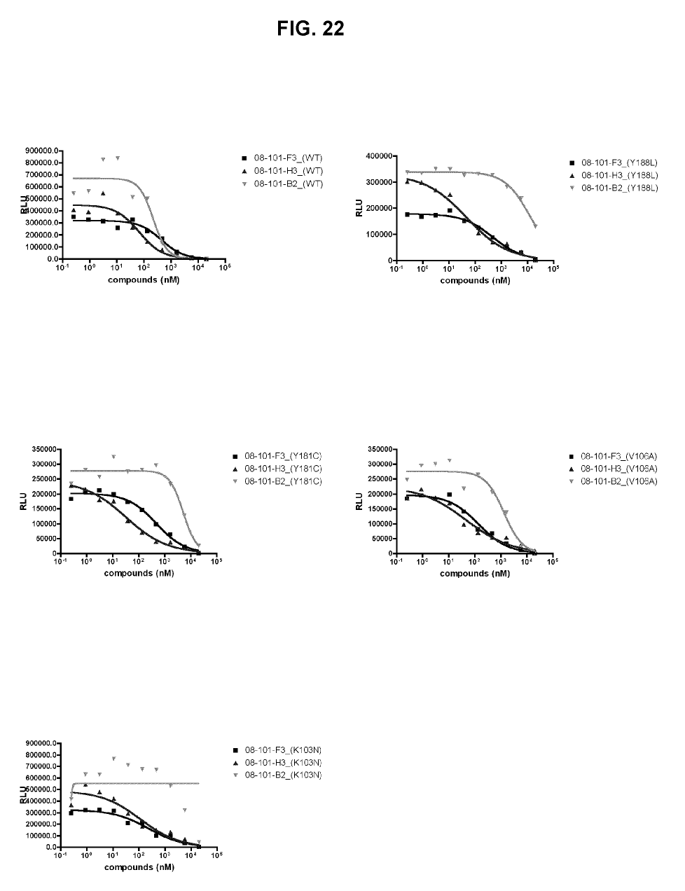

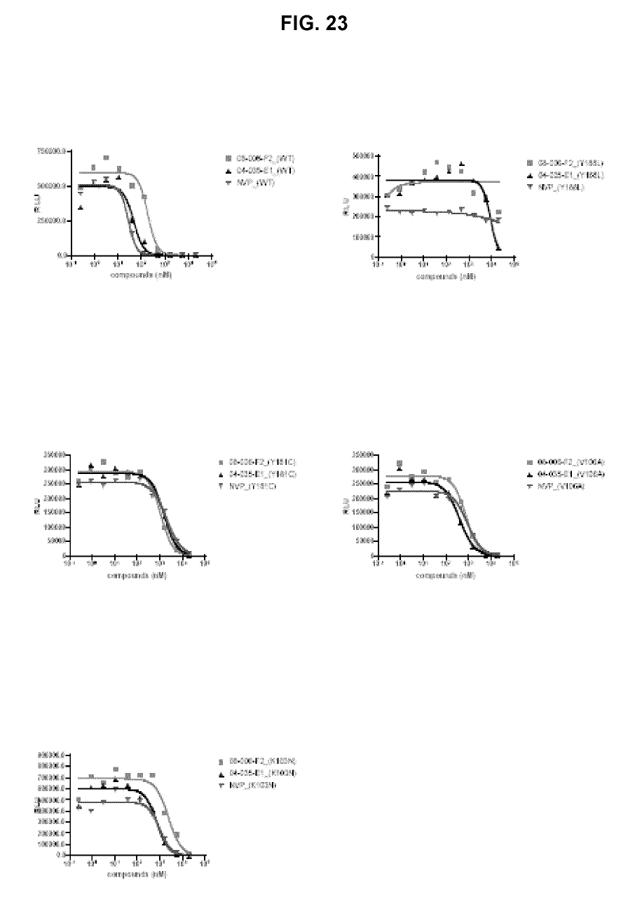

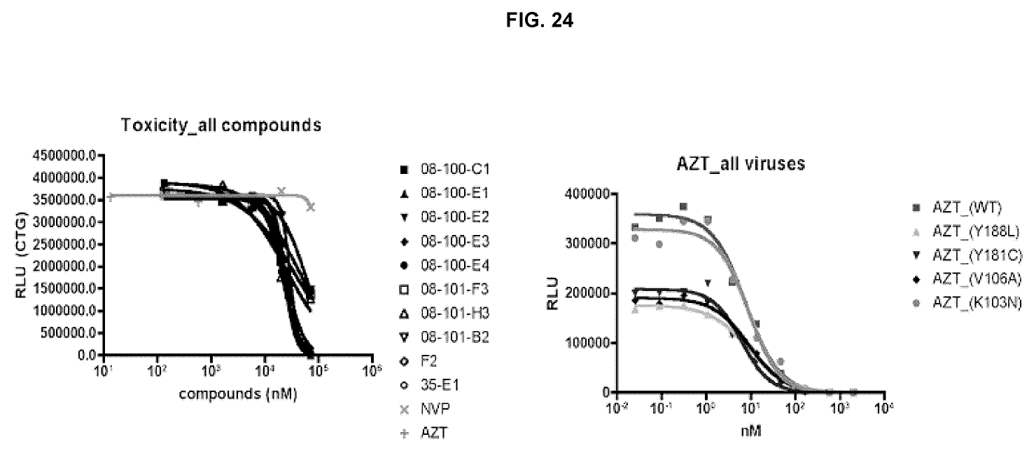

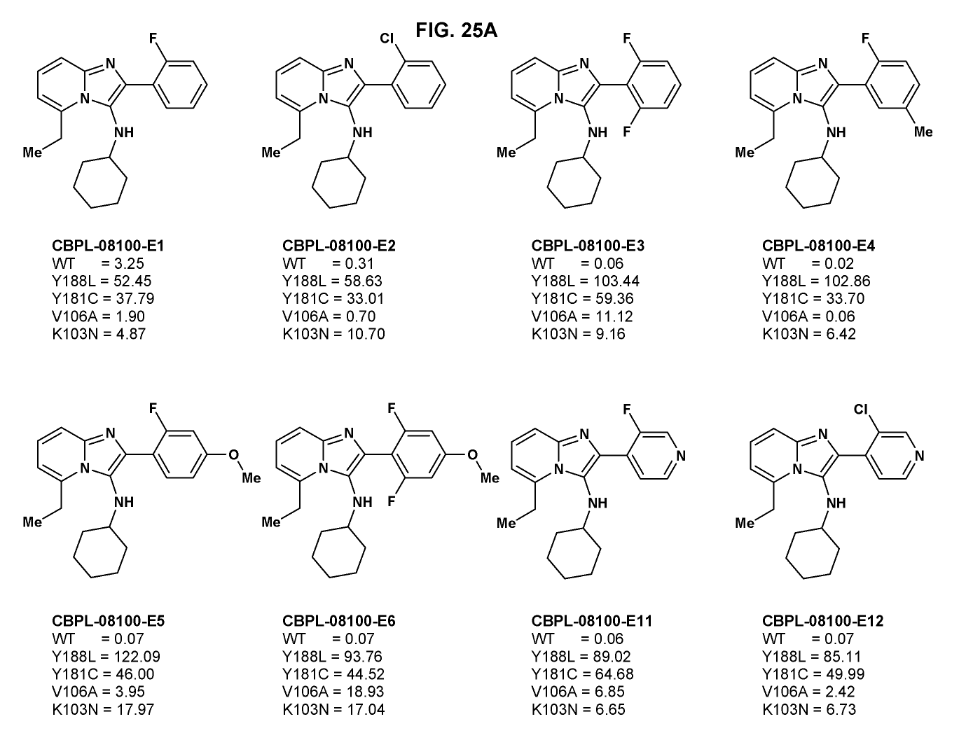

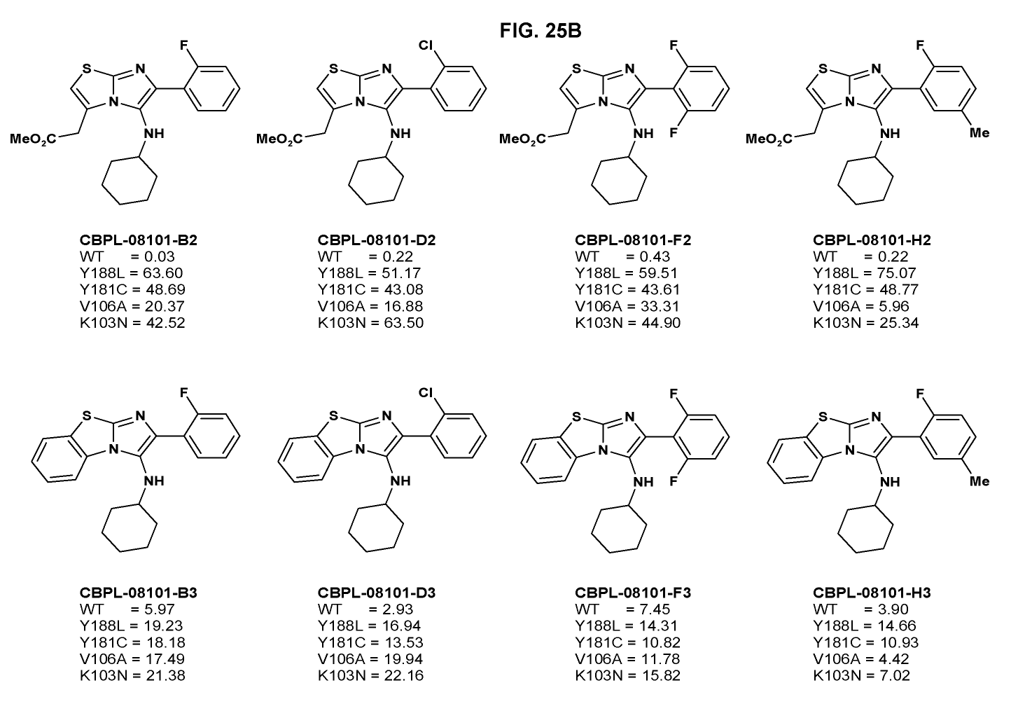

Using the methods described in Example 5, a dose-response curve (IC₅₀) for each wild-type and mutant vector was obtained for the eight compounds that provided the best results, as well as the control compounds (08-006-F2, 04-035-E1, NVP, and AZT). The results are shown in FIGS. 21-24 and Tables 3 and 4. As shown in FIG. 22, compounds 08-101-H3 and 08-101-F3 (see FIG. 25 for chemical structures) significantly reduced reverse transcriptase activity, even in the RT mutants resistant to other NNRTIs. Dose response curves were repeated in three replicates for compounds 08-101-H3 and 08-101-F3 to confirm the results (Table 4). FIG. 24 (left panel) shows the toxicity response curve of several compounds using 293T human cells and the methods described above, with the ATP content measured by the CellTiter-Glo kit (Promega) 24 hours after compound addition. FIG. 24 (right panel) shows inhibition of wild-type and NNRTI mutant viruses by another control compound, AZT. These results demonstrate that the viruses can be efficiently inhibited by the NRTI compound AZT. It was observed that compounds 08-101-B3, 08-101-D3, 08-101-F3, 08-101-H3 have at least some false-positive luciferase inhibitory activity. Therefore, a part of the observed inhibitory effect is likely due to luciferase inhibition by the compound in addition to RT inhibition.

TABLE 3
EC50 of NNRTIs for wild-type and mutant HIV reverse transcriptases

| | Virus genotype | | | | | |
|---|---|---|---|---|---|---|
| | WT | | Y188L | | Y181C | |
| | EC50 | | EC50 | | EC50 | |
| Compound | (nM) | 95% conf. int. | (nM) | 95% conf. int. | (nM) | 95% conf. int. |
| 08-100-C1 | 43.49 | 27.76 to 68.14 | 14653.00 | 10510 to 20429 | 1182.00 | 813.0 to 1717 |
| 08-100-E1 | 58.88 | 50.08 to 69.22 | 8760.00 | 5538 to 13857 | 1853.00 | 1120 to 3066 |
| 08-100-E2 | 86.13 | 64.61 to 114.8 | 3929.00 | 1753 to 8805 | 2130.00 | 1294 to 3507 |
| 08-100-E3 | 97.69 | 72.98 to 130.7 | 4289.00 | 2096 to 8776 | 2368.00 | 1372 to 4089 |
| 08-100-E4 | 12.76 | 3.583 to 45.41 | 7720.00 | 4823 to 12358 | 2025.00 | 1771 to 2316 |
| 08-101-F3 | 436.70 | 232.6 to 819.8 | 473.50 | 284.0 to 789.7 | 467.30 | 288.9 to 755.7 |
| 08-101-H3 | 66.28 | 25.41 to 172.9 | 46.48 | 18.32 to 118.0 | 32.16 | 13.52 to 76.48 |
| 08-101-B2 | 217.70 | 82.93 to 571.4 | 12429.00 | 9631 to 16041 | 5031.00 | 3004 to 8427 |
| F2 | 185.30 | 108.1 to 317.8 | na | na | 1074.00 | 739.9 to 1560 |
| 35-E1 | 46.24 | 22.84 to 93.65 | 9001.00 | 4738 to 17098 | 1533.00 | 1029 to 2284 |
| NVP | 27.39 | 20.04 to 37.43 | 278969.00 | 4737 to 1.643e+007 | 1995.00 | 1427 to 2790 |
| AZT | 7.70 | 5.238 to 11.32 | 9.35 | 7.322 to 11.95 | 6.12 | 3.884 to 9.654 |

| | Virus genotype | | | | | |
|---|---|---|---|---|---|---|
| | V106A | | K103N | | Toxicity | |
| | EC50 | | EC50 | | EC50 | |
| Compound | (nM) | 95% conf. int. | (nM) | 95% conf. int. | (nM) | 95% conf. int. |
| 08-100-C1 | 689.30 | 414.2 to 1147 | 746.70 | 464.3 to 1201 | 41297.00 | 22690 to 75163 |
| 08-100-E1 | 277.70 | 190.8 to 404.0 | 593.40 | 356.4 to 988.1 | 22591.00 | 15255 to 33455 |
| 08-100-E2 | 150.60 | 127.3 to 178.2 | 854.60 | 485.9 to 1503 | 22278.00 | 16229 to 30581 |
| 08-100-E3 | 494.20 | 355.7 to 686.4 | 565.60 | 328.0 to 975.2 | 25174.00 | 18900 to 33531 |
| 08-100-E4 | 146.80 | 110.5 to 195.1 | 515.80 | 287.6 to 925.1 | 20896.00 | 15825 to 27592 |
| 08-101-F3 | 140.40 | 68.43 to 288.1 | 236.50 | 87.05 to 642.6 | 34616.00 | 12974 to 92353 |
| 08-101-H3 | 47.06 | 8.405 to 263.5 | 112.50 | 18.35 to 689.3 | 24260.00 | 4256 to 138266 |
| 08-101-B2 | 1304.00 | 628.1 to 2708 | na | | 56652.00 | 37963 to 84541 |
| F2 | 763.90 | 471.1 to 1239 | 2356.00 | 1126 to 4928 | 29214.00 | 13459 to 63411 |
| 35-E1 | 398.30 | 202.9 to 781.9 | 719.80 | 373.5 to 1387 | 24055.00 | 12879 to 44930 |
| NVP | 888.30 | 573.6 to 1376 | 977.00 | 513.9 to 1857 | 105833.00 | |
| AZT | 9.14 | 6.521 to 12.80 | 8.76 | 5.361 to 14.31 | na | na |

TABLE 4
EC50 of NNRTIs for wild-type and mutant HIV reverse transcriptases

| compound | virus | EC50 (nM) | SE(logEC50) | Hill slope | EC70 | EC90 | EC95 |
|---|---|---|---|---|---|---|---|
| 08-101-H3 | 1a (WT) | 12.98 | 0.11 | −0.59 | 54.88 | 545.71 | 1945.97 |
| | 3a (Y188L) rep1 | 28.59 | 0.08 | −0.59 | 120.02 | 1180.13 | 4182.07 |
| | 3a (Y188L) rep2 | 19.95 | 0.07 | −0.70 | 67.27 | 466.46 | 1362.49 |
| | 4a (Y181C) | 17.66 | 0.08 | −0.55 | 82.98 | 976.30 | 3821.09 |
| | 5a (V106A) | 27.91 | 0.15 | −0.61 | 112.69 | 1041.27 | 3565.26 |
| | 6a (K103N) | 65.81 | 0.14 | −0.83 | 181.59 | 914.89 | 2239.24 |
| 08-101-F3 | 1a (WT) | 91.41 | 0.08 | −0.95 | 223.54 | 929.20 | 2044.57 |
| | 3a (Y188L) rep1 | 193.50 | 0.07 | −0.90 | 494.68 | 2206.88 | 5049.73 |
| | 3a (Y188L) rep2 | 95.25 | 0.08 | −0.84 | 260.83 | 1298.32 | 3156.46 |
| | 4a (Y181C) | 119.80 | 0.09 | −0.88 | 313.98 | 1457.35 | 3408.61 |
| | 5a (V106A) | 248.20 | 0.11 | −1.40 | 454.81 | 1193.69 | 2036.34 |
| | 6a (K103N) | 275.20 | 0.17 | −1.12 | 585.22 | 1947.04 | 3787.41 |
| 08-100-E4 | 1a (WT) | 35.47 | 0.07 | −0.91 | 89.97 | 396.40 | 900.79 |
| 08-100-C4 | 1a (WT) | 79.17 | 0.06 | −1.16 | 164.46 | 527.10 | 1004.35 |

### Example 7

This example describes methods that can be used to confirm the efficacy of the disclosed compounds in vivo in an animal model to, for example confirm the ability of the NNRTIs to substantially reduce or inhibit the activity of HIV-1 reverse transcriptase, HIV infection and replication. Although particular animals and methods are provided, one skilled in the art will appreciate that other animals, doses, and modes of administration can be used.

Pigtail or rhesus macaques (e.g., 6 to 12 months of age) are infected with a chimeric simian-human immunodeficiency viruses (SHIVs), which includes SIV and HIV-1 reverse transcriptase. In one example the chimeric virus is RT-SHIVₘₙₑ, which is inhibited by all currently FDA approved RT inhibitors (see Ambrose et al., J. Virol. 78:13553-61, 2004). Animals are sedated (intramuscular injection of 3 to 6 mg/kg tiletamine-zolazepam or 10 to 15 mg/kg ketamine) and then infected intravenously with virus, such as 10³ to 10⁸ (e.g., 10⁵) infectious units of virus.

Viral loads can be monitored using routine methods. For example, viral RNA can be extracted from plasma and quantitative RT-PCR performed to determine the number of viral genomes per ml of plasma. Lymphocyte subsets (CD3, CD4, CD8, and CD20) are measured by staining whole blood and analyzed by flow cytometry. Initial plasma virus peaks are routinely observed 1 to 4 weeks post infection. CD4+ T cell counts are typically 700 to 1700 cells/μL prior to infection, and decline to below 500 or even below 200 cells/μL after infection. Animals that develop untreatable symptoms associated with AIDS (e.g., moderate to severe weight loss and respiratory or gastrointestinal pathologies) can be euthanized.

Animals can be divided into treated and untreated control groups. The treated group receives therapeutically effective amounts of one or more of the disclosed NNRTIs, such as a range of doses (e.g. 10 to 1000 mg of the NNRTI). In some examples, the disclosed NNRTIs are administered with other therapeutic agents, such as at least one or two NRTIs and/or additional antiviral agents. For example the treated group can orally receive from about 0.1 to about 50 mg/kg, such as from about 2 to about 15 mg/kg, or from about 1 to about 20 mg/kg daily during the trial period.

The untreated control group does not receive therapeutic agents (e.g., receives only a pharmaceutically acceptable carrier or other placebo).

In some examples the animals also include a third group (positive control), which are treated with NNRTIs known to be effective, such as FDA approved NNRTIs. For example the positive control group can receive three doses of efavirenz (EFV) from 200 mg capsules. In one example oral administration is performed during week 13, days 1, 2 and 4. At week 17, animals are given daily doses of tenofovir disoproxil fumarate (TDF) (20 mg/kg) and emtricitabine (FTC) (50 mg/kg) subcutaneously and EFV (200 mg in food) orally for up to 20 weeks. Drug administration can be discontinued for 2 to 4 weeks until plasma viral loads reach 3000 copies/mL or higher. All three drugs are then restarted until 45 weeks post-infection (3 to 5 additional weeks). In another examples, oral administration of 200 mg EFV is performed daily beginning at week 6 post infection, for example until at least 30, at least 40, or at least 52 weeks post-infection. Other exemplary positive control administrations are described in Hofman et al. (Antimicrob. Agents Chemother. 48:3483-90, 2004; hereby incorporated by reference as to the methods). EFV levels can be measured in plasma samples by high-performance liquid chromatography.

Animals are monitored for the presence of viral RNA, and lymphocyte counts, to confirm the efficacy of the NNRTIs, to obtain a dose-response curve, or to identify the IC₅₀. Viral RNA is extracted from plasma and quantitative RT-PCR performed to determine the number of viral genomes per ml of plasma. Lymphocyte subsets (CD3, CD4, CD8, and CD20) are measured by staining whole blood and analyzed by flow cytometry. NNRTIs that decrease viral RNA to less than 400 copies of viral RNA per ml blood and/or a CD4+ count of greater than 200 CD4+ cells per microliter typically are considered to be efficacious in vivo.

### Example 8

This example describes a particular method that can be used to treat HIV in a human subject by administration of one or more of the disclosed NNRTIs. Although particular methods, dosages, and modes of administrations are provided, one skilled in the art will appreciate that variations can be made without substantially affecting the efficacy of treatment.

Based upon the teaching disclosed herein, HIV, such as HIV type 1 (HIV-I) or HIV type 2 (HIV-II), can be treated by administering a therapeutically effective amount of one or more of the disclosed NNRTIs (such as 04-035-E1, 04-034-E1, 04-034-E2), which in turn in reduces or eliminates HIV infection, replication or a combination thereof.

Briefly, the method can include screening subjects to determine if they are infected with HIV, such as HIV-I or HIV-II. Subjects infected with HIV are selected. In one example, subjects having increased levels of HIV antibodies in their blood (for example as detected with an enzyme-linked immunosorbent assay, Western blot, immunofluorescence assay, or nucleic acid testing, including viral RNA or proviral DNA amplification methods) or HIV RNA in their plasma (for example detected using RT-PCR) are selected. In one example, a clinical trial would include half of the subjects following a currently established protocol for treatment of HIV (such as a HAART). The other half would receive a currently established protocol for treatment of HIV (such as treatment with HAART) in combination with administration of one or more of the NNRTIs provided herein. In another example, a clinical trial would include half of the subjects following the established protocol for treatment of HIV (such as a HAART). The other half would receive one or more of the NNRTIs provided herein.

### Screening Subjects

In particular examples, the subject is first screened to determine if it is infected with HIV, such as a mutant HIV (for example HIV with one or more of the following mutations: L74V, V75I, A98G, L100I, K101E/D/C, K103N, V106A/M, V108I/M, E138K, Q145M, Y181C/I, Y188L/C/H, G190S/A/E, M230L, P225H, P236L, Y318F, and N348I). Examples of methods that can be used to screen for HIV include a combination of measuring a subject's CD4+ T cell count and the level of HIV antibodies or RNA in serum.

In some examples, HIV testing consists of initial screening with an enzyme-linked immunosorbent assay (ELISA) to detect antibodies to HIV, such as to HIV-1. Specimens with a nonreactive result from the initial ELISA are considered HIV-negative unless new exposure to an infected partner or partner of unknown HIV status has occurred. Specimens with a reactive ELISA result are retested in duplicate. If the result of either duplicate test is reactive, the specimen is reported as repeatedly reactive and undergoes confirmatory testing with a more specific supplemental test (e.g., Western blot or an immunofluorescence assay (IFA)). Specimens that are repeatedly reactive by ELISA and positive by IFA or reactive by Western blot are considered HIV-positive and indicative of HIV infection. Specimens that are repeatedly ELISA-reactive occasionally provide an indeterminate Western blot result, which may be either an incomplete antibody response to HIV in an infected person, or nonspecific reactions in an uninfected person. IFA can be used to confirm infection in these ambiguous cases. In some instances, a second specimen will be collected more than a month later and retested for subjects with indeterminate Western blot results. In some examples, nucleic acid testing (e.g., viral RNA or proviral DNA amplification method) is used to diagnosis HIV infection.

The detection of HIV in a subject's blood is indicative that the subject is infected with HIV and is a candidate for receiving the one or more of the NNRTIs provided herein. Moreover, detection of a CD4+ T cell count below 350 per microliter, such as 200 cells per microliter, is also indicative that the subject is likely to be infected with HIV.

Pre-screening is not required prior to administration of the therapeutic compositions disclosed herein (such as those that include one or more of the NNRTIs provided herein).

Subjects also may be screened to determine if they are infected with a resistant strain of HIV, in particular a strain resistant to one or more reverse transcriptase inhibitors. Dozens of mutant strains have been characterized as resistant to NNRTI compounds, including L74V, V75I, A98G, L100I, K101E/D/C, K103N, V106A/M, V1081I/M, E138K, Q145M, Y181C/I, Y188L/C/H, G190S/A/E, M230L, P225H, P236L, Y318F, N348I, and combinations of such mutations. In particular, the Y181C and K103N mutants currently are believed to be the most difficult to treat using current treatment protocols. The present compounds are particularly useful in treating subjects infected with these resistant strains. Subjects infected by a resistant strain can be identified by their lack of response to a drug treatment protocol, or with more specificity using phenotypic and/or genotypic characterization methods. Thus, one method for identifying subjects for treatment includes collecting a biological sample from an HIV-infected subject; and determining whether the biological sample comprises nucleic acid encoding HIV reverse transcriptase having a mutation. As is known to those of skill in the art, reverse transcriptase-polymerase chain reaction-based methods can be used to amplify viral RNA isolated from viral particles present in the serum of HIV-infected individuals and to determine whether the RNA contains mutations that would confer resistance. For example, as described in U.S. Pat. No. 7,037,644, which is incorporated herein by reference, strains having nucleic acid encoding HIV reverse transcriptase having a mutation at codon 181 and 227 had an increase in delavirdine susceptibility and a significant decrease in nevirapine susceptibility as well as an increase in efavirenz susceptibility. The mutated codon 227 codes for a leucine and mutated codon 181 codes for a cysteine. Similarly, strains having nucleic acid encoding HIV reverse transcriptase having a mutation at codon 106 and 181 and 227 were observed to have a moderate decrease in delavirdine susceptibility and a substantial decrease in nevirapine susceptibility and a slight decrease in efavirenz susceptibility. The mutated codon 106 codes for an alanine, codon 181 codes for a cysteine and codon 227 codes for a leucine. In another example, strains having nucleic acid encoding HIV reverse transcriptase having a mutation at codons 103 alone or in combination with a mutation at codon 188 had a substantial decrease in delavirdine susceptibility and a substantial decrease in nevirapine susceptibility and a substantial decrease in efavirenz susceptibility. The mutated codon 188 codes for a leucine and codon 103 codes for an asparagine. Additional, known and future emerging mutant strains can be identified for treatment with the present compounds using similar techniques as is known to those of skill in the art.

### Pre-Treatment of Subjects

In particular examples, the subject is treated prior to administration of a therapeutic agent that includes one or more of the NNRTIs provided herein. However, such pre-treatment is not always required, and can be determined by a skilled clinician. For example, the subject can be treated with an established protocol for treatment of HIV (such as a currently established HAART protocol).

### Administration of Therapeutic Compositions

Following subject selection, a therapeutically effective dose of one or more of the NNRTIs provided herein is administered to the subject (such as an adult human or a newborn infant either at risk for contracting HIV or known to be infected with HIV). For example, a therapeutic effective dose of a composition including one or more of the NNRTIs provided herein is administered to the subject to reduce or inhibit HIV replication. Additional antiviral agents, such as other NNRTIs, NRTIs (such as AZT), PIs and other anti-viral agents, can also be administered to the subject simultaneously or prior to or following administration of the disclosed NNRTIs. Administration can be achieved by any method known in the art, such as by oral administration although other modes of administration can be selected, such as including without limitation intravenous administration.

In some particular examples, the therapeutic composition includes at least one presently disclosed inhibitor compound, such as 04-034-A1; 04-034-B10; 04-034-E1; 04-034-E2; 04-034-E10; 04-034-H1; 04-034-H2; 04-034-H9; 04-034-H10; 04-035-E1; 04-036-E1; 04-036-E10; 04-037-E1; or from library CBPL-08-100 and with reference to the structures in FIG. 25, compound E1; E2; E3; E4; ES; E6; E11; E12; or from library CBPL-08-101 compound B2; D2; F2; H2; B3; D3; F3; H3; or combinations thereof. In one specific example, the composition is administered orally at from 10 to about 300 mg/kg of inhibitor every day for a selected time, such as from one to ten weeks or, if a positive result is obtained (for example, less than 400 copies of viral RNA per ml blood and a CD4+ count of greater than 200 CD4+ cells per microliter), the composition can continue to e administered, depending upon the particular stage of HIV. In an example, the therapeutic agents are administered continuously (e.g., one, two or three times daily, daily, two or three times a week).

The amount of the composition administered to prevent, reduce, inhibit, and/or treat HIV or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., HIV infection or AIDS) in a subject without causing a substantial cytotoxic effect in the subject. An effective amount can be readily determined by one skilled in the art, for example using routine trials establishing dose response curves. In addition, particular exemplary dosages are provided herein. The therapeutic compositions can be administered in a single dose delivery, via continuous delivery over an extended time period, in a repeated administration protocol (for example, by a daily, weekly, or monthly repeated administration protocol). In one example, therapeutic agents that include one or more of the NNRTIs provided herein are administered orally to a human. As such, these compositions may be formulated with a pharmaceutically acceptable carrier.

Administration of the therapeutic compositions can be taken long term (for example over a period of months or years).

### Assessment

Following the administration of one or more therapies, subjects having HIV (for example, HIV-I or HIV-II) can be monitored for reductions in HIV levels, increases in CD4+ T cell count, or reductions in one or more clinical symptoms associated with HIV or AIDS. In particular examples, subjects are analyzed one or more times, for example starting at least 7 days or at least 14 days following treatment. Subjects can be monitored using any method known in the art. For example, biological samples from the subject, including blood, can be obtained and alterations in HIV or CD4+ T cell levels evaluated. In certain situations when the disclosed NNRTIs either administered alone or in a cocktail including other antiviral agents yield a decrease viral RNA levels by at least about 2-orders of magnitude, or an increase in CD4+ cell levels to at least 200 CD4+ cells per microliter of blood, within 1 week of therapy, the compound or would be considered to be efficacious in vivo.

### Additional Treatments

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50%, or at least 70% in HIV infection, HIV replication or combination thereof. A partial response may also be an increase in CD4+ T cell count such as at least 350 T cells/mL.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A compound according to the formula 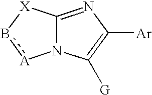
wherein A is N, O, S or CR¹;
B is N or CR²
X is CH; CR, where R is H, cyano, halogen, aliphatic, particularly haloalkyl and lower aliphatic, —OR₉, —NR¹⁰R¹¹, or is an atom or atoms in a fused ring; O; or S; 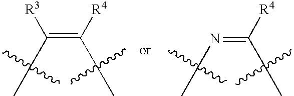
R¹, R², R³ and R⁴ independently are selected from H; cyano; halogen; haloalkyl; lower aliphatic; —OR₉; and —NR¹⁰R¹¹; and two of R¹, R², R³ and R⁴ together may optionally form a fused ring;
Ar is a 5 or 6 membered aromatic ring of the formula 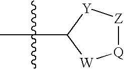
Y is S; N or CR⁵;
Z is S; N; CR⁶; 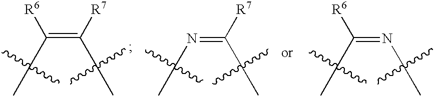
Q is S; N or CR⁸
W is S; N or CR⁹
R⁵-R⁹ independently are selected from H; cyano; halogen; haloalkyl; lower alkyl; —OR¹⁰; —SR¹¹; —NR¹²R¹³; and wherein two of R⁵-R⁹ together optionally may form a fused ring;
R¹⁰, R¹¹, R¹² and R¹³ independently are H, alkyl or acyl;
G is selected from —NR¹⁴R¹⁵ or —N═R¹⁶;
R¹⁴ and R¹⁵ independently are selected from H; aralkyl; lower alkyl; aryl; acyl; —C(O)OR¹⁷; —C(O)NR¹⁸R¹⁹; —S(O)₂R²⁰; or together with one of R¹, R² or R³ forms a ring;
R¹⁶ is aralkyl and optionally together with one of R¹, R² or R³ forms a ring;
R¹⁷ is lower alkyl, aralkyl or aryl;
R¹⁸ and R₁₉ independently are selected from H; aralkyl; lower alkyl and aryl;
R²⁰ is aryl; or
a salt thereof.

2. The compound of claim 1, according to the formula

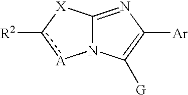

3. The compound of claim 1, according to the formula

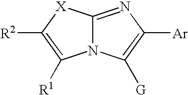

4. The compound of claim 3, according to the formula

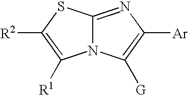

5. The compound of claim 1, according to the formula

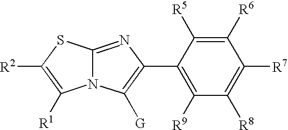

6. The compound of claim 1, according to the formula

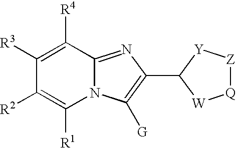

6. (canceled)

7. The compound of claim 5, according to the formula

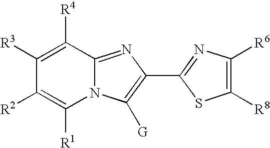

8. The compound of claim 1, wherein Ar represents

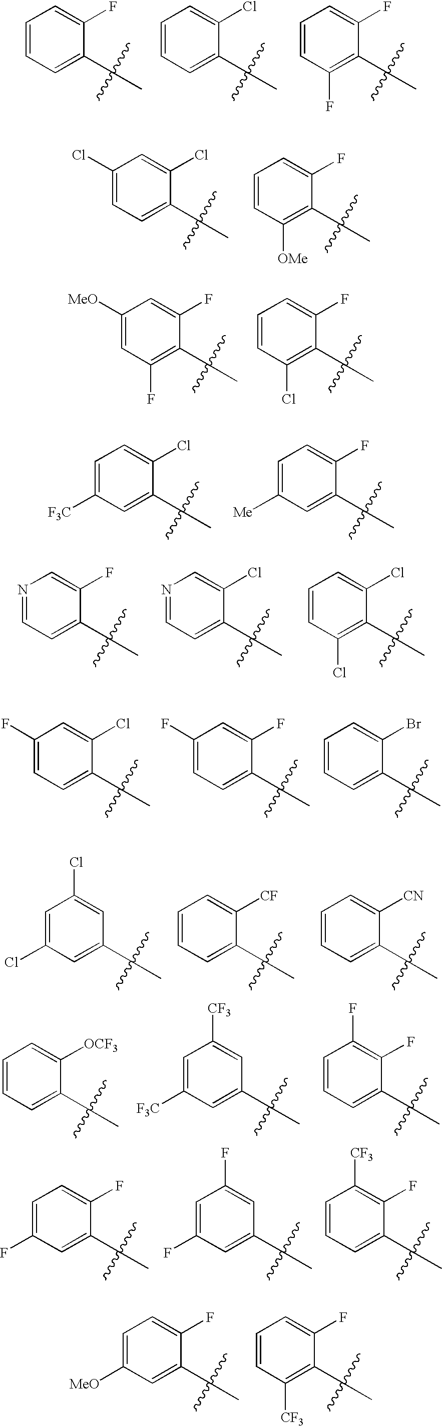

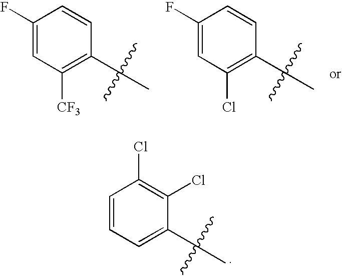

9. (canceled)

10. The compound of claim 1 according to the formula

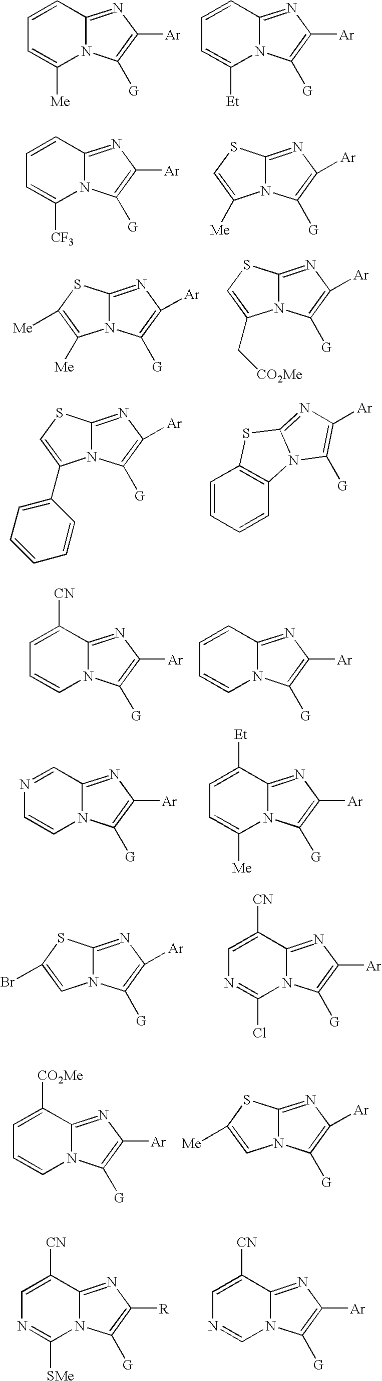

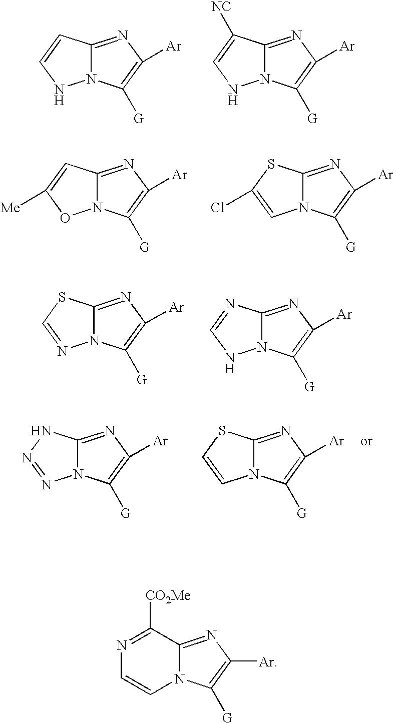

11. (canceled)

12. The compound of claim 1 wherein G comprises an optionally substituted aliphatic or aromatic ring.

13. The compound of claim 12, wherein G comprises a cycloalkyl group or an alicyclic group.

14. (canceled)

15. The compound of claim 12, wherein G comprises an aromatic ring optionally substituted with 0, 1, 2, 3 or 4 substituents selected from halo, alkyl, alkylthio, alkoxy, alkoxycarbonyl, arylalkyloxycarbonyl, aryloxycarbonyl, cycloheteroalkyl, carbamoyl, haloalkyl, dialkylamino, sulfamoyl groups and substituted versions thereof.

16. The compound of claim 12, wherein G has the formula 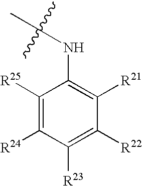
wherein R²¹, R²², R²³, R²⁴ and R²⁵ independently are H, halo, alkyl, alkylthio, alkoxy, alkoxycarbonyl, arylalkyloxycarbonyl, aryloxycarbonyl, cycloheteroalkyl, carbamoyl, haloalkyl, dialkylamino, sulfamoyl groups and substituted versions thereof.

17. The compound of claim 16, wherein at least one of R²¹, R²², R²³, R²⁴ and R²⁵ is lower alkyl or a halo.

18. The compound of claim 1, wherein G is

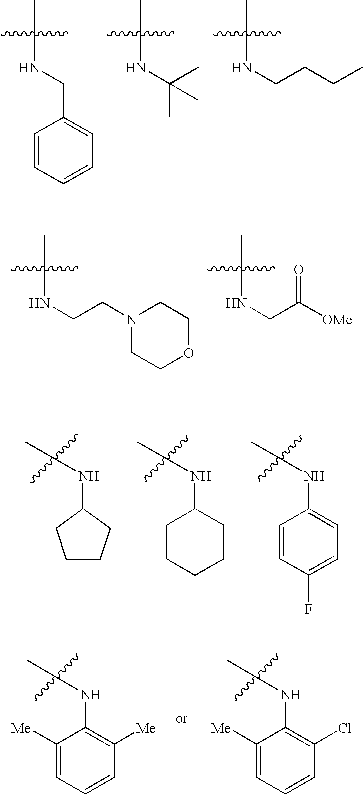

19. (canceled)

20. The compound of claim 1, according to the formula

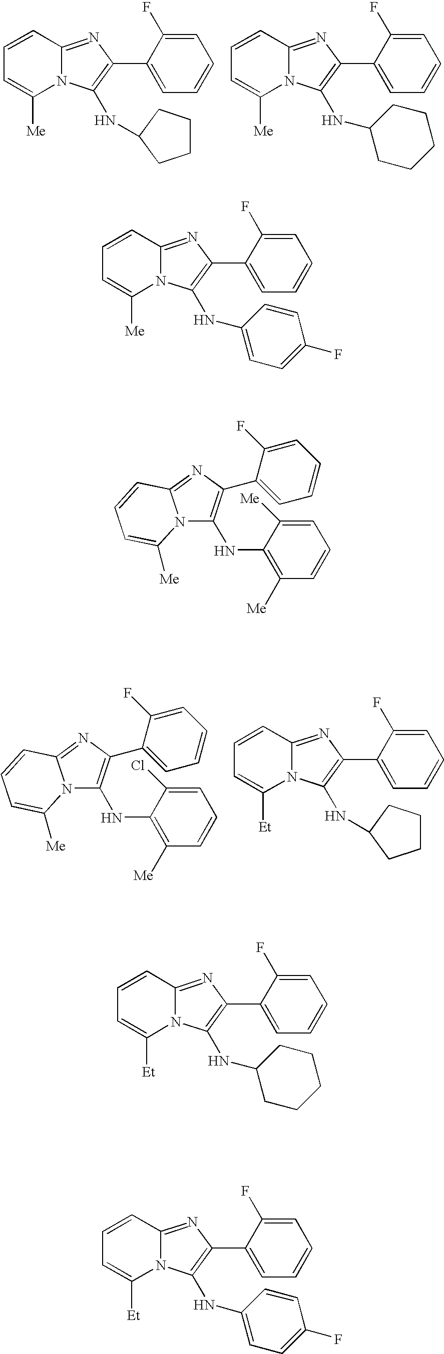

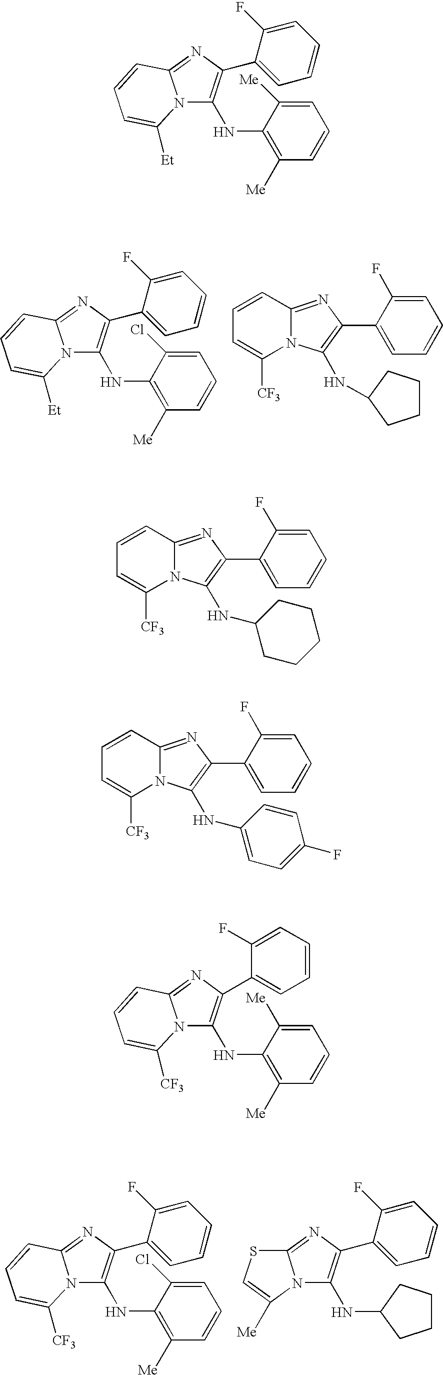

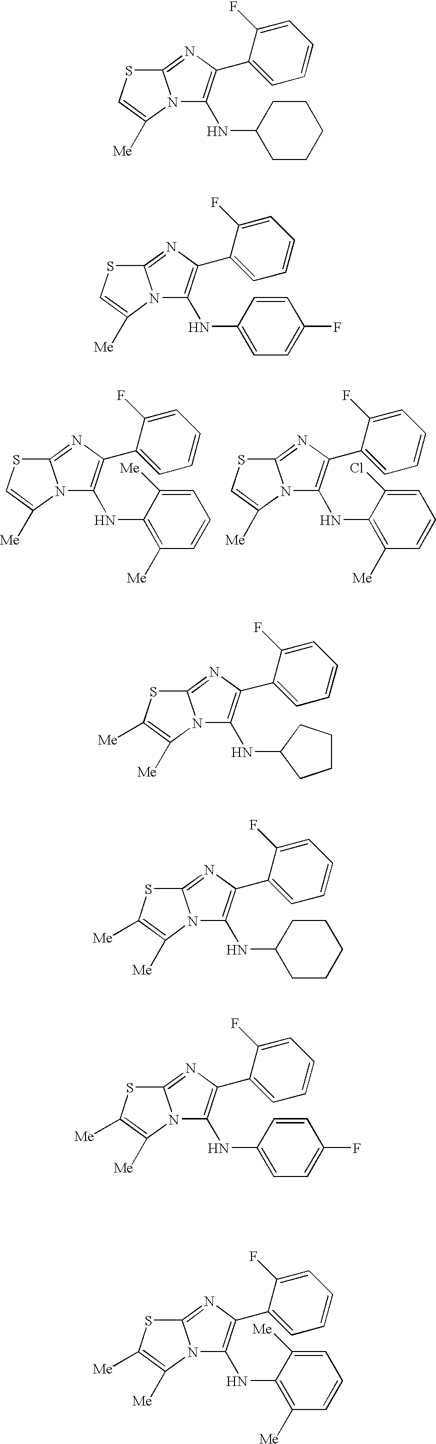

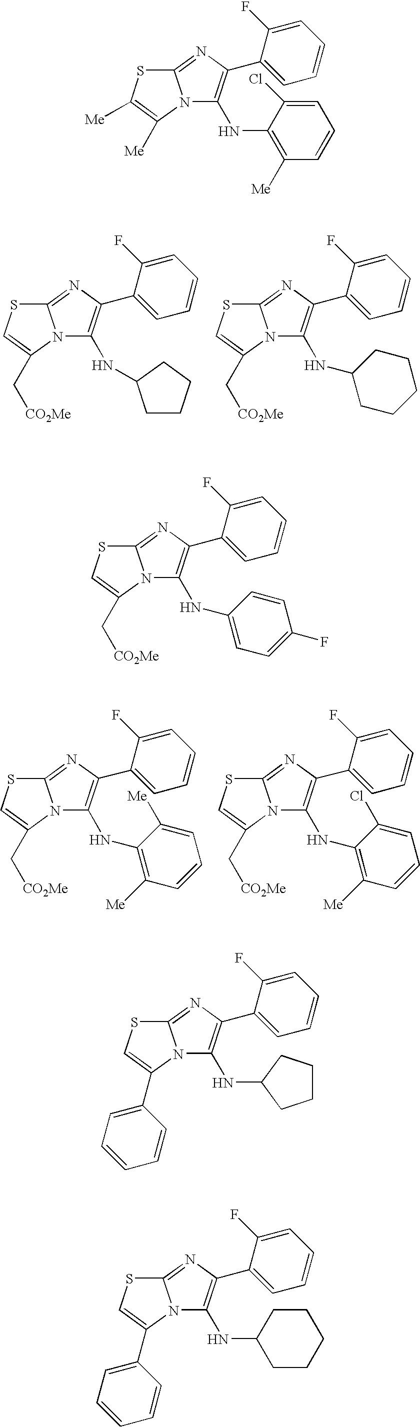

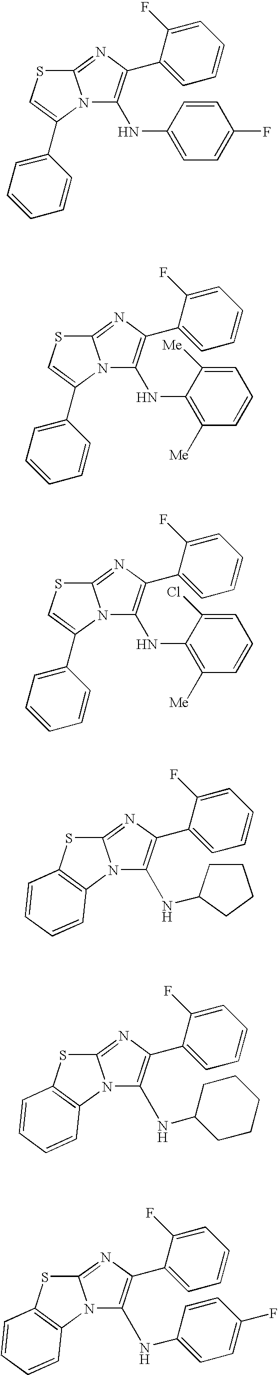

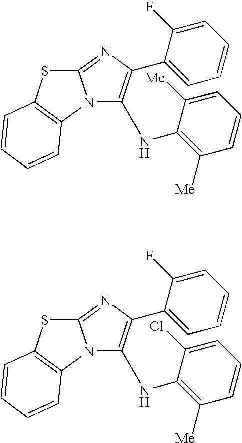

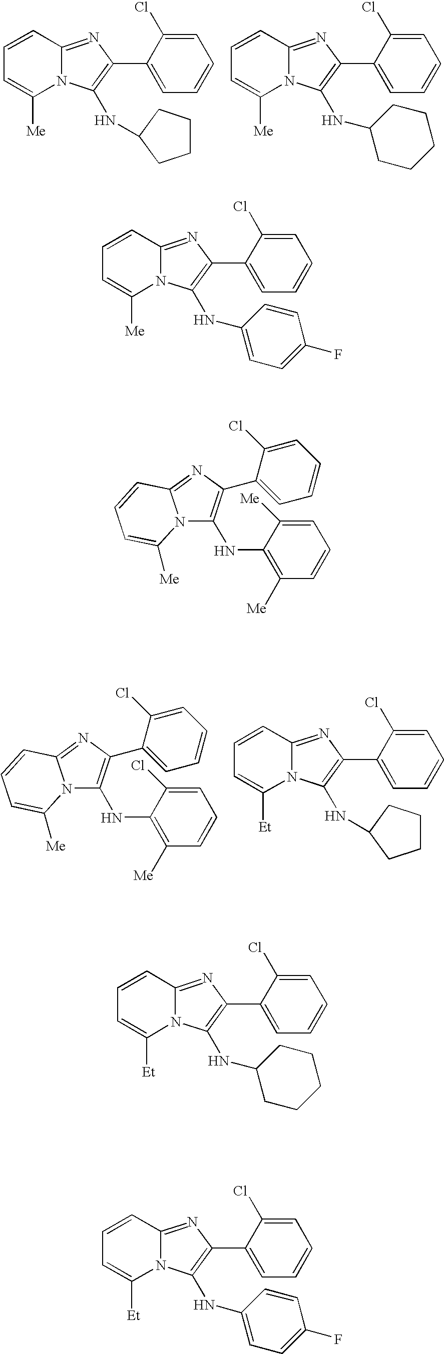

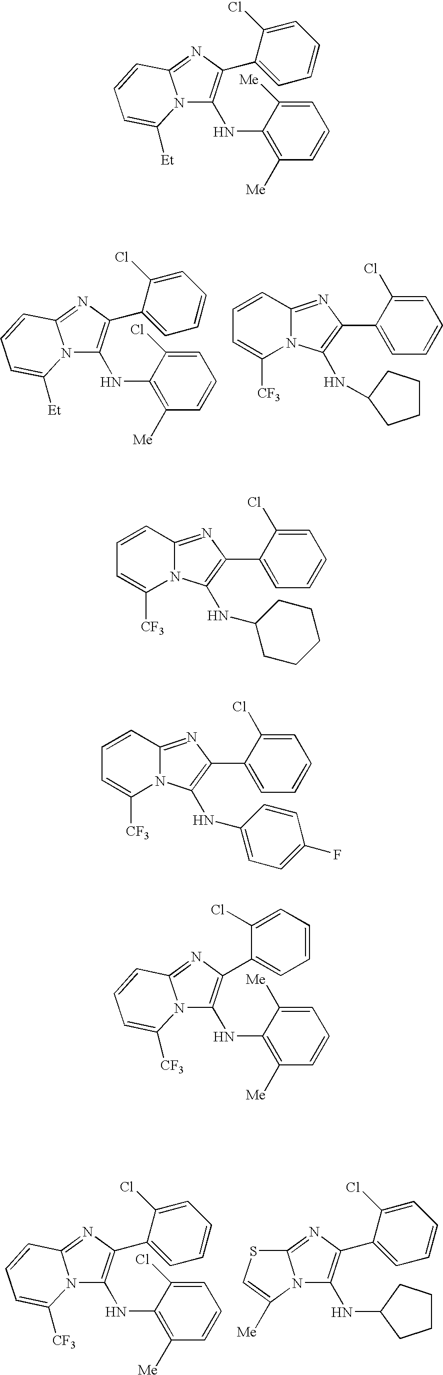

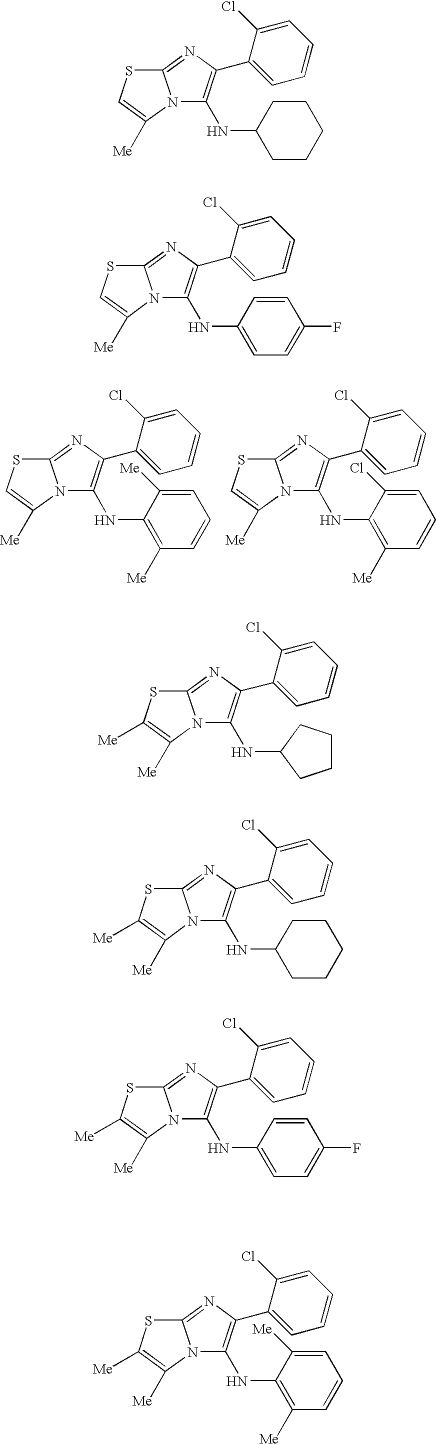

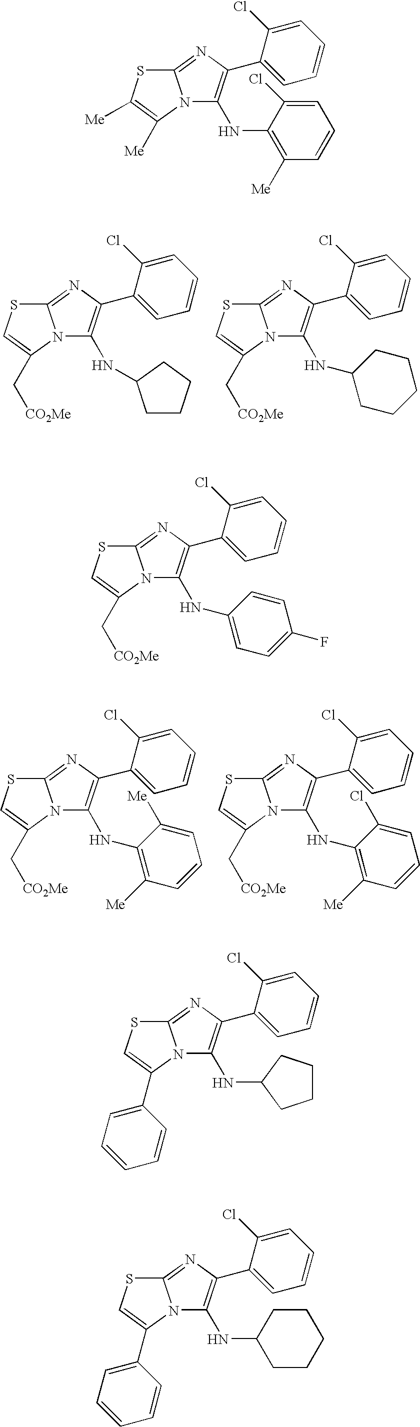

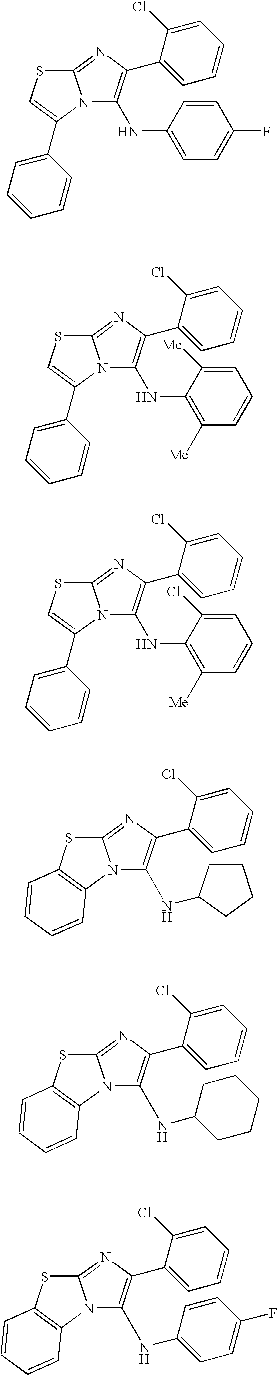

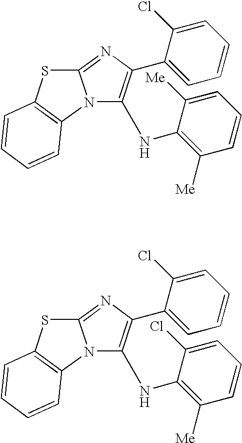

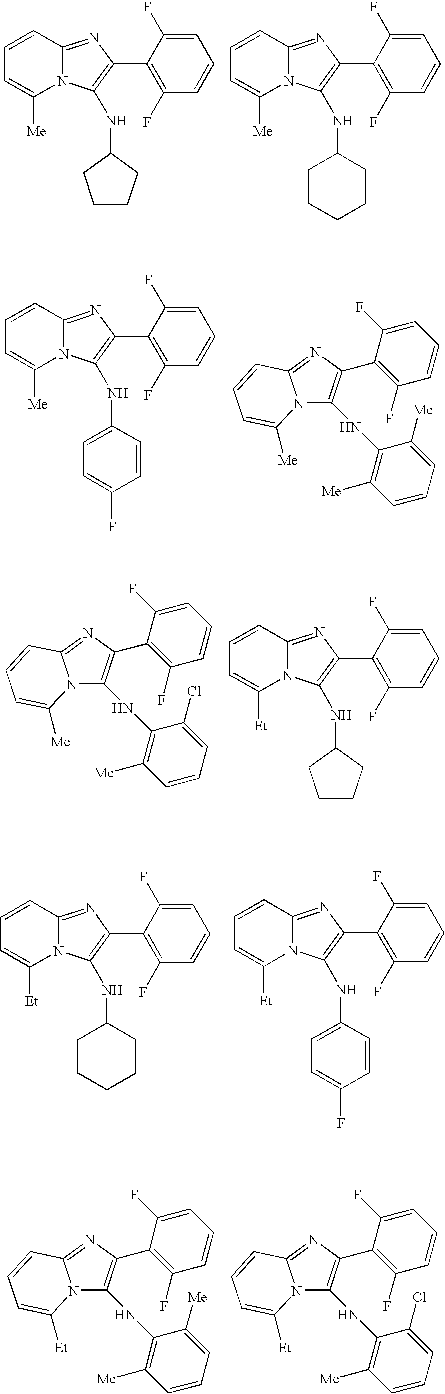

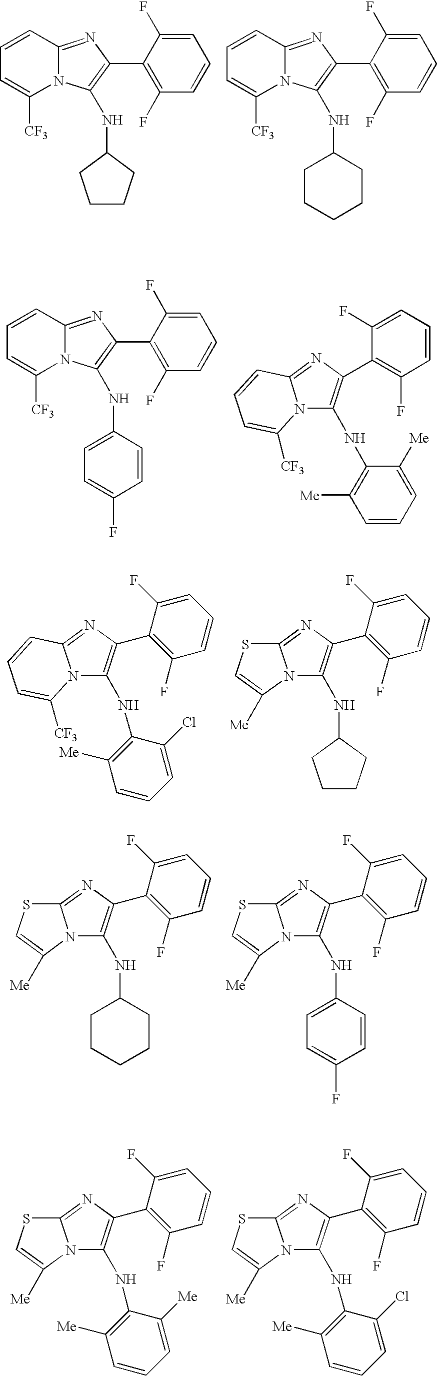

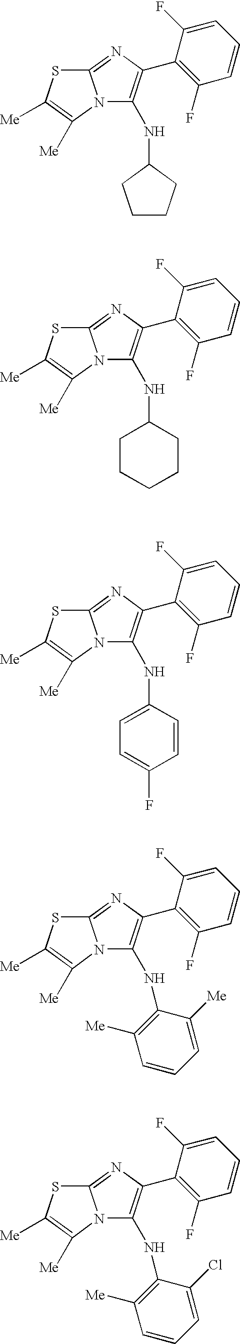

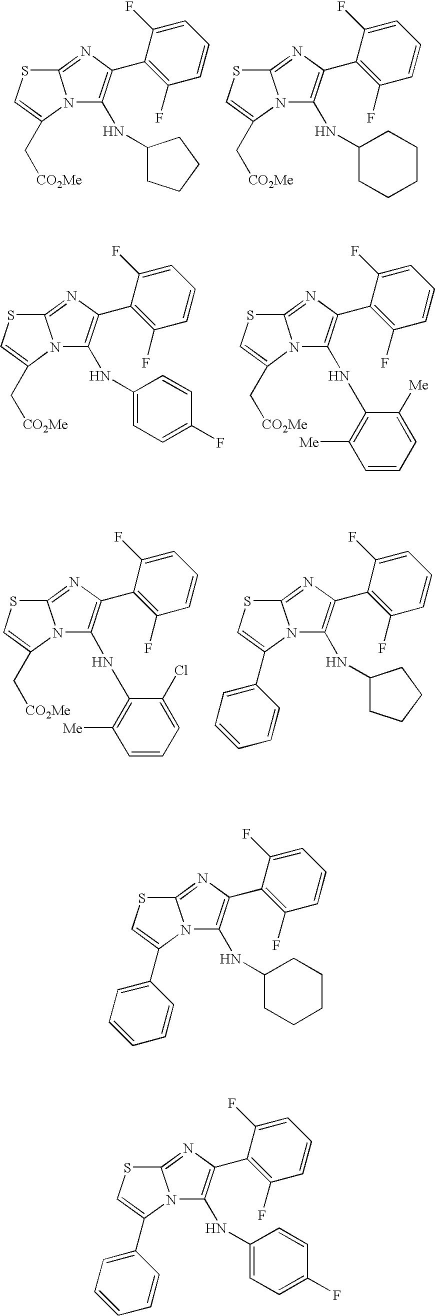

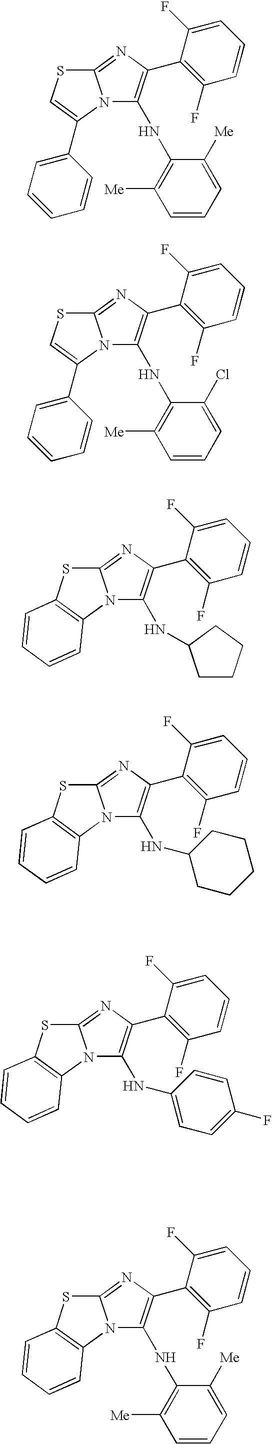

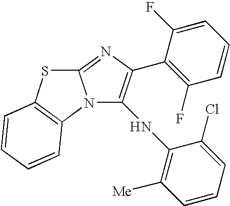

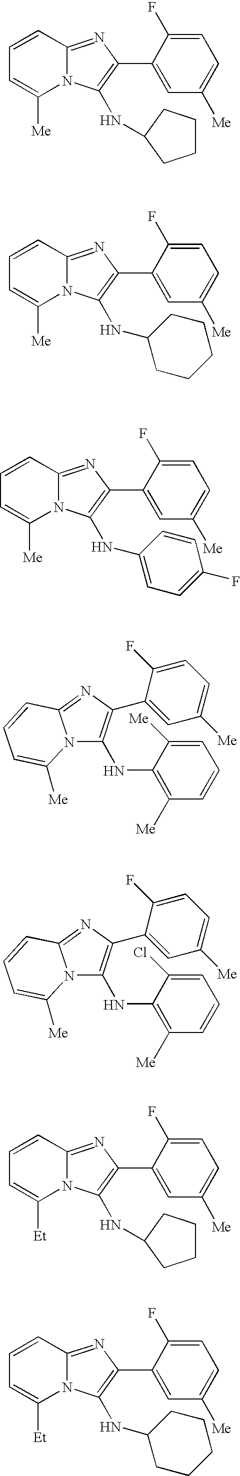

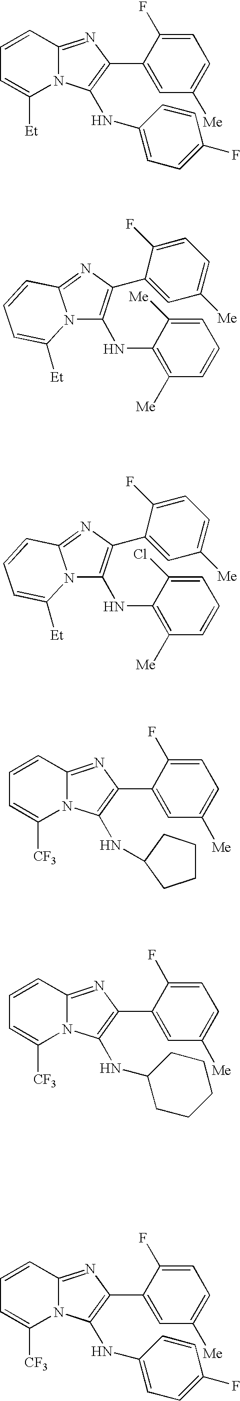

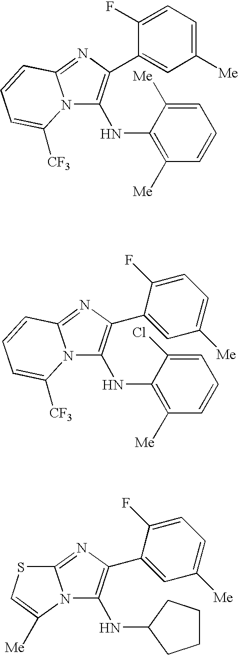

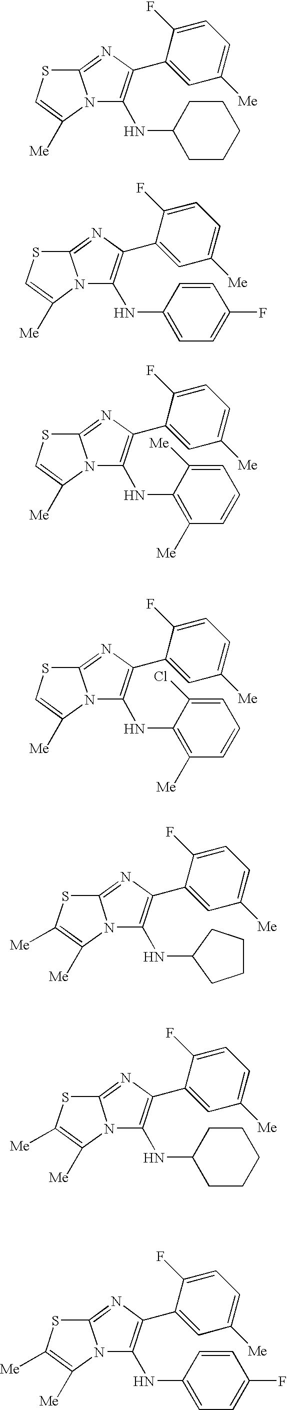

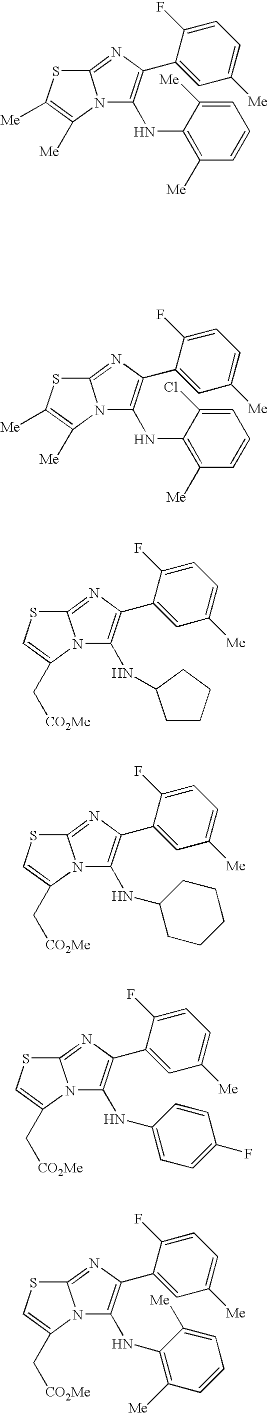

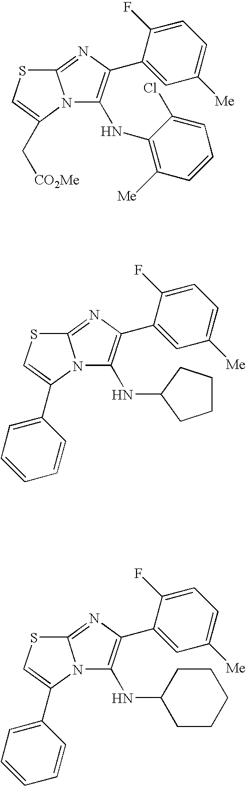

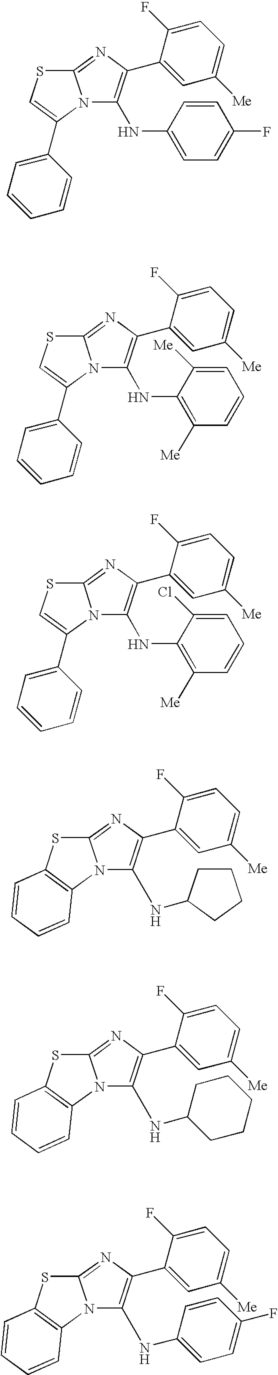

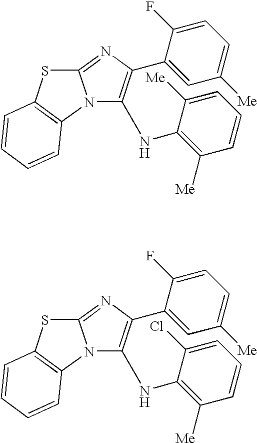

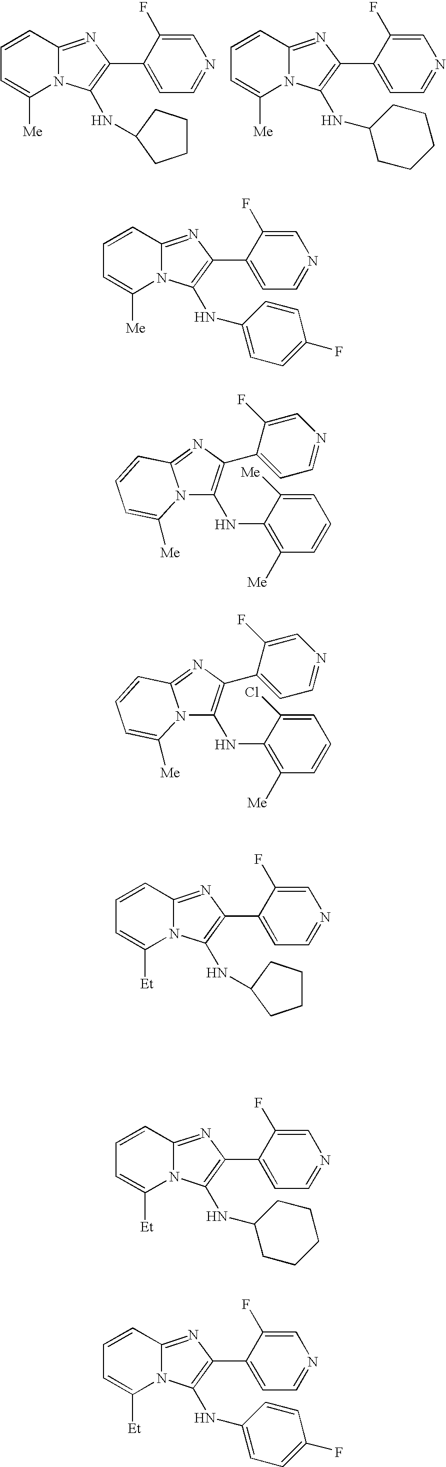

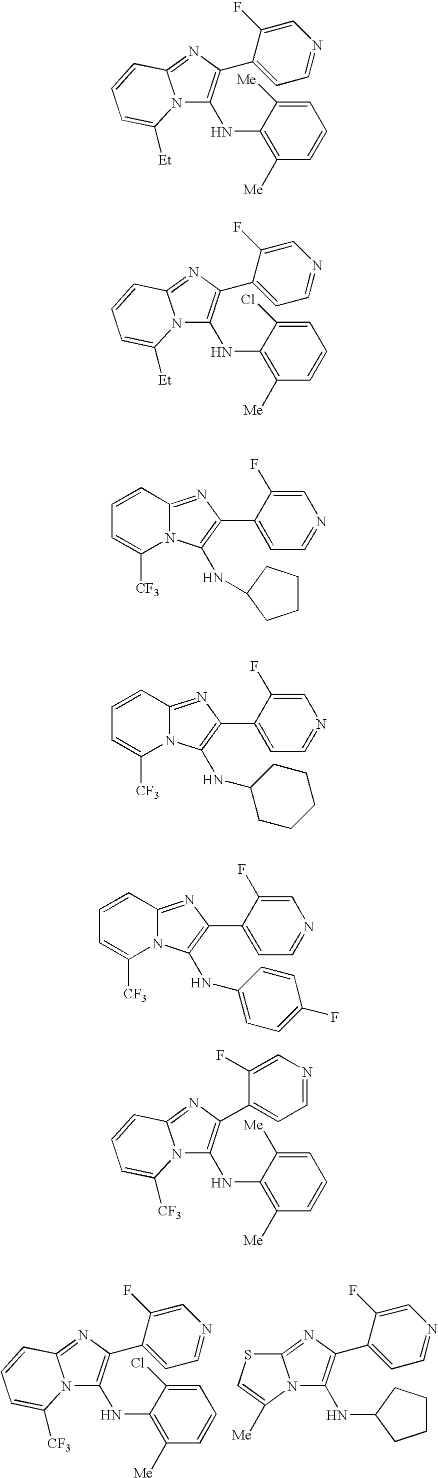

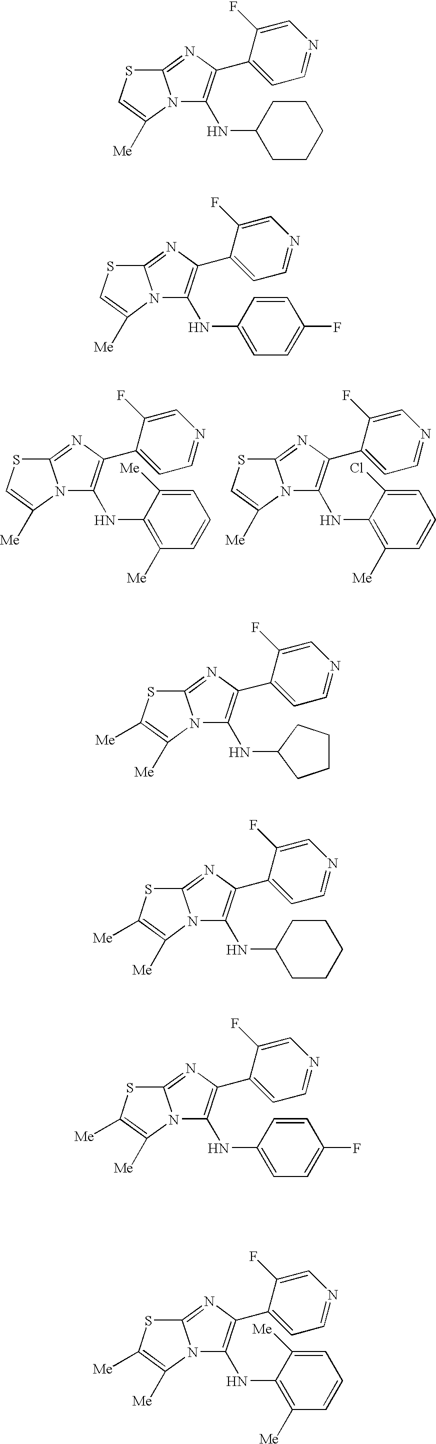

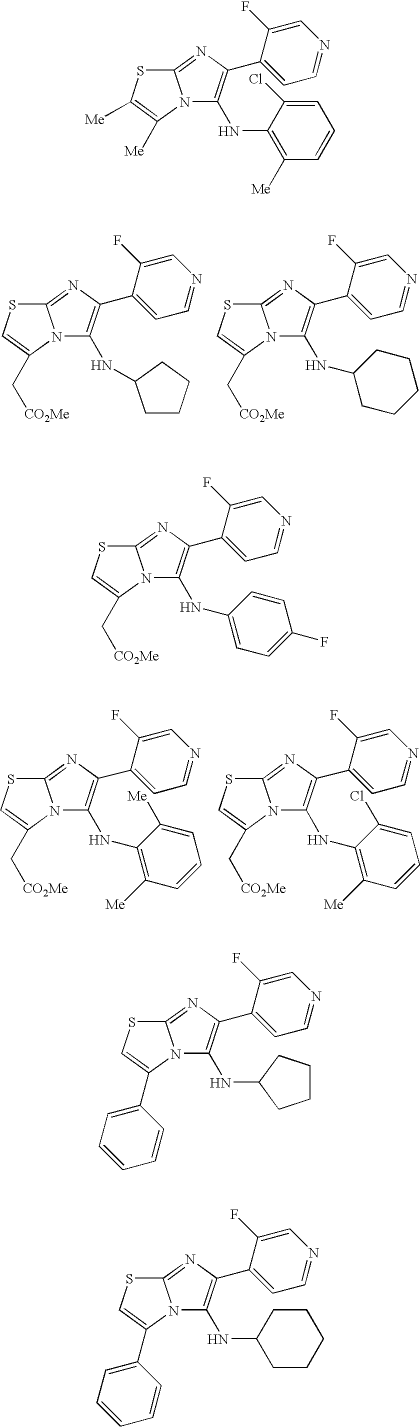

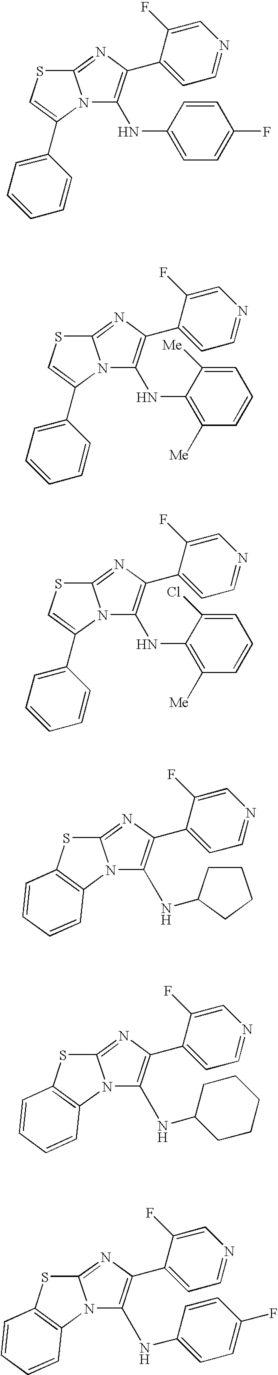

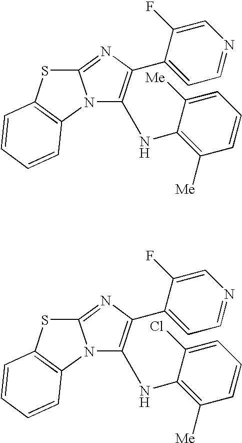

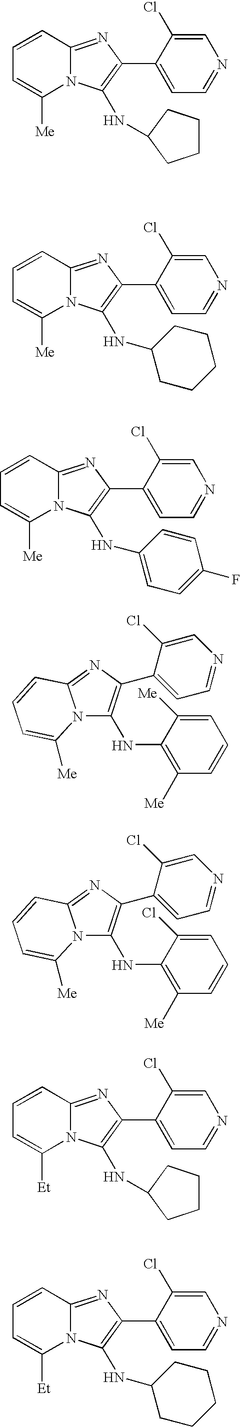

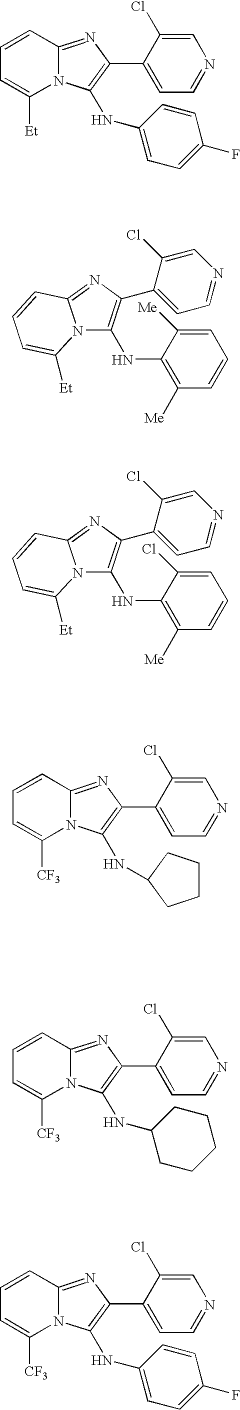

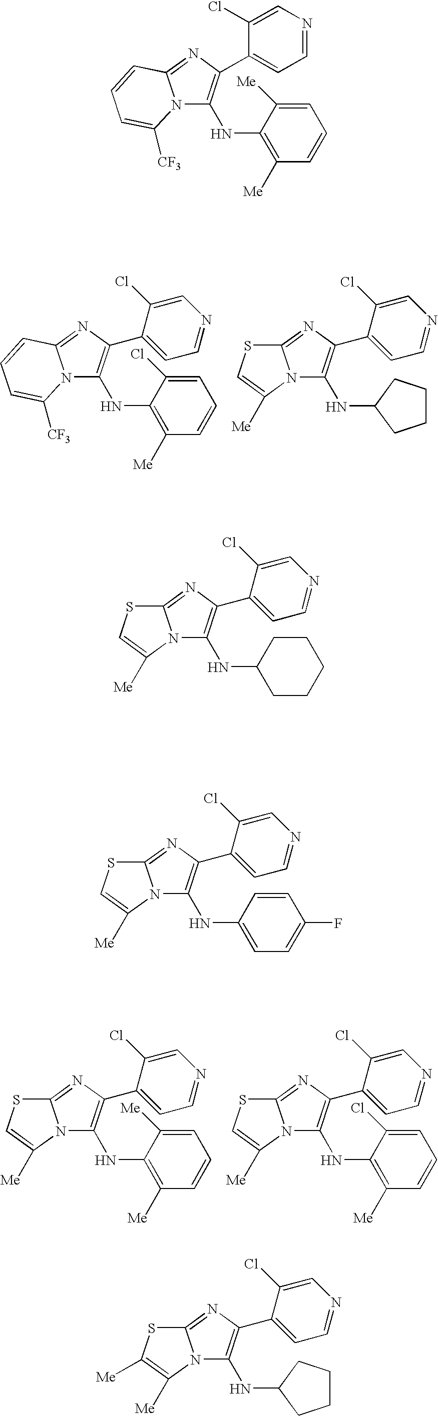

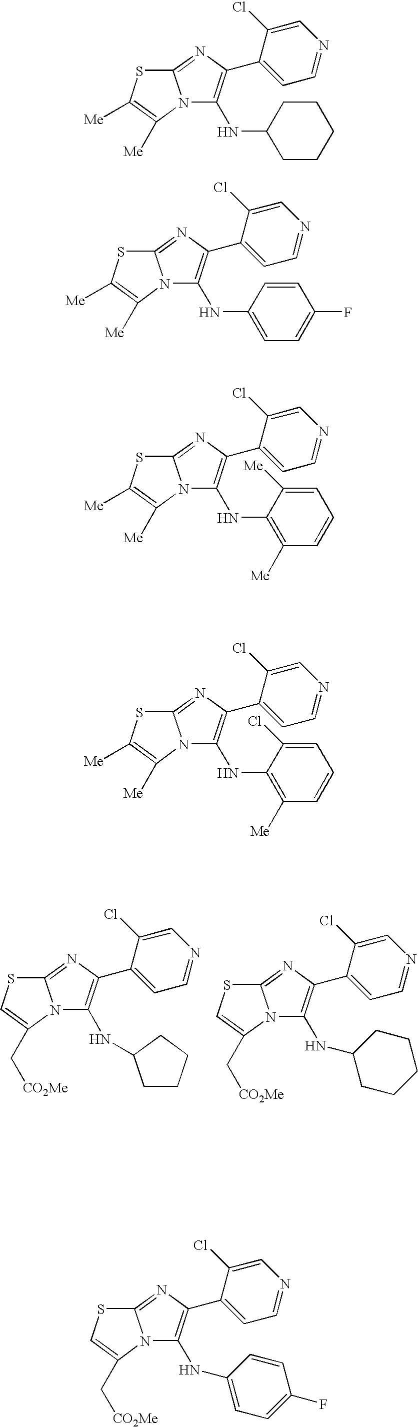

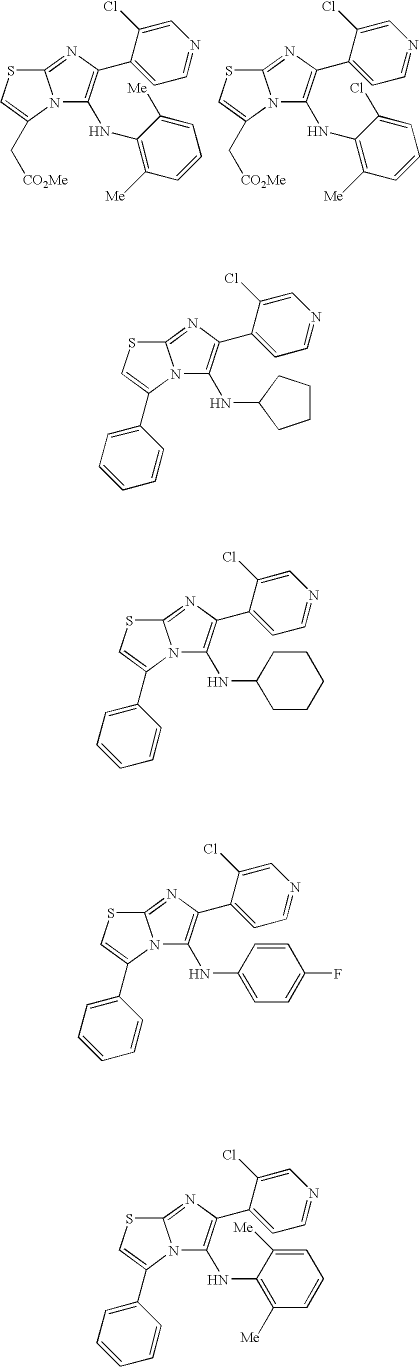

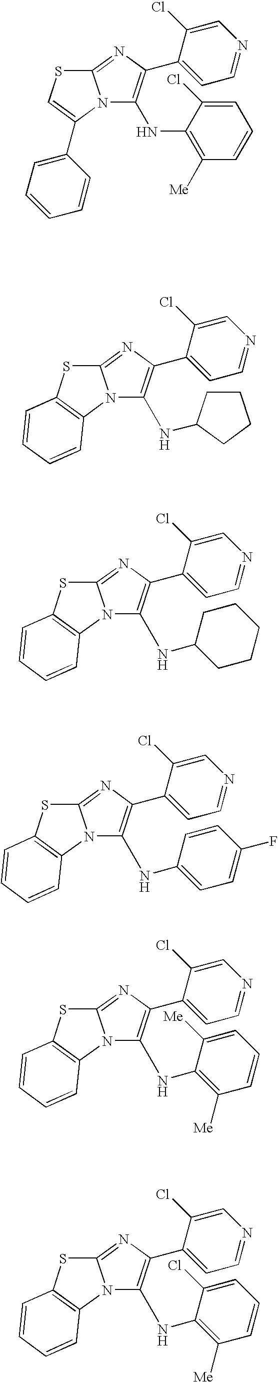

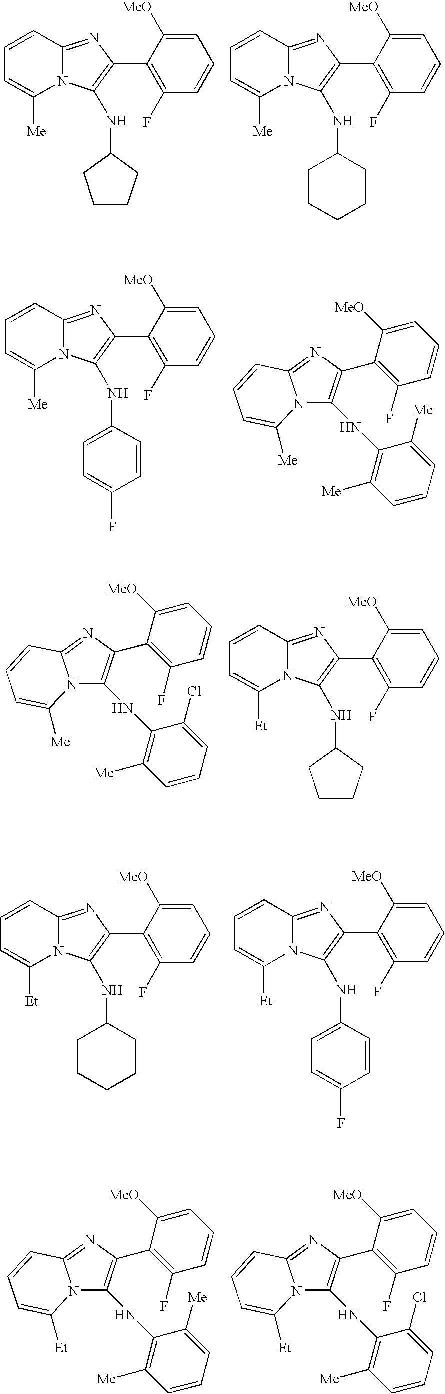

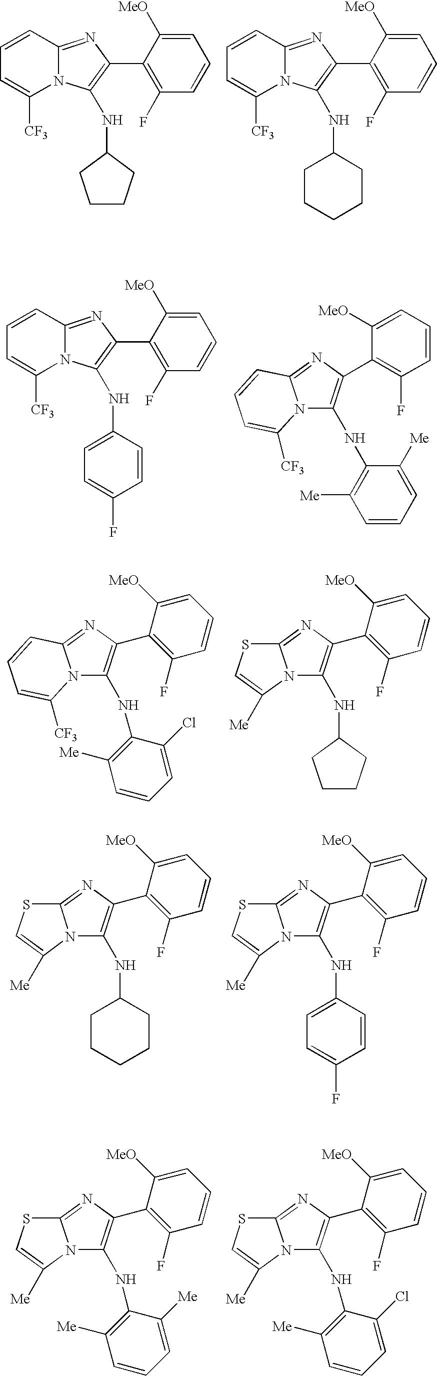

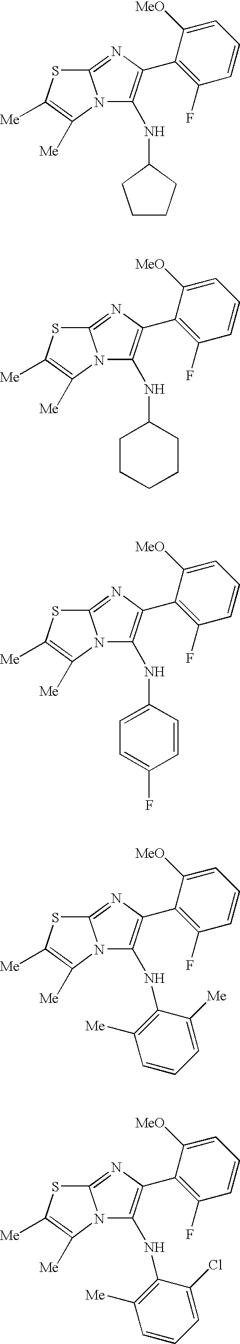

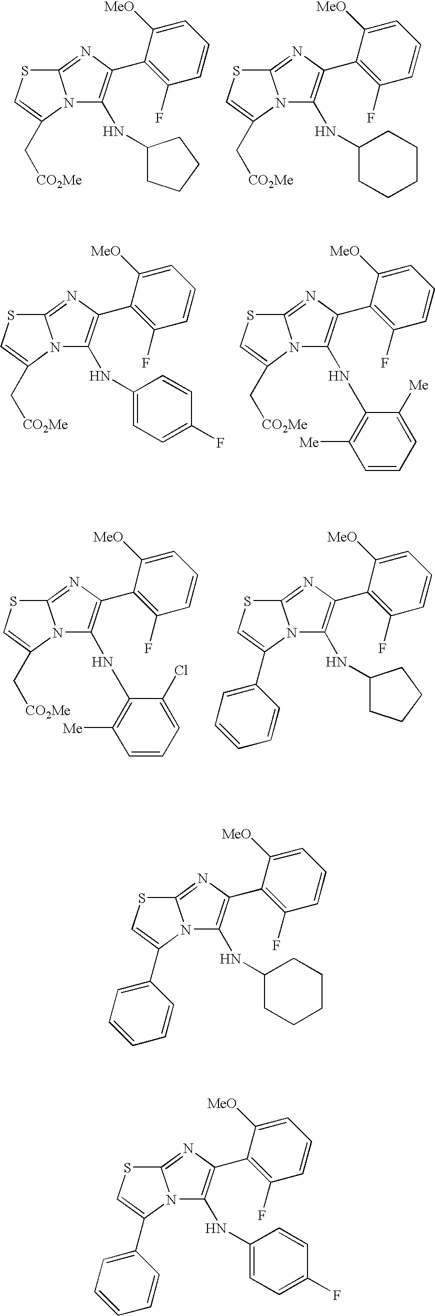

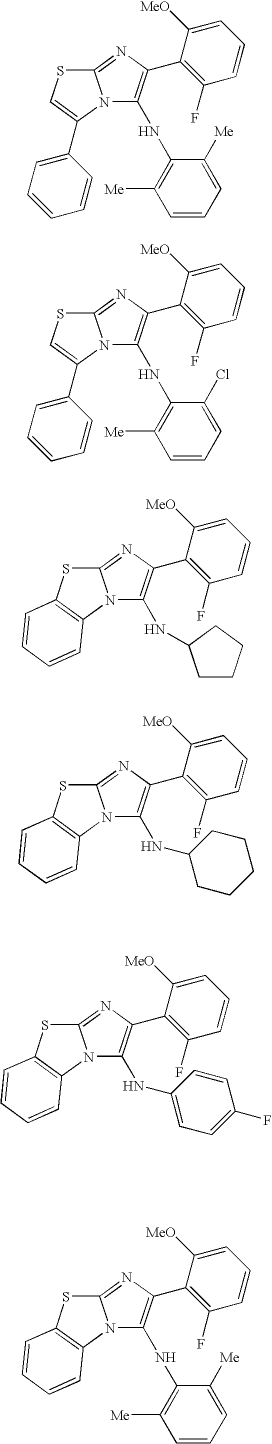

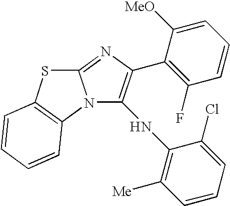

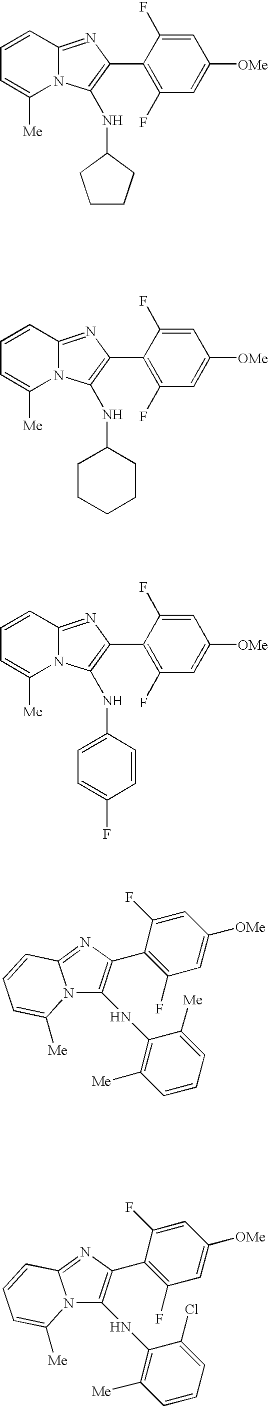

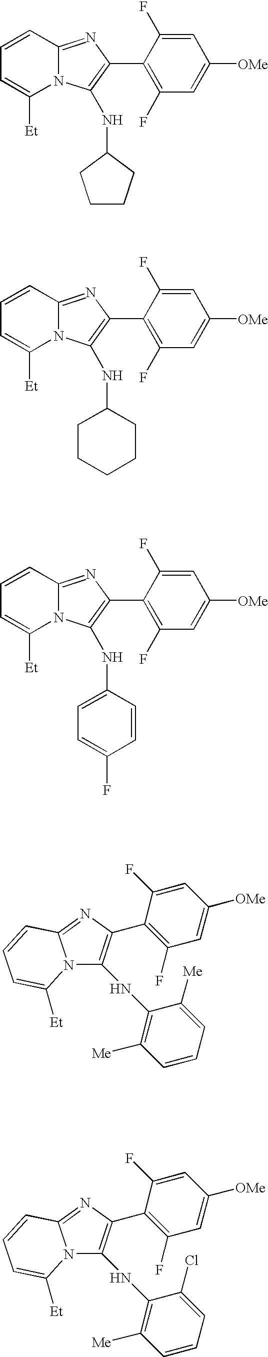

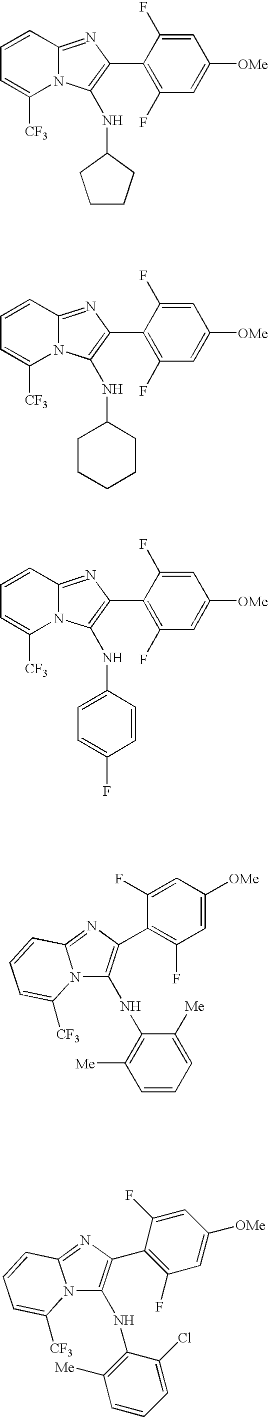

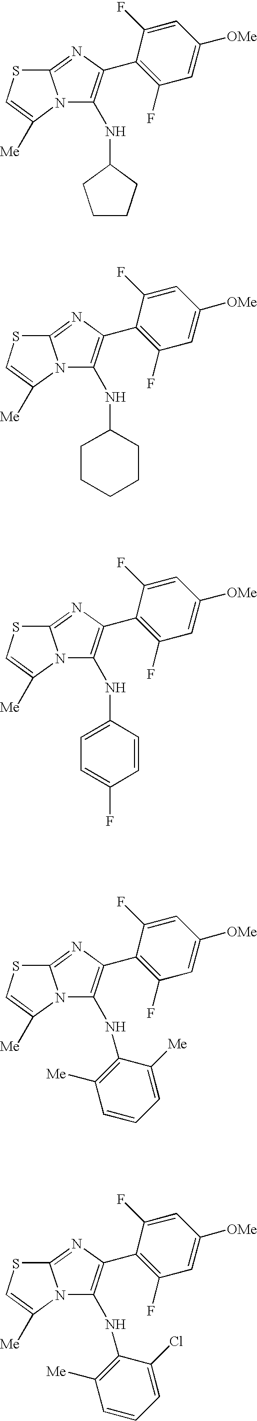

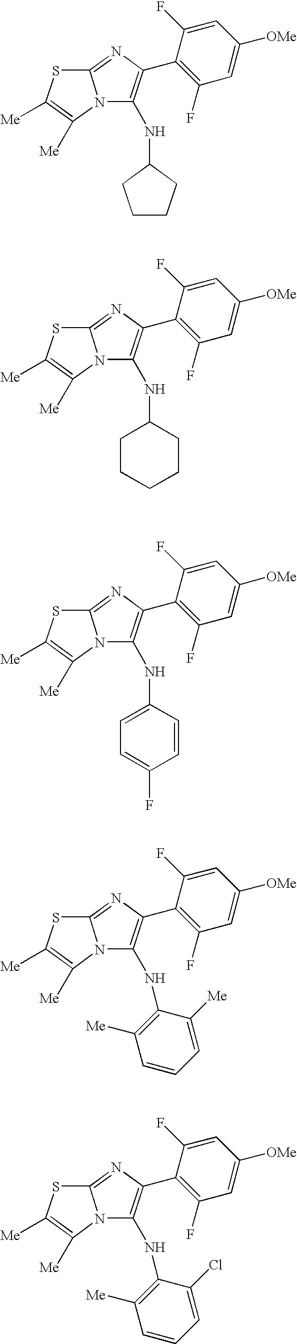

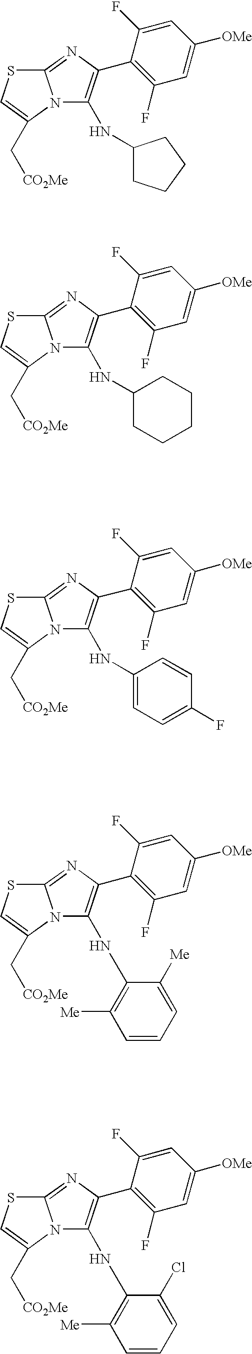

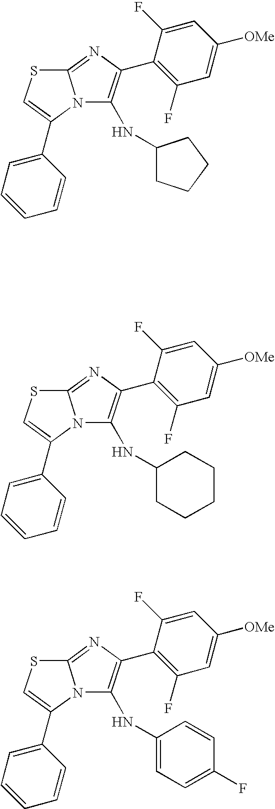

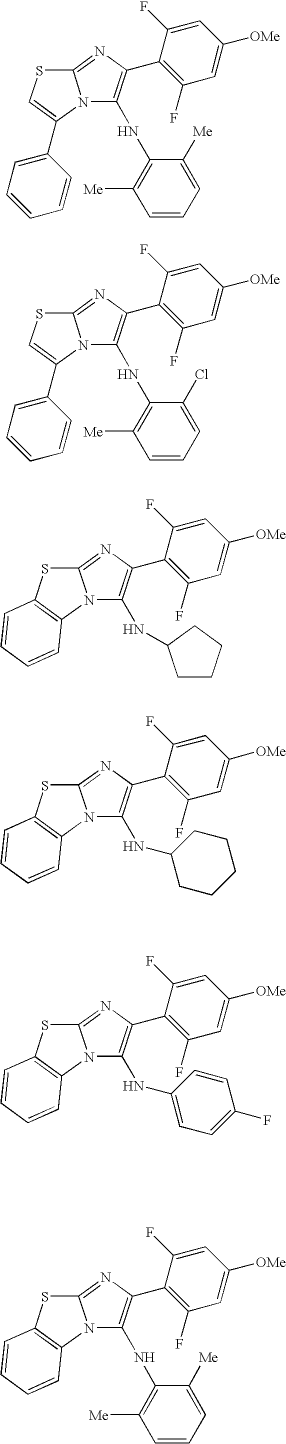

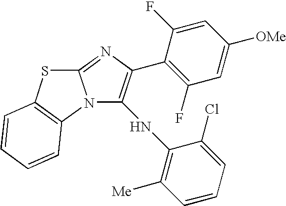

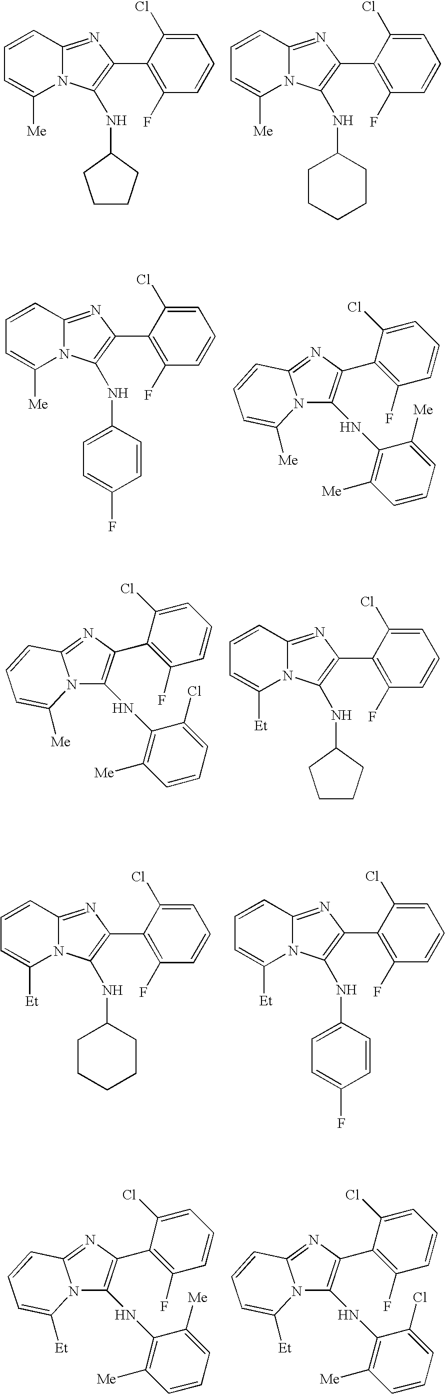

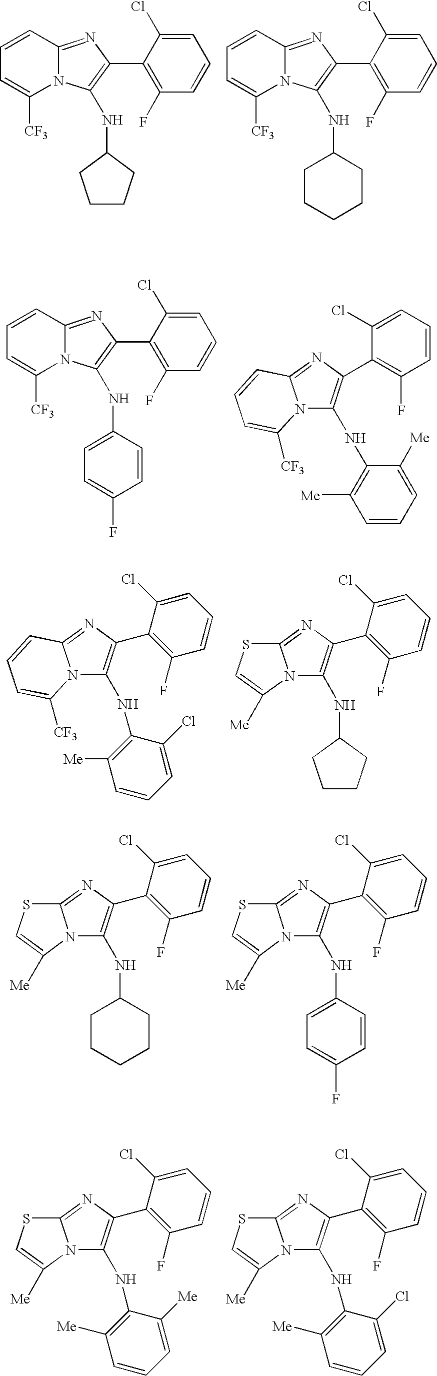

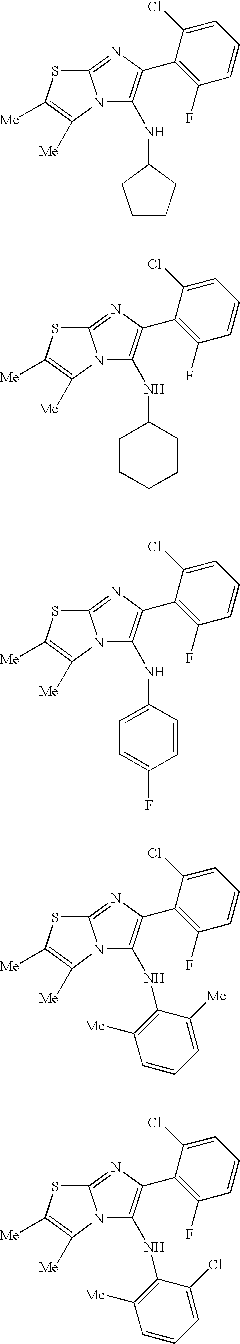

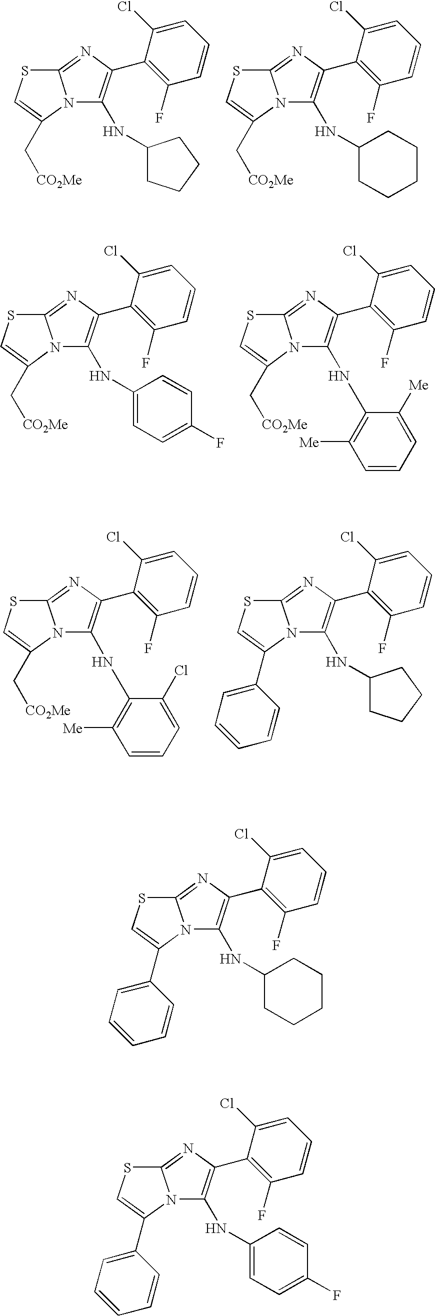

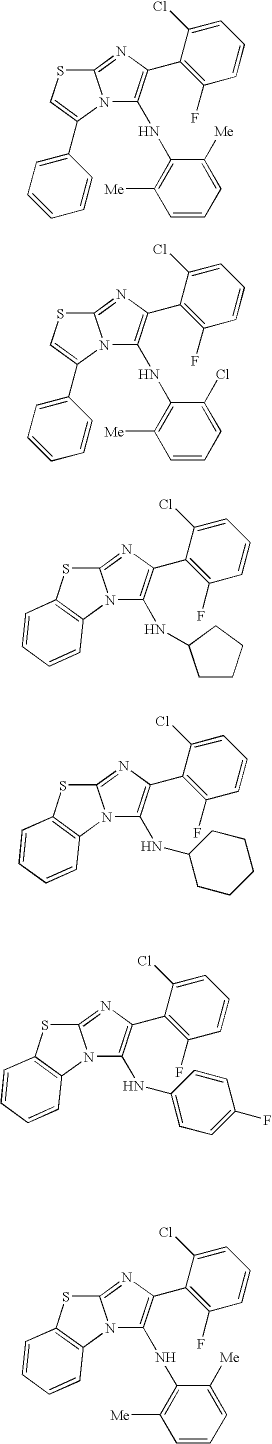

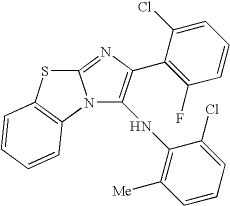

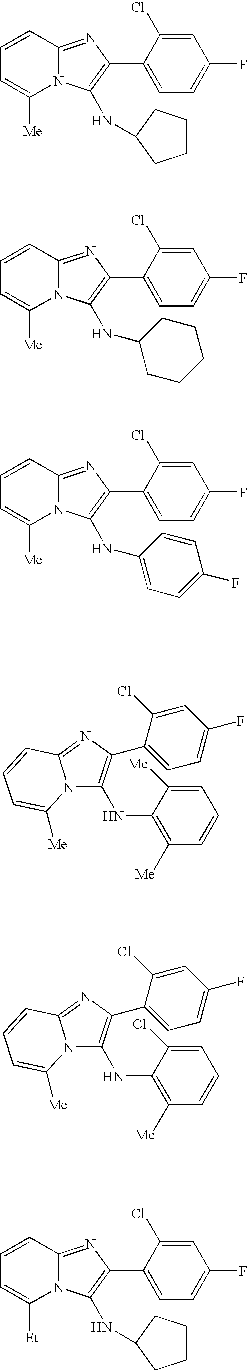

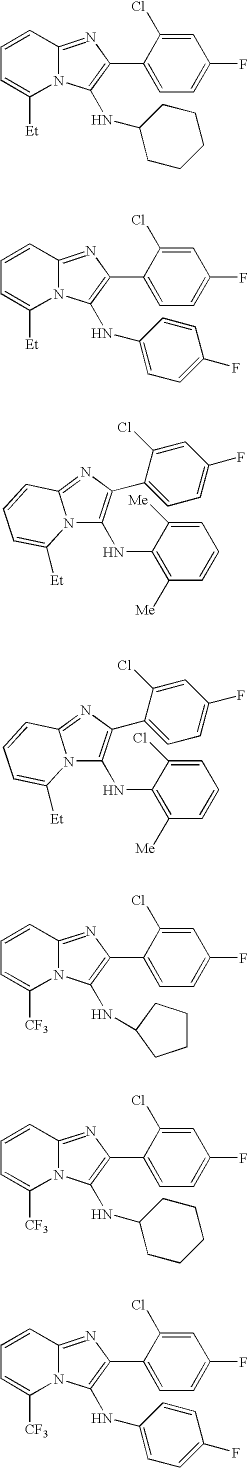

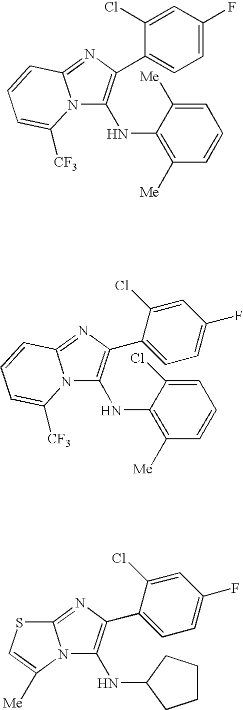

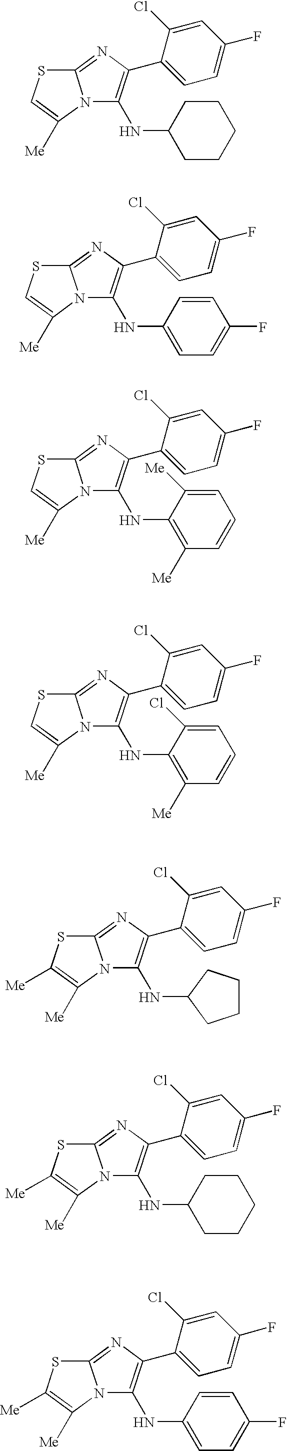

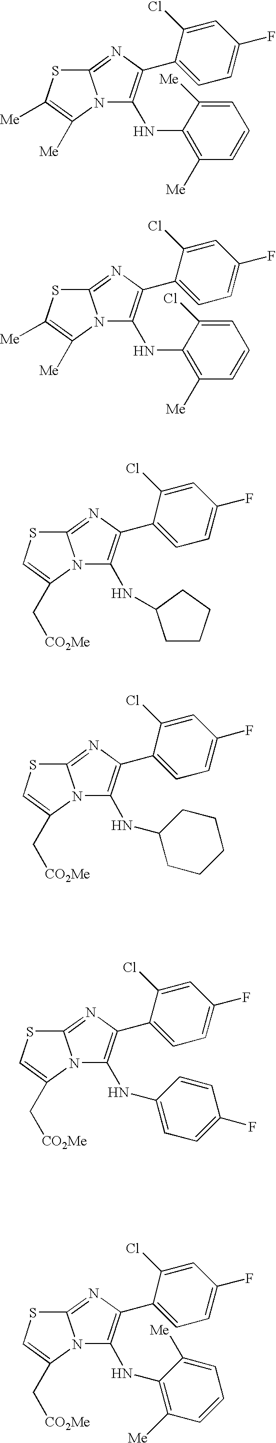

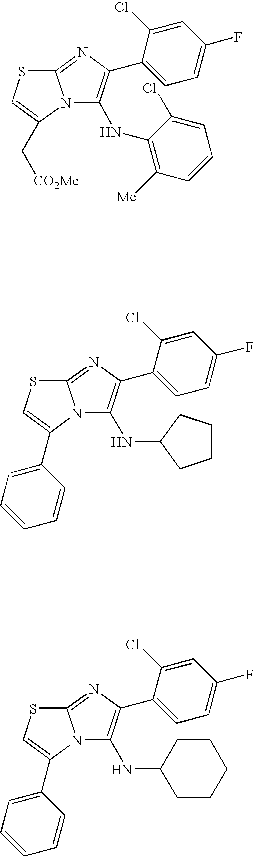

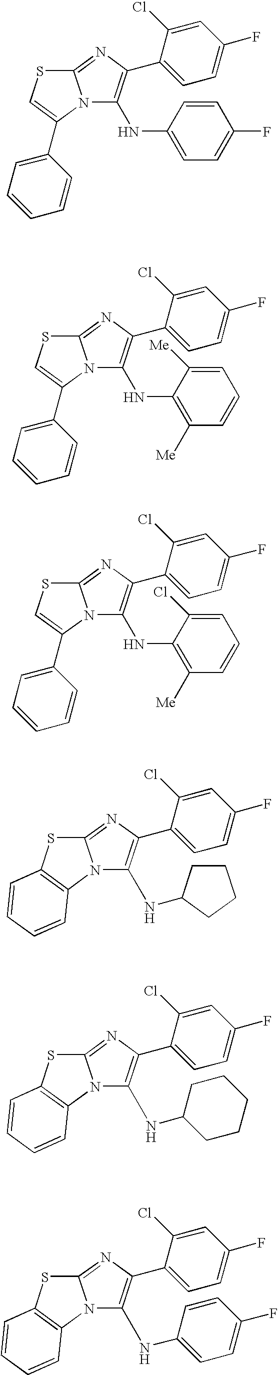

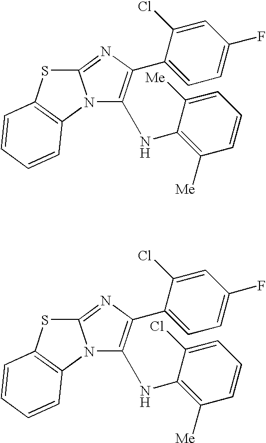

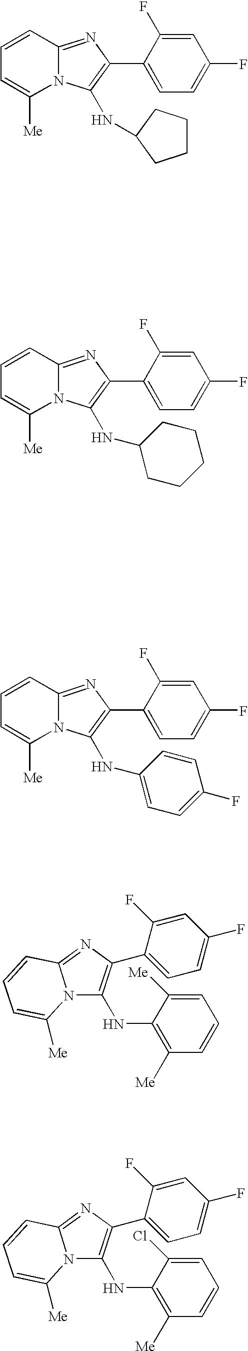

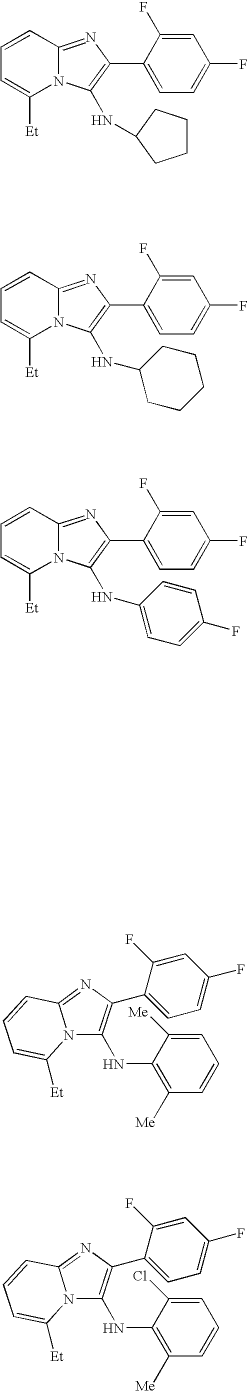

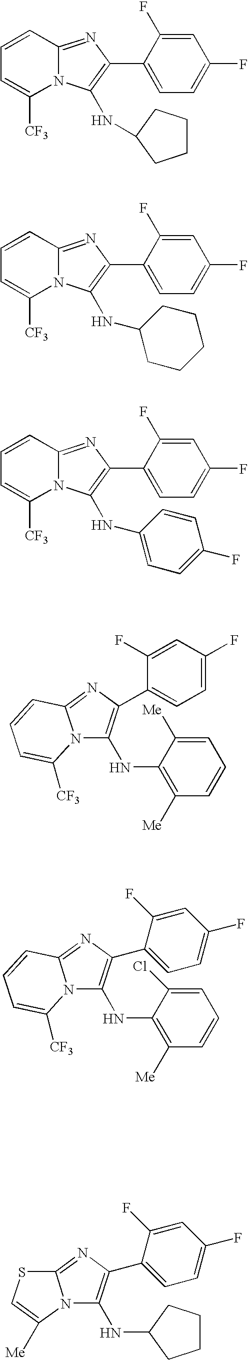

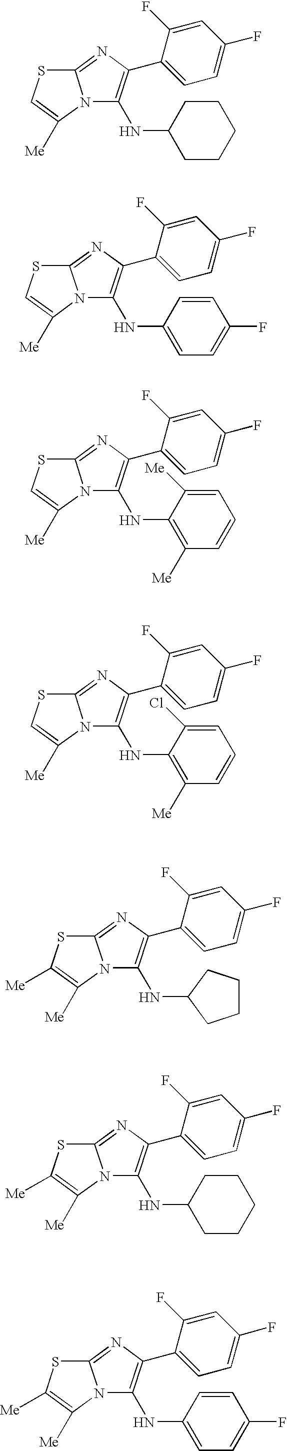

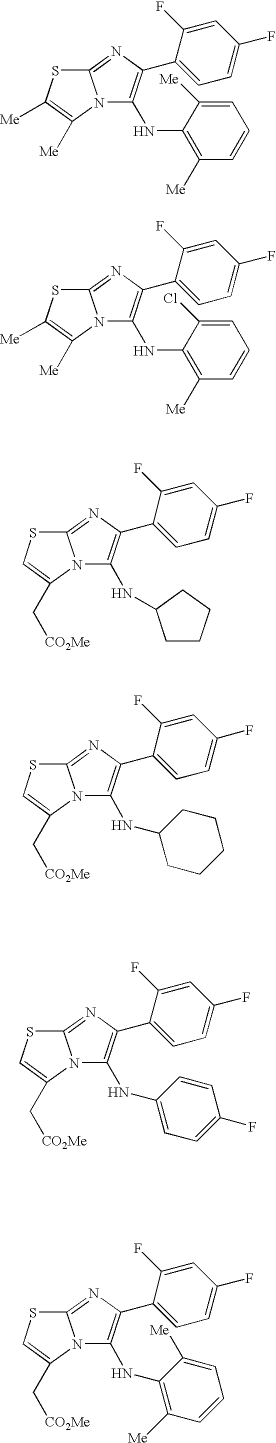

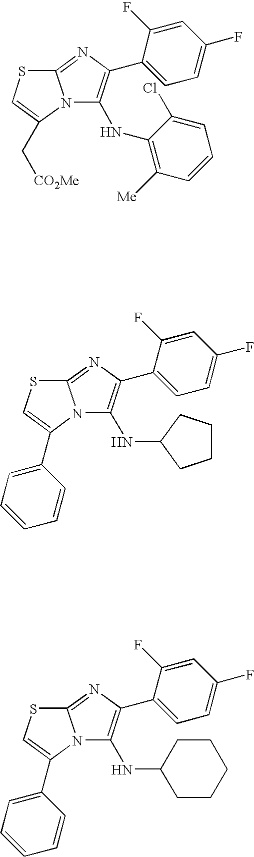

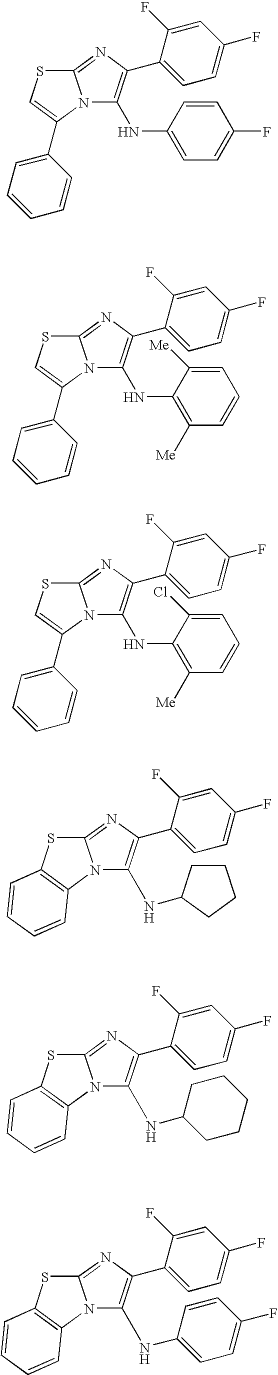

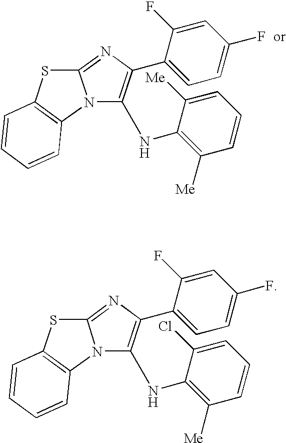

21. The compound of claim 1, according to the formula

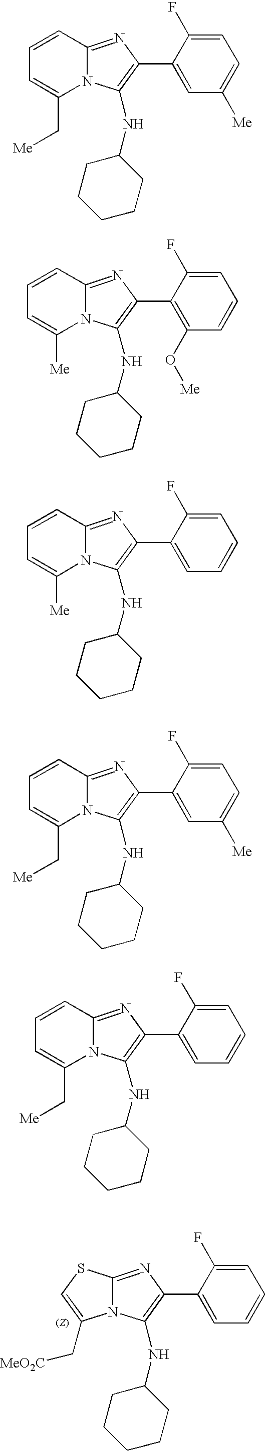

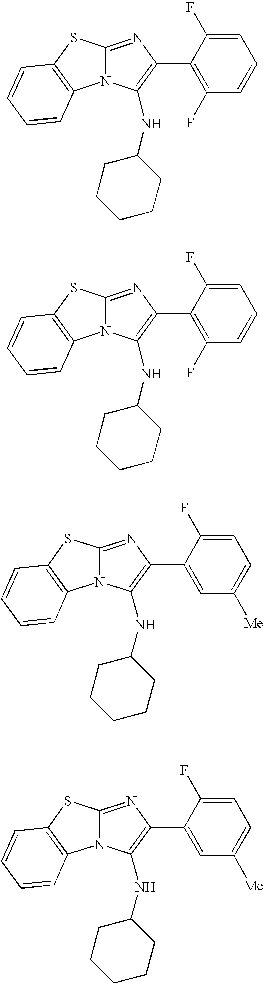

22. A method for making a compound of claim 1, according to the scheme 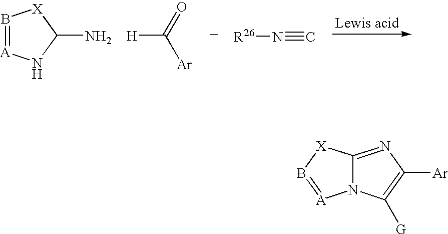
A is N, O, S or CR¹;
B is N or CR²
X is CH; CR, where R is H, cyano, halogen, aliphatic, particularly haloalkyl and lower aliphatic, —OR₉, —NR¹⁰R¹¹, or is an atom or atoms in a fused ring; O; or S; 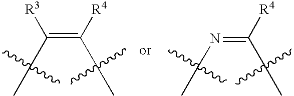
R¹, R², R³ and R⁴ independently are selected from H; cyano; halogen; haloalkyl; lower aliphatic; —OR₉; and —NR¹⁰R¹¹; and two of R¹, R², R³ and R⁴ together may optionally form a fused ring;
Ar is a 5 or 6 membered aromatic ring of the formula 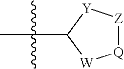
Y is S; N or CR⁵;
Z is S; N; CR⁶; 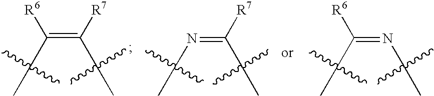
Q is S; N or CR⁸
W is S; N or CR⁹
R⁵-R⁹ independently are selected from H; cyano; halogen; haloalkyl; lower alkyl; —OR¹⁰; —SR¹¹; —NR¹²R¹³; and wherein two of R⁵-R⁹ together optionally may form a fused ring;
R¹⁰, R¹¹, R¹² and R¹³ independently are H, alkyl or acyl;
G is selected from —NR¹⁴R¹⁵ or —N═R¹⁶;
R¹⁴ and R¹⁵ independently are selected from H; aralkyl; lower alkyl; aryl; acyl;
—C(O)OR¹⁷; —C(O)NR¹⁸R¹⁹; —S(O)₂R²⁰; or together with one of R¹, R² or R³ forms a ring;
R¹⁶ is aralkyl and optionally together with one of R¹, R² or R³ forms a ring;
R¹⁷ is lower alkyl, aralkyl or aryl;
R¹⁸ and R₁₉ independently are selected from H; aralkyl; lower alkyl and aryl; and
R²⁰ is aryl.

23. A method of inhibiting a reverse transcriptase, comprising contacting the reverse transcriptase with a therapeutically effective amount of one or more of the compounds of claim 1.

24. The method of claim 23, wherein the reverse transcriptase is a human immunodeficiency virus (HIV)-1 reverse transcriptase.

25. The method of claim 24, wherein the reverse transcriptase has a mutation.

26. The method of claim 25, wherein the mutation confers resistance to at least one reverse transcriptase inhibitor.

27. The method of claim 26, wherein the at least one reverse transcriptase inhibitor is stavudine or zidovudine.

28. (canceled)

29. A method for inhibiting human immunodeficiency virus (HIV) infection, comprising:
contacting a cell with a therapeutically effective amount of one or more of the compounds of claim 1, thereby inhibiting HIV infection.

30. The method of claim 29, wherein contacting the cell comprises administering the one or more compounds to a mammalian subject.

31. The method of claim 29, further comprising administering a therapeutically effective amount of an HIV nucleoside reverse transcriptase inhibitor, an HIV non-nucleoside reverse transcriptase inhibitor other than those described in claim 1, an HIV protease inhibitor, a viral fusion inhibitor, an RNAse H inhibitor, an integrase inhibitor, a maturation inhibitor, or combinations thereof.

32.-40. (canceled)

41. A method for inhibiting virus replication in a cell infected with a resistant strain of HIV comprising administering to the infected cell a virus replication inhibiting amount of a compound of claim 1.

42. The method of claim 41, wherein the resistant HIV strain is a clinical isolate obtained from an infected individual who is not responding or has not responded to at least one treatment course.

43. The method of claim 41, wherein administering to an infected cell comprises administering to a human.

44. (canceled)

45. A method for treating a subject infected with a resistant strain of HIV, comprising:
identifying the subject infected with the resistant strain of HIV; and
administering a therapeutically effective amount of a compound of claim 1.

46. (canceled)

47. (canceled)

48. A pharmaceutical composition comprising a therapeutically effective amount of any of the compounds of claim 1 and a pharmaceutically acceptable carrier.

49. The composition according to claim 48, in an amount sufficient for a single dose regimen.

50. (canceled)

51. The compound of claim 5, according to the formula [chemical structure]
